(19) United States
(12) Patent Application Publication
(10) Pub. No.: US 2026/0234129 A1
(43) Pub. Date: Aug. 13, 2026

(54) NOVEL IMIDAZOLE DERIVATIVE AND USE THEREOF

(71) Applicants: PELEMED CO., LTD., Gwangiu (KR); GWANGJU INSTITUTE OF SCIENCE AND TECHNOLOGY, Gwangju (KR)

(72) Inventors: Soo Yeon JANG, Seoul (KR); Jin Hee PARK, Seoul (KR); Myung Jin KIM, Seoul (KR); Yong Chul KIM, Gwangju (KR); Bong Ki KO, Incheon (KR); Yong Soo JANG, Busan (KR)

(73) Assignees: PELEMED CO., LTD., Gwangiu (KR); GWANGJU INSTITUTE OF SCIENCE AND TECHNOLOGY, Gwangju (KR)

(21) Appl. No.: 19/471,478

(22) PCT Filed: Apr. 4, 2024
(86) PCT No.: PCT/KR2024/004438

§ 371 Date: Oct. 1, 2025

(30) Foreign Application Priority Data
Apr. 4, 2023 (KR) 10-2023-0044392

(51) Int. Cl. C07D 403/04; A61K 31/4184; A61K 31/439; A61K 31/454; A61K 31/496; A61K 31/5377; A61P 35/00; C07D 401/14; C07D 403/14; C07D 405/14; C07D 487/04; C07D 487/08; C07D 487/10

(52) U.S. Cl. CPC C07D 403/04; A61K 31/4184; A61K 31/439; A61K 31/454; A61K 31/496; A61K 31/5377; A61P 35/00; C07D 401/14; C07D 403/14; C07D 405/14; C07D 487/04; C07D 487/08; C07D 487/10

(57) ABSTRACT

The present invention relates to a novel imidazole derivative and a pharmaceutical composition for treating protein kinase-associated diseases, comprising same as an active ingredient. More specifically, the present invention relates to a novel imidazole derivative and a pharmaceutical composition comprising same as an active ingredient, the derivative inhibiting protein kinase activity so as to prevent or treat cancer diseases. In addition, the compound and the pharmaceutical composition, comprising same as an active ingredient, of the present invention, effectively inhibit FLT3 kinase activity, and thus can be effectively used for preventing or treating mutant FLT3-associated diseases, particularly acute myeloid leukemia.

### TECHNICAL FIELD

The present invention relates to a novel imidazole derivative and a pharmaceutical composition for the treatment of protein kinase-associated diseases containing it as an active ingredient. More specifically, the present invention relates to a novel imidazole derivative that can prevent or treat cancer by inhibiting the activity of protein kinases, and a pharmaceutical composition containing the same as an active ingredient.

### BACKGROUND OF THE INVENTION

Protein kinase is an enzyme that catalyzes the phosphorylation of proteins, and is also called protein phosphorylation enzyme by catalyzing the action of binding phosphate groups to hydroxy groups located on residues such as serine, threonine, tyrosine, and histidine of target proteins(phosphorylation).

To date, more than 500 types of protein kinases have been discovered. These can be classified into 5 categories according to the type of amino acid residues phosphorylated in the target protein: (1) serine/threonine (Ser/Thr) protein kinases, (2) tyrosine (Tyr) protein kinases, (3) histidine protein kinases, (4) tryptophan protein kinases, and (5) aspartyl/glutamyl protein kinases.

Protein kinases play an important role in growth factor signal transduction that induces cell cycle, cell survival, cell growth, differentiation, and proliferation. If their activities are not properly regulated, intracellular signal transduction becomes overactivated, leading to uncontrolled cell division and proliferation. In addition, abnormal activation due to gene mutations, amplification, and overexpression of protein kinases is associated with the development and progression of various tumors, playing a critical role in the growth and metastasis of cancer cells. Examples of such protein kinases include ARK5, FLT3, YES1, ABL1, KIT, and RET.

Fms-like tyrosine kinase 3(FLT3) is a protein belonging to class III of receptor-type tyrosine kinases. FLT3 is expressed in normal CD34-positive human bone marrow progenitor cells and dendritic cells, and plays an important role in the proliferation and differentiation of these cells (Brown Pet et al., European Journal of Cancer, 40th, pp. 707-721, 2004). In addition, the ligand of FLT3(FL) is expressed in bone marrow stromal cells and T cells, influences the development of various hematopoietic lineages, and is one of the cytokines that stimulate the proliferation of hepatocytes, progenitor cells, dendritic cells, and natural killer cells through interactions with other growth factors. FLT3 is also a target antigen for acute myeloid leukemia (AML), known to be overexpressed in AML patient blasts compared to healthy cells, and to be expressed in the majority of patient cells (Carow et al., Feb. 2, 1996 Blood: 87(3); Birg et al., November 1992 Blood: 80(10)). In particular, FLT3 is a frequently mutated gene in AML patients, with such mutations associated with poor prognosis (Abu-Duhier et al., British Journal of Haematology 2000 October; 111(1):190-5, Yamamoto et al., Apr. 15, 2001; Blood:97 (8)).

In addition, the association between FLT3 kinase and pancreatic and esophageal cancer has been confirmed, as there have been recent reports that FLT3 may be an important biomarker related to the prognosis of pancreatic cancer (Ger et al., Anticancer Research 38: 5759-5765 (2018), and that the ligand of FLT3 (FL) promotes resistance in esophageal squamous cell carcinoma (Zhu et al., Frontiers in Pharmacology, May 2021, Vol 12, Article 659735). Regarding FLT3 inhibitors, several inhibitors are in clinical stages, but they are incomplete and transient in treating AML patients, and drug resistance was developed rapidly in AML patients treated with these FLT3 inhibitors (Molecular Cancer Therapeutics, 2017, 16 (6): 991-1001).

Therefore, there is a need for methods to address drug resistance caused by FLT3 mutations and to effectively treat AML patients.

### DISCLOSURE OF THE INVENTION

### Problems to be Solved

In order to solve the above problems, the inventors made research efforts to derive novel compounds with inhibitory activity against protein kinases and discovered that a series of novel imidazole derivative compounds have excellent inhibitory activity against protein kinases. In particular, the present inventors completed the present invention by identifying that a series of novel imidazole derivative compounds effectively inhibit FLT3 and shows excellent inhibitory activity against mutant FLT3.

The object of the present invention is to provide a pharmaceutical composition for the prevention or treatment of cancer by inhibiting the activity of protein kinases, comprising a novel imidazole derivative compound or a pharmaceutically acceptable salt thereof.

Another object of the present invention is to provide a pharmaceutical composition for the prevention or treatment of FLT3-associated diseases, comprising a novel imidazole derivative compound or a pharmaceutically acceptable salt thereof, and use of the novel compounds for the prevention or treatment of FLT3-associated diseases.

### Means for Solving the Problems

According to one embodiment of the present invention, a compound represented by the following formula 1 or a pharmaceutically acceptable salt thereof is provided.

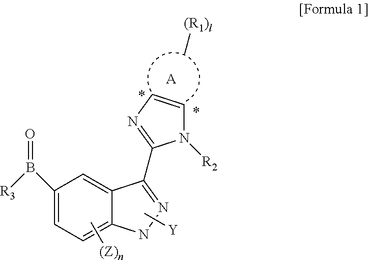

wherein in Formula 1
A is absent, or if present, is (6-14 membered) aryl or (5-12 membered) heteroaryl,
if the A is absent, R₁ is optionally bonded to the “*” position,
if the A is present, R₁ is optionally substituted on A,
R₁ is each independently selected from the group consisting of hydrogen, halogen, C₁₋₁₀ alkyl, C₁₋₁₀ haloalkyl, (3-10 membered) cycloalkyl, C₁₋₁₀ alkylene-(3-10 membered) cycloalkyl, (3-10 membered) heterocyclyl, C₁₋₁₀ alkylene-(3-10 membered) heterocyclyl, (6-14 membered) aryl, C₀₋₁₀ alkylene-(6-14 membered) aryl, (5-12 membered) heteroaryl, C₁₋₁₀ alkylene-(5-12 membered) heteroaryl, C₁₋₁₀ alkoxy, C₁₋₁₀ alkylene-C₁₋₆ alkoxy, O-(6-14 membered) aryl, C₁₋₁₀ alkylene-O-(6-14 membered)aryl, O—C₁₋₁₀ alkylene-C₁₋₆ alkoxy, O—C₁₋₁₀ alkylene-NR₁ₐR_{1b}, C(O)—NR₁ₐR_{1b}, C₁₋₁₀ alkylene-C(O)—NR₁ₐR_{1b}, C₁₋₆ haloalkoxy, C₁₋₁₀ alkylene-C₁₋₆ haloalkoxy, CN, C₁₋₁₀ alkylene-CN, OH, NO₂, NR₁ₐR_{1b}, OC(O)—NR₁ₐR_{1b}, C(O)R₁ₐ, C(O)OR₁ₐ, NR₁ₐC(O)R_{1b}, NR₁ₐC(O)OR_{1b}, NR₁ₐSO₂R_{1b} and SO₂NR₁ₐR_{1b},
wherein (3-10 membered) cycloalkyl, (3-10 membered) heterocyclyl, (6-14 membered) aryl, and (5-12 membered) heteroaryl are each independently unsubstituted or optionally substituted with at least one of (3-10 membered) cycloalkyl, (3-10 membered) heterocycyl, C₁₋₆ alkyl, C₁₋₆ alkoxy, halogen, C₁₋₆ haloalkyl, NH₂, NO₂, OH, or NR₁ₐR_{1b},
the R₁ₐ and R_{1b} are each independently selected from hydrogen, C₁₋₆ alkyl or C₁₋₆ haloalkyl, or the R₁ₐ and R_{1b} are optionally bonded to the same nitrogen atom to form (3-10 membered) heterocyclyl or (5-12 membered) heteroaryl within the substituent,
1 is an integer from 0 to 2 when A is absent, and an integer from 0 to 6 when A is present,
R₂ is each independently selected from the group consisting of hydrogen, C₁₋₁₀ alkyl, halogen, C₁₋₆ haloalkyl, C₁₋₆ alkoxy, C₁₋₁₀ alkylene-C₁₋₆ alkoxy, C₁₋₆ haloalkoxy, CN, OH, C₁₋₁₀ alkylene-OH, NO₂, C₁₋₆ alkylamino, C₁₋₁₀ alkylene-NR₂ₐR_{2b}, (6-14 membered) aryl, (5-12 membered) heteroaryl, (3-10 membered) cycloalkyl, (3-10 membered) heterocyclyl, and C₁₋₁₀ alkylene-(3-10 membered) heterocyclyl,
wherein (6-14 membered) aryl, (5-12 membered) heteroaryl, (3-10 membered) cycloalkyl and (3-10 membered) heterocyclyl are each independently unsubstituted or optionally substituted with at least one of C₁₋₆ alkyl, halogen, C₁₋₆ haloalkyl, C₁₋₆ alkoxy or C₁₋₆ haloalkoxy,
wherein R₂ₐ and R_{2b} are each independently selected from hydrogen, C₁₋₆ alkyl or C₁₋₆ haloalkyl, or the R₂ₐ and R_{2b} are optionally bonded together to the same nitrogen atom to form (3-10 membered) heterocyclyl or (5-12 membered) heteroaryl within the substituent,
B is carbon or sulfur,
R₃ is NR₃ₐR_{3b},
wherein R₃ₐ and R_{3b} are each independently selected from the group consisting of hydrogen, OH, C₁₋₁₀ alkyl, C₁₋₆ haloalkyl, C(O)C₁₋₆ alkyl,

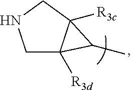

(3-10 membered) cycloalkyl, (6-14 membered) aryl, (5-12 membered) heteroaryl and (3-10 membered) heterocyclyl,
or the R₃ₐ and R_{3b} are optionally boned to the same nitrogen atom to form (3-10 membered) heterocyclyl or (5-12 membered) heteroaryl within the substituent,
wherein R_{3c} and R_{3d} are each independently selected from hydrogen, C₁₋₆ alkyl or C₁₋₆ haloalkyl,
wherein C₁₋₁₀ alkyl, (3-10 membered) cycloalkyl, (3-10 membered) heterocyclyl, (6-14 membered) aryl, and (5-12 membered) heteroaryl are each independently unsubstituted or optionally substituted with R₄,
R₄ is selected from the group consisting of OH, C₁₋₆ alkyl, C₁₋₆ alkoxy, halogen, C₁₋₆ haloalkyl, C(O)C₁₋₆ alkyl, (6-14 membered) aryl, (3-10 membered) cycloalkyl, (3-10 membered) heterocyclyl, (5-12 membered) heteroaryl and NR₄ₐR_{4b},
wherein R₄ₐ and R_{4b} are each independently selected from hydrogen, C₁₋₆ alkyl, C₁₋₆ haloalkyl, C(O)C₁₋₆ alkyl, SO₂(C₁₋₆)alkyl, or C(O)C₁₋₆ haloalkyl, or the R₄ₐ and R_{4b} are optionally boned to the same nitrogen atom to form (3-10 membered) heterocyclyl or (5-12 membered) heteroaryl within the substituent,
wherein C₁₋₆ alkyl, (6-14 membered)aryl, (3-10 membered)cycloalkyl, and (3-10 membered)heterocyclyl are each independently unsubstituted or optionally substituted with at least one of C₁₋₆ alkyl, amino, halogen, C₁₋₆ haloalkyl, C(O)C₁₋₆ alkyl, C₁₋₆ alkoxy, or C₁₋₆ haloalkoxy,
Y is selected from the group consisting of hydrogen, C₁₋₆ alkyl, C₁₋₆ haloalkyl, C₁₋₆ alkoxy, C(O)C₁₋₆ alkyl, C(O)OC₁₋₆ alkyl and C(O)N—C₁₋₆ alkyl,
Z is selected from the group consisting of hydrogen, C₁₋₆ alkyl, halogen, C₁₋₆ haloalkyl, C₁₋₆ alkoxy, NH₂, OH, CN, COOH, C(O)C₁₋₆ alkyl, C(O)OC₁₋₆ alkyl and C(O)N—C₁₋₆ alkyl, wherein heterocyclyl and heteroaryl each independently comprise at least one of N, O, or S in the ring,
n is an integer from 0 to 3.

The compounds disclosed herein may prevent or treat cancer by inhibiting the activity of protein kinases.

The compounds and pharmaceutical compositions disclosed herein may prevent or treat FLT3-associated diseases, for example, by administering the compounds or compositions to the subject.

### ADVANTAGE OF THE INVENTION

The imidazole derivative compound of the present invention is a novel compound that may be useful for the prevention or treatment of cancer by inhibiting the activity of protein kinases.

The novel compound of the present invention may also be useful for the prevention or treatment of mutant FLT3-associated diseases, especially acute myeloid leukemia (AML) by effectively inhibiting FLT3 activity.

### DETAILED DESCRIPTION OF THE INVENTION

### Definition of Terms

Unless defined otherwise, all technical terms used herein have the same meaning as commonly understood by a person of ordinary skill in the art to which this invention belongs. Also, numerical values recited herein are intended to include the meaning of “about” unless explicitly stated otherwise.

Definitions of moieties and substituents as used herein are provided below. Unless otherwise specified, each moiety is defined as follows, and is used with the same meaning as commonly understood by those skilled in the art.

The substituents, as used herein, are intended to include both further substituted and unsubstituted instances.

As used herein, the term “alkyl” is a hydrocarbon having primary, secondary, tertiary, and/or quaternary carbon atoms, including saturated aliphatic groups, which may be straight-chain, branched, cyclic, or combinations thereof. For example, an alkyl group may have 1 to 8 carbon atoms (i.e., C₁₋₈ alkyl), 1 to 6 carbon atoms (i.e., C₁₋₆ alkyl), or 1 to 3 carbon atoms (i.e., C₁₋₃ alkyl). Examples of suitable alkyl groups may include methyl (Me, —CH₃), ethyl (Et, —C₂H₅), 1-propyl (n-Pr, —CH₂CH₂CH₃), 2-propyl (i-Pr, —CH(CH₃)₂), 1-butyl (n-Bu, —CH₂CH₂CH₂CH₃), 2-methyl-1-propyl (i-Bu, —CH₂CH(CH₃)₂), 2-butyl (s-Bu, —CH(CH₃)CH₂CH₃), 2-methyl-2-propyl (t-Bu, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—CH(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃), and octyl (—(CH₂)₇CH₃) but is not limited thereto. As used herein, the terms “halo”, “halogen” refer to bromo, chloro, fluoro, or iodo.

As used herein, the term “alkoxy” refers to an —O-(alkyl) group, including —OCH₃, —OCH₂CH₃, —O(CH₂)₂CH₃, —O(CH₂)₃CH₃, —O(CH₂)₄CH₃, —O(CH₂)SCH₃, and the like, but is not limited thereto, wherein “alkyl” is as defined above.

As used herein, the terms “haloalkyl” and “haloalkoxy” refer to alkyl and alkoxy groups, in which one or more hydrogen atoms are each replaced by a halogen atom. For example, haloalkyl includes —CF₃, —CHF₂, —CH₂F, —CBr₃, —CHBr₂, —CH₂Br, —CCl₃, —CHCl₂, —CH₂Cl, —CI₃, —CHI₂, —CH₂I, —CH₂—CF₃, —CH₂—CHF₃, —CH₂—CH₂F, —CH₂—CBr₃, —CH₂—CHBr₂, —CH₂—CH₂Br, —CH₂—CCl₃, —CH₂—CHCl₂, —CH₂—CH₂Cl, —CH₂—CI₃, —CH₂—CHI₂, —CH₂—CH₂I and the like, but is not limited thereto. Wherein, alkyl and halogen are each as defined above.

As used herein, the term “amine” or “amino” refers to a substituted or unsubstituted amine group represented by the form —NRR′, wherein R and R′ are each independently selected from hydrogen, alkyl, alkenyl, alkynyl, aryl, arylalkyl, cycloalkyl, heteroaryl, and heterocyclyl groups.

The term “amino” also includes a corresponding quaternary ammonium salt of any amino group, such as —[N(R1)(Rm)(Rn)]⁺. Examples of amino groups include aminoalkyl groups in which at least one of R1, Rm, or Rn is an alkyl group. In certain embodiments, R1, Rm, and Rn are selected from hydrogen or alkyl.

As used herein, the term “alkylamino” refers to a concept that includes both mono-alkylamino and di-alkylamino groups.

As used herein, the term “mono-alkylamino” refers to an —NH(alkyl) group, including —NHCH₃, —NHCH₂CH₃, —NH(CH₂)₂CH₃, —NH(CH₂)₃CH₃, —NH(CH₂)₄CH₃, —NH(CH₂)SCH₃, and the like, wherein “alkyl” is as defined above.

As used herein, the term “di-alkylamino” refers to an —N(alkyl)(alkyl) group, including —N(CH₃)₂, —N(CH₂CH₃)₂, —N((CH₂)₂CH₃)₂, —N(CH₃)(CH₂CH₃), and the like, wherein each “alkyl” is independently as defined above.

As used herein, the term “cycloalkyl” refers to a monocyclic or polycyclic saturated ring having carbon and hydrogen atoms and no carbon-carbon multiple bonds. The cycloalkyl group may contain from 3 to 10 carbon atoms in a monocyclic ring, from 7 to 12 carbon atoms in a bicyclic ring, or up to about 20 carbon atoms in a polycyclic ring. Examples of cycloalkyl groups comprise, (C₃-C₂₀) cycloalkyl, (C₃-C₁₀) cycloalkyl or more preferably (C₃-C₇) cycloalkyl (e.g., cyclopropyl, cyclobutyl, cyclopentyl, cyclohexyl, and cycloheptyl) but are not limited thereto. Cycloalkyl groups may be optionally substituted. In one embodiment, the cycloalkyl group is monocyclic.

As used herein, the terms “heterocyclyl(hetero ring)” refer to a monovalent or divalent, saturated or partially saturated non-aromatic ring structure, preferably a 3- to 10-membered ring, more preferably a 3- to 7-membered ring, in which the ring structure contains at least 1 heteroatom, preferably 1 to 4 heteroatoms, more preferably 1 to 2 heteroatom(s). Examples of the suitable heteroatoms include, but are not limited to, oxygen, sulfur, and nitrogen. The term “heterocycloalkyl” also includes polycyclic ring systems having two or more cyclic rings in which two or more carbons are common to two adjacent rings, wherein at least one of the rings is heterocyclic and the other cyclic ring may be, for example, cycloalkyl, cycloalkenyl, cycloalkynyl, aryl, heteroaryl, and/or heterocyclyl. Bicyclic and polycyclic/heterocyclic ring systems may be fused, bridged, or spiro ring systems. Exemplary heterocycloalky groups include azetidinyl, dihydropyridyl, dihydroindolyl, tetrahydropyridyl (piperidinyl), tetrahydrothiophenyl, sulfur-oxidized tetrahydrothiophenyl, indolenyl, piperidinyl, 4-piperidinyl, pyrrolidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, 6H-1,2,5-thiadiazinyl, pyranyl, chromenyl, xanthenyl, phenoxatinyl, 2H-pyrrolyl, 3H-indolyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, quinuclidinyl, morpholinyl, and oxazolidinyl(each of which may be substituted or unsubstituted), but are not limited thereto.

As used herein, the term “alkylene” may refer to an alkylene group having 1 to 20 carbon atoms, 1 to 16 carbon atoms, 1 to 12 carbon atoms, 1 to 10 carbon atoms, or 1 to 4 carbon atoms, unless otherwise specified. The alkylene group may be a straight-chain, branched, or cyclic alkylene group, and may optionally be substituted with one or more substituents.

As used herein, the term “aryl” includes substituted or unsubstituted monovalent or divalent aromatic hydrocarbon groups, which may be monocyclic, bicyclic or polycyclic, in which each atom of the ring is carbon. Preferably, the aryl ring is a 6- to 20-membered ring, a 6- to 14-membered ring, a 6- to 10-membered ring, or more preferably a 6-membered ring. An aryl group may be a polycyclic ring system having two or more cyclic rings in which two or more carbons are common to two adjacent rings, wherein at least one of the rings may be aromatic, and the other cyclic ring may be, for example, a cycloalkyl, cycloalkenyl, cycloalkynyl, aryl, heteroaryl, and/or heterocycloalkyl. Aryl groups include benzene, naphthalene, phenanthrene, anthracene, indene, indane, phenol, and aniline, but are not limited thereto.

As used herein, the term “heteroaryl” refers to a substituted or unsubstituted monovalent or divalent aromatic group, which is monocyclic, bicyclic or polycyclic, containing one or more heteroatoms in the ring. Non-limiting examples of suitable heteroatoms that may be contained in the aromatic ring include, oxygen, sulfur and nitrogen. In polycyclic heteroaryl ring systems, the ring system has at least two cyclic rings in which at least two carbon atom are common to two adjacent rings, wherein at least one of the rings is heteroaromatic and the other cyclic rings may be, for example, cycloalkyl, cycloalkenyl, cycloalkynyl, aryl, heteroaryl, and/or heterocyclyl. Representative examples of heteroaryl groups include triazolyl, tetrazolyl, oxadiazolyl, pyridyl, furyl, benzofuranyl, quinolinyl, pyrrolyl, indolyl, oxazolyl, benzoxazolyl, imidazolyl, benzimidazolyl, isoxazolyl, pyrazolyl, pyridazinyl, pyrimidinyl, pyrazinyl, triazinyl, cinnolinyl, phthalazinyl, quinazolinyl, pyrimidyl, oxetanyl, azepinyl, piperazinyl, morpholinyl, dioxanyl, and oxazolyl(each of which may be unsubstituted or substituted), but are not limited to.

The term “substituted” used in the present invention for alkyl, alkoxy, alkoxyalkyl, alkylamino, aryl, heteroaryl, heterocycloalkyl, or cycloalkyl refers to alkyl, alkoxy, alkoxyalkyl, alkylamino, aryl, heteroaryl, heterocycloalkyl, or cycloalkyl, in which one or more hydrogen atoms are each independently replaced by a non-hydrogen substituent. A substituent may include any of the substituents described herein, for example, halogen, hydroxyl, alkyl, amino, nitroalkyl, alkoxy, carbonyl (e.g., carboxyl, alkoxycarbonyl, formyl, or acyl), phosphoryl, phosphate, phosphonate, amido, amidin, imine, cyano, azido, sulfate, sulfonate, sulfamoyl, sulfonamido, sulfonyl, heterocyclyl, aralkyl, or aromatic or heteroaromatic moieties, but are not limited thereto. It will be understood by those skilled in the art that a substituted moiety on the hydrocarbon chain may itself be optionally substituted.

Unless otherwise specified, the site of attachment of a substituent as used herein may be at any suitable position of the substituent.

As used herein, “adjacent substituents are bonded to each other to form a ring” means combining with adjacent substituents to form a substituted or unsubstituted aryl, heteroaryl, cycloalkyl, heterocycloalkyl, or condensed ring thereof.

The phrase “pharmaceutically acceptable” is an expression used in the art to indicate that a substance or composition must be chemically and/or toxicologically compatible with the other components of the formulation and/or the mammal to be treated therewith.

“Pharmaceutically acceptable salt” or “salt” are used herein to refer to an acid or base addition salt that is suitable for or compatible with the treatment of patients. An exemplary inorganic acid that forms a suitable salt may be hydrochloric acid, hydrobromic acid, sulfuric acid and phosphoric acid, as well as a metal salt, for example sodium monohydrogen orthophosphate and potassium hydrogen sulfate. An exemplary organic acid that forms a suitable salt may be mono-, di- and tricarboxylic acids, for example, trifluoroacetic acid, glycolic acid, lactic acid, pyruvic acid, malonic acid, succinic acid, glutaric acid, fumaric acid, malic acid, tartaric acid, citric acid, ascorbic acid, maleic acid, benzoic acid, phenylacetic acid, cinnamic acid and salicylic acid, as well as sulfonic acid, for example p-toluene sulfonic acid and methanesulfonic acid. Mono- or di-acid salts may be formed, and such salts may exist in hydrated, solvated or substantially anhydrous form. In general, acid addition salts of the compounds of the present invention are more soluble in water and various hydrophilic organic solvents compared to free base forms thereof, and generally exhibit higher melting points. The selection of suitable salts is known to those skilled in the art. Other non-pharmaceutically acceptable salts, for example, oxalates may be used in the isolation of the compounds of the present invention, for example for laboratory use or for subsequent conversion into pharmaceutically acceptable acid addition salts. An exemplary inorganic base that form a suitable salt may be lithium, sodium, potassium, calcium, magnesium, or barium hydroxides. An exemplary organic base that form a suitable salt may be aliphatic, cycloaliphatic or aromatic organic amines, for example, methylamine, trimethylamine and picoline or ammonia. Thus, in some embodiments, contemplated salts of the present invention may be alkyl, dialkyl, trialkyl or tetra-alkyl ammonium salts. In certain embodiments, contemplated salts of the present invention may be L-arginine, benethamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts, but is not limited thereto. In certain embodiments, contemplated salts of the present invention may be Na, Ca, K, Mg, Zn or other metal salts, but is not limited thereto.

The selection of appropriate salts is known to those skilled in the art.

Pharmaceutically acceptable acid addition salts may also exist as various solvates, for example, solvates with water, methanol, ethanol, dimethylformamide, and the like. Mixtures of these solvates may also be prepared. The source of such solvates may be from the solvent used for crystallization, inherently present in the solvent for manufacture or crystallization, or accidentally introduced through such a solvent.

As used herein, the term “IC₅₀” refers to the concentration of an inhibitor or compound that achieves 50% inhibition of inflammation.

As used herein, the term “GI₅₀” refers to the concentration of an inhibitor or compound that produces 50% inhibition relative to the maximum inhibition of cell proliferation.

As used herein, the terms “subject”, “patient” refer to a warm-blooded animal, for example, pig, cow, chicken, horse, guinea pig, mouse, rat, gerbil, cat, rabbit, dog, monkey, chimpanzee and human.

As used herein, the terms “treat”, “treating”, “treatment”, “improve” and “improving” refer to any objective or subjective parameter, e.g., decline; remission; reducing symptoms or making the symptom, injury, pathology or condition more tolerable by a patient; reducing the frequency or duration of a symptom or condition; or, in some circumstances, any indication of success in treating or ameliorating an injury, pathology, condition, or symptom (e.g., pain), including preventing the onset of the symptom or condition. Treatment or improvement of symptoms may be based on any objective or subjective parameter, for example, the results of a physical examination.

### Imidazole Derivative Compound

The present invention provides a novel imidazole derivative compound.

In one embodiment, the present invention relates to an imidazole derivative compound represented by the following Formula 1 or a pharmaceutically acceptable salt thereof.

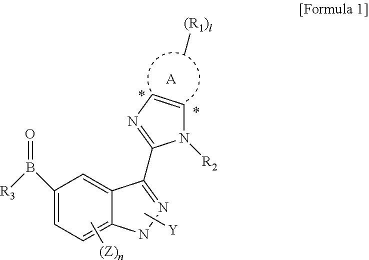

wherein in Formula 1,
A is absent, or if present, is (6-14 membered) aryl or (5-12 membered) heteroaryl,
if the A is absent, R₁ is optionally bonded to the “*” position,
if the A is present, R₁ may be substituted on A,
R₁ is independently selected from the group consisting of hydrogen, halogen, C₁₋₁₀ alkyl, C₁₋₁₀ haloalkyl, (3-10 membered) cycloalkyl, C₁₋₁₀ alkylene-(3-10 membered) cycloalkyl, (3-10 membered) heterocyclyl, C₁₋₁₀ alkylene-(3-10 membered) heterocyclyl, (6-14 membered) aryl, C₀₋₁₀ alkylene-(6-14 membered) aryl, (5-12 membered) heteroaryl, C₁₋₁₀ alkylene-(5-12 membered) heteroaryl, C₁₋₁₀ alkoxy, C₁₋₁₀ alkylene-C₁₋₆ alkoxy, O-(6-14 membered) aryl, C₁₋₁₀ alkylene-O-(6-14 membered)aryl, O—C₁₋₁₀ alkylene-C₁₋₆ alkoxy, O—C₁₋₁₀ alkylene-NR₁ₐR_{1b}, C(O)—NR₁ₐR_{1b}, C₁₋₁₀ alkylene-C(O)—NR₁ₐR_{1b}, C₁₋₆ haloalkoxy, C₁₋₁₀ alkylene-C₁₋₆ haloalkoxy, CN, C₁₋₁₀ alkylene-CN, OH, NO₂, NR₁ₐR_{1b}, OC(O)—NR₁ₐR_{1b}, C(O)R₁ₐ, C(O)OR₁ₐ, NR₁ₐC(O)R_{1b}, NR₁ₐC(O)OR_{1b}, NR₁ₐSO₂R_{1b} and SO₂NR₁ₐR_{1b},
wherein (3-10 membered) cycloalkyl, (3-10 membered) heterocyclyl, (6-14 membered) aryl, and (5-12 membered) heteroaryl may each independently be unsubstituted or substituted with at least one of (3-10 membered) cycloalkyl, (3-10 membered) heterocycyl, C₁₋₆ alkyl, C₁₋₆ alkoxy, halogen, C₁₋₆ haloalkyl, NH₂, NO₂, OH, or NR₁ₐR_{1b}, wherein R₁ₐ and R_{1b} are each independently selected from hydrogen, C₁₋₆ alkyl or C₁₋₆ haloalkyl, or the R₁ₐ and R_{1b} may be bonded to the same nitrogen atom to form (3-10 membered) heterocyclyl or (5-12 membered) heteroaryl within the substituent,
1 is an integer from 0 to 2 when A is absent, and an integer from 0 to 6 when A is present,
R₂ is each independently selected from the group consisting of hydrogen, C₁₋₁₀ alkyl, halogen, C₁₋₆ haloalkyl, C₁₋₆ alkoxy, C₁₋₁₀ alkylene-C₁₋₆ alkoxy, C₁₋₆ haloalkoxy, CN, OH, C₁₋₁₀ alkylene-OH, NO₂, C₁₋₆ alkylamino, C₁₋₁₀ alkylene-NR₂ₐR_{2b}, (6-14 membered) aryl, (5-12 membered) heteroaryl, (3-10 membered) cycloalkyl, (3-10 membered) heterocyclyl, and C₁₋₁₀ alkylene-(3-10 membered) heterocyclyl,
wherein (6-14 membered) aryl, (5-12 membered) heteroaryl, (3-10 membered) cycloalkyl and (3-10 membered) heterocyclyl may each independently be unsubstituted or substituted with at least one of C₁₋₆ alkyl, halogen, C₁₋₆ haloalkyl, C₁₋₆ alkoxy or C₁₋₆ haloalkoxy,
wherein R₂ₐ and R_{2b} are independently selected from hydrogen, C₁₋₆ alkyl or C₁₋₆ haloalkyl, or the R₂ₐ and R_{2b} may be bonded together to the same nitrogen atom to form (3-10 membered) heterocyclyl or (5-12 membered) heteroaryl within the substituent,
B is carbon or sulfur,
R₃ is NR₃ₐR_{3b},
wherein R₃ₐ and R_{3b} are independently selected from the group consisting of hydrogen, OH, C₁₋₁₀ alkyl, C₁₋₆ haloalkyl, C(O)C₁₋₆ alkyl,

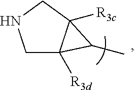

(3-10 membered) cycloalkyl, (6-14 membered) aryl, (5-12 membered) heteroaryl and (3-10 membered) heterocyclyl,
or wherein R₃ₐ and R_{3b} may be boned to the same nitrogen atom to form (3-10 membered) heterocyclyl or (5-12 membered) heteroaryl within the substituent,
wherein R_{3c} and R_{3d} are each independently selected from hydrogen, C₁₋₆ alkyl or C₁₋₆ haloalkyl, wherein C₁₋₁₀ alkyl, (3-10 membered) cycloalkyl, (3-10 membered) heterocyclyl, (6-14 membered) aryl, and (5-12 membered) heteroaryl may each independently be unsubstituted or substituted with R₄,
R₄ is selected from the group consisting of OH, C₁₋₆ alkyl, C₁₋₆ alkoxy, halogen, C₁₋₆ haloalkyl, C(O)C₁₋₆ alkyl, (6-14 membered) aryl, (3-10 membered) cycloalkyl, (3-10 membered) heterocyclyl, (5-12 membered) heteroaryl and NR₄ₐR_{4b},
wherein R₄ₐ and R_{4b} are each independently selected from hydrogen, C₁₋₆ alkyl, C₁₋₆ haloalkyl, C(O)C₁₋₆ alkyl, SO₂(C₁₋₆)alkyl, or C(O)C₁₋₆ haloalkyl, or the R₄ₐ and R_{4b} may be boned to the same nitrogen atom to form (3-10 membered) heterocyclyl or (5-12 membered) heteroaryl within the substituent,
wherein C₁₋₆ alkyl, (6-14 membered)aryl, (3-10 membered)cycloalkyl, and (3-10 membered)heterocyclyl may each independently be unsubstituted or substituted with at least one of C₁₋₆ alkyl, amino, halogen, C₁₋₆ haloalkyl, C(O)C₁₋₆ alkyl, C₁₋₆ alkoxy, or C₁₋₆ haloalkoxy,
Y is selected from the group consisting of hydrogen, C₁₋₆ alkyl, C₁₋₆ haloalkyl, C₁₋₆ alkoxy, C(O)C₁₋₆ alkyl, C(O)OC₁₋₆ alkyl and C(O)N—C₁₋₆ alkyl,
Z is selected from the group consisting of hydrogen, C₁₋₆ alkyl, halogen, C₁₋₆ haloalkyl, C₁₋₆ alkoxy, NH₂, OH, CN, COOH, C(O)C₁₋₆ alkyl, C(O)OC₁₋₆ alkyl and C(O)N—C₁₋₆ alkyl,
wherein heterocyclyl and heteroaryl each independently comprise at least one of N, O, or S in the ring,
n may be an integer from 0 to 3.

In one specific embodiment of the present invention, A is as defined in claim 1,
1 is an integer from 0 to 2 both when A is absent and present,
R₁ is each independently selected from the group consisting of hydrogen, halogen, C₁₋₁₀alkyl, C₁₋₁₀ haloalkyl, (3-10 membered) heterocyclyl, C₁₋₁₀ alkylene-(3-10 membered) heterocyclyl, (6-14 membered) aryl, (5-12 membered) heteroaryl, C₁₋₁₀ alkoxy, C₁₋₁₀ alkylene-C₁₋₆ alkoxy, C₁₋₁₀ alkylene-O-(6-14 membered) aryl, O—C₁₋₁₀ alkylene-C₁₋₆ alkoxy, O—C₁₋₁₀ alkylene-NR₁ₐR_{1b}, C(O)—NR₁ₐR_{1b}, C₁₋₁₀ alkylene-C(O)—NR₁ₐR_{1b} and CN.
wherein (3-10 membered) heterocyclyl, (6-14 membered) aryl, and (5-12 membered) heteroaryl may each independently be unsubstituted or substituted with at least one of (3-10 membered) heterocyclyl, C₁₋₆ alkyl, C₁₋₆ alkoxy, halogen, C₁₋₆ haloalkyl, NH₂, NO₂, OH, or NR₁ₐR_{1b},
wherein R₁ₐ and R_{1b} are each independently hydrogen or C₁₋₆ alkyl, or the R₁ₐ and R_{1b} may be bonded to the same nitrogen atom to form (3-10 membered) heterocyclyl within the substituent,
R₂ is each independently selected from the group consisting of hydrogen, C₁₋₁₀ alkyl, C₁₋₁₀ alkylene-C₁₋₆ alkoxy, C₁₋₁₀ alkylene-OH, C₁₋₁₀ alkylene-NR₂ₐR_{2b}, and C₁₋₁₀ alkylene-(3-10 membered) heterocyclyl,
wherein (3-10 membered) cycloalkyl may each independently be unsubstituted or substituted with C₁₋₆ alkyl,
wherein R₂ₐ and R_{2b} are independently selected from hydrogen or C₁₋₆ alkyl, or the R₂ₐ and R_{2b} may be bonded to the same nitrogen atom to form (3-10 membered) heterocyclyl within the substituent,
B is carbon or sulfur,
R₃ is —NR₃ₐR_{3b},
wherein R₃ₐ and R_{3b} are independently selected from the group consisting of hydrogen, C₁₋₁₀ alkyl,

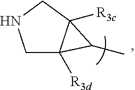

(3-10 membered) cycloalkyl, (6-14 membered) aryl, and (3-10 membered) heterocyclyl,
or wherein R₃ₐ and R_{3b} may be boned to the same nitrogen atom to form (3-10 membered) heterocyclyl within the substituent,
wherein R_{3c} and R_{3d} are each independently selected from hydrogen, C₁₋₆ alkyl,
wherein C₁₋₁₀ alkyl, (3-10 membered) cycloalkyl, (6-14 membered) aryl, and (3-10 membered) heterocyclyl may each independently be unsubstituted or substituted with R₄,
R₄ is selected from the group consisting of OH, C₁₋₆ alkyl, C₁₋₆ haloalkyl, C(O)C₁₋₆ alkyl, (6-14 membered) aryl, (3-10 membered) cycloalkyl, (3-10 membered) heterocyclyl and NR₄ₐR_{4b},
wherein R₄ₐ and R_{4b} are each independently selected from hydrogen, C₁₋₆ alkyl, or SO₂(C₁₋₆)alkyl, or the R₄ₐ and R_{4b} may be boned to the same nitrogen atom to form (3-10 membered) heterocyclyl within the substituent,
wherein C₁₋₆ alkyl, (6-14 membered)aryl, (3-10 membered)cycloalkyl, and (3-10 membered)heterocyclyl may each independently be unsubstituted or substituted with at least one of C₁₋₆ alkyl, amino, halogen, C₁₋₆ haloalkyl, or C(O)C₁₋₆ alkyl,
Y is hydrogen, and n may be 0.

In one specific embodiment of the present invention, a compound represented by the following formula 2 or a pharmaceutically acceptable salt thereof is provided.

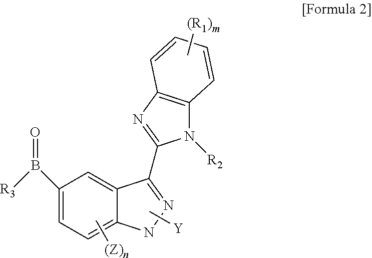

wherein in Formula 2,
R₁ is each independently selected from the group consisting of hydrogen, halogen, C₁₋₁₀ alkyl, C₁₋₁₀ haloalkyl, (3-10 membered) cycloalkyl, C₁₋₁₀ alkylene-(3-10 membered) cycloalkyl, (3-10 membered) heterocyclyl, C₁₋₁₀ alkylene-(3-10 membered) heterocyclyl, (6-14 membered) aryl, C₀₋₁₀ alkylene-(6-14 membered) aryl, (5-12 membered) heteroaryl, C₁₋₁₀ alkylene-(5-12 membered) heteroaryl, C₁₋₁₀ alkoxy, C₁₋₁₀ alkylene-C₁₋₆ alkoxy, 0-(6-14 membered) aryl, C₁₋₁₀ alkylene-O-(6-14 membered)aryl, O—C₁₋₁₀ alkylene-C₁₋₆ alkoxy, O—C₁₋₁₀ alkylene-NR₁ₐR_{1b}, C(O)—NR₁ₐR_{1b}, C₁₋₁₀ alkylene-C(O)—NR₁ₐR_{1b}, C₁₋₆ haloalkoxy, C₁₋₁₀ alkylene-C₁₋₆ haloalkoxy, CN, C₁₋₁₀ alkylene-CN, OH, NO₂, NR₁ₐR_{1b}, OC(O)—NR₁ₐR_{1b}, C(O)R₁ₐ, C(O)OR₁ₐ, NR₁ₐC(O)R_{1b}, NR₁ₐC(O)OR_{1b}, NR₁ₐSO₂R_{1b} and SO₂NR₁ₐR_{1b},
wherein (3-10 membered) cycloalkyl, (3-10 membered) heterocyclyl, (6-14 membered) aryl, and (5-12 membered) heteroaryl may each independently be unsubstituted or substituted with at least one of (3-10 membered) cycloalkyl, (3-10 membered) heterocycyl, C₁₋₆ alkyl, C₁₋₆ alkoxy, halogen, C₁₋₆ haloalkyl, NH₂, NO₂, OH, or NR₁ₐR_{1b},
wherein R₁ₐ and R_{1b} are each independently selected from hydrogen, C₁₋₆ alkyl or C₁₋₆ haloalkyl, or the R₁ₐ and R_{1b} may be bonded to the same nitrogen atom to form (3-10 membered) heterocyclyl or (5-12 membered) heteroaryl within the substituent,
m is an integer from 0 to 4,
R₂ is each independently selected from the group consisting of hydrogen, C₁₋₁₀ alkyl, halogen, C₁₋₆ haloalkyl, C₁₋₆ alkoxy, C₁₋₁₀ alkylene-C₁₋₆ alkoxy, C₁₋₆ haloalkoxy, CN, OH, C₁₋₁₀ alkylene-OH, NO₂, C₁₋₆ alkylamino, C₁₋₁₀ alkylene-NR₂ₐR_{2b}, (6-14 membered) aryl, (5-12 membered) heteroaryl, (3-10 membered) cycloalkyl, (3-10 membered) heterocyclyl, and C₁₋₁₀ alkylene-(3-10 membered) heterocyclyl,
wherein (6-14 membered) aryl, (5-12 membered) heteroaryl, (3-10 membered) cycloalkyl and (3-10 membered) heterocyclyl may each independently be unsubstituted or substituted with at least one of C₁₋₆ alkyl, halogen, C₁₋₆ haloalkyl, C₁₋₆ alkoxy or C₁₋₆ haloalkoxy,
wherein R₂ₐ and R_{2b} are independently selected from hydrogen, C₁₋₆ alkyl or C₁₋₆ haloalkyl, or the R₂ₐ and R_{2b} may be bonded to the same nitrogen atom to form (3-10 membered) heterocyclyl or (5-12 membered) heteroaryl within the substituent,
B is carbon or sulfur,
R₃ is —NR₃ₐR_{3b},
wherein R₃ₐ and R_{3b} are independently selected from the group consisting of hydrogen, OH, C₁₋₁₀ alkyl, C₁₋₆ haloalkyl, C(O)C₁₋₆ alkyl,

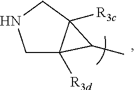

(3-10 membered) cycloalkyl, (6-14 membered) aryl, (5-12 membered) heteroaryl and (3-10 membered) heterocyclyl,
or wherein R₃ₐ and R_{3b} may be boned to the same nitrogen atom to form (3-10 membered) heterocyclyl or (5-12 membered) heteroaryl within the substituent,
wherein R_{3c} and R_{3d} are each independently selected from hydrogen, C₁₋₆ alkyl or C₁-6 haloalkyl,
wherein C₁₋₁₀ alkyl, (3-10 membered) cycloalkyl, (3-10 membered) heterocyclyl, (6-14 membered) aryl, and (5-12 membered) heteroaryl may each independently be unsubstituted or substituted with R₄,
R₄ is selected from the group consisting of OH, C₁₋₆ alkyl, C₁₋₆ alkoxy, halogen, C₁₋₆ haloalkyl, C(O)C₁₋₆ alkyl, (6-14 membered) aryl, (3-10 membered) cycloalkyl, (3-10 membered) heterocyclyl, (5-12 membered) heteroaryl and NR₄ₐR_{4b},
wherein R₄ₐ and R_{4b} are each independently selected from hydrogen, C₁₋₆ alkyl, C₁₋₆ haloalkyl, C(O)C₁₋₆ alkyl, SO₂(C₁₋₆)alkyl, or C(O)C₁₋₆ haloalkyl, or the R₄ₐ and R_{4b} may be boned to the same nitrogen atom to form (3-10 membered) heterocyclyl or (5-12 membered) heteroaryl within the substituent,
wherein C₁₋₆ alkyl, (6-14 membered)aryl, (3-10 membered)cycloalkyl, and (3-10 membered)heterocyclyl may each independently be unsubstituted or substituted with at least one of C₁₋₆ alkyl, amino, halogen, C₁₋₆ haloalkyl, C(O)C₁₋₆ alkyl, C₁₋₆ alkoxy, or C₁₋₆ haloalkoxy,
Y is selected from the group consisting of hydrogen, C₁₋₆ alkyl, C₁₋₆ haloalkyl, C₁₋₆ alkoxy, C(O)C₁₋₆ alkyl, C(O)OC₁₋₆ alkyl and C(O)N—C₁₋₆ alkyl,
Z is selected from the group consisting of hydrogen, C₁₋₆ alkyl, halogen, C₁₋₆ haloalkyl, C₁₋₆ alkoxy, NH₂, OH, CN, COOH, C(O)C₁₋₆ alkyl, C(O)OC₁₋₆ alkyl and C(O)N—C₁₋₆ alkyl,
wherein heterocyclyl and heteroaryl each independently comprise at least one of N, O, or S in the ring,
n may be an integer from 0 to 3.

In one specific embodiment of the present invention, in Formula 2, R₁ is each independently selected from the group consisting of hydrogen, halogen, C₁₋₁₀ alkyl, C₁₋₁₀ haloalkyl, (3-10 membered) heterocyclyl, C₁₋₁₀ alkylene-(3-10 membered) heterocyclyl, (6-14 membered) aryl, (5-12 membered) heteroaryl, C₁₋₁₀ alkoxy, O—C₁₋₁₀ alkylene-C₁₋₆ alkoxy, O—C₁₋₁₀ alkylene-NR₁ₐR_{1b}, C(O)—NR₁ₐR_{1b} and CN.
wherein (3-10 membered) heterocyclyl, (6-14 membered) aryl, and (5-12 membered) heteroaryl may each independently be unsubstituted or substituted with at least one of (3-10 membered) heterocyclyl, C₁₋₆ alkyl, C₁₋₆ alkoxy, halogen, C₁₋₆ haloalkyl, NH₂, NO₂, OH, or NR₁ₐR_{1b},
wherein R₁ₐ and R_{1b} are each independently hydrogen or C₁₋₆ alkyl, or the R₁ₐ and R_{1b} may be bonded to the same nitrogen atom to form (3-10 membered) heterocyclyl within the substituent,
m is an integer from 0 to 2,
R₂ is each independently selected from the group consisting of hydrogen, C₁₋₁₀ alkyl, C₁₋₁₀ alkylene-C₁₋₆ alkoxy, C₁₋₁₀ alkylene-OH, C₁₋₁₀ alkylene-NR₂ₐR_{2b}, and C₁₋₁₀ alkylene-(3-10 membered) heterocyclyl, wherein (3-10 membered) heterocyclyl may be unsubstituted or substituted with C₁₋₆ alkyl,
wherein R₂ₐ and R_{2b} are independently selected from hydrogen, C₁₋₆ alkyl, or C₁₋₆ haloalkyl, or the R₂ₐ and R_{2b} may be bonded to the same nitrogen atom to form (3-10 membered) heterocyclyl within the substituent,
B is carbon,
R₃ is —NR₃ₐR_{3b},
wherein R₃ₐ and R_{3b} are independently selected from the group consisting of hydrogen, C₁₋₁₀ alkyl,

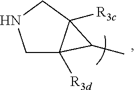

(3-10 membered) cycloalkyl, (6-14 membered) aryl, and (3-10 membered) heterocyclyl,
or the R₃ₐ and R_{3b} may be boned to the same nitrogen atom to form (3-10 membered) heterocyclyl within the substituent,
wherein R_{3c} and R_{3d} are each independently selected from hydrogen, C₁₋₆ alkyl,
wherein C₁₋₁₀ alkyl, (3-10 membered) cycloalkyl, (6-14 membered) aryl and (3-10 membered) heterocyclyl may each independently be unsubstituted or substituted with R₄,
R₄ is selected from the group consisting of OH, C₁₋₆ alkyl, C₁₋₆ haloalkyl, C(O)C₁₋₆ alkyl, (6-14 membered) aryl, (3-10 membered) cycloalkyl, (3-10 membered) heterocyclyl and NR₄ₐR_{4b},
wherein R₄ₐ and R_{4b} are each independently selected from hydrogen, C₁₋₆ alkyl or C(O)C₁₋₆ alkyl, or the R₄ₐ and R_{4b} may be boned to the same nitrogen atom to form (3-10 membered) heterocyclyl within the substituent,
wherein C₁₋₆ alkyl, (6-14 membered)aryl, (3-10 membered)cycloalkyl, and (3-10 membered)heterocyclyl may each independently be unsubstituted or substituted with at least one of C₁₋₆ alkyl, amino, halogen, and C(O)C₁₋₆ alkyl,
Y is hydrogen, and n may be 0.

Representative compounds of Formula 2 comprise, but are not limited to, compounds selected from the group consisting of Examples 1 to 80 and Examples 104 to 112 in Tables 1 to 3 below.

TABLE 1

| Example | name |
|---|---|
| 1 | (3-(1H-benzo[d]imidazol-2-yl)-1H-indazol-5-yl)(4-phenylpiperazin-1- |
| | yl)methanone |
| 2 | (3-(1H-benzo[d]imidazol-2-yl)-1H-indazol-5-yl)(4-methylpiperazin-1- |
| | yl)methanone |
| 3 | (3-(1H-benzo[d]imidazol-2-yl)-1H-indazol-5-yl)(4-(4-fluorophenyl)piperazin-1- |
| | yl)methanone |
| 4 | (3-(1H-benzo[d]imidazol-2-yl)-1H-indazol-5-yl)(4-(2-fluorophenyl)piperazin-1- |
| | yl)methanone |
| 5 | (3-(1H-benzo[d]imidazol-2-yl)-1H-indazol-5-yl)(4-(3-chlorophenyl)piperazin- |
| | 1-yl)methanone |
| 6 | (3-(1H-benzo[d]imidazol-2-yl)-1H-indazol-5-yl)(4-(2-chlorophenyl)piperazin- |
| | 1-yl)methanone |
| 7 | N-(3-aminopropyl)-3-(1H-benzo[d]imidazol-2-yl)-1H-indazole-5-carboxamide |
| 8 | N-(2-(4-aminopiperidin-1-yl)ethyl)-3-(1H-benzo[d]imidazol-2-yl)-1H-indazole- |
| | 5-carboxamide |
| 9 | 3-(5,6-difluoro-1H-benzo[d]imidazol-2-yl)-N-(3-(dimethylamino)propyl)-1H- |
| | indazole-5-carboxamide |
| 10 | 3-(6-cyano-1H-benzo[d]imidazol-2-yl)-N-(3-(dimethylamino)propyl)-1H- |
| | indazole-5-carboxamide |
| 11 | N-(3-(dimethylamino)propyl)-3-(6-methoxy-1H-benzo[d]imidazol-2-yl)-1H- |
| | indazole-5-carboxamide |
| 12 | N-(3-(dimethylamino)propyl)-3-(6-methyl-1H-benzo[d]imidazol-2-yl)-1H- |
| | indazole-5-carboxamide |
| 13 | 3-(6-(pyrrolidin-1-yl)-1H-benzo[d]imidazol-2-yl)-N-(4,4,4-trifluorobutyl)-1H- |
| | indazole-5-carboxamide |
| 14 | N-((3,3-difluorocyclobutyl)methyl)-3-(6-(pyrrolidin-1-yl)-1H- |
| | benzo[d]imidazol-2-yl)-1H-indazole-5-carboxamide |
| 15 | N-(3-aminopropyl)-3-(6-methyl-1H-benzo[d]imidazol-2-yl)-1H-indazole-5- |
| | carboxamide |
| 16 | N-(3-aminopropyl)-3-(6-(pyrrolidin-1-yl)-1H-benzo[d]imidazol-2-yl)-1H- |
| | indazole-5-carboxamide |
| 17 | N-(3-aminopropyl)-3-(6-(piperidin-1-yl)-1H-benzo[d]imidazol-2-yl)-1H- |
| | indazole-5-carboxamide |
| 18 | N-(3-aminopropyl)-3-(6-(4-methylpiperazin-1-yl)-1H-benzo[d]imidazol-2-yl)- |
| | 1H-indazole-5-carboxamide |
| 19 | N-(3-aminopropyl)-3-(6-((3S,5R)-3,5-dimethylpiperazin-1-yl)-1H- |
| | benzo[d]imidazol-2-yl)-1H-indazole-5-carboxamide |
| 20 | N-(3-aminopropyl)-3-(6-morpholino-1H-benzo[d]imidazol-2-yl)-1H-indazole- |
| | 5-carboxamide |
| 21 | N-(3-aminopropyl)-3-(6-(morpholine-4-carbonyl)-1H-benzo[d]imidazol-2-yl)- |
| | 1H-indazole-5-carboxamide |
| 22 | N-(3-aminopropyl)-3-(6-methoxy-1H-benzo[d]imidazol-2-yl)-1H-indazole-5- |
| | carboxamide |
| 23 | N-(3-aminopropyl)-3-(6-(2-methoxyethoxy)-1H-benzo[d]imidazol-2-yl)-1H- |
| | indazole-5-carboxamide |
| 24 | N-(3-aminopropyl)-3-(6-(2-(dimethylamino)ethoxy)-1H-benzo[d]imidazol-2- |
| | yl)-1H-indazole-5-carboxamide |
| 25 | N-(3-aminopropyl)-3-(7-fluoro-1H-benzo[d]imidazol-2-yl)-1H-indazole-5- |
| | carboxamide |
| 26 | N-(3-aminopropyl)-3-(6-(morpholinomethyl)-1H-benzo[d]imidazol-2-yl)-1H- |
| | indazole-5-carboxamide |
| 27 | N-(3-aminopropyl)-3-(6-chloro-1H-benzo[d]imidazol-2-yl)-1H-indazole-5- |
| | carboxamide |
| 28 | N-(3-aminopropyl)-3-(6-bromo-1H-benzo[d]imidazol-2-yl)-1H-indazole-5- |
| | carboxamide |
| 29 | N-(3-aminopropyl)-3-(6-(trifluoromethyl)-1H-benzo[d]imidazol-2-yl)-1H- |
| | indazole-5-carboxamide |
| 30 | N-((1S,3S)-3-aminocyclopentyl)-3-(6-(pyrrolidin-1-yl)-1H-benzo[d]imidazol-2- |
| | yl)-1H-indazole-5-carboxamide |
| 31 | N-((1S,3S)-3-aminocyclopentyl)-3-(6-(piperidin-1-yl)-1H-benzo[d]imidazol-2- |
| | yl)-1H-indazole-5-carboxamide |
| 32 | N-((1S,3S)-3-aminocyclopentyl)-3-(6-methoxy-1H-benzo[d]imidazol-2-yl)-1H- |
| | indazole-5-carboxamide |
| 33 | N-(3-acetamidopropyl)-3-(6-(piperidin-1-yl)-1H-benzo[d]imidazol-2-yl)-1H- |
| | indazole-5-carboxamide |
| 34 | (3-(6-fluoro-1H-benzo[d]imidazol-2-yl)-1H-indazol-5-yl)(4-(4-methylpiperazin- |
| | 1-yl) piperidin-1-yl)methanone |
| 35 | N-(2-(dimethylamino)ethyl)-3-(6-fluoro-1H-benzo[d]imidazol-2-yl)-1H- |
| | indazole-5-carboxamide |
| 36 | N-(3-(dimethylamino)propyl)-3-(6-fluoro-1H-benzo[d]imidazol-2-yl)-1H- |
| | indazole-5-carboxamide |
| 37 | N-(2-aminoethyl)-3-(6-fluoro-1H-benzo[d]imidazol-2-yl)-1H-indazole-5- |
| | carboxamide |
| 38 | N-(3-aminopropyl)-3-(6-fluoro-1H-benzo[d]imidazol-2-yl)-1H-indazole-5- |
| | carboxamide |
| 39 | N-(4-aminobutyl)-3-(6-fluoro-1H-benzo[d]imidazol-2-yl)-1H-indazole-5- |
| | carboxamide |
| 40 | 3-(6-fluoro-1H-benzo[d]imidazol-2-yl)-N-(piperidin-4-yl)-1H-indazole-5- |
| | carboxamide |

TABLE 2

| Example | name |
|---|---|
| 41 | (R)-3-(6-fluoro-1H-benzo[d]imidazol-2-yl)-N-(piperidin-3-yl)-1H-indazole-5- |
| | carboxamide |
| 42 | N-(2-aminoethyl)-3-(6-morpholino-1H-benzo[d]imidazol-2-yl)-1H-indazole-5- |
| | carboxamide |
| 43 | N-(4-aminobutyl)-3-(6-morpholino-1H-benzo[d]imidazol-2-yl)-1H-indazole- |
| | 5-carboxamide |
| 44 | N-(3-(methylamino)propyl)-3-(6-morpholino-1H-benzo[d]imidazol-2-yl)-1H- |
| | indazole-5-carboxamide |
| 45 | N-(3-amino-2-methylpropyl)-3-(6-morpholino-1H-benzo[d]imidazol-2-yl)-1H- |
| | indazole-5-carboxamide |
| 46 | N-(azetidin-3-yl)-3-(6-morpholino-1H-benzo[d]imidazol-2-yl)-1H-indazole-5- |
| | carboxamide |
| 47 | N-(azetidin-3-ylmethyl)-3-(6-morpholino-1H-benzo[d]imidazol-2-yl)-1H- |
| | indazole-5-carboxamide |
| 48 | N-(2-(azetidin-3-yl)ethyl)-3-(6-morpholino-1H-benzo[d]imidazol-2-yl)-1H- |
| | indazole-5-carboxamide |
| 49 | (3-(6-morpholino-1H-benzo[d]imidazol-2-yl)-1H-indazol-5-yl)(2,6- |
| | diazaspiro[3.3]heptan-2-yl)methanone |
| 50 | N-((1S, 3R)-3-aminocyclopentyl)-3-(6-morpholino-1H-benzo[d]imidazol-2-yl)- |
| | 1H-indazole-5-carboxamide |
| 51 | N-((1S,3S)-3-aminocyclopentyl)-3-(6-morpholino-1H-benzo[d]imidazol-2-yl)- |
| | 1H-indazole-5-carboxamide |
| 52 | (R)-3-(6-morpholino-1H-benzo[d]imidazol-2-yl)-N-(pyrrolidin-3-yl)-1H- |
| | indazole-5-carboxamide |
| 53 | (S)-3-(6-morpholino-1H-benzo[d]imidazol-2-yl)-N-(pyrrolidin-3-yl)-1H- |
| | indazole-5-carboxamide |
| 54 | (R)-3-(6-morpholino-1H-benzo[d]imidazol-2-yl)-N-(pyrrolidin-3-ylmethyl)- |
| | 1H-indazole-5-carboxamide |
| 55 | (S)-3-(6-morpholino-1H-benzo[d]imidazol-2-yl)-N-(pyrrolidin-3-ylmethyl)- |
| | 1H-indazole-5-carboxamide |
| 56 | 3-(6-morpholino-1H-benzo[d]imidazol-2-yl)-N-(piperidin-3-ylmethyl)-1H- |
| | indazole-5-carboxamide |
| 57 | 3-(6-morpholino-1H-benzo[d]imidazol-2-yl)-N-(piperidin-4-ylmethyl)-1H- |
| | indazole-5-carboxamide |
| 58 | 3-(6-morpholino-1H-benzo[d]imidazol-2-yl)-N-(piperidin-4-yl)-1H-indazole- |
| | 5-carboxamide |
| 59 | N-((1s, 4s)-4-aminocyclohexyl)-3-(6-morpholino-1H-benzo[d]imidazol-2-yl)- |
| | 1H-indazole-5-carboxamide |
| 60 | 1-(6-(3-(6-morpholino-1H-benzo[d]imidazol-2-yl)-1H-indazole-5-carbonyl)- |
| | 2,6-diazaspiro[3.3]heptan-2-yl)ethan-1-one |
| 61 | N-((1-acetylazetidin-3-yl)methyl)-3-(6-morpholino-1H-benzo[d]imidazol-2- |
| | yl)-1H-indazole-5-carboxamide |
| 62 | N-(2-(dimethylamino)ethyl)-3-(6-morpholino-1H-benzo[d]imidazol-2-yl)-1H- |
| | indazole-5-carboxamide |
| 63 | N-(3-(dimethylamino)propyl)-3-(6-morpholino-1H-benzo[d]imidazol-2-yl)- |
| | 1H-indazole-5-carboxamide |
| 64 | (3-(6-morpholino-1H-benzo[d]imidazol-2-yl)-1H-indazol-5-yl)(piperazin-1- |
| | yl)methanone |
| 65 | ((1R, 4R)-2,5-diazabicyclo[2.2.1]heptan-2-yl)(3-(6-morpholino-1H- |
| | benzo[d]imidazol-2-yl)-1H-indazol-5-yl)methanone |
| 66 | ((IS, 4S)-2,5-diazabicyclo[2.2.1]heptan-2-yl)(3-(6-morpholino-1H- |
| | benzo[d]imidazol-2-yl)-1H-indazol-5-yl)methanone |
| 67 | (1,4-diazepan-1-yl)(3-(6-morpholino-1H-benzo[d]imidazol-2-yl)-1H-indazol- |
| | 5-yl)methanone |
| 68 | (3-(aminomethyl)piperidin-1-yl)(3-(6-morpholino-1H-benzo[d]imidazol-2-yl)- |
| | 1H-indazol-5-yl)methanone |
| 69 | (4-aminopiperidin-1-yl)(3-(6-morpholino-1H-benzo[d]imidazol-2-yl)-1H- |
| | indazol-5-yl)methanone |
| 70 | (4-(methylamino)piperidin-1-yl)(3-(6-morpholino-1H-benzo[d]imidazol-2-yl)- |
| | 1H-indazol-5-yl)methanone |
| 71 | N-((1R, 5S, 6r)-3-azabicyclo[3.1.0]hexan-6-yl)-3-(6-morpholino-1H- |
| | benzo[d]imidazol-2-yl)-1H-indazole-5-carboxamide |
| 72 | N-(4-aminophenyl)-3-(6-morpholino-1H-benzo[d]imidazol-2-yl)-1H-indazole- |
| | 5-carboxamide |
| 73 | N-(3-aminopropyl)-3-(6-(furan-3-yl)-1H-benzo[d]imidazol-2-yl)-1H-indazole- |
| | 5-carboxamide |
| 74 | N-((1S, 3S)-3-aminocyclopentyl)-3-(6-(furan-3-yl)-1H-benzo[d]imidazol-2-yl)- |
| | 1H-indazole-5-carboxamide |
| 75 | N-(azetidin-3-ylmethyl)-3-(6-(furan-3-yl)-1H-benzo[d]imidazol-2-yl)-1H- |
| | indazole-5-carboxamide |
| 76 | N-(3-aminopropyl)-3-(6-phenyl-1H-benzo[d]imidazol-2-yl)-1H-indazole-5- |
| | carboxamide |
| 77 | N-(3-aminopropyl)-3-(6-(pyridin-4-yl)-1H-benzo[d]imidazol-2-yl)-1H- |
| | indazole-5-carboxamide |
| 78 | N-(3-aminopropyl)-3-(6-(4-(dimethylamino)phenyl)-1H-benzo[d]imidazol-2- |
| | yl)-1H-indazole-5-carboxamide |
| 79 | N-(3-aminopropyl)-3-(6-(4-methoxyphenyl)-1H-benzo[d]imidazol-2-yl)-1H- |
| | indazole-5-carboxamide |
| 80 | N-((1S,3S)-3-aminocyclopentyl)-3-(6-(furan-2-yl)-1H-benzo[d]imidazol-2-yl)- |
| | 1H-indazole-5-carboxamide |

TABLE 3

| Example | name |
|---|---|
| 104 | N-(3-(dimethylamino)propyl)-3-(6-fluoro-1-isopropyl-1H-benzo[d]imidazol-2- |
| | yl)-1H-indazole-5-carboxamide |
| 105 | N-(3-aminopropyl)-3-(6-fluoro-1-isopropyl-1H-benzo[d]imidazol-2-yl)-1H- |
| | indazole-5-carboxamide |
| 106 | N-(3-aminopropyl)-3-(6-fluoro-1-(2-methoxyethyl)-1H-benzo[d]imidazol-2- |
| | yl)-1H-indazole-5-carboxamide |
| 107 | N-(3-aminopropyl)-3-(1-(2-(dimethylamino)ethyl)-6-fluoro-1H- |
| | benzo[d]imidazol-2-yl)-1H-indazole-5-carboxamide |
| 108 | N-(3-aminopropyl)-3-(6-fluoro-1-(2-(4-methylpiperazin-1-yl)ethyl)-1H- |
| | benzo[d]imidazol-2-yl)-1H-indazole-5-carboxamide |
| 109 | N-(3-aminopropyl)-3-(6-fluoro-1-(2-hydroxyethyl)-1H-benzo[d]imidazol-2- |
| | yl)-1H-indazole-5-carboxamide |
| 110 | N-(3-aminopropyl)-3-(6-fluoro-1H-benzo[d]imidazol-2-yl)-1H-indazole-6- |
| | carboxamide |
| 111 | 3-(6-fluoro-1H-benzo[d]imidazol-2-yl)-N-(3-hydroxypropyl)-1H-indazole-5- |
| | carboxamide |
| 112 | N-(1-(2-aminoethyl)piperidin-4-yl)-3-(1H-benzo[d]imidazol-2-yl)-1H- |
| | indazole-5-carboxamide |

In one specific embodiment of the present invention, the present invention relates to a compound represented by the following formula 3 or a pharmaceutically acceptable salt thereof.

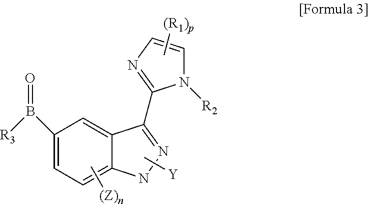

wherein in Formula 3,
R₁ is each independently selected from the group consisting of hydrogen, halogen, C₁₋₁₀ alkyl, C₁₋₁₀ haloalkyl, (3-10 membered) cycloalkyl, C₁₋₁₀ alkylene-(3-10 membered) cycloalkyl, (3-10 membered) heterocyclyl, C₁₋₁₀ alkylene-(3-10 membered) heterocyclyl, (6-14 membered) aryl, C₀₋₁₀ alkylene-(6-14 membered) aryl, (5-12 membered) heteroaryl, C₁₋₁₀ alkylene-(5-12 membered) heteroaryl, C₁₋₁₀ alkoxy, C₁₋₁₀ alkylene-C₁₋₆ alkoxy, 0-(6-14 membered) aryl, C₁₋₁₀ alkylene-O-(6-14 membered)aryl, O—C₁₋₁₀ alkylene-C₁₋₆ alkoxy, O—C₁₋₁₀ alkylene-NR₁ₐR_{1b}, C(O)—NR₁ₐR_{1b}, C₁₋₁₀ alkylene-C(O)—NR₁ₐR_{1b}, C₁₋₆ haloalkoxy, C₁₋₁₀ alkylene-C₁₋₆ haloalkoxy, CN, C₁₋₁₀ alkylene-CN, OH, NO₂, NR₁ₐR_{1b}, OC(O)—NR₁ₐR_{1b}, C(O)R₁ₐ, C(O)OR₁ₐ, NR₁ₐC(O)R_{1b}, NR₁ₐC(O)OR_{1b}, NR₁ₐSO₂R_{1b} and SO₂NR₁ₐR_{1b},
wherein (3-10 membered) cycloalkyl, (3-10 membered) heterocyclyl, (6-14 membered) aryl, and (5-12 membered) heteroaryl may each independently be unsubstituted or substituted with at least one of (3-10 membered) cycloalkyl, (3-10 membered) heterocycyl, C₁₋₆ alkyl, C₁₋₆ alkoxy, halogen, C₁₋₆ haloalkyl, NH₂, NO₂, OH, or NR₁ₐR_{1b},
wherein R₁ₐ and R_{1b} are each independently selected from hydrogen, C₁₋₆ alkyl or C₁₋₆ haloalkyl, or the R₁ₐ and R_{1b} may be bonded to the same nitrogen atom to form (3-10 membered) heterocyclyl or (5-12 membered) heteroaryl within the substituent,
p is an integer from 0 to 2,
R₂ is each independently selected from the group consisting of hydrogen, C₁₋₁₀ alkyl, halogen, C₁₋₆ haloalkyl, C₁₋₆ alkoxy, C₁₋₁₀ alkylene-C₁₋₆ alkoxy, C₁₋₆ haloalkoxy, CN, OH, C₁₋₁₀ alkylene-OH, NO₂, C₁₋₆ alkylamino, C₁₋₁₀ alkylene-NR₂ₐR_{2b}, (6-14 membered) aryl, (5-12 membered) heteroaryl, (3-10 membered) cycloalkyl, (3-10 membered) heterocyclyl, and C₁₋₁₀ alkylene-(3-10 membered) heterocyclyl,
wherein (6-14 membered) aryl, (5-12 membered) heteroaryl, (3-10 membered) cycloalkyl and (3-10 membered) heterocyclyl may each independently be unsubstituted or substituted with at least one of C₁₋₆ alkyl, halogen, C₁₋₆ haloalkyl, C₁₋₆ alkoxy or C₁₋₆ haloalkoxy,
the R₂ₐ and R_{2b} are each independently selected from hydrogen, C₁₋₆ alkyl or C₁₋₆ haloalkyl, or the R₂ₐ and R_{2b} may be bonded to the same nitrogen atom to form (3-10 membered) heterocyclyl or (5-12 membered) heteroaryl within the substituent,
B is carbon or sulfur,
R₃ is —NR₃ₐR_{3b},
wherein R₃ₐ and R_{3b} are each independently selected from the group consisting of hydrogen, OH, C₁₋₁₀ alkyl, C₁₋₆ haloalkyl, C(O)C₁₋₆ alkyl,

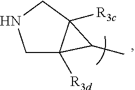

(3-10 membered) cycloalkyl, (6-14 membered) aryl, (5-12 membered) heteroaryl and (3-10 membered) heterocyclyl,
or the R₃ₐ and R_{3b} may be boned to the same nitrogen atom to form (3-10 membered) heterocyclyl or (5-12 membered) heteroaryl within the substituent,
wherein R_{3c} and R_{3d} are each independently selected from hydrogen, C₁₋₆ alkyl or C₁₋₆ haloalkyl,
wherein C₁₋₁₀ alkyl, (3-10 membered) cycloalkyl, (3-10 membered) heterocyclyl, (6-14 membered) aryl, and (5-12 membered) heteroaryl may each independently be unsubstituted or substituted with R₄,
R₄ is selected from the group consisting of OH, C₁₋₆ alkyl, C₁₋₆ alkoxy, halogen, C₁₋₆ haloalkyl, C(O)C₁₋₆ alkyl, (6-14 membered) aryl, (3-10 membered) cycloalkyl, (3-10 membered) heterocyclyl, (5-12 membered) heteroaryl and NR₄ₐR_{4b},
wherein R₄ₐ and R_{4b} are each independently selected from hydrogen, C₁₋₆ alkyl, C₁₋₆ haloalkyl, C(O)C₁₋₆ alkyl, SO₂(C₁₋₆)alkyl, or C(O)C₁₋₆ haloalkyl, or the R₄ₐ and R_{4b} may be boned to the same nitrogen atom to form (3-10 membered) heterocyclyl or (5-12 membered) heteroaryl within the substituent,
wherein (6-14 membered)aryl, (3-10 membered)cycloalkyl, and (3-10 membered)heterocyclyl may each independently be unsubstituted or substituted with at least one of C₁₋₆ alkyl, amino, halogen, C₁₋₆ haloalkyl, C(O)C₁₋₆ alkyl, C₁₋₆ alkoxy, or C₁₋₆ haloalkoxy,
Y is selected from the group consisting of hydrogen, C₁₋₆ alkyl, C₁₋₆ haloalkyl, C₁₋₆ alkoxy, C(O)C₁₋₆ alkyl, C(O)OC₁₋₆ alkyl and C(O)N—C₁₋₆ alkyl,
Z is selected from the group consisting of hydrogen, C₁₋₆ alkyl, halogen, C₁₋₆ haloalkyl, C₁₋₆ alkoxy, NH₂, OH, CN, COOH, C(O)C₁₋₆ alkyl, C(O)OC₁₋₆ alkyl and C(O)N—C₁₋₆ alkyl,
wherein heterocyclyl and heteroaryl each independently comprise at least one of N, O, or S in the ring,
n may be an integer from 0 to 3.

In one specific embodiment of the present invention, in Formula 3, R₁ is each independently selected from the group consisting of hydrogen, (6-14 membered) aryl, C₁₋₁₀ alkylene-C₁₋₆ alkoxy, C₁₋₁₀ alkylene-O-(6-14 membered) aryl, and C(O)—NR₁ₐR_{1b},
wherein (6-14 membered) aryl may each independently be unsubstituted or substituted with at least one of (3-10 membered) cycloalkyl, (3-10 membered) heterocyclyl, C₁₋₆ alkyl, C₁₋₆ alkoxy, halogen, C₁₋₆ haloalkyl, NH₂, NO₂, OH, or NR₁ₐR_{1b},
wherein R₁ₐ and R_{1b} are each independently hydrogen or C₁₋₆ alkyl, or the R₁ₐ and R_{1b} may be bonded to the same nitrogen atom to form (3-10 membered) heterocyclyl within the substituent,
p is an integer from 0 to 2,
R₂ is each independently hydrogen or C₁₋₁₀ alkyl,
B is carbon,
R₃ is —NR₃ₐR_{3b},
wherein R₃ₐ and R_{3b} are each independently hydrogen or C₁₋₁₀ alkyl,
or the R₃ₐ and R_{3b} may be boned to the same nitrogen atom to form (3-10 membered) heterocyclyl within the substituent,
wherein C₁₋₁₀ alkyl and (3-10 membered) heterocyclyl may each independently be unsubstituted or substituted with R₄,
R₄ is selected from the group consisting of C₁₋₆ alkyl, (6-14 membered) aryl, (3-10 membered) heterocyclyl, and NR₄ₐR_{4b},
wherein R₄ₐ and R_{4b} are each independently selected from hydrogen, C₁₋₆ alkyl, or SO₂(C₁₋₆)alkyl, or the R₄ₐ and R_{4b} may be boned to the same nitrogen atom to form (3-10 membered) heterocyclyl within the substituent,
wherein (6-14 membered)aryl, and (3-10 membered)heterocyclyl may each independently be unsubstituted or substituted with at least one of C₁₋₆ alkyl, amino, halogen, or C₁₋₆ haloalkyl,
Y is hydrogen, and n may be 0.

Representative compounds of Formula 3 include, but are not limited to, compounds selected from the group consisting of Examples 81 to 103 and Examples 113 to 115 in Table 4 below.

TABLE 4

| Example | name |
|---|---|
| 81 | N-(3-aminopropyl)-3-(5-(4-fluorophenyl)-1H-imidazol-2-yl)-1H-indazole-5- |
| | carboxamide |
| 82 | N-(3-aminopropyl)-3-(5-(3-fluorophenyl)-1H-imidazol-2-yl)-1H-indazole-5- |
| | carboxamide |
| 83 | N-(3-aminopropyl)-3-(5-(2-fluorophenyl)-1H-imidazol-2-yl)-1H-indazole-5- |
| | carboxamide |
| 84 | N-(3-aminopropyl)-3-(5-(3,4-difluorophenyl)-1H-imidazol-2-yl)-1H-indazole- |
| | 5-carboxamide |
| 85 | N-(3-aminopropyl)-3-(5-(4-chloro-3-fluorophenyl)-1H-imidazol-2-yl)-1H- |
| | indazole-5-carboxamide |
| 86 | N-(3-aminopropyl)-3-(5-(4-(trifluoromethyl)phenyl)-1H-imidazol-2-yl)-1H- |
| | indazole-5-carboxamide |
| 87 | N-(3-aminopropyl)-3-(5-(3,5-dichloro-2-methoxyphenyl)-1H-imidazol-2-yl)- |
| | 1H-indazole-5-carboxamide |
| 88 | N-(3-aminopropyl)-3-(5-(3-methoxyphenyl)-1H-imidazol-2-yl)-1H-indazole- |
| | 5-carboxamide |
| 89 | N-(3-aminopropyl)-3-(5-(3-nitrophenyl)-1H-imidazol-2-yl)-1H-indazole-5- |
| | carboxamide |
| 90 | N-(3-aminopropyl)-3-(5-(4-morpholinophenyl)-1H-imidazol-2-yl)-1H- |
| | indazole-5-carboxamide |
| 91 | N-(3-aminopropyl)-3-(5-(3-morpholinophenyl)-1H-imidazol-2-yl)-1H- |
| | indazole-5-carboxamide |
| 92 | N-(3-aminopropyl)-3-(5-(3-hydroxyphenyl)-1H-imidazol-2-yl)-1H-indazole-5- |
| | carboxamide |
| 93 | 3-(5-(3-aminophenyl)-1H-imidazol-2-yl)-N-(3-aminopropyl)-1H-indazole-5- |
| | carboxamide |
| 94 | N-(2-(methylsulfonamido)ethyl)-3-(4-phenyl-1H-imidazol-2-yl)-1H-indazole- |
| | 5-carboxamide |
| 95 | N-(2-(dimethylamino)ethyl)-3-(4-phenyl-1H-imidazol-2-yl)-1H-indazole-5- |
| | carboxamide |
| 96 | (3-(4-phenyl-1H-imidazol-2-yl)-1H-indazol-5-yl)(4-phenylpiperazin-1- |
| | yl)methanone |
| 97 | (4-(4-fluorophenyl)piperazin-1-yl)(3-(4-phenyl-1H-imidazol-2-yl)-1H- |
| | indazol-5-yl)methanone |
| 98 | (3-(4-phenyl-1H-imidazol-2-yl)-1H-indazol-5-yl)(4-(3- |
| | (trifluoromethyl)phenyl)piperazin-1-yl)methanone |
| 99 | (4-methylpiperazin-1-yl)(3-(4-phenyl-1H-imidazol-2-yl)-1H-indazol-5- |
| | yl)methanone |
| 100 | (4-(2-fluorophenyl)piperazin-1-yl)(3-(4-phenyl-1H-imidazol-2-yl)-1H- |
| | indazol-5-yl)methanone |
| 101 | (4-(3-chlorophenyl)piperazin-1-yl)(3-(4-phenyl-1H-imidazol-2-yl)-1H- |
| | indazol-5-yl)methanone |
| 102 | (4-(2-chlorophenyl)piperazin-1-yl)(3-(4-phenyl-1H-imidazol-2-yl)-1H- |
| | indazol-5-yl)methanone |
| 103 | N-(2-(4-aminopiperidin-1-yl)ethyl)-3-(4-phenyl-1H-imidazol-2-yl)-1H- |
| | indazole-5-carboxamide |
| 113 | N-(3-aminopropyl)-3-(4-carbamoyl-5-phenyl1-1H-imidazol-2-yl)-1H-indazole- |
| | 5-carboxamide |
| 114 | N-(3-aminopropyl)-3-(5-(methoxymethyl)-1H-imidazol-2-yl)-1H-indazole-5- |
| | carboxamide |
| 115 | N-(3-aminopropyl)-3-(5-(phenoxymethyl)-1H-imidazol-2-yl)-1H-indazole-5- |
| | carboxamide |

In one specific embodiment of the present invention, the present invention relates to a pharmaceutical composition for the prevention or treatment of cancer diseases, comprising the imidazole derivative compound of the present invention or a pharmaceutically acceptable salt thereof.

In one specific embodiment of the present invention, the present invention relates to a pharmaceutical composition for the prevention or treatment of cancer diseases, which prevents or treats cancer by inhibiting the activity of protein kinase.

In one specific embodiment of the present invention, the present invention relates to a pharmaceutical composition for the prevention or treatment of cancer diseases, wherein protein kinase is at least one selected from the group consisting of ABL1(E255K)-phosphorylated, ABL1(F3171)-phosphorylated, ABL1(F317L)-phosphorylated, ABL1(H396P)-nonphosphorylated, ABL1(H396P)-phosphorylated, ABL1(M351T)-phosphorylated, ABL1(Q252H)-nonphosphorylated, ABL1(Q252H)-phosphorylated, ABL1(T3151)-nonphosphorylated, ABL1(T3151)-phosphorylated, ABL1(Y253F)-phosphorylated, ABL1-nonphosphorylated, ABL1-phosphorylated, ARK5, AURKA, AURKB, AURKC, AXL, CDK2, CDK3, CDK4-cycllinDl, CDK4-cyclinD3, CDK5, CDK7, DYRK₁ₐ, DYRK_{1b}, DYRK2, ERK1, ERK2, ERK3, ERK4, ERK8, FLT3, FLT3(D835H), FLT3(D835V), FLT3(D835Y), FLT3(ITD), FLT3(ITD, D835V), FLT3(ITD, F691L), FLT3(K663Q), FLT3(N841I), FLT3(R834Q), FLT3-autoinhibited, GSK3A, GSK3B, HASPIN, HIPK1, HIPK2, HIPK3, HIPK4, IRAK1, IRAK3, IRAK4, JAK1(JHldomain-catalytic), JAK1(JH2domain-pseudokinase), JAK2(JH1domain-catalytic), JAK3(JH1domain-catalytic), KIT, KIT(A829P), KIT(D816H), KIT(D816V), KIT(L576P), KIT(V559D), KIT(V559D, T670I), KIT(V559D, V654A), MAP3K1, MAP3K15, MAP3K2, MAP3K3, MEKI, MEK2, MEK3, MEK4, MEK5, MEK6, PAKI, PAK2, PAK3, PAK4, PAK4, PAK5, PAK6, PAK7, PIM1, PIM2, PIM3, RET, RET(M918T), RET(V804L), RET(V804M), ROCKI, ROCK2, SRC, TRKA, TRKB, TRKC and YES1.

In one specific embodiment of the present invention, the present invention relates to a pharmaceutical composition for the prevention or treatment of cancer diseases, wherein protein kinase is at least one selected from the group consisting of ARK5, FLT3, YES1, ABL1, KIT and RET.

In one specific embodiment of the present invention, the present invention relates to a pharmaceutical composition for the prevention or treatment of cancer diseases, wherein protein kinase is an FMS-like tyrosine kinase 3 (FLT3).

In one specific embodiment of the present invention, the present invention relates to a pharmaceutical composition for the prevention or treatment of cancer diseases. The cancer diseases comprise multiple myeloma, malignant plasma cell neoplasm, Hodgkin lymphoma, nodular lymphocyte predominant Hodgkin lymphoma, Culler disease and myelomatosis, plasma cell leukemia, plasmacytoma, B-cell prolymphocytic leukemia, hairy cell leukemia, B-cell non-Hodgkin lymphoma (NHL), acute myeloid leukemia (AML), chronic lymphocytic leukemia (CLL), acute lymphoblastic leukemia (ALL), chronic myeloid leukemia (CML), follicular lymphoma, Burkitt lymphoma, marginal zone lymphoma, mantle cell lymphoma, large cell lymphoma, precursor B-lymphoblastic lymphoma, myeloid leukemia, Waldenstrom's macroglobulinemia, diffuse large B-cell lymphoma, follicular lymphoma, marginal zone lymphoma, mucosa-associated lymphoid tissue lymphoma, small lymphocytic lymphoma, mantle cell lymphoma, Burkitt lymphoma, primary mediastinal (thymic) large B-cell lymphoma, lymphoplasmacytic lymphoma, Waldenstrom macroglobulinemia, lymph node marginal zone B-cell lymphoma, splenic marginal zone lymphoma, intravascular large B-cell lymphoma, primary effusion lymphoma, lymphomatoid granulomatosis, T-cell/histiocyte-rich large B-cell lymphoma, primary central nervous system lymphoma, primary cutaneous diffuse large B-cell lymphoma (leg type), EBV-positive diffuse large B-cell lymphoma of the elderly, inflammation-associated diffuse large B-cell lymphoma, intravascular large B-cell lymphoma, ALK-positive large B-cell lymphoma, plasmablastic lymphoma, HHV8-associated multicentric Castleman disease-associated large B-cell lymphoma, unclassified B-cell lymphoma with intermediate features between diffuse large B-cell lymphoma and Burkitt lymphoma, or unclassified B-cell lymphoma with intermediate features between diffuse large B-cell lymphoma and classical Hodgkin lymphoma.

In one specific embodiment of the present invention, the present invention relates to a pharmaceutical composition for the prevention or treatment of cancer diseases, wherein cancer diseases is leukemia.

In one specific embodiment of the present invention, the present invention relates to a pharmaceutical composition for the prevention or treatment of cancer diseases, wherein leukemia is acute myeloid leukemia (AML).

In one specific embodiment of the present invention, the present invention relates to a pharmaceutical composition for the prevention or treatment of cancerous diseases, wherein acute myeloid leukemia (AML) expresses mutant FLT3. In one specific embodiment of the present invention, the present invention relates to a pharmaceutical composition for the prevention or treatment of Fms-like tyrosine kinase 3 (FLT3)-associated diseases, comprising the imidazole derivative compound of the present invention or a pharmaceutically acceptable salt thereof.

### Therapeutic Use

The novel imidazole derivative compound according to the present invention or a pharmaceutically acceptable salt thereof, inhibit the activity of protein kinases and exhibit preventive or therapeutic effects against cancer related to these activities.

In addition, the novel imidazole derivative compound according to the present invention or a pharmaceutically acceptable salt thereof, inhibit the activity of FLT3, particularly mutant FLT3, and exhibit preventive or therapeutic effects against diseases related to these activities.

The mutant FLT3 may have a mutation in the tyrosine kinase domain (TKD) of the FLT3 amino acid sequence (FLT3-TKD). The mutant FLT3 may further comprise an internal tandem duplication (ITD) within the gene. Examples of the mutant FLT3 comprise, but are not limited to, one or more of FLT3-ITD, FLT3-D835Y, FLT3-F691L, FLT3-F691L/D835Y, FLT3-ITD/D835Y, or FLT3-ITD/F691L.

In one embodiment, the compound of the present invention may be used to treat FLT3-associated diseases. In some embodiments, the FLT3-associated disease comprises malignant cells expressing FLT3, for example, cancer. In a specific embodiment, the cancer is of hematopoietic origin, such as lymphoma or leukemia. In a more specific embodiment, the cancer includes, but is not limited thereto, multiple myeloma, malignant plasma cell neoplasm, Hodgkin lymphoma, nodular lymphocyte predominant Hodgkin lymphoma, Culler disease and myelomatosis, plasma cell leukemia, plasmacytoma, B-cell prolymphocytic leukemia, hairy cell leukemia, B-cell non-Hodgkin lymphoma (NHL), acute myeloid leukemia (AML), chronic lymphocytic leukemia (CLL), acute lymphoblastic leukemia (ALL), chronic myeloid leukemia (CML), follicular lymphoma, Burkitt lymphoma, marginal zone lymphoma, mantle cell lymphoma, large cell lymphoma, precursor B-lymphoblastic lymphoma, myeloid leukemia, Waldenstrom's macroglobulinemia, diffuse large B-cell lymphoma, follicular lymphoma, marginal zone lymphoma, mucosa-associated lymphoid tissue lymphoma, small lymphocytic lymphoma, mantle cell lymphoma, Burkitt lymphoma, primary mediastinal (thymic) large B-cell lymphoma, lymphoplasmacytic lymphoma, Waldenstrom macroglobulinemia, lymph node marginal zone B-cell lymphoma, splenic marginal zone lymphoma, intravascular large B-cell lymphoma, primary effusion lymphoma, lymphomatoid granulomatosis, T-cell/histiocyte-rich large B-cell lymphoma, primary central nervous system lymphoma, primary cutaneous diffuse large B-cell lymphoma (leg type), EBV-positive diffuse large B-cell lymphoma of the elderly, inflammation-associated diffuse large B-cell lymphoma, intravascular large B-cell lymphoma, ALK-positive large B-cell lymphoma, plasmablastic lymphoma, HHV8-associated multicentric Castleman disease-associated large B-cell lymphoma, unclassified B-cell lymphoma with intermediate features between diffuse large B-cell lymphoma and Burkitt lymphoma, unclassified B-cell lymphoma with intermediate features between diffuse large B-cell lymphoma and classical Hodgkin lymphoma, or other hematopoietic cell-associated cancers. Preferably, the cancer is acute myeloid leukemia (AML), more preferably, AML expressing mutant FLT3.

As used herein, the term “acute myeloid leukemia (AML)” is a cancer of the myeloid blood cell lineage characterized by the rapid proliferation of abnormal blood cells derived from the bone marrow, which interferes with normal blood cells. The main symptoms of AML include fatigue, shortness of breath, easy bruising or bleeding, and frequent infections. Although several putative causes of AML have been identified, the definitive cause of AML remains unknown. The compounds of the present invention, particularly those possessing mutant FLT3 inhibitory activity, have demonstrated potent antiproliferative activity in FLT3-ITD-expressing MV4-11 cell lines, FLT3/F691L-expressing BaF3-ITD cell lines, and FLT/D835Y-expressing BaF3-ITD cell lines, confirming their potential as AML therapeutics.

Accordingly, the present invention provides a pharmaceutical composition for inhibiting FLT3 kinase, comprising a compound according to the present invention or a pharmaceutically acceptable salt thereof as an active ingredient. In one specific embodiment, the pharmaceutical composition is intended for the prevention or treatment of FLT3-associated diseases, more specifically, for the prevention or treatment of AML.

The present invention also provides a method for preventing or treating FLT3-associated diseases, comprising administering a compound according to the present invention or a pharmaceutically acceptable salt thereof, or the pharmaceutical composition to a subject in need thereof.

The present invention also provides a method of inhibiting FLT3, comprising administering to a subject diagnosed with AML or at risk of developing AML a compound according to the present invention or a pharmaceutically acceptable salt thereof, or the pharmaceutical composition thereof, in a pharmaceutically effective amount.

The present invention further provides a method of inhibiting FLT3, the pharmaceutical composition exhibits potent antiproliferative activity in FLT3-ITD-expressing MV4-11 cell lines, FLT3/F691L-expressing BaF3-ITD cell lines, and FLT/D835Y-expressing BaF3-ITD cell lines.

Furthermore, the present invention provides the use of compounds according to the present invention or pharmaceutically acceptable salts thereof for the inhibition of FLT3, or for the prevention or treatment of FLT3-associated diseases. The present invention also provides the use of compounds according to the present invention or pharmaceutically acceptable salts thereof for the manufacture of a medicament for the prevention or treatment of AML.

Hereinafter, the present invention will be described in more detail through examples. These examples are only intended to more specifically explain the present invention, and it will be apparent to those skilled in the art that the scope of the present invention is not limited by these examples according to the gist of the present invention.

### General Synthesis Procedure

Compound according to the present invention may be prepared from readily available starting materials using modifications to the specific synthetic protocols well known to those skilled in the art as described below.

### General Synthesis Procedure A

The corresponding acid was dissolved in DMF, and 1-(3-dimethylaminopropyl)-3-ethylcarbodiimide hydrochloride (2.0 eq), 1-hydroxybenzotriazole (2.0 eq), and triethylamine (3.0 eq) were added, and then the corresponding amine was added, and the solution was stirred at room temperature for 12 hours. Upon completion of the reaction, the solution was extracted with water and ethyl acetate to obtain the organic layer, which was dried over sodium sulfate (Na₂SO₄) and filtered. The solvent was removed under reduced pressure, and the residue was purified by column chromatography to afford the product.

### General Synthesis Procedure B

The starting material obtained from General Synthesis Procedure A was dissolved in 10% trifluoroacetic acid in DCM and the solution was stirred at room temperature overnight. Upon completion of the reaction, the solvent was removed under reduced pressure, and the residue was extracted with saturated aqueous sodium bicarbonate (NaHCO₃) solution and ethyl acetate to obtain the organic layer. The organic layer was dried over sodium sulfate (Na₂SO₄) and filtered. Thereafter, the solvent was removed under reduced pressure, and the residue was purified by column chromatography to afford the product.

### General Synthesis Procedure C

Methyl 3-formyl-1H-indazole-5-carboxylate was dissolved in methanol (MeOH), and substituted benzene-1,2-diamine (1.5 eq) was added. The solution was stirred under reflux for 12 hours, after which the solvent was removed completely under reduced pressure. The residue was extracted with water and ethyl acetate, and the organic layer was dried over sodium sulfate (Na₂SO₄) and filtered. Thereafter, the solvent was removed under reduced pressure, and the residue was purified by column chromatography to afford the product.

### General Synthesis Procedure D

The ester derivative obtained from General Synthesis Procedure C was dissolved in methanol, and an equal volume of 5 N aqueous NaOH solution was added. The solution was stirred at room temperature for 12 hours. Upon completion of the reaction, methanol was completely removed, and 1 N aqueous HCl solution was added dropwise to neutralize the solution. The resulting solid was collected by filtration to afford the product.

### General Synthesis Procedure E

Methyl 3-formyl-1H-indazole-5-carboxylate was dissolved in methanol, and substituted benzene-1,2-diamine (1.5 eq) was added. The solution was stirred under reflux for 12 hours, and upon confirming completion of the reaction, 5 N aqueous NaOH solution (10.0 eq) was added. The solution was stirred at room temperature for an additional 12 hours. Upon completion of the reaction, methanol was completely removed, and 1 N aqueous HCl solution was added dropwise to neutralize the solution. The resulting solid was collected by filtration to afford the product.

### General Synthesis Procedure F

The starting material was dissolved in DMF and cooled to 0° C. Thereafter, TEA (1.1 eq) and acetyl chloride (1.1 eq) were added, and the solution was stirred for 1 hour. Upon completion of the reaction, the solution was extracted with water and ethyl acetate, and the organic layer was dried over sodium sulfate (Na₂SO₄) and filtered. The solvent was removed under reduced pressure, and the residue was purified by column chromatography to afford the product.

### General Synthesis Procedure G

The starting material was dissolved in toluene/H₂O (3:1), and boronic acid (1.5 eq) and potassium carbonate (K₂CO₃) (2.3 eq) were added. After degassing, Pd(PPh₃)₄(0.07 eq) was added, and the solution was heated at 60° C. for 3 hours. Upon completion of the reaction, the solution was extracted with water and ethyl acetate, and the organic layer was dried over sodium sulfate (Na₂SO₄) and filtered. Thereafter, the solvent was removed under reduced pressure, and the residue was purified by column chromatography to afford the product.

### General Synthesis Procedure H

The derivative obtained from General Synthesis Procedure G was dissolved in conc HCl(aq)/Methanol (1:3) and the solution was stirred at room temperature for 12 hours. Upon completion of the reaction, the solvent was completely removed under reduced pressure, and the residue was dissolved in saturated aqueous sodium bicarbonate (NaHCO₃) solution and extracted with ethyl acetate. The organic layer was dried over sodium sulfate (Na₂SO₄) and filtered. Thereafter, the solvent was removed under reduced pressure, and the residue was purified by column chromatography to afford the product.

### General Synthesis Procedure I

Methyl 3-formyl-TH-indazole-5-carboxylate was dissolved in methanol, and 2-oxo-2-substituted phenylacetaldehyde (1.0 eq) and ammonium acetate (5.0 eq) were added, and the solution was stirred at room temperature for 1 hour. Upon completion of the reaction, the solvent was completely removed under reduced pressure, and the residue was extracted with water and ethyl acetate. Thereafter, the organic layer was dried over sodium sulfate (Na₂SO₄) and filtered. Thereafter, the solvent was removed under reduced pressure, and the residue was purified by column chromatography to afford the product.

### EXAMPLES

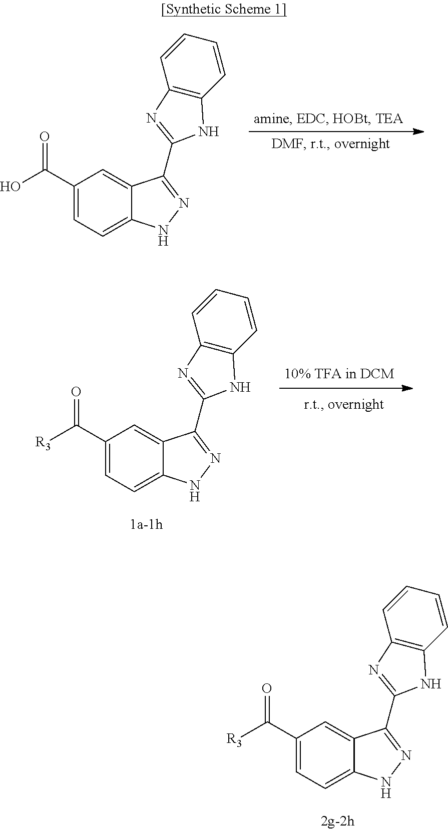

### Example 1: (3-(1H-benzo[d]imidazol-2-yl)-1H-indazol-5-yl)(4-phenylpiperazin-1-yl)methanone (la)

Compound (1a) of Example 1 was obtained by using N-phenylpiperazine according to General Synthesis Procedure A (yellow oil, yield: 45%).

¹H NMR (400 MHz, DMSO-d₆) δ 8.63-8.65 (m, 1H), 7.71-7.74 (m, 1H), 7.49-7.58 (m, 2H), 7.15-7.27 (m, 5H), 6.95-7.00 (m, 2H), 6.78-6.84 (m, 1H), 3.56-3.86 (m, 3H), 3.15-3.27 (m, 4H). MS (ESI): [M+H]⁺=422.95

### Example 2: (3-(1H-benzo[d]imidazol-2-yl)-1H-indazol-5-yl)(4-methylpiperazin-1-yl)methanone (1b)

Compound (1b) of Example 2 was obtained by using N-methylpiperazine according to General Synthesis Procedure A (yellow oil, yield: 33%).

¹H NMR (400 MHz, METHANOL-d₄) δ 8.60 (s, 1H), 7.63-7.84 (m, 2H), 7.51-7.63 (m, 2H), 7.26-7.32 (m, 2H), 3.76-3.95 (m, 2H), 3.53-3.76 (m, 2H), 2.42-2.67 (m, 4H), 2.36 (s, 3H). MS (ESI): [M−H]⁺=359.99

### Example 3: (3-(1H-benzo[d]imidazol-2-yl)-1H-indazol-5-yl)(4-(4-fluorophenyl)piperazin-1-yl)methanone (1c)

Compound (1c) of Example 3 was obtained by using N-(4-fluorophenyl)piperazine according to General Synthesis Procedure A (yellow oil, yield: 84%).

¹H NMR (400 MHz, METHANOL-d₄) δ 8.62-8.65 (m, 1H), 7.71-7.75 (m, 1H), 7.62-7.70 (m, 2H), 7.58 (dd, J=1.60, 8.70 Hz, 1H), 7.26-7.31 (m, 2H), 6.96-7.05 (m, 4H), 3.73-4.02 (m, 4H), 3.06-3.29 (m, 4H). MS (ESI): [M+H]⁺=441.01

### Example 4: (3-(1H-benzo[d]imidazol-2-yl)-1H-indazol-5-yl)(4-(2-fluorophenyl)piperazin-1-yl)methanone (1d)

Compound (1d) of Example 4 was obtained by using N-(2-fluorophenyl)piperazine according to General Synthesis Procedure A (yellow oil, yield: 80%).

¹H NMR (400 MHz, DMSO-d₆) δ 8.63-8.65 (m, 1H), 7.69-7.78 (m, 2H), 7.48-7.58 (m, 2H), 7.05-7.27 (m, 5H), 6.97-7.03 (m, 1H), 3.60-3.89 (m, 4H), 2.94-3.15 (m, 4H). MS (ESI): [M+H]⁺=440.94

### Example 5: (3-(1H-benzo[d]imidazol-2-yl)-1H-indazol-5-yl)(4-(3-chlorophenyl)piperazin-1-yl)methanone (1e)

Compound (1e) of Example 5 was obtained by using N-(3-chlorophenyl)piperazine according to General Synthesis Procedure A (yellow oil, yield: 20%).

¹H NMR (400 MHz, METHANOL-d₄) δ 8.64-8.67 (m, 1H), 7.64-7.77 (m, 3H), 7.57-7.64 (m, 1H), 7.36-7.41 (m, 1H), 7.26-7.32 (m, 3H), 7.17-7.22 (m, 1H), 7.01-7.08 (m, 1H), 3.00-3.25 (m, 4H). MS (ESI): [M+H]⁺=456.97

### Example 6: (3-(1H-benzo[d]imidazol-2-yl)-1H-indazol-5-yl)(4-(2-chlorophenyl)piperazin-1-yl)methanone (1f)

Compound (1f) of Example 6 was obtained by using N-(2-chlorophenyl)piperazine according to General Synthesis Procedure A (yellow oil, yield: 25%).

¹H NMR (400 MHz, METHANOL-d₄) δ 8.64 (t, J=1.14 Hz, 1H), 7.59-7.76 (m, 4H), 7.27-7.31 (m, 2H), 7.17-7.23 (m, 1H), 6.99-7.02 (m, 1H), 6.91-6.96 (m, 1H), 6.81-6.86 (m, 1H), 3.75-3.98 (m, 4H), 3.18-3.29 (m, 4H). MS (ESI): [M+H]⁺=456.97

### tert-butyl(3-(3-(1H-benzo[d]imidazol-2-yl)-1H-indazole-5-carboxamido)propyl)carbamate (1g)

Compound Ig was obtained by using N-Boc-propane-1,3-diamine according to General Synthesis Procedure A (yellow oil, yield: 35%).

MS (ESI): [M+H]⁺=435.1

### tert-butyl (1-(2-(3-(1H-benzo[d]imidazol-2-yl)-1H-indazole-5-carboxamido)ethyl)piperidin-4-yl)carbamate (1h)

Compound 1 h was obtained by using 1-(2-aminoethyl)-N-Boc-piperidine-4-amine according to General Synthesis Procedure A (yellow oil, yield: 83%).

¹H NMR (400 MHz, METHANOL-d₄) δ 9.02 (dd, J=0.92, 1.60 Hz, 1H), 7.94-8.00 (m, 1H), 7.69-7.76 (m, 1H), 7.66-7.69 (m, 1H), 7.58-7.63 (m, 1H), 7.27-7.33 (m, 2H), 4.23-4.35 (m, 1H), 3.55-3.68 (m, 2H), 2.99-3.09 (m, 2H), 2.65-2.75 (m, 2H), 2.20-2.30 (m, 2H), 1.84-1.95 (m, 2H), 1.49-1.58 (m, 2H), 1.43 (s, 9H). MS (ESI): [M+H]⁺=504.04

### Example 7: N-(3-aminopropyl)-3-(1H-benzo[d]imidazol-2-yl)-1H-indazole-5-carboxamide (2g)

Compound (2g) of Example 7 was obtained from compound lg according to General Synthesis Procedure B (colorless oil, yield: 89%).

¹H NMR (400 MHz, METHANOL-d₄) δ 9.02 (dd, J=0.92, 1.60 Hz, 1H), 7.96 (dd, J=1.60, 8.93 Hz, 1H), 7.65-7.72 (m, 3H), 7.27-7.34 (m, 2H), 3.55 (t, J=6.75 Hz, 2H), 2.83 (t, J=7.10 Hz, 2H), 1.79-1.92 (m, 2H). MS (ESI): [M+H]⁺=335.2

### Example 8: N-(2-(4-aminopiperidin-1-yl)ethyl)-3-(1H-benzo[d]imidazol-2-yl)-1H-indazole-5-carboxamide (2h)

Compound (2h) of Example 8 was obtained from compound 1h according to General Synthesis Procedure B (white solid, yield: 90%).

¹H NMR (400 MHz, METHANOL-d₄) δ 9.01 (dd, J=0.80, 1.72 Hz, 1H), 7.95 (dd, J=1.72, 8.82 Hz, 1H), 7.65-7.72 (m, 3H), 7.26-7.33 (m, 2H), 3.63 (t, J=6.98 Hz, 2H), 3.04 (d, J=12.14 Hz, 2H), 2.60-2.74 (m, 3H), 2.10-2.23 (m, 2H), 1.77-1.94 (m, 2H), 1.41-1.56 (m, 2H). MS (ESI): [M+H]⁺=404.20

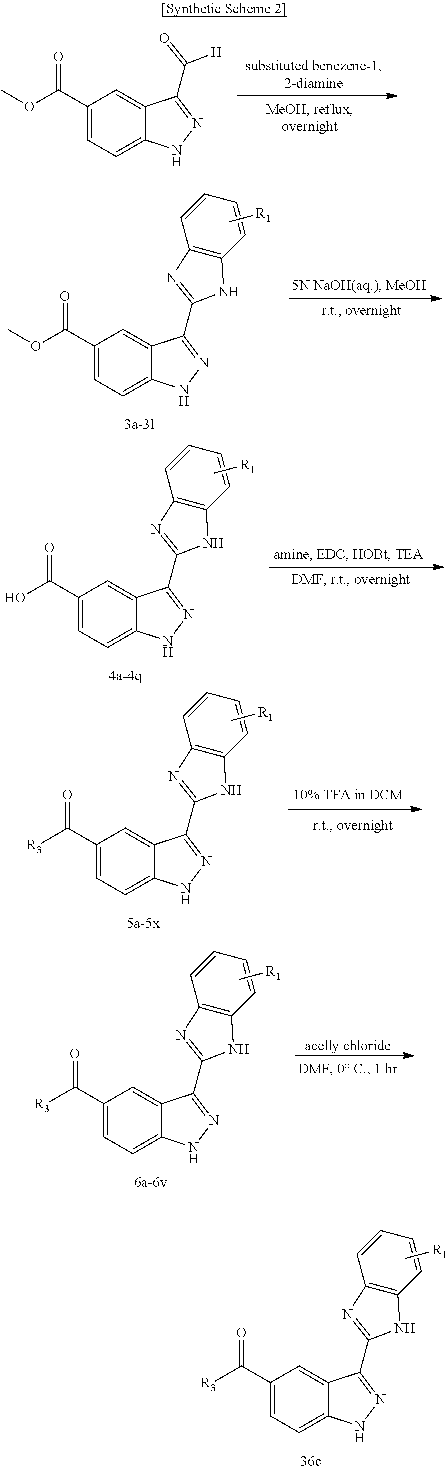

### methyl 3-(6-methyl-1H-benzo[d]imidazol-2-yl)-1H-indazole-5-carboxylate (3a)

Compound 3a was obtained by using 6-methyl benzene-1,2-diamine according to General Synthesis Procedure C (yellow solid, yield: 33%).

¹H NMR (400 MHz, DMSO-d₆) δ 12.83-13.12 (m, 1H), 9.21 (dd, J=0.69, 1.60 Hz, 1H), 8.03 (dd, J=1.60, 8.70 Hz, 1H), 7.74 (dd, J=0.80, 8.82 Hz, 1H), 7.56-7.71 (m, 1H), 7.27-7.46 (m, 1H), 7.01-7.11 (m, 1H), 3.91-3.97 (m, 3H), 2.45 (s, 3H). MS (ESI): [M+H]⁺=307.4

### methyl 3-(6-(pyrrolidin-1-yl)-1H-benzo[d]imidazol-2-yl)-1H-indazole-5-carboxylate (3b)

Compound 3b was obtained by using 6-pyrrolidin-1-yl benzene-1,2-diamine according to General Synthesis Procedure C (yellow solid, yield: 18%).

¹H NMR (400 MHz, METHANOL-d₄) δ 9.22 (s, 1H), 8.10 (dd, J=1.49, 8.82 Hz, 1H), 7.65 (dd, J=0.69, 8.70 Hz, 1H), 7.57 (d, J=9.16 Hz, 1H), 6.72-6.76 (m, 2H), 3.98 (s, 3H), 3.36-3.39 (m, 4H), 2.08 (td, J=3.35, 6.35 Hz, 4H). MS (ESI): [M+H]⁺=362.5

### methyl 3-(6-(piperidin-1-yl)-1H-benzo[d]imidazol-2-yl)-1H-indazole-5-carboxylate (3c)

Compound 3c was obtained by using 6-piperidin-1-yl benzene-1,2-diamine according to General Synthesis Procedure C (brown oil, yield: 39%).

Mixture of two tautomers. ¹H NMR (400 MHz, DMSO-d₆) δ 13.77-13.88 (m, 1H), 12.69-12.84 (m, 1H), 9.12-9.21 (m, 1H), 7.99 (dd, J=1.60, 8.71 Hz, 1H), 7.69 (d, J=8.94 Hz, 1H), 7.30-7.61 (m, 1H), 6.91-7.02 (m, 1H), 6.85-7.26 (m, 1H), 3.90 (s, 3H), 3.03-3.14 (m, 4H), 1.59-1.71 (m, 4H), 1.45-1.56 (m, 2H). MS (ESI): [M+H]⁺=376.3.

### methyl 3-(6-(4-methylpiperazin-1-yl)-1H-benzo[d]imidazol-2-yl)-1H-indazole-5-carboxylate (3d)

Compound 3d was obtained by using 6-(4-methylpiperazin-1-yl) benzene-1,2-diamine according to General Synthesis Procedure C (yellow solid, yield: 60%).

¹H NMR (400 MHz, CHLOROFORM-d) δ 11.89 (br. s., 1H), 10.53 (br. s., 1H), 9.35 (s, 1H), 8.07 (d, J=8.70 Hz, 1H), 7.73 (br. s., 1H), 7.43 (dd, J=0.57, 7.67 Hz, 1H), 6.98 (br. s., 1H), 6.84 (br. s., 1H), 3.89 (s, 3H), 3.20 (br. s., 4H), 2.61 (br. s., 4H), 2.38 (s, 3H). MS (ESI): [M+H]⁺=391.5.

### methyl 3-(6-((3S,5R)-3,5-dimethylpiperazin-1-yl)-1H-benzo[d]imidazol-2-yl)-1H-indazole-5-carboxylate (3e)

Compound 3e was obtained by using 6-((3S,5R)-dimethylpiperazin-1-yl) benzene-1,2-diamine according to General Synthesis Procedure C (brown solid, yield: 41%).

¹H NMR (400 MHz, METHANOL-d₄) δ9.22 (dd, J=0.80, 1.49 Hz, 1H), 8.09 (dd, J=1.60, 8.93 Hz, 1H), 7.64 (dd, J=0.92, 8.93 Hz, 1H), 7.57-7.68 (m, 1H), 7.19 (br. s, 1H), 7.10 (dd, J=2.29, 8.93 Hz, 1H), 3.97 (s, 3H), 3.58 (dd, J=1.83, 11.68 Hz, 2H), 3.07-3.17 (m, 2H), 2.39 (t, J=11.20 Hz, 2H), 1.21 (d, J=6.41 Hz, 6H). MS (ESI): [M+H]⁺=405.5.

### methyl 3-(6-morpholino-1H-benzo[d]imidazol-2-yl)-1H-indazole-5-carboxylate (3f)

Compound 3f was obtained by using 6-morpholin-4-yl benzene-1,2-diamine according to General Synthesis Procedure C (yellow solid, yield: 58%).

Mixture of two tautomers. ¹H NMR (400 MHz, DMSO-d₆) δ 13.85-13.93 (m, 1H), 12.81-12.92 (m, 1H), 9.18-9.23 (m, 1H), 8.01-8.05 (m, 1H), 7.71-7.76 (m, 1H), 7.37-7.68 (m, 1H), 6.98-7.07 (m, 1H), 6.92-7.32 (m, 1H), 3.91-3.96 (m, 3H), 3.75-3.82 (m, 4H), 3.09-3.15 (m, 4H). MS (ESI): [M+H]⁺=378.7.

### methyl 3-(6-(morpholine-4-carbonyl)-1H-benzo[d]imidazol-2-yl)-1H-indazole-5-carboxylate (3g)

Compound 3g was obtained by using 6-morpholinomethanoyl benzene-1,2-diamine according to General Synthesis Procedure C (white solid, yield: 37%).

¹H NMR (400 MHz, METHANOL-d₄) δ 9.25 (dd, J=0.80, 1.49 Hz, 1H), 8.12 (dd, J=1.60, 8.70 Hz, 1H), 7.90 (br. s., 1H), 7.68 (dd, J=0.80, 8.82 Hz, 2H), 7.39 (d, J=8.01 Hz, 1H), 3.99 (s, 3H), 3.74 (br. s., 8H). MS (ESI): [M+H]⁺=406.0.

### methyl 3-(6-methoxy-1H-benzo[d]imidazol-2-yl)-1H-indazole-5-carboxylate (3h)

Compound 3 h was obtained by using 6-methoxy benzene-1,2-diamine according to General Synthesis Procedure C (yellow solid, yield: 33%).

¹H NMR (400 MHz, METHANOL-d₄) δ 9.20 (dd, J=0.92, 1.60 Hz, 1H), 8.09 (dd, J=1.60, 8.93 Hz, 1H), 7.64 (dd, J=0.92, 8.70 Hz, 1H), 7.59 (br. s, 1H), 7.15 (br. s., 1H), 6.93 (dd, J=2.40, 8.82 Hz, 1H), 3.97 (s, 3H), 3.88 (s, 3H). MS (ESI): [M+H]⁺=323.6.

### methyl 3-(6-(2-methoxyethoxy)-1H-benzo[d]imidazol-2-yl)-1H-indazole-5-carboxylate (3i)

Compound 3i was obtained by using 6-(2-methoxyethoxy) benzene-1,2-diamine according to General Synthesis Procedure C (light brown solid, yield: 74%).

¹H NMR (400 MHz, METHANOL-d₄) δ 9.21 (dd, J=0.92, 1.60 Hz, 1H), 8.10 (dd, J=1.49, 8.82 Hz, 1H), 7.65 (dd, J=0.92, 8.93 Hz, 1H), 7.62 (br. s, 1H), 7.14 (br. s, 1H), 6.97 (dd, J=2.06, 8.70 Hz, 3H), 4.16-4.23 (m, 2H), 3.98 (s, 3H), 3.78-3.82 (m, 2H), 3.46 (s, 3H).

MS (ESI): [M+H]⁺=367.5

### methyl 3-(6-(2-(dimethylamino)ethoxy)-1H-benzo[d]imidazol-2-yl)-1H-indazole-5-carboxylate (3j)

Compound 3j was obtained by using 6-(2-methoxy-N,N-dimethylethane-1-amine) benzene-1,2-diamine according to General Synthesis Procedure C (brown oil, yield: 37%).

¹H NMR (400 MHz, METHANOL-d₄) δ 9.18 (dd, J=0.80, 1.49 Hz, 1H), 8.06 (dd, J=1.49, 8.82 Hz, 1H), 7.61 (dd, J=0.80, 8.82 Hz, 1H), 7.56 (d, J=7.56 Hz, 1H), 7.17 (br. s., 1H), 6.94 (dd, J=2.40, 8.82 Hz, 1H), 4.15 (t, J=5.50 Hz, 2H), 3.95 (s, 3H), 2.80 (t, J=5.38 Hz, 2H), 2.36 (s, 6H). MS (ESI): [M+H]⁺=380.6.

### methyl 3-(7-fluoro-1H-benzo[d]imidazol-2-yl)-1H-indazole-5-carboxylate (3k)

Compound 3k was obtained by using 7-fluorobenzene-1,2-diamine according to General Synthesis Procedure C (brown solid, yield: 39%).

¹H NMR (400 MHz, DMSO-d₆) δ 9.28 (dd, J=0.69, 1.60 Hz, 1H), 8.08 (dd, J=1.60, 8.93 Hz, 1H), 7.79 (dd, J=0.80, 8.82 Hz, 1H), 7.50-7.56 (m, 1H), 7.27 (dt, J=4.81, 8.01 Hz, 1H), 7.11 (ddd, J=0.69, 8.01, 10.99 Hz, 1H), 3.94 (s, 3H). MS (ESI): [M+H]⁺=311.2

### methyl 3-(6-(morpholinomethyl)-1H-benzo[d]imidazol-2-yl)-1H-indazole-5-carboxylate (3l)

Compound 3l was obtained by using 6-morpholinomethyl benzene-1,2-diamine according to General Synthesis Procedure C (yellow oil, yield: 66%).

¹H NMR (400 MHz, CHLOROFORM-d) δ 11.16 (br. s, 1H), 10.27 (br. s, 1H), 9.41 (s, 1H), 8.15 (dd, J=1.60, 8.70 Hz, 1H), 7.75 (br. s, 1H), 7.53 (d, J=8.93 Hz, 1H), 7.48 (br. s, 1H), 7.27-7.29 (m, 1H), 3.97 (s, 3H), 3.72-3.75 (m, 4H), 3.65 (s, 2H), 2.49-2.54 (m, 4H).

MS (ESI): [M+H]⁺=391.6.

### 3-(6-methyl-1H-benzo[d]imidazol-2-yl)-1H-indazole-5-carboxylic acid (4a)

Compound 4a was obtained from compound 3a according to General Synthesis Procedure D (brown solid, yield: 93%).

¹H NMR (400 MHz, METHANOL-d₄) δ 9.16 (t, J=1.14 Hz, 1H), 8.19 (dd, J=1.49, 8.82 Hz, 1H), 7.77 (dd, J=0.80, 8.82 Hz, 1H), 7.69 (d, J=8.47 Hz, 1H), 7.59-7.60 (m, 1H), 7.35-7.39 (m, 1H), 2.55 (s, 3H). MS (ESI): [M+H]⁺=293.4.

### 3-(6-(pyrrolidin-1-yl)-1H-benzo[d]imidazol-2-yl)-1H-indazole-5-carboxylic acid (4b)

Compound 4b was obtained from compound 3b according to General Synthesis Procedure D (brown solid, yield: 85%).

¹H NMR (400 MHz, METHANOL-d₄) δ 9.11 (s, 1H), 8.19 (dd, J=1.37, 8.93 Hz, 1H), 7.76 (d, J=9.16 Hz, 1H), 7.61 (d, J=8.93 Hz, 1H), 6.91 (dd, J=2.18, 9.04 Hz, 1H), 6.74 (d, J=2.06 Hz, 1H), 3.34-3.41 (m, 4H), 2.06-2.13 (m, 4H). MS (ESI): [M+H]⁺=348.3

### 3-(6-(piperidin-1-yl)-1H-benzo[d]imidazol-2-yl)-1H-indazole-5-carboxylic acid (4c)

Compound 4c was obtained from compound 3c according to General Synthesis Procedure D (black solid, yield: 77%).

Mixture of two tautomers. ¹H NMR (400 MHz, DMSO-d₆) δ 12.66-12.89 (m, 1H), 9.16-9.22 (m, 1H), 8.01 (dd, J=1.37, 8.70 Hz, 1H), 7.69 (d, J=8.93 Hz, 1H), 7.33-7.65 (m, 1H), 6.93-7.04 (m, 1H), 6.91-7.31 (m, 1H), 3.06-3.15 (m, 4H), 1.63-1.74 (m, 4H), 1.50-1.60 (m, 2H). MS (ESI): [M+H]⁺=362.1.

### 3-(6-(4-methylpiperazin-1-yl)-1H-benzo[d]imidazol-2-yl)-1H-indazole-5-carboxylic acid (4d)

Compound 4d was obtained from compound 3d according to General Synthesis Procedure D (yellowish solid, yield: 85%).

¹H NMR (400 MHz, DMSO-d₆) δ 12.72-12.87 (m, 1H), 9.16-9.24 (m, 1H), 7.98-8.04 (m, 1H), 7.66-7.72 (m, 1H), 7.35-7.66 (m, 1H), 6.95-7.05 (m, 1H), 6.91-7.32 (m, 1H), 3.12-3.19 (m, 4H), 2.53-2.59 (m, 4H), 2.25-2.31 (m, 3H). MS (ESI): [M+H]⁺=377.4.

### 3-(6-((3S,5R)-3,5-dimethylpiperazin-1-yl)-1H-benzo[d]imidazol-2-yl)-1H-indazole-5-carboxylic acid (4e)

Compound 4e was obtained from compound 3e according to General Synthesis Procedure D (light orange solid, yield: 90%).

¹H NMR (400 MHz, METHANOL-d₄) δ 9.14 (t, J=1.14 Hz, 1H), 8.12 (dd, J=1.49, 8.82 Hz, 1H), 7.58-7.66 (m, 2H), 7.26 (br. s., 1H), 7.11 (dd, J=2.18, 8.82 Hz, 1H), 3.80 (dd, J=2.18, 12.94 Hz, 2H), 3.48-3.61 (m, 3H), 2.71-2.83 (m, 2H), 1.42 (d, J=6.64 Hz, 6H). MS (ESI): [M+H]⁺=391.5.

### 3-(6-morpholino-1H-benzo[d]imidazol-2-yl)-1H-indazole-5-carboxylic acid (4f)

Compound 4f was obtained from compound 3f according to General Synthesis Procedure D (brown solid, yield: 86%).

¹H NMR (400 MHz, DMSO-d₆) δ 12.92 (br. s, 1H), 9.19 (s, 1H), 8.01 (dd, J=1.60, 8.70 Hz, 1H), 7.70 (dd, J=0.69, 8.70 Hz, 1H), 7.61 (br. s, 1H), 6.87-7.12 (m, 2H), 3.75-3.84 (m, 4H), 3.09-3.17 (m, 4H). MS (ESI): [M+H]⁺=363.8.

### 3-(6-(morpholine-4-carbonyl)-1H-benzo[d]imidazol-2-yl)-1H-indazole-5-carboxylic acid (4g)

Compound 4g was obtained from compound 3g according to General Synthesis Procedure D (white solid, yield: 99%).

¹H NMR (400 MHz, METHANOL-d₄) δ 9.26 (dd, J=0.60, 1.50 Hz, 1H), 8.14 (dd, J=1.49, 8.82 Hz, 1H), 7.74-7.84 (m, 2H), 7.67 (dd, J=0.57, 8.82 Hz, 1H), 7.39 (dd, J=1.37, 8.24 Hz, 1H), 3.73 (br. s., 8H). MS (ESI): [M+H]⁺=392.6.

### 3-(6-methoxy-1H-benzo[d]imidazol-2-yl)-1H-indazole-5-carboxylic acid (4h)

Compound 4 h was obtained from compound 3 h according to General Synthesis Procedure D (brown solid, yield: 98%).

¹H NMR (400 MHz, METHANOL-d₄) δ 9.12 (dd, J=0.80, 1.40 Hz, 1H), 8.22 (dd, J=1.37, 8.93 Hz, 1H), 7.81 (dd, J=0.80, 8.82 Hz, 1H), 7.74 (dd, J=0.57, 9.04 Hz, 1H), 7.30 (d, J=2.29 Hz, 1H), 7.23 (dd, J=2.40, 9.04 Hz, 1H), 3.94 (s, 3H). MS (ESI): [M+H]⁺=309.0

### 3-(6-(2-methoxyethoxy)-1H-benzo[d]imidazol-2-yl)-1H-indazole-5-carboxylic acid (4i)

Compound 4i was obtained from compound 3i according to General Synthesis Procedure D (brown solid, yield: 84%).

¹H NMR (400 MHz, METHANOL-d₄) δ 9.22 (dd, J=0.92, 1.60 Hz, 1H), 8.12 (dd, J=1.49, 8.82 Hz, 1H), 7.64 (dd, J=0.92, 8.70 Hz, 1H), 7.59 (d, J=8.93 Hz, 1H), 7.21 (d, J=2.29 Hz, 1H), 6.97 (dd, J=2.29, 8.70 Hz, 1H), 4.18-4.22 (m, 2H), 3.78-3.83 (m, 2H), 3.46 (s, 3H). MS (ESI): [M+H]⁺=353.4.

### 3-(6-(2-(dimethylamino)ethoxy)-1H-benzo[d]imidazol-2-yl)-1H-indazole-5-carboxylic acid (4j)

Compound 4j was obtained from compound 3j according to General Synthesis Procedure D (brown solid, yield: 87%).

¹H NMR (400 MHz, METHANOL-d₄) δ 9.18 (dd, J=0.80, 1.49 Hz, 1H), 8.12 (dd, J=1.49, 8.82 Hz, 1H), 7.56-7.66 (m, 2H), 7.30 (d, J=2.52 Hz, 1H), 7.04 (dd, J=2.40, 8.82 Hz, 1H), 4.39-4.44 (m, 2H), 3.53-3.60 (m, 2H), 2.96 (s, 6H). MS (ESI): [M+H]⁺=366.0.

### 3-(7-fluoro-1H-benzo[d]imidazol-2-yl)-1H-indazole-5-carboxylic acid (4k)

Compound 4k was obtained from compound 3k according to General Synthesis Procedure D (brown solid, yield: 99%).

¹H NMR (400 MHz, DMSO-d₆) δ 9.18-9.22 (m, 1H), 8.04 (ddd, J=0.69, 1.60, 8.70 Hz, 1H), 7.74 (td, J=0.77, 8.76 Hz, 1H), 7.41 (d, J=8.01 Hz, 1H), 7.20-7.28 (m, 1H), 7.06 (dd, J=8.01, 11.22 Hz, 1H). MS (ESI): [M+H]⁺=297.3

### 3-(6-(morpholinomethyl)-1H-benzo[d]imidazol-2-yl)-1H-indazole-5-carboxylic acid (41)

Compound 41 was obtained from compound 31 according to General Synthesis Procedure D (light brown solid, yield: 92%).

¹H NMR (400 MHz, DMSO-d₆) δ 11.41 (br. s, 1H), 9.21 (dd, J=0.80, 1.49 Hz, 1H), 8.05 (dd, J=1.37, 8.70 Hz, 1H), 8.01 (s, 1H), 7.75-7.80 (m, 2H), 7.57 (dd, J=1.26, 8.36 Hz, 1H), 4.48 (d, J=4.81 Hz, 2H), 3.90-3.97 (m, 2H), 3.78-3.86 (m, 2H), 3.25 (d, J=11.91 Hz, 2H), 3.06-3.15 (m, 2H). MS (ESI): [M+H]⁺=377.5

### 3-(6-chloro-1H-benzo[d]imidazol-2-yl)-1H-indazole-5-carboxylic acid (4m)

Compound 4m was obtained by using 6-chlorobenzene-1,2-diamine according to General Synthesis Procedure E (brown solid, yield: 82%).

¹H NMR (400 MHz, DMSO-d₆) δ 9.18 (s, 1H), 8.03 (dd, J=1.60, 8.70 Hz, 1H), 7.83 (d, J=8.70 Hz, 1H), 7.73 (d, J=8.93 Hz, 1H), 7.48-7.57 (m, 1H), 7.26 (ddd, J=2.06, 8.59, 13.40 Hz, 1H). MS (ESI): [M+H]⁺=313.04

### 3-(6-bromo-1H-benzo[d]imidazol-2-yl)-1H-indazole-5-carboxylic acid (4n)

Compound 4n was obtained by using 6-bromobenzene-1,2-diamine according to General Synthesis Procedure E (brown solid, yield: 95%).

¹H NMR (400 MHz, DMSO-d₆) δ 9.22-9.31 (m, 1H), 8.07 (td, J=1.03, 11.22 Hz, 1H), 7.80-7.84 (m, 1H), 7.74 (d, J=0.69 Hz, 1H), 7.49-7.53 (m, 1H), 7.34-7.39 (m, 1H). MS (ESI): [M+H]⁺=356.68

### 3-(6-(trifluoromethyl)-1H-benzo[d]imidazol-2-yl)-1H-indazole-5-carboxylic acid (4o)

Compound 4o was obtained by using 6-trifluoromethyl benzene-1,2-diamine according to General Synthesis Procedure E (brown solid, yield: 84%).

¹H NMR (400 MHz, DMSO-d₆) δ 9.20 (s, 1H), 8.04-8.07 (m, 1H), 7.83-7.89 (m, 1H), 7.71-7.77 (m, 2H), 7.49-7.57 (m, 1H). MS (ESI): [M+H]⁺=346.91

### 3-(5,6-difluoro-1H-benzo[d]imidazol-2-yl)-1H-indazole-5-carboxylic acid (4p)

Compound 4p was obtained by using 5,6-difluorobenzene-1,2-diamine according to General Synthesis Procedure E (brown solid, yield: 87%).

¹H NMR (400 MHz, DMSO-d₆) δ 9.17 (s, 1H), 7.97-8.05 (m, 1H), 7.63-7.77 (m, 3H). MS (ESI): [M+H]⁺=314.6

### 3-(6-cyano-1H-benzo[d]imidazol-2-yl)-1H-indazole-5-carboxylic acid (4q)

Compound 4q was obtained by using 6-cyanobenzene-1,2-diamine according to General Synthesis Procedure E (orange solid, yield: 77%).

¹H NMR (400 MHz, DMSO-d₆) δ 9.09-9.16 (m, 1H), 8.21-8.44 (m, 1H), 7.99-8.21 (m, 1H), 7.52-7.82 (m, 3H). MS (ESI): [M+H]⁺=303.7. tert-butyl (3-(3-(6-methyl-1H-benzo[d]imidazol-2-yl)-1H-indazole-5-carboxamido)propyl) carbamate (5a) Compound 5a was obtained from compound 4a by using N-Boc-propane-1,3-diamine according to General Synthesis Procedure A (light brown solid, yield: 81%).

¹H NMR (400 MHz, METHANOL-d₄) δ 8.99 (dd, J=0.69, 1.60 Hz, 1H), 7.96 (dd, J=1.72, 8.82 Hz, 1H), 7.66 (dd, J=0.92, 8.93 Hz, 1H), 7.34-7.64 (m, 2H), 7.14 (d, J=8.20 Hz, 1H), 3.50 (t, J=6.90 Hz, 2H), 3.20 (t, J=6.75 Hz, 2H), 2.50 (s, 3H), 1.85 (quin, J=6.90 Hz, 2H), 1.43 (s, 9H). MS (ESI): [M+H]⁺=449.6.

### tert-butyl (3-(3-(6-(pyrrolidin-1-yl)-1H-benzo[d]imidazol-2-yl)-1H-indazole-5-carboxamido) propyl)carbamate (5b)

Compound 5b was obtained from compound 4b by using tert-butyl (3-aminopropyl)carbamate according to General Synthesis Procedure A (yellow solid, yield: 50%).

¹H NMR (400 MHz, METHANOL-d₄) δ 8.97-8.99 (m, 1H), 7.95 (dd, J=1.60, 8.70 Hz, 1H), 7.64 (dd, J=0.69, 8.70 Hz, 1H), 7.55 (d, J=9.16 Hz, 1H), 6.69-6.77 (m, 2H), 3.50 (t, J=6.87 Hz, 2H), 3.34-3.39 (m, 4H), 3.19 (dt, J=1.26, 6.47 Hz, 2H), 2.07 (td, J=3.35, 6.35 Hz, 4H), 1.84 (quin, J=6.70 Hz, 2H), 1.43 (s, 9H). MS (ESI): [M+H]⁺=504.3.

### tert-butyl (3-(3-(6-(piperidin-1-yl)-1H-benzo[d]imidazol-2-yl)-1H-indazole-5-carboxamido) propyl)carbamate (5c)

Compound 5c was obtained from compound 4c by using tert-butyl (3-aminopropyl)carbamate according to General Synthesis Procedure A (brown solid, yield: 38%).

Mixture of two tautomers. ¹H NMR (400 MHz, DMSO-d₆) δ 13.68 (br. s, 1H), 12.69 (br. s., 1H), 8.97 (s, 1H), 8.58 (t, J=5.50 Hz, 1H), 7.91 (dd, J=1.60, 8.71 Hz, 1H), 7.66 (d, J=8.94 Hz, 1H), 7.32-7.63 (m, 1H), 6.97-7.28 (m, 1H), 6.92-6.97 (m, 1H), 6.83 (t, J=5.70 Hz, 1H), 3.26-3.30 (m, 2H), 3.06-3.16 (m, 4H), 3.01 (q, J=6.50 Hz, 2H), 1.62-1.74 (m, 6H), 1.50-1.60 (m, 2H), 1.38 (s, 9H). MS (ESI): [M+H]⁺=518.3.

### tert-butyl (3-(3-(6-(4-methylpiperazin-1-yl)-1H-benzo[d]imidazol-2-yl)-1H-indazole-5-carboxamido)propyl)carbamate (5d)

Compound 5d was obtained from compound 4d by using tert-butyl (3-aminopropyl)carbamate according to General Synthesis Procedure A (yellowish solid, yield: 52%).

¹H NMR (400 MHz, METHANOL-d₄) δ 8.99 (dd, J=0.69, 1.60 Hz, 1H), 7.96 (dd, J=1.60, 8.80 Hz, 1H), 7.66 (dd, J=0.92, 8.93 Hz, 1H), 7.64 (br. s, OH), 7.11 (d, J=8.70 Hz, 1H), 7.14 (br. s, OH), 3.50 (t, J=6.87 Hz, 2H), 3.23-3.29 (m, 4H), 3.19 (t, J=6.64 Hz, 2H), 2.67-2.73 (m, 4H), 2.39 (s, 3H), 1.84 (quin, J=6.90 Hz, 2H), 1.43 (s, 9H). MS (ESI): [M+H]⁺=533.4.

### tert-butyl (3-(3-(6-((3S,5R)-3,5-dimethylpiperazin-1-yl)-1H-benzo[d]imidazol-2-yl)-1H-indazole-5-carboxamido)propyl)carbamate (5e)

Compound 5e was obtained from compound 4e by using tert-butyl (3-aminopropyl)carbamate according to General Synthesis Procedure A (yellow solid, yield: 34%).

¹H NMR (400 MHz, METHANOL-d₄) δ 8.99 (dd, J=0.57, 1.49 Hz, 1H), 7.96 (dd, J=1.72, 8.82 Hz, 1H), 7.66 (dd, J=0.50, 8.93 Hz, 1H), 7.60 (br. s, 1H), 7.19 (br. s., 1H), 7.11 (dd, J=2.06, 8.70 Hz, 1H), 3.57 (dd, J=1.60, 11.45 Hz, 2H), 3.50 (t, J=6.87 Hz, 2H), 3.19 (t, J=6.60 Hz, 2H), 3.05-3.13 (m, 2H), 2.37 (t, J=11.00 Hz, 2H), 1.84 (quin, J=6.58 Hz, 2H), 1.44 (s, 9H), 1.20 (d, J=6.64 Hz, 6H). MS (ESI): [M+H]⁺=547.4

### tert-butyl (3-(3-(6-morpholino-1H-benzo[d]imidazol-2-yl)-1H-indazole-5-carboxamido)propyl)carbamate (5f)

Compound 5f was obtained from compound 4f by using tert-butyl (3-aminopropyl)carbamate according to General Synthesis Procedure A (yellow solid, yield: 67%).

Mixture of two tautomers. ¹H NMR (400 MHz, DMSO-d₆) δ 13.70-13.76 (m, 1H), 12.74-12.83 (m, 1H), 8.96-9.00 (m, 1H), 8.56-8.62 (m, 1H), 7.91 (dd, J=1.72, 8.82 Hz, 1H), 7.65-7.69 (m, 1H), 7.36-7.65 (m, 1H), 6.96-7.05 (m, 1H), 6.92-7.31 (m, 1H), 6.84 (t, J=5.50 Hz, 1H), 3.75-3.81 (m, 4H), 3.28-3.31 (m, 2H), 3.09-3.15 (m, 4H), 3.01 (q, J=6.40 Hz, 2H), 1.67 (quin, J=7.10 Hz, 2H), 1.38 (s, 9H). MS (ESI): [M+H]⁺=520.4

### tert-butyl (3-(3-(6-(morpholine-4-carbonyl)-1H-benzo[d]imidazol-2-yl)-1H-indazole-5-carboxamido)propyl)carbamate (5g)

Compound 5g was obtained from compound 4g by using tert-butyl (3-aminopropyl)carbamate according to General Synthesis Procedure A (colorless oil, yield: 44%).

¹H NMR (400 MHz, METHANOL-d₄) δ 9.01 (dd, J=0.92, 1.60 Hz, 1H), 7.99 (dd, J=1.60, 8.93 Hz, 1H), 7.78-7.84 (m, 2H), 7.72 (dd, J=0.69, 8.93 Hz, 1H), 7.45 (dd, J=1.37, 8.24 Hz, 1H), 3.74 (br. s., 8H), 3.51 (t, J=6.87 Hz, 2H), 3.20 (t, J=6.87 Hz, 2H), 1.84 (quin, J=6.60 Hz, 2H), 1.44 (s, 9H). MS (ESI): [M+H]⁺=548.5

### tert-butyl (3-(3-(6-methoxy-1H-benzo[d]imidazol-2-yl)-1H-indazole-5-carboxamido)propyl) carbamate (5h)

Compound 5 h was obtained from compound 4 h by using tert-butyl (3-aminopropyl)carbamate according to General Synthesis Procedure A (yellowish solid, yield: 72%).

¹H NMR (400 MHz, METHANOL-d₄) δ 8.95-9.02 (m, 1H), 7.92-7.98 (m, 1H), 7.65 (d, J=8.70 Hz, 1H), 7.61 (br. s, 1H), 7.12 (br. s., 1H), 6.94 (dd, J=1.60, 8.70 Hz, 1H), 3.88 (s, 3H), 3.50 (t, J=6.87 Hz, 2H), 3.19 (t, J=6.75 Hz, 2H), 1.84 (quin, J=6.75 Hz, 2H), 1.43 (s, 9H). MS (ESI): [M+H]⁺=465.2

### tert-butyl (3-(3-(6-(2-methoxyethoxy)-1H-benzo[d]imidazol-2-yl)-1H-indazole-5-carboxamido)propyl)carbamate (5i)

Compound 5i was obtained from compound 4i by using tert-butyl (3-aminopropyl)carbamate according to General Synthesis Procedure A (white solid, yield: 69%).

¹H NMR (400 MHz, METHANOL-d₄) δ 8.98 (dd, J=0.69, 1.60 Hz, 1H), 7.95 (dd, J=1.60, 8.70 Hz, 1H), 7.66 (dd, J=0.80, 8.82 Hz, 1H), 7.39-7.71 (m, OH), 7.08-7.36 (m, 1H), 6.97 (dd, J=1.60, 8.70 Hz, 1H), 4.17-4.23 (m, 2H), 3.78-3.83 (m, 2H), 3.50 (t, J=6.87 Hz, 2H), 3.46 (s, 3H), 3.19 (t, J=6.75 Hz, 2H), 1.84 (quin, J=6.75 Hz, 2H), 1.43 (s, 9H). MS (ESI): [M+H]⁺=509.4

### tert-butyl (3-(3-(6-(2-(dimethylamino)ethoxy)-1H-benzo[d]imidazol-2-yl)-1H-indazole-5-carboxamido)propyl)carbamate (5j)

Compound 5j was obtained from compound 4j by using tert-butyl (3-aminopropyl)carbamate according to General Synthesis Procedure A (yellowish solid, yield: 55%).

¹H NMR (400 MHz, METHANOL-d₄) δ 8.99 (dd, J=0.69, 1.60 Hz, 1H), 7.95 (dd, J=1.72, 8.82 Hz, 1H), 7.66 (dd, J=0.92, 8.93 Hz, 1H), 7.60 (br. s., 1H), 7.20 (br. s., 1H), 6.98 (dd, J=2.40, 8.82 Hz, 1H), 4.19 (t, J=5.50 Hz, 2H), 3.50 (t, J=6.98 Hz, 2H), 3.19 (t, J=6.75 Hz, 2H), 2.84 (t, J=5.50 Hz, 2H), 2.39 (s, 6H), 1.84 (quin, J=6.75 Hz, 2H), 1.43 (s, 9H). MS (ESI): [M+H]⁺=522.7

### tert-butyl (3-(3-(7-fluoro-1H-benzo[d]imidazol-2-yl)-1H-indazole-5-carboxamido)propyl) carbamate (5k)

Compound 5k was obtained from compound 4k by using tert-butyl (3-aminopropyl)carbamate according to General Synthesis Procedure A (light brown solid, yield: 53%).

¹H NMR (400 MHz, METHANOL-d₄) δ 9.03 (s, 1H), 7.98 (dd, J=1.72, 8.82 Hz, 1H), 7.68 (dd, J=0.92, 8.70 Hz, 1H), 7.41 (br. s., 1H), 7.26 (dt, J=4.81, 8.13 Hz, 1H), 7.02 (ddd, J=0.69, 8.41, 10.13 Hz, 1H), 3.51 (t, J=6.87 Hz, 2H), 3.19 (t, J=6.75 Hz, 2H), 1.84 (quin, J=6.75 Hz, 2H), 1.43 (s, 9H). MS (ESI): [M+H]⁺=453.4

### tert-butyl (3-(3-(6-(morpholinomethyl)-1H-benzo[d]imidazol-2-yl)-1H-indazole-5-carboxamido)propyl)carbamate (51)

Compound 51 was obtained from compound 41 by using tert-butyl (3-aminopropyl)carbamate according to General Synthesis Procedure A (yellowish oil, yield: 50%).

¹H NMR (400 MHz, METHANOL-d₄) δ 9.01 (dd, J=0.92, 1.83 Hz, 1H), 7.96 (dd, J=1.83, 8.70 Hz, 1H), 7.76 (br. s, 1H), 7.67 (dd, J=0.92, 8.70 Hz, 1H), 7.58 (br. s, 1H), 7.32 (d, J=8.24 Hz, 1H), 3.70-3.74 (m, 4H), 3.69 (s, 2H), 3.51 (t, J=6.87 Hz, 2H), 3.20 (t, J=6.64 Hz, 2H), 2.49-2.55 (m, 4H), 1.84 (quin, J=6.90 Hz, 2H), 1.43 (s, 9H). MS (ESI): [M+H]⁺=534.8

### tert-butyl (3-(3-(6-chloro-1H-benzo[d]imidazol-2-yl)-1H-indazole-5-carboxamido)propyl)carbamate (5m)

Compound 5m was obtained from compound 4m by using tert-butyl (3-aminopropyl)carbamate according to General Synthesis Procedure A (yellow oil, yield: 20%).

¹H NMR (400 MHz, METHANOL-d₄) δ 8.97-9.01 (m, 1H), 7.94-7.99 (m, 1H), 7.48-7.75 (m, 3H), 7.20-7.31 (m, 1H), 3.39-3.56 (m, 2H), 3.13-3.22 (m, 2H), 1.76-1.88 (m, 2H), 1.43 (s, 9H). MS (ESI): [M+H]⁺=469.6

### tert-butyl (3-(3-(6-bromo-1H-benzo[d]imidazol-2-yl)-1H-indazole-5-carboxamido)propyl)carbamate (5n)

Compound 5n was obtained from compound 4n by using tert-butyl (3-aminopropyl)carbamate according to General Synthesis Procedure A (yellow oil, yield 35%).

¹H NMR (400 MHz, METHANOL-d₄) δ 8.97-9.03 (m, 1H), 7.95-8.00 (m, 1H), 7.74-7.95 (m, 1H), 7.65-7.71 (m, 1H), 7.46-7.65 (m, 1H), 7.37-7.44 (m, 1H), 3.45-3.53 (m, 2H), 3.14-3.23 (m, 2H), 1.77-1.88 (m, 2H), 1.46 (s, 9H). MS (ESI): [M+H]⁺=513.2

### tert-butyl (3-(3-(6-(trifluoromethyl)-1H-benzo[d]imidazol-2-yl)-1H-indazole-5-carboxamido)propyl)carbamate (5o)

Compound 5o was obtained from compound 4o by using tert-butyl (3-aminopropyl)carbamate according to General Synthesis Procedure A (yellow oil, yield: 33%).

¹H NMR (400 MHz, METHANOL-d₄) δ 9.01-9.06 (m, 1H), 7.94-8.14 (m, 2H), 7.74-7.94 (m, 1H), 7.66-7.73 (m, 1H), 7.54-7.61 (m, 1H), 3.51 (t, J=6.90 Hz, 1H), 3.20 (t, J=6.90 Hz, 2H), 1.78-1.90 (m, 2H), 1.42 (s, 9H). MS (ESI): [M+H]⁺=502.82

### Example 9: 3-(5,6-difluoro-1H-benzo[d]imidazol-2-yl)-N-(3-(dimethylamino)propyl)-1H-indazole-5-carboxamide (5p)

Compound (5p) of Example 9 was obtained from compound 4p by using N,N-dimethylpropane-1,3-diamine according to General Synthesis Procedure A (yellow solid, yield: 34%).

¹H NMR (400 MHz, METHANOL-d₄) δ 8.95 (dd, J=0.80, 1.72 Hz, 1H), 7.94 (dd, J=1.60, 8.70 Hz, 1H), 7.66 (dd, J=0.92, 8.70 Hz, 1H), 7.48-7.55 (m, 2H), 3.49 (t, J=6.98 Hz, 2H), 2.50 (t, J=7.00 Hz, 2H), 2.33 (s, 6H), 1.84-1.95 (m, 2H). MS (ESI): [M+H]⁺=399.0

### Example 10: 3-(6-cyano-1H-benzo[d]imidazol-2-yl)-N-(3-(dimethylamino)propyl)-1H-indazole-5-carboxamide (5q)

Compound (5q) of Example 10 was obtained from compound 4q by using N,N-dimethylpropane-1,3-diamine according to General Synthesis Procedure A (white solid, yield: 55%).

¹H NMR (400 MHz, METHANOL-d₄) δ 8.94-9.01 (m, 1H), 8.00-8.07 (m, 1H), 7.94-7.98 (m, 1H), 7.73-7.79 (m, 1H), 7.67 (dd, J=0.80, 8.82 Hz, 1H), 7.54-7.59 (m, 1H), 3.50 (t, J=6.98 Hz, 2H), 2.39-2.58 (m, 2H), 2.32 (s, 6H), 1.81-1.93 (m, 2H). MS (ESI): [M+H]⁺=388.0

### Example 11: N-(3-(dimethylamino)propyl)-3-(6-methoxy-1H-benzo[d]imidazol-2-yl)-1H-indazole-5-carboxamide (5r)

Compound (5r) of Example 11 was obtained from compound 4a by using N,N-dimethylpropane-1,3-diamine according to General Synthesis Procedure A (yellow oil, yield: 45%).

¹H NMR (400 MHz, METHANOL-d₄) δ 8.97 (dd, J=0.92, 1.60 Hz, 1H), 7.95 (dd, J=1.60, 8.70 Hz, 1H), 7.66 (dd, J=0.80, 8.82 Hz, 1H), 7.52-7.62 (m, 1H), 7.09-7.22 (m, 1H), 6.94 (dd, J=2.40, 8.82 Hz, 1H), 3.88 (s, 3H), 3.44-3.54 (m, 2H), 2.42-2.55 (m, 2H), 2.32 (s, 6H), 1.81-1.99 (m, 2H). MS (ESI): [M+H]⁺=393.0

### Example 12: N-(3-(dimethylamino)propyl)-3-(6-methyl-1H-benzo[d]imidazol-2-yl)-1H-indazole-5-carboxamide (5s)

Compound (5s) of Example 12 was obtained from compound 4 h by using N,N-dimethylpropane-1,3-diamine according to General Synthesis Procedure A (white solid, yield: 52%).

¹H NMR (400 MHz, METHANOL-d₄) δ 8.96-9.01 (m, 1H), 7.93-8.00 (m, 1H), 7.64-7.71 (m, 1H), 7.41-7.64 (m, 2H), 7.11-7.17 (m, 1H), 3.43-3.54 (m, 2H), 2.52-2.62 (m, 2H), 2.47-2.52 (m, 3H), 2.30-2.42 (m, 6H), 1.85-1.99 (m, 2H). MS (ESI): [M+H]⁺=377.3

### tert-butyl ((1S,3S)-3-(3-(6-(pyrrolidin-1-yl)-1H-benzo[d]imidazol-2-yl)-1H-indazole-5-carboxamido)cyclopentyl)carbamate (5t)

Compound 5t was obtained from compound 4b by using tert-butyl ((1S,3S)-3-aminocyclopentyl)carbamate according to General Synthesis Procedure A (yellow solid, yield: 49%).

¹H NMR (400 MHz, METHANOL-d₄) δ 8.96 (dd, J=0.92, 1.60 Hz, 1H), 7.94 (dd, J=1.60, 8.70 Hz, 1H), 7.64 (dd, J=0.92, 8.93 Hz, 1H), 7.56 (d, J=9.39 Hz, 1H), 6.72-6.76 (m, 2H), 4.52 (quin, J=7.60 Hz, 1H), 4.11 (quin, J=6.40 Hz, 1H), 3.33-3.39 (m, 4H), 2.16-2.30 (m, 2H), 2.05-2.10 (m, 4H), 1.98-2.04 (m, 2H), 1.66-1.76 (m, 1H), 1.51-1.60 (m, 1H), 1.46 (s, 9H). MS (ESI): [M+H]⁺=530.5

### tert-butyl ((1S,3S)-3-(3-(6-(piperidin-1-yl)-1H-benzo[d]imidazol-2-yl)-1H-indazole-5-carboxamido)cyclopentyl) carbamate (5u)

Compound 5u was obtained from compound 4c by using tert-butyl ((1S,3S)-3-aminocyclopentyl)carbamate according to General Synthesis Procedure A (brown solid, yield: 68%).

¹H NMR (400 MHz, METHANOL-d₄) δ 8.95-8.99 (m, 1H), 7.95 (dd, J=1.60, 8.90 Hz, 1H), 7.51-7.71 (m, 2H), 7.07-7.27 (m, 2H), 4.52 (quin, J=7.27 Hz, 1H), 4.12 (quin, J=6.70 Hz, 1H), 3.14-3.20 (m, 4H), 2.15-2.30 (m, 2H), 1.97-2.04 (m, 2H), 1.77-1.83 (m, 4H), 1.66-1.75 (m, 1H), 1.60-1.65 (m, 2H), 1.50-1.59 (m, 1H), 1.46 (s, 9H). MS (ESI): [M+H]⁺=544.7

### tert-butyl ((1S,3S)-3-(3-(6-methoxy-1H-benzo[d]imidazol-2-yl)-1H-indazole-5-carboxamido) cyclopentyl)carbamate (5v)

Compound 5v was obtained from compound 4 h by using tert-butyl ((1S,3S)-3-aminocyclopentyl)carbamate according to General Synthesis Procedure A (yellowish solid, yield: 77%).

¹H NMR (400 MHz, METHANOL-d₄) δ 8.96 (dd, J=0.70, 1.60 Hz, 1H), 7.94 (dd, J=1.60, 8.70 Hz, 1H), 7.65 (dd, J=0.80, 8.82 Hz, 1H), 7.59 (d, J=8.70 Hz, 1H), 7.18 (br. s, 1H), 6.94 (dd, J=2.29, 8.70 Hz, 1H), 4.52 (quin, J=7.10 Hz, 1H), 4.11 (quin, J=6.90 Hz, 1H), 3.88 (s, 3H), 2.13-2.33 (m, 2H), 1.96-2.06 (m, 2H), 1.65-1.76 (m, 1H), 1.50-1.61 (m, 1H), 1.47 (s, 9H). MS (ESI): [M+H]⁺=491.5

### Example 13: 3-(6-(pyrrolidin-1-yl)-1H-benzo[d]imidazol-2-yl)-N-(4,4,4-trifluorobutyl)-1H-indazole-5-carboxamide (5w)

Compound (5w) of Example 13 was obtained from compound 4b by using 4,4,4-trifluorobutan-1-amine according to General Synthesis Procedure A (yellow solid, yield: 52%).

¹H NMR (400 MHz, METHANOL-d₄) δ 8.98 (dd, J=0.92, 1.60 Hz, 1H), 7.95 (dd, J=1.72, 8.82 Hz, 1H), 7.65 (dd, J=0.80, 8.82 Hz, 1H), 7.57 (br. s., 1H), 6.64-6.80 (m, 2H), 3.54 (t, J=6.98 Hz, 2H), 3.34-3.39 (m, 4H), 2.26-2.39 (m, 2H), 2.05-2.10 (m, 4H), 1.90-1.99 (m, 2H). MS (ESI): [M+H]⁺=457.5

### Example 14: N-((3,3-difluorocyclobutyl)methyl)-3-(6-(pyrrolidin-1-yl)-1H-benzo[d]imidazol-2-yl)-1H-indazole-5-carboxamide (5×)

Compound (5×) of Example 14 was obtained from compound 4b by using (3,3-difluorocyclobutyl)methanamin according to General Synthesis Procedure A (yellow solid, yield: 51%).

¹H NMR (400 MHz, METHANOL-d₄) δ 8.98 (dd, J=0.69, 1.60 Hz, 1H), 7.95 (dd, J=1.72, 8.82 Hz, 1H), 7.65 (dd, J=0.69, 8.93 Hz, 1H), 7.57 (br. s., 1H), 6.63-6.79 (m, 2H), 3.59 (d, J=6.87 Hz, 2H), 3.34-3.39 (m, 4H), 2.66-2.77 (m, 2H), 2.50-2.60 (m, 1H), 2.37-2.48 (m, 2H), 2.08 (td, J=3.38, 6.53 Hz, 4H). MS (ESI): [M+H]⁺=451.4

### Example 15: N-(3-aminopropyl)-3-(6-methyl-1H-benzo[d]imidazol-2-yl)-1H-indazole-5-carboxamide (6a)

Compound (6a) of Example 15 was obtained from compound 5a according to General Synthesis Procedure B (yellow solid, yield: 33%).

¹H NMR (400 MHz, METHANOL-d₄) δ 9.00 (dd, J=0.92, 1.60 Hz, 1H), 7.96 (dd, J=1.60, 8.93 Hz, 1H), 7.67 (dd, J=0.80, 8.82 Hz, 1H), 7.57 (d, J=8.01 Hz, 1H), 7.47 (s, 1H), 7.11-7.17 (m, 1H), 3.56 (t, J=6.75 Hz, 2H), 2.85 (t, J=7.10 Hz, 2H), 2.50 (s, 3H), 1.89 (t, J=6.98 Hz, 2H). MS (ESI): [M+H]⁺=349.6

### Example 16: N-(3-aminopropyl)-3-(6-(pyrrolidin-1-yl)-1H-benzo[d]imidazol-2-yl)-1H-indazole-5-carboxamide (6b)

Compound (6b) of Example 16 was obtained from compound 5b according to General Synthesis Procedure B (yellow solid, yield: 33%).

¹H NMR (400 MHz, METHANOL-d₄) δ 8.98 (dd, J=0.80, 1.72 Hz, 1H), 7.95 (dd, J=1.60, 8.93 Hz, 1H), 7.65 (dd, J=0.80, 8.82 Hz, 1H), 7.55 (d, J=9.39 Hz, 1H), 6.71-6.76 (m, 2H), 3.55 (t, J=6.75 Hz, 2H), 3.34-3.38 (m, 4H), 2.82 (t, J=6.98 Hz, 2H), 2.08 (td, J=3.32, 6.64 Hz, 4H), 1.87 (quin, J=6.87 Hz, 2H). MS (ESI): [M+H]⁺=404.3

### Example 17: N-(3-aminopropyl)-3-(6-(piperidin-1-yl)-1H-benzo[d]imidazol-2-yl)-1H-indazole-5-carboxamide (6c)

Compound (6c) of Example 17 was obtained from compound 5c according to General Synthesis Procedure B (yellow solid, yield: 79%).

¹H NMR (400 MHz, METHANOL-d₄) δ 8.99 (dd, J=0.80, 1.72 Hz, 1H), 7.95 (dd, J=1.60, 8.70 Hz, 1H), 7.66 (dd, J=0.80, 8.82 Hz, 1H), 7.59 (d, J=8.70 Hz, 1H), 7.23 (d, J=2.06 Hz, 1H), 7.11 (dd, J=2.29, 8.70 Hz, 1H), 3.55 (t, J=6.75 Hz, 2H), 3.15-3.19 (m, 4H), 2.82 (t, J=6.98 Hz, 2H), 1.87 (quin, J=6.90 Hz, 2H), 1.76-1.83 (m, 4H), 1.59-1.66 (m, J=5.70 Hz, 2H). MS (ESI): [M+H]⁺=418.1

### Example 18: N-(3-aminopropyl)-3-(6-(4-methylpiperazin-1-yl)-1H-benzo[d]imidazol-2-yl)-1H-indazole-5-carboxamide (6d)

Compound (6d) of Example 18 was obtained from compound 5d according to General Synthesis Procedure B (yellow solid, yield: 29%).

¹H NMR (400 MHz, METHANOL-d₄, TFA salt) δ 8.99-9.02 (m, 1H), 8.01 (d, J=8.70 Hz, 1H), 7.75 (d, J=8.70 Hz, 1H), 7.71 (d, J=8.47 Hz, 1H), 7.32 (br. s., 1H), 7.26 (dd, J=1.83, 8.70 Hz, 1H), 3.88 (br. s., 2H), 3.65 (br. s., 2H), 3.59 (t, J=6.60 Hz, 2H), 3.20 (br. s., 1H), 3.07 (t, J=7.21 Hz, 2H), 3.01 (s, 3H), 1.98-2.08 (m, 3H), 1.83 (br. s., 1H), 1.54 (br. s., 1H). MS (ESI): [M+H]⁺=433.5

### Example 19: N-(3-aminopropyl)-3-(6-((3S,5R)-3,5-dimethylpiperazin-1-yl)-1H-benzo[d]imidazol-2-yl)-1H-indazole-5-carboxamide (6e)

Compound (6e) of Example 19 was obtained from compound 5e according to General Synthesis Procedure B (yellow solid, yield: 31%).

¹H NMR (400 MHz, METHANOL-d₄, TFA salt) δ 9.01-9.04 (m, 1H), 7.97-8.02 (m, 1H), 7.73 (td, J=0.70, 8.93 Hz, 1H), 7.68 (d, J=8.93 Hz, 1H), 7.30 (d, J=2.06 Hz, 1H), 7.23 (dd, J=2.18, 8.82 Hz, 1H), 3.88 (dd, J=2.29, 13.05 Hz, 2H), 3.55-3.65 (m, 4H), 3.07 (t, J=7.33 Hz, 2H), 2.81 (dd, J=11.45, 13.05 Hz, 2H), 2.04 (quin, J=6.60 Hz, 2H), 1.44 (d, J=6.64 Hz, 6H). MS (ESI): [M+H]⁺=447.1

### Example 20: N-(3-aminopropyl)-3-(6-morpholino-1H-benzo[d]imidazol-2-yl)-1H-indazole-5-carboxamide (6f)

Compound (6f) of Example 19 was obtained from compound 5f according to General Synthesis Procedure B (yellowish solid, yield: 49%).

¹H NMR (400 MHz, DMSO-d₆) δ 8.98 (s, 1H), 8.68 (t, J=5.50 Hz, 1H), 7.90 (dd, J=1.72, 8.82 Hz, 1H), 7.66 (dd, J=0.69, 8.70 Hz, 1H), 7.58 (br. s, 1H), 6.87-7.12 (m, 2H), 3.76-3.83 (m, 4H), 3.35-3.41 (m, 2H), 3.08-3.14 (m, 4H), 2.63 (t, J=6.64 Hz, 2H), 1.64 (quin, J=6.75 Hz, 2H). MS (ESI): [M+H]⁺=420.4

### Example 21: N-(3-aminopropyl)-3-(6-(morpholine-4-carbonyl)-1H-benzo[d]imidazol-2-yl)-1H-indazole-5-carboxamide (6g)

Compound (6g) of Example 21 was obtained from compound 5g according to General Synthesis Procedure B (white solid, yield: 27%).

¹H NMR (400 MHz, METHANOL-d₄, TFA salt) δ 9.05 (dd, J=0.90, 1.60 Hz, 1H), 7.97 (dd, J=1.60, 8.70 Hz, 1H), 7.77 (br. s, 2H), 7.70 (dd, J=0.80, 8.82 Hz, 1H), 7.40 (d, J=8.24 Hz, 1H), 3.73 (br. s, 8H), 3.59 (t, J=6.53 Hz, 2H), 3.07 (t, J=7.33 Hz, 2H), 2.03 (quin, J=6.93 Hz, 2H). MS (ESI): [M+H]⁺=448.6

### Example 22: N-(3-aminopropyl)-3-(6-methoxy-1H-benzo[d]imidazol-2-yl)-1H-indazole-5-carboxamide (6h)

Compound (6h) of Example 22 was obtained from compound 5 h according to General Synthesis Procedure B (white solid, yield: 62%).

¹H NMR (400 MHz, METHANOL-d₄) δ 8.99 (dd, J=0.69, 1.60 Hz, 1H), 7.95 (dd, J=1.60, 8.93 Hz, 1H), 7.66 (dd, J=0.57, 8.82 Hz, 1H), 7.58 (d, J=8.70 Hz, 1H), 7.17 (d, J=2.29 Hz, 1H), 6.94 (dd, J=2.40, 8.82 Hz, 1H), 3.88 (s, 3H), 3.55 (t, J=6.75 Hz, 2H), 2.84 (t, J=6.98 Hz, 2H), 1.88 (quin, J=6.87 Hz, 2H). MS (ESI): [M+H]⁺=365.1

### Example 23: N-(3-aminopropyl)-3-(6-(2-methoxyethoxy)-1H-benzo[d]imidazol-2-yl)-1H-indazole-5-carboxamide (6i)

Compound (6i) of Example 23 was obtained from compound 5i according to General Synthesis Procedure B (white solid, yield: 59%).

¹H NMR (400 MHz, METHANOL-d₄, TFA salt) δ 8.99 (dd, J=0.80, 1.60 Hz, 1H), 7.95 (dd, J=1.60, 8.93 Hz, 1H), 7.66 (dd, J=0.80, 8.82 Hz, 1H), 7.59 (d, J=8.70 Hz, 1H), 7.20 (d, J=2.52 Hz, 1H), 6.98 (dd, J=2.40, 8.82 Hz, 1H), 4.18-4.22 (m, 2H), 3.78-3.83 (m, 2H), 3.55 (t, J=6.75 Hz, 2H), 3.46 (s, 3H), 2.83 (t, J=6.98 Hz, 2H), 1.87 (quin, J=6.93 Hz, 2H). MS (ESI): [M+H]⁺=409.6

### Example 24: N-(3-aminopropyl)-3-(6-(2-(dimethylamino)ethoxy)-1H-benzo[d]imidazol-2-yl)-1H-indazole-5-carboxamide (6j)

Compound (6j) of Example 24 was obtained from compound 5j according to General Synthesis Procedure B (yellowish solid, yield: 43%).

¹H NMR (400 MHz, METHANOL-d₄, TFA salt) δ 9.01 (dd, J=0.80, 1.72 Hz, 1H), 7.96 (dd, J=1.60, 8.70 Hz, 1H), 7.67 (dd, J=0.80, 8.82 Hz, 1H), 7.59 (d, J=8.93 Hz, 1H), 7.20 (d, J=2.06 Hz, 1H), 6.98 (dd, J=2.40, 8.82 Hz, 1H), 4.19 (t, J=5.50 Hz, 2H), 3.57 (t, J=6.60 Hz, 2H), 2.99 (t, J=7.30 Hz, 2H), 2.84 (t, J=5.38 Hz, 2H), 2.39 (s, 6H), 1.98 (quin, J=6.60 Hz, 2H). MS (ESI): [M+H]⁺=422.6

### Example 25: N-(3-aminopropyl)-3-(7-fluoro-1H-benzo[d]imidazol-2-yl)-1H-indazole-5-carboxamide (6k)

Compound (6k) of Example 25 was obtained from compound 5k according to General Synthesis Procedure B (brown solid, yield: 39%).

¹H NMR (400 MHz, METHANOL-d₄) δ 9.07 (dd, J=0.69, 1.60 Hz, 1H), 7.99 (dd, J=1.72, 8.82 Hz, 1H), 7.70 (dd, J=0.80, 8.82 Hz, 1H), 7.45 (d, J=8.01 Hz, 1H), 7.27 (dt, J=4.81, 8.13 Hz, 1H), 7.03 (dd, J=8.01, 10.99 Hz, 1H), 3.59 (t, J=6.53 Hz, 2H), 3.06 (t, J=7.33 Hz, 2H), 2.03 (quin, J=6.90 Hz, 2H). MS (ESI): [M+H]⁺=353.5

### Example 26: N-(3-aminopropyl)-3-(6-(morpholinomethyl)-1H-benzo[d]imidazol-2-yl)-1H-indazole-5-carboxamide (6l)

Compound (6l) of Example 26 was obtained from compound 51 according to General Synthesis Procedure B (white solid, yield: 50%).

¹H NMR (400 MHz, METHANOL-d₄) δ 9.01 (dd, J=0.80, 1.72 Hz, 1H), 7.96 (dd, J=1.72, 8.82 Hz, 1H), 7.63-7.69 (m, 1H), 7.32 (dd, J=1.60, 8.24 Hz, 1H), 3.69-3.74 (m, 4H), 3.68 (s, 2H), 3.55 (t, J=6.75 Hz, 2H), 2.81 (t, J=6.98 Hz, 2H), 2.49-2.56 (m, 4H), 1.86 (quin, J=6.90 Hz, 2H). MS (ESI): [M+H]⁺=433.9

### Example 27: N-(3-aminopropyl)-3-(6-chloro-1H-benzo[d]imidazol-2-yl)-1H-indazole-5-carboxamide (6m)

Compound (27) of Example 27 was obtained from compound 5m according to General Synthesis Procedure B (yellow oil, yield: 20%).

¹H NMR (400 MHz, METHANOL-d₄, TFA salt) δ 8.99-9.02 (m, 1H), 7.94-7.99 (m, 1H), 7.66-7.71 (m, 2H), 7.63 (d, J=8.24 Hz, 1H), 7.28 (dd, J=1.95, 8.59 Hz, 1H), 3.58 (t, J=6.64 Hz, 2H), 3.02 (t, J=7.21 Hz, 2H), 1.95-2.03 (m, 2H). MS (ESI): [M+H]⁺=369.1

### Example 28: N-(3-aminopropyl)-3-(6-bromo-1H-benzo[d]imidazol-2-yl)-1H-indazole-5-carboxamide (6n)

Compound (6n) of Example 28 was obtained from compound 5n according to General Synthesis Procedure B (yellow oil, yield: 35%).

¹H NMR (400 MHz, METHANOL-d₄, TFA salt) δ 8.98-9.02 (m, 1H), 7.92-7.98 (m, 1H), 7.83 (d, J=1.83 Hz, 1H), 7.68 (dd, J=0.80, 8.82 Hz, 1H), 7.58 (d, J=8.47 Hz, 1H), 7.36-7.43 (m, 1H), 3.56 (t, J=6.64 Hz, 2H), 2.93 (t, J=7.10 Hz, 2H), 1.88-2.00 (m, 2H). MS (ESI): [M+H]⁺=413.2

### Example 29: N-(3-aminopropyl)-3-(6-(trifluoromethyl)-1H-benzo[d]imidazol-2-yl)-1H-indazole-5-carboxamide (60)

Compound (6o) of Example 29 was obtained from compound 5o according to General Synthesis Procedure B (white solid, yield: 75%).

¹H NMR (400 MHz, DMSO-d₆) δ 9.00 (dd, J=0.80, 1.49 Hz, 1H), 8.02 (s, 1H), 7.93 (dd, J=1.60, 8.70 Hz, 1H), 7.83 (d, J=8.47 Hz, 1H), 7.72 (dd, J=0.57, 8.82 Hz, 1H), 7.56 (dd, J=1.49, 8.59 Hz, 1H), 3.38 (t, J=6.75 Hz, 2H), 2.69 (t, J=6.80 Hz, 2H), 1.70-1.77 (m, 2H). MS (ESI): [M+H]⁺=402.85

### Example 30: N-((1S,3S)-3-aminocyclopentyl)-3-(6-(pyrrolidin-1-yl)-1H-benzo[d]imidazol-2-yl)-1H-indazole-5-carboxamide (6t)

Compound (6t) of Example 30 was obtained from compound 5t according to General Synthesis Procedure B (yellow solid, yield: 78%).

¹H NMR (400 MHz, METHANOL-d₄) δ 8.91-8.97 (m, 1H), 7.94 (dd, J=1.72, 8.82 Hz, 1H), 7.64 (dd, J=0.80, 8.82 Hz, 1H), 7.55 (d, J=8.70 Hz, 1H), 6.68-6.76 (m, 2H), 4.59 (quin, J=6.90 Hz, 1H), 3.59 (quin, J=6.87 Hz, 1H), 3.33-3.39 (m, 4H), 2.26-2.37 (m, 1H), 2.15-2.25 (m, 1H), 2.01-2.12 (m, 5H), 1.87-1.99 (m, 1H), 1.74 (d, J=5.95 Hz, 1H), 1.49 (d, J=0.69 Hz, 1H). MS (ESI): [M+H]⁺=429.3

### Example 31: N-((1S,3S)-3-aminocyclopentyl)-3-(6-(piperidin-1-yl)-1H-benzo[d]imidazol-2-yl)-1H-indazole-5-carboxamide (6u)

Compound (6u) of Example 31 was obtained from compound 5u according to General Synthesis Procedure B (yellow solid, yield: 25%).

¹H NMR (400 MHz, METHANOL-d₄) δ 8.96 (dd, J=0.92, 1.60 Hz, 1H), 7.94 (dd, J=1.60, 8.93 Hz, 1H), 7.65 (dd, J=0.80, 8.82 Hz, 1H), 7.59 (d, J=8.93 Hz, 1H), 7.23 (s, 1H), 7.10 (dd, J=2.29, 8.70 Hz, 1H), 4.58 (quin, J=7.20 Hz, 1H), 3.60 (quin, J=6.93 Hz, 1H), 3.13-3.20 (m, 4H), 2.26-2.36 (m, 1H), 2.15-2.26 (m, 1H), 2.01-2.10 (m, 1H), 1.89-1.98 (m, 1H), 1.75-1.83 (m, 4H), 1.69-1.75 (m, 1H), 1.58-1.67 (m, 2H), 1.44-1.55 (m, 1H). MS (ESI): [M+H]⁺=444.1

### Example 32: N-((1S,3S)-3-aminocyclopentyl)-3-(6-methoxy-1H-benzo[d]imidazol-2-yl)-1H-indazole-5-carboxamide (6v)

Compound (6v) of Example 32 was obtained from compound 5v according to General Synthesis Procedure B (white solid, yield: 64%).

¹H NMR (400 MHz, METHANOL-d₄) δ 8.96 (dd, J=0.80, 1.72 Hz, 1H), 7.95 (dd, J=1.60, 8.93 Hz, 1H), 7.66 (dd, J=0.80, 8.82 Hz, 1H), 7.59 (d, J=8.93 Hz, 1H), 7.18 (d, J=2.29 Hz, 1H), 6.94 (dd, J=2.40, 8.82 Hz, 1H), 4.59 (td, J=7.24, 14.14 Hz, 1H), 3.89 (s, 3H), 3.59 (quin, J=6.93 Hz, 1H), 2.27-2.36 (m, 1H), 2.15-2.26 (m, 1H), 2.01-2.10 (m, 1H), 1.87-1.99 (m, 1H), 1.68-1.80 (m, 1H), 1.44-1.55 (m, 1H). MS (ESI): [M+H]⁺=391.1

### Example 33: N-(3-acetamidopropyl)-3-(6-(piperidin-1-yl)-1H-benzo[d]imidazol-2-yl)-1H-indazole-5-carboxamide (36c)

Compound (36c) of Example 33 was obtained from compound 6c according to General Synthesis Procedure F (white solid, yield: 17%).

¹H NMR (400 MHz, METHANOL-d₄) δ 8.99 (dd, J=0.69, 1.60 Hz, 1H), 7.95 (dd, J=1.72, 8.82 Hz, 1H), 7.65 (dd, J=0.92, 8.93 Hz, 1H), 7.60 (br. s, 1H), 7.10 (dd, J=1.14, 8.70 Hz, 1H), 7.18 (br. s, 1H), 3.50 (t, J=6.98 Hz, 2H), 3.33-3.34 (m, 1H), 3.11-3.21 (m, 4H), 1.97 (s, 3H), 1.87 (quin, J=6.90 Hz, 2H), 1.79 (td, J=5.70, 11.28 Hz, 4H), 1.59-1.68 (m, 2H). MS (ESI): [M+H]⁺=460.5

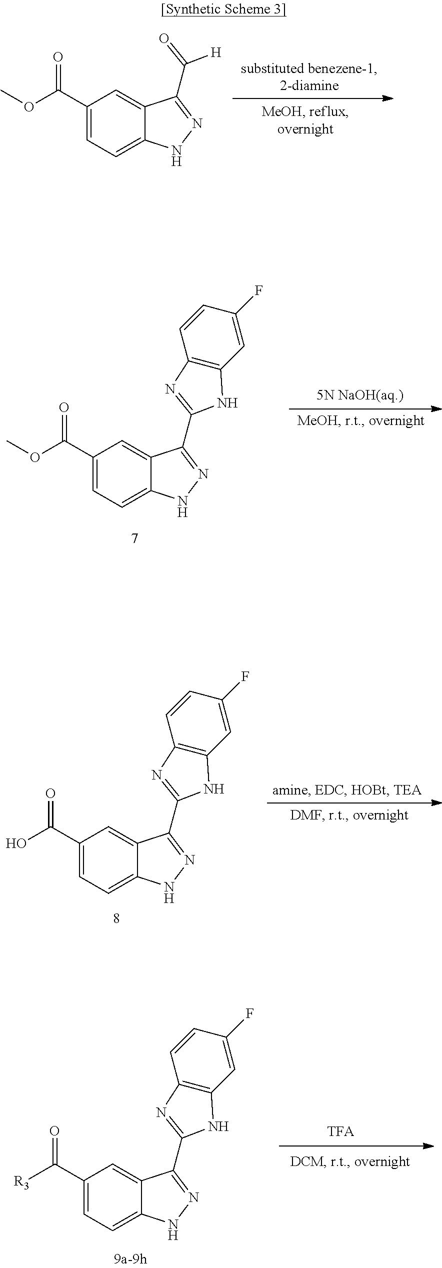

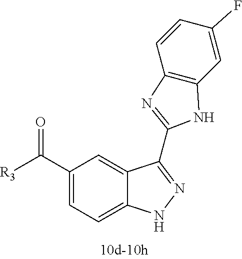

### methyl 3-(6-fluoro-1H-benzo[d]imidazol-2-yl)-1H-indazole-5-carboxylate (7)

Compound 7 was obtained by using 4-fluorobenzene-1,2-diamine according to General Synthesis Procedure C (light orange solid, yield: 24%).

¹H NMR (400 MHz, METHANOL-d₄) δ 9.22 (d, J=1.83 Hz, 1H), 8.11 (dd, J=1.80, 8.55 Hz, 1H), 7.66 (d, J=7.93 Hz, 2H), 7.37 (br. s., 1H), 7.08 (dt, J=2.44, 9.16 Hz, 1H), 3.98 (s, 3H). MS (ESI): [M+H]⁺=311.4

### 3-(6-fluoro-1H-benzo[d]imidazol-2-yl)-1H-indazole-5-carboxylic acid (8)

Compound 8 was obtained from compound 7 according to General Synthesis Procedure D (light brown solid, yield: 83%).

¹H NMR (400 MHz, DMSO-d₆) δ 14.19 (br. s., 1H), 9.18 (d, J=2.44 Hz, 1H), 8.04 (dd, J=1.53, 8.85 Hz, 1H), 7.76 (d, J=9.77 Hz, 1H), 7.70 (dd, J=4.88, 8.55 Hz, 1H), 7.46-7.53 (m, 1H), 7.17 (dt, J=2.44, 9.46 Hz, 1H). MS (ESI): [M+H]⁺=296.9

### Example 34: (3-(6-fluoro-1H-benzo[d]imidazol-2-yl)-1H-indazol-5-yl)(4-(4-methylpiperazin-1-yl) piperidin-1-yl)methanone (9a)

Compound (9a) of Example 34 was obtained from compound 8 by using 1-methyl-4-(piperidin-4-yl)piperazine according to General Synthesis Procedure A (light brown solid, yield: 64%).

¹H NMR (400 MHz, DMSO-d₆) δ 8.55-8.57 (m, 1H), 7.68 (dd, J=0.80, 8.59 Hz, 1H), 7.62 (br. s, 1H), 7.48 (dd, J=1.60, 8.70 Hz, 1H), 7.38 (br. s, 1H), 7.06 (ddd, J=2.52, 8.87, 9.90 Hz, 1H), 4.11 (br. s, 2H), 2.95-3.14 (m, 4H), 2.30-2.35 (m, 4H), 2.16 (s, 3H), 1.76-1.84 (m, 2H), 1.39-1.52 (m, 2H). MS (ESI): [M+H]⁺=462.5

### Example 35: N-(2-(dimethylamino)ethyl)-3-(6-fluoro-1H-benzo[d]imidazol-2-yl)-1H-indazole-5-carboxamide (9b)

Compound (9b) of Example 35 was obtained from compound 8 by using N,N-dimethylethane-1,2-diamine according to General Synthesis Procedure A (yellowish solid, yield: 39%).

¹H NMR (400 MHz, METHANOL-d₄) δ 8.94-9.05 (m, 1H), 7.93-7.99 (m, 1H), 7.58-7.71 (m, 2H), 7.29-7.42 (m, 1H), 7.04-7.12 (m, 1H), 3.62 (t, J=6.87 Hz, 2H), 2.67 (t, J=6.98 Hz, 2H), 2.38 (s, 6H) MS (ESI): [M+H]⁺=367.2

### Example 36: N-(3-(dimethylamino)propyl)-3-(6-fluoro-1H-benzo[d]imidazol-2-yl)-1H-indazole-5-carboxamide (9c)

Compound (9c) of Example 36 was obtained from compound 8 by using N,N-dimethylpropane-1,3-diamine according to General Synthesis Procedure A (yellowish solid, yield: 48%).

¹H NMR (400 MHz, METHANOL-d₄) δ 8.94-8.97 (m, 1H), 7.92 (dd, J=1.72, 8.82 Hz, 1H), 7.58-7.66 (m, 2H), 7.33 (dd, J=2.29, 9.16 Hz, 1H), 7.05 (ddd, J=2.52, 8.82, 9.73 Hz, 1H), 3.42-3.56 (m, 2H), 2.42-2.54 (m, 2H), 2.28 (s, 6H), 1.77-1.90 (m, 2H). MS (ESI): [M+H]⁺=381.0

### tert-butyl (2-(3-(6-fluoro-1H-benzo[d]imidazol-2-yl)-1H-indazole-5-carboxamido)ethyl) carbamate (9d)

Compound 9d was obtained from compound 8 by using 2-aminoethyl tert-butylcarbamate according to General Synthesis Procedure A (white solid, yield: 42%).

¹H NMR (400 MHz, METHANOL-d₄) δ 8.97-9.05 (m, 1H), 7.92-8.00 (m, 1H), 7.51-7.80 (m, 2H), 7.20-7.48 (m, 1H), 7.03-7.15 (m, 1H), 3.52-3.56 (m, 2H), 3.33-3.36 (m, 2H), 1.38-1.43 (m, 9H). MS (ESI): [M+H]⁺=439.5

### tert-butyl (3-(3-(6-fluoro-1H-benzo[d]imidazol-2-yl)-1H-indazole-5-carboxamido)propyl) carbamate (9e)

Compound 9e was obtained from compound 8 by using 3-aminopropyl tert-butylcarbamate according to General Synthesis Procedure A (light brown solid, yield: 37%).

¹H NMR (400 MHz, DMSO-d₆) δ 8.97 (dd, J=0.80, 1.49 Hz, 1H), 8.62 (t, J=5.72 Hz, 1H), 7.90-7.95 (m, 2H), 7.69 (dd, J=0.69, 8.70 Hz, 1H), 7.65 (br. s., 1H), 7.44 (br. s., 1H), 7.09 (ddd, J=2.52, 8.76, 9.79 Hz, 1H), 6.84 (t, J=5.72 Hz, 1H), 3.28-3.34 (m, 2H), 3.00 (q, J=6.72 Hz, 2H), 1.67 (quin, J=6.81 Hz, 2H), 1.36 (s, 9H). MS (ESI): [M+H]⁺=452.96

### tert-butyl (4-(3-(6-fluoro-1H-benzo[d]imidazol-2-yl)-1H-indazole-5-carboxamido)butyl) carbamate (9f)

Compound 9f was obtained from compound 8 by using 4-aminobutyl tert-butylcarbamate according to General Synthesis Procedure A (white solid, yield: 35%).

¹H NMR (400 MHz, METHANOL-d₄) δ 8.92-9.02 (m, 1H), 7.93-7.99 (m, 1H), 7.61-7.75 (m, 2H), 7.40-7.51 (m, 1H), 7.01-7.15 (m, 1H), 3.43-3.51 (m, 2H), 3.09-3.15 (m, 2H), 1.66-1.74 (m, 2H), 1.57-1.64 (m, 2H), 1.42 (d, J=4.35 Hz, 9H). MS (ESI): [M+H]⁺=467.2

### tert-butyl 4-(3-(6-fluoro-1H-benzo[d]imidazol-2-yl)-1H-indazole-5-carboxamido)piperidine-1-carboxylate (9g)

Compound 9g was obtained from compound 8 by using tert-butyl 4-aminopiperidine-1-carboxylate according to General Synthesis Procedure A (yellow oil, yield: 49%). MS (ESI): [M+H]⁺=479.2

### tert-butyl (R)-3-(3-(6-fluoro-1H-benzo[d]imidazol-2-yl)-1H-indazole-5-carboxamido)piperidine-1-carboxylate (9h)

Compound 9 h was obtained from compound 8 by using tert-butyl (R)-3-aminopiperidine-1-carboxylate according to General Synthesis Procedure A (brown oil, yield: 55%).

¹H NMR (400 MHz, DMSO-d₆) δ 8.89-8.98 (m, 1H), 8.38-8.48 (m, 1H), 7.85-7.93 (m, 1H), 7.61-7.69 (m, 1H), 7.43-7.61 (m, 1H), 6.98-7.12 (m, 1H), 3.87-4.10 (m, 1H), 3.72-3.87 (m, 2H), 3.24-3.28 (m, 1H), 2.66-2.78 (m, 1H), 1.84-1.92 (m, 1H), 1.65-1.75 (m, 1H), 1.48-1.61 (m, 1H), 1.29-1.45 (m, 10H). MS (ESI): [M+H]⁺=479.2

### Example 37: N-(2-aminoethyl)-3-(6-fluoro-1H-benzo[d]imidazol-2-yl)-1H-indazole-5-carboxamide (10d)

Compound (10d) of Example 37 was obtained from compound 9d according to General Synthesis Procedure B (white solid, yield: 42%).

¹H NMR (400 MHz, DMSO-d₆) δ 8.98 (dd, J=0.69, 1.60 Hz, 1H), 8.59 (t, J=5.61 Hz, 1H), 7.95 (dd, J=1.60, 8.70 Hz, 1H), 7.69 (dd, J=0.80, 8.82 Hz, 1H), 7.49 (br. s, 2H), 7.06-7.14 (m, 1H), 3.31-3.36 (m, 3H), 2.74 (t, J=6.64 Hz, 2H). MS (ESI): [M+H]⁺=338.8

### Example 38: N-(3-aminopropyl)-3-(6-fluoro-1H-benzo[d]imidazol-2-yl)-1H-indazole-5-carboxamide (10e)

Compound (10e) of Example 38 was obtained from compound 9e according to General Synthesis Procedure B (yellowish solid, yield: 42%).

¹H NMR (400 MHz, DMSO-d₆) δ 8.97-9.03 (m, 1H), 8.73 (t, J=5.61 Hz, 1H), 7.92 (dd, J=1.60, 8.70 Hz, 1H), 7.69 (dd, J=0.80, 8.82 Hz, 2H), 7.09 (ddd, J=2.52, 8.70, 9.85 Hz, 1H), 3.34-3.43 (m, 2H), 2.65 (t, J=6.64 Hz, 2H), 1.66 (t, J=6.75 Hz, 2H). MS (ESI): [M+H]⁺=353.2

### Example 39: N-(4-aminobutyl)-3-(6-fluoro-1H-benzo[d]imidazol-2-yl)-1H-indazole-5-carboxamide (10f)

Compound (10f) of Example 39 was obtained from compound 9f according to General Synthesis Procedure B (white solid, yield: 35%).

¹H NMR (400 MHz, METHANOL-d₄) δ 8.97-9.00 (m, 1H), 7.95 (dd, J=1.60, 8.70 Hz, 1H), 7.62-7.69 (m, 2H), 7.37 (dd, J=2.52, 9.16 Hz, 1H), 7.04-7.11 (m, 1H), 3.48 (t, J=6.87 Hz, 2H), 2.75 (t, J=7.10 Hz, 2H), 1.69-1.78 (m, 2H), 1.58-1.68 (m, 2H). MS (ESI): [M+H]⁺=366.9

### Example 40: 3-(6-fluoro-1H-benzo[d]imidazol-2-yl)-N-(piperidin-4-yl)-1H-indazole-5-carboxamide (10g)

Compound (10g) of Example 40 was obtained from compound 9g according to General Synthesis Procedure B (yellowish solid, yield: 41%).

¹H NMR (400 MHz, DMSO-d₆) δ 8.93-8.97 (m, 1H), 8.41-8.48 (m, 1H), 7.90-7.96 (m, 1H), 7.58-7.71 (m, 2H), 7.37-7.55 (m, 1H), 7.02-7.13 (m, 1H), 3.82-3.93 (m, 1H), 2.93-3.02 (m, 2H), 2.51-2.60 (m, 2H), 1.72-1.82 (m, 2H), 1.38-1.52 (m, 2H). MS (ESI): [M+H]⁺=379.0

### Example 41: (R)-3-(6-fluoro-1H-benzo[d]imidazol-2-yl)-N-(piperidin-3-yl)-1H-indazole-5-carboxamide (10h)

Compound (10h) of Example 41 was obtained from compound 9 h according to General Synthesis Procedure B (white solid, yield: 46%).

¹H NMR (400 MHz, METHANOL-d₄) δ 8.96-9.01 (m, 1H), 7.96 (dd, J=1.60, 8.70 Hz, 1H), 7.63-7.72 (m, 2H), 7.32-7.43 (m, 1H), 7.09 (ddd, J=2.52, 8.82, 9.73 Hz, 1H), 4.00-4.15 (m, 1H), 3.18-3.29 (m, 1H), 2.94-3.02 (m, 1H), 2.51-2.67 (m, 2H), 2.04-2.19 (m, 1H), 1.82-1.94 (m, 1H), 1.55-1.77 (m, 2H). MS (ESI): [M+H]⁺=378.9

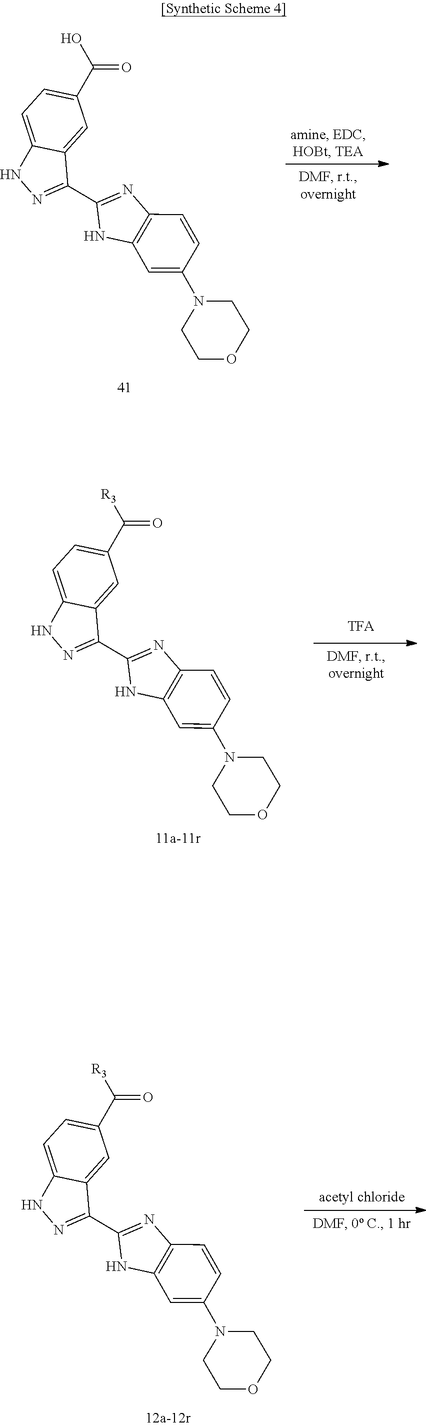

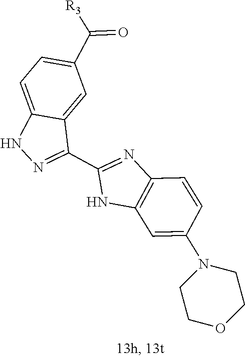

### tert-butyl (2-(3-(6-morpholino-1H-benzo[d]imidazol-2-yl)-1H-indazole-5-carboxamido)ethyl)carbamate (11a)

Compound 11a was obtained from compound 4f by using tert-butyl (2-aminoethyl)carbamate according to General Synthesis Procedure A (yellow oil, yield: 65%).

¹H NMR (400 MHz, METHANOL-d₄) δ 9.00 (s, 1H), 7.93-7.99 (m, 1H), 7.57-7.73 (m, 2H), 6.99-7.20 (m, 2H), 3.82-3.93 (m, 4H), 3.49-3.59 (m, 2H), 3.33-3.45 (m, 2H), 3.10-3.23 (m, 4H), 1.40 (s, 9H). MS (ESI): [M+H]⁺=506.2

### tert-butyl (4-(3-(6-morpholino-1H-benzo[d]imidazol-2-yl)-1H-indazole-5-carboxamido)butyl)carbamate (l1b)

Compound 11b was obtained from compound 4f by using tert-butyl (4-aminobutyl)carbamate according to General Synthesis Procedure A (yellow oil, yield: 55%).

¹H NMR (400 MHz, METHANOL-d₄) δ 8.98 (dd, J=0.92, 1.60 Hz, 1H), 7.92-7.99 (m, 1H), 7.65 (dd, J=0.80, 8.82 Hz, 2H), 7.03-7.18 (m, 2H), 3.82-3.92 (m, 4H), 3.48 (t, J=6.98 Hz, 2H), 3.16-3.22 (m, 4H), 3.08-3.14 (m, 2H), 1.67-1.76 (m, 2H), 1.58-1.67 (m, 2H), 1.42 (s, 9H). MS (ESI): [M+H]⁺=534.2

### tert-butyl methyl(3-(3-(6-morpholino-1H-benzo[d]imidazol-2-yl)-1H-indazole-5-carboxamido)propyl)carbamate (tic)

Compound 11c was obtained from compound 4f by using tert-butyl (3-aminopropyl)(methyl)carbamate according to General Synthesis Procedure A (yellow oil, yield: 45%).

¹H NMR (400 MHz, METHANOL-d₄) δ 8.98-9.02 (m, 1H), 7.93-8.00 (m, 1H), 7.53-7.72 (m, 2H), 7.04-7.20 (m, 2H), 3.84-3.95 (m, 4H), 3.43-3.52 (m, 2H), 3.35-3.43 (m, 2H), 3.13-3.23 (m, 4H), 2.89 (s, 3H), 1.82-1.99 (m, 2H), 1.40 (s, 9H). MS (ESI): [M+H]⁺=534.8

### tert-butyl (2-methyl-3-(3-(6-morpholino-1H-benzo[d]imidazol-2-yl)-1H-indazole-5-carboxamido)propyl)carbamate (11d)

Compound 11d was obtained from compound 4f by using tert-butyl (3-amino-2-methylpropyl)carbamate according to General Synthesis Procedure A (brown oil, yield: 56%).

¹H NMR (400 MHz, METHANOL-d₄) δ 8.94-9.04 (m, 1H), 7.92-7.98 (m, 1H), 7.45-7.74 (m, 2H), 7.03-7.33 (m, 2H), 3.82-3.91 (m, 4H), 3.36-3.42 (m, 2H), 3.15-3.22 (m, 4H), 3.03-3.15 (m, 2H), 1.92-2.07 (m, 1H), 1.40 (s, 9H), 0.97-1.07 (m, 3H). MS (ESI): [M+H]⁺=534.4

### tert-butyl 3-(3-(6-morpholino-1H-benzo[d]imidazol-2-yl)-1H-indazole-5-carboxamido)azetidine-1-carboxylate (11e)

Compound 11e was obtained from compound 4f by using tert-butyl 3-aminoazetidine-1-carboxylat according to General Synthesis Procedure A (brown oil, yield: 50%).

¹H NMR (400 MHz, METHANOL-d₄) δ 9.00-9.06 (m, 1H), 7.90-7.99 (m, 1H), 7.52-7.73 (m, 2H), 6.98-7.22 (m, 2H), 4.77-4.82 (m, 1H), 4.28-4.36 (m, 2H), 3.98-4.09 (m, 2H), 3.85-3.93 (m, 4H), 3.14-3.22 (m, 4H), 1.48 (s, 9H). MS (ESI): [M+H]⁺=518.2

### tert-butyl 3-((3-(6-morpholino-1H-benzo[d]imidazol-2-yl)-1H-indazole-5-carboxamido)methyl)azetidine-1-carboxylate (11f)

Compound 11f was obtained from compound 4f by using tert-butyl 3-(aminomethyl)azetidine-1-carboxylate according to General Synthesis Procedure A (brown oil, yield: 30%).

¹H NMR (400 MHz, METHANOL-d₄) δ 8.93-8.97 (m, 1H), 7.90-7.95 (m, 1H), 7.61-7.69 (m, 2H), 6.99-7.13 (m, 2H), 4.00-4.07 (m, 2H), 3.85-3.89 (m, 4H), 3.73-3.79 (m, 2H), 3.63-3.66 (m, 2H), 3.13-3.19 (m, 4H), 2.87-2.94 (m, 1H), 1.39 (s, 9H). MS (ESI): [M+H]⁺=532.9

### tert-butyl 3-(2-(3-(6-morpholino-1H-benzo[d]imidazol-2-yl)-1H-indazole-5-carboxamido)ethyl)azetidine-1-carboxylate (11g)

Compound 11g was obtained from compound 4f by using tert-butyl 3-(2-aminoethyl)azetidine-1-carboxylate according to General Synthesis Procedure A (brown oil, yield: 32%).

¹H NMR (400 MHz, METHANOL-d₄) δ 8.92-9.03 (m, 1H), 7.91-7.96 (m, 1H), 7.57-7.76 (m, 2H), 7.00-7.27 (m, 2H), 4.01-4.12 (m, 2H), 3.83-3.92 (m, 4H), 3.60-3.68 (m, 2H), 3.41-3.50 (m, 2H), 3.12-3.23 (m, 4H), 2.65-2.77 (m, 1H), 1.90-2.03 (m, 2H), 1.40 (s, 9H). MS (ESI): [M+H]⁺=546.4

### tert-butyl 6-(3-(6-morpholino-1H-benzo[d]imidazol-2-yl)-1H-indazole-5-carbonyl)-2,6-diazaspiro[3.3]heptane-2-carboxylate (11h)

Compound 11 h was obtained from compound 4f by using tert-butyl 2,6-diazaspiro[3.3]heptane-2-carboxylate according to General Synthesis Procedure A (brown oil, yield: 23%).

¹H NMR (400 MHz, METHANOL-d₄) δ 8.69-8.80 (m, 1H), 7.75 (dd, J=1.49, 8.82 Hz, 1H), 7.65 (dd, J=0.80, 8.82 Hz, 2H), 6.96-7.25 (m, 2H), 4.65 (br. s., 2H), 4.34 (br. s., 2H), 4.12 (s, 4H), 3.76-3.94 (m, 4H), 3.06-3.20 (m, 4H), 1.43 (s, 9H). MS (ESI): [M+H]⁺=544.1

### tert-butyl ((1R,3S)-3-(3-(6-morpholino-1H-benzo[d]imidazol-2-yl)-1H-indazole-5-carboxamido)cyclopentyl)carbamate (11i)

Compound 11i was obtained from compound 4f by using tert-butyl ((1R,3S)-3-aminocyclopentyl)carbamate according to General Synthesis Procedure A (brown oil, yield: 53%).

¹H NMR (400 MHz, METHANOL-d₄) δ 8.91-9.02 (m, 1H), 7.92-7.97 (m, 1H), 7.58-7.71 (m, 2H), 7.14-7.24 (m, 1H), 7.02-7.14 (m, 1H), 4.33-4.46 (m, 1H), 3.92-4.00 (m, 1H), 3.81-3.91 (m, 4H), 3.04-3.25 (m, 4H), 2.43-2.57 (m, 1H), 1.96-2.18 (m, 2H), 1.69-1.88 (m, 2H), 1.55-1.62 (m, 1H), 1.42 (s, 9H). MS (ESI): [M+H]⁺=546.4

### tert-butyl ((1S,3S)-3-(3-(6-morpholino-1H-benzo[d]imidazol-2-yl)-1H-indazole-5-carboxamido)cyclopentyl)carbamate (11j)

Compound 11j was obtained from compound 4f by using tert-butyl ((1S,3S)-3-aminocyclopentyl)carbamate according to General Synthesis Procedure A (brown oil, yield: 56%).

¹H NMR (400 MHz, METHANOL-d₄) δ 8.95-8.98 (m, 1H), 7.91-7.98 (m, 1H), 7.57-7.68 (m, 2H), 7.14-7.20 (m, 1H), 7.04-7.13 (m, 1H), 4.46-4.59 (m, 1H), 4.05-4.18 (m, 1H), 3.84-3.91 (m, 4H), 3.12-3.23 (m, 4H), 2.15-2.27 (m, 2H), 1.98-2.04 (m, 2H), 1.65-1.73 (m, 1H), 1.51-1.59 (m, 1H), 1.45 (s, 9H). MS (ESI): [M+H]⁺=546.6

### tert-butyl (R)-3-(3-(6-morpholino-1H-benzo[d]imidazol-2-yl)-1H-indazole-5-carboxamido)pyrrolidine-1-carboxylate (11k)

Compound 11k was obtained from compound 4f by using tert-butyl (R)-3-aminopyrrolidine-1-carboxylate according to General Synthesis Procedure A (orange oil, yield: 45%).

¹H NMR (400 MHz, METHANOL-d₄) δ 9.00 (s, 1H), 7.93-7.99 (m, 1H), 7.56-7.74 (m, 2H), 7.00-7.21 (m, 2H), 4.56-4.70 (m, 1H), 3.80-3.93 (m, 4H), 3.72-3.80 (m, 1H), 3.57-3.67 (m, 1H), 3.45-3.54 (m, 1H), 3.35-3.44 (m, 1H), 3.15-3.25 (m, 4H), 2.23-2.38 (m, 1H), 2.06-2.14 (m, 1H), 1.46 (s, 9H). MS (ESI): [M+H]⁺=532.3

### tert-butyl (S)-3-(3-(6-morpholino-1H-benzo[d]imidazol-2-yl)-1H-indazole-5-carboxamido)pyrrolidine-1-carboxylate (11l)

Compound 11l was obtained from compound 4f by using tert-butyl (R)-3-aminopyrrolidine-1-carboxylate according to General Synthesis Procedure A (brown oil, yield: 47%).

¹H NMR (400 MHz, METHANOL-d₄) δ 8.99 (s, 1H), 7.96 (dd, J=1.72, 8.83 Hz, 1H), 7.56-7.74 (m, 2H), 6.99-7.20 (m, 2H), 4.58-4.66 (m, 1H), 3.86-3.92 (m, 4H), 3.72-3.81 (m, 1H), 3.56-3.64 (m, 1H), 3.45-3.53 (m, 1H), 3.37-3.44 (m, 1H), 3.17-3.23 (m, 4H), 2.24-2.36 (m, 1H), 2.03-2.19 (m, 1H), 1.52 (s, 9H). MS (ESI): [M+H]⁺=532.3

### tert-butyl (S)-3-((3-(6-morpholino-1H-benzo[d]imidazol-2-yl)-1H-indazole-5-carboxamido)methyl)pyrrolidine-1-carboxylate (11m)

Compound 11m was obtained from compound 4f by using tert-butyl (S)-3-(aminomethyl)pyrrolidine-1-carboxylate according to General Synthesis Procedure A (brown oil, yield: 41%).

¹H NMR (400 MHz, METHANOL-d₄) δ 8.94-8.99 (m, 1H), 7.94 (d, J=8.93 Hz, 1H), 7.54-7.68 (m, 2H), 7.13-7.20 (m, 1H), 7.08 (dd, J=2.29, 8.70 Hz, 1H), 3.79-3.91 (m, 4H), 3.40-3.61 (m, 4H), 3.33-3.40 (m, 1H), 3.10-3.20 (m, 5H), 2.48-2.66 (m, 1H), 1.99-2.15 (m, 1H), 1.71-1.83 (m, 1H), 1.45 (s, 9H). MS (ESI): [M+H]⁺=546.4

### tert-butyl (R)-3-((3-(6-morpholino-1H-benzo[d]imidazol-2-yl)-1H-indazole-5-carboxamido)methyl)pyrrolidine-1-carboxylate (tin)

Compound 1In was obtained from compound 4f by using tert-butyl (R)-3-(aminomethyl)pyrrolidine-1-carboxylate according to General Synthesis Procedure A (brown oil, yield: 44%).

¹H NMR (400 MHz, METHANOL-d₄) δ 8.97-9.02 (m, 1H), 7.91-7.98 (m, 1H), 7.62-7.69 (m, 1H), 7.54-7.62 (m, 1H), 7.13-7.20 (m, 1H), 7.06-7.12 (m, 1H), 3.86-3.92 (m, 4H), 3.33-3.63 (m, 5H), 3.12-3.22 (m, 5H), 2.53-2.63 (m, 1H), 1.98-2.15 (m, 1H), 1.72-1.85 (m, 1H), 1.46 (s, 9H). MS (ESI): [M+H]⁺=546.4

### tert-butyl 3-((3-(6-morpholino-1H-benzo[d]imidazol-2-yl)-1H-indazole-5-carboxamido)methyl)piperidine-1-carboxylate (11o)

Compound 11o was obtained from compound 4f by using tert-butyl 3-(aminomethyl)piperidine-1-carboxylate according to General Synthesis Procedure A (brown oil, yield: 42%).

¹H NMR (400 MHz, DMSO-d₆) δ 8.95-9.02 (m, 1H), 7.87-7.95 (m, 1H), 7.60-7.72 (m, 2H), 6.90-7.06 (m, 2H), 3.77-3.81 (m, 4H), 3.16-3.24 (m, 2H), 3.04-3.16 (m, 4H), 2.73-2.82 (m, 1H), 1.57-1.84 (m, 4H), 1.14-1.44 (m, 13H). MS (ESI): [M+H]⁺=560.4

### tert-butyl 4-((3-(6-morpholino-1H-benzo[d]imidazol-2-yl)-1H-indazole-5-carboxamido)methyl)piperidine-1-carboxylate (11p)

Compound 11p was obtained from compound 4f by using tert-butyl 4-(aminomethyl)piperidine-1-carboxylate according to General Synthesis Procedure A (brown oil, yield: 53%).

¹H NMR (400 MHz, METHANOL-d₄) δ 8.97-9.00 (m, 1H), 7.93-7.98 (m, 1H), 7.54-7.72 (m, 2H), 7.02-7.25 (m, 2H), 4.05-4.15 (m, 2H), 3.80-3.96 (m, 4H), 3.33-3.39 (m, 2H), 3.14-3.21 (m, 4H), 2.65-2.87 (m, 2H), 1.86-1.94 (m, 1H), 1.75-1.85 (m, 2H), 1.44 (s, 9H), 1.07-1.29 (m, 2H). MS (ESI): [M+H]⁺=560.9

### tert-butyl 4-(3-(6-morpholino-1H-benzo[d]imidazol-2-yl)-1H-indazole-5-carboxamido)piperidine-1-carboxylate (11q)

Compound 11q was obtained from compound 4f by using tert-butyl 4-aminopiperidine-1-carboxylate according to General Synthesis Procedure A (brown oil, yield: 47%).

¹H NMR (400 MHz, METHANOL-d₄) δ 8.93-9.00 (m, 1H), 7.92-7.99 (m, 1H), 7.62-7.71 (m, 2H), 7.04-7.16 (m, 2H), 4.08-4.18 (m, 4H), 3.84-3.92 (m, 5H), 3.17-3.22 (m, 4H), 2.01-2.05 (m, 2H), 1.56-1.62 (m, 2H), 1.50 (s, 9H). MS (ESI): [M+H]⁺=546.2

### tert-butyl ((1s,4s)-4-(3-(6-morpholino-1H-benzo[d]imidazol-2-yl)-1H-indazole-5-carboxamido)cyclohexyl)carbamate (11r)

Compound 11r was obtained from compound 4f by using tert-butyl ((1s,4s)-4-aminocyclohexyl)carbamate according to General Synthesis Procedure A (brown oil, yield: 56%).

¹H NMR (400 MHz, METHANOL-d₄) δ 8.93-9.00 (m, 1H), 7.96 (dd, J=1.60, 8.70 Hz, 1H), 7.66 (dd, J=0.69, 8.70 Hz, 2H), 6.99-7.19 (m, 2H), 3.93-4.10 (m, 1H), 3.84-3.93 (m, 4H), 3.62-3.68 (m, 1H), 3.18-3.23 (m, 4H), 1.75-1.89 (m, 8H), 1.47 (s, 9H). MS (ESI): [M+H]⁺=560.1

### Example 42: N-(2-aminoethyl)-3-(6-morpholino-1H-benzo[d]imidazol-2-yl)-1H-indazole-5-carboxamide (12a)

Compound (12a) of Example 42 was obtained from compound 11a according to General Synthesis Procedure B (yellow oil, yield: 75%).

¹H NMR (400 MHz, METHANOL-d₄) δ 8.99-9.04 (m, 1H), 7.92-8.01 (m, 1H), 7.63-7.70 (m, 1H), 7.61 (d, J=8.93 Hz, 1H), 7.19 (s, 1H), 7.10 (dd, J=2.18, 8.82 Hz, 1H), 3.84-3.93 (m, 4H), 3.57 (t, J=6.18 Hz, 2H), 3.16-3.21 (m, 4H), 2.96 (t, J=6.18 Hz, 2H). MS (ESI): [M+H]⁺=406.5

### Example 43: N-(4-aminobutyl)-3-(6-morpholino-1H-benzo[d]imidazol-2-yl)-1H-indazole-5-carboxamide (12b)

Compound (12b) of Example 43 was obtained from compound 11b according to General Synthesis Procedure B (yellow oil, yield: 73%).

¹H NMR (400 MHz, METHANOL-d₄) δ 8.99 (dd, J=0.92, 1.60 Hz, 1H), 7.93-7.99 (m, 1H), 7.66 (dd, J=0.80, 8.82 Hz, 1H), 7.61 (d, J=8.93 Hz, 1H), 7.20 (d, J=2.06 Hz, 1H), 7.10 (dd, J=2.29, 8.93 Hz, 1H), 3.84-3.94 (m, 4H), 3.47-3.53 (m, 2H), 3.14-3.24 (m, 4H), 2.74-2.86 (m, 2H), 1.63-1.78 (m, 4H). MS (ESI): [M+H]⁺=434.7

### Example 44: N-(3-(methylamino)propyl)-3-(6-morpholino-1H-benzo[d]imidazol-2-yl)-1H-indazole-5-carboxamide (12c)

Compound (12c) of Example 44 was obtained from compound I1c according to General Synthesis Procedure B (yellow solid, yield: 43%).

¹H NMR (400 MHz, METHANOL-d₄) δ 8.96-9.00 (m, 1H), 7.92-7.98 (m, 1H), 7.66 (dd, J=0.80, 8.82 Hz, 1H), 7.60 (d, J=8.70 Hz, 1H), 7.18 (d, J=2.06 Hz, 1H), 7.09 (dd, J=2.29, 8.70 Hz, 1H), 3.82-3.92 (m, 4H), 3.52 (t, J=6.87 Hz, 2H), 3.14-3.20 (m, 4H), 2.70 (t, J=7.21 Hz, 2H), 2.42 (s, 3H), 1.85-1.94 (m, 2H). MS (ESI): [M+H]⁺=434.0

### Example 45: N-(3-amino-2-methylpropyl)-3-(6-morpholino-1H-benzo[d]imidazol-2-yl)-1H-indazole-5-carboxamide (12d)

Compound (12d) of Example 45 was obtained from compound lid according to General Synthesis Procedure B (yellow oil, yield: 33%).

¹H NMR (400 MHz, METHANOL-d₄) δ 8.94-9.00 (m, 1H), 7.91-7.97 (m, 1H), 7.57-7.67 (m, 2H), 7.17 (d, J=2.29 Hz, 1H), 7.07 (dd, J=2.29, 8.70 Hz, 1H), 3.87 (dd, J=3.89, 5.72 Hz, 4H), 3.40-3.46 (m, 2H), 3.15-3.23 (m, 4H), 2.64-2.74 (m, 1H), 2.58 (dd, J=6.41, 12.82 Hz, 1H), 1.88-1.98 (m, 1H), 0.99-1.07 (m, 3H). MS (ESI): [M+H]⁺=434.0

### Example 46: N-(azetidin-3-yl)-3-(6-morpholino-1H-benzo[d]imidazol-2-yl)-1H-indazole-5-carboxamide (12e)

Compound (12e) of Example 46 was obtained from compound 11e according to General Synthesis Procedure B (yellow oil, yield: 42%).

¹H NMR (400 MHz, METHANOL-d₄) δ 9.04 (dd, J=0.92, 1.60 Hz, 1H), 7.99 (dd, J=1.60, 8.70 Hz, 1H), 7.67-7.74 (m, 1H), 7.62 (dd, J=0.46, 8.93 Hz, 1H), 7.20 (d, J=2.06 Hz, 1H), 7.13 (dd, J=2.29, 8.93 Hz, 1H), 4.91-5.00 (m, 1H), 4.36-4.46 (m, 4H), 3.84-3.97 (m, 4H), 3.13-3.24 (m, 4H). MS (ESI): [M+H]⁺=417.3

### Example 47: N-(azetidin-3-ylmethyl)-3-(6-morpholino-1H-benzo[d]imidazol-2-yl)-1H-indazole-5-carboxamide (12f)

Compound (12f) of Example 47 was obtained from compound 11f according to General Synthesis Procedure B (yellow oil, yield: 45%).

¹H NMR (400 MHz, METHANOL-d₄) δ 8.97-9.00 (m, 1H), 7.92-7.98 (m, 1H), 7.65-7.70 (m, 1H), 7.57-7.63 (m, 1H), 7.16-7.21 (m, 1H), 7.10 (dd, J=2.29, 8.93 Hz, 1H), 3.87-3.92 (m, 4H), 3.73-3.83 (m, 2H), 3.65-3.71 (m, 2H), 3.59-3.65 (m, 2H), 3.17-3.23 (m, 4H), 3.09-3.17 (m, 1H). MS (ESI): [M+H]⁺=432.5

### Example 48: N-(2-(azetidin-3-yl)ethyl)-3-(6-morpholino-1H-benzo[d]imidazol-2-yl)-1H-indazole-5-carboxamide (12g)

Compound (12g) of Example 48 was obtained from compound 11g according to General Synthesis Procedure B (yellow oil, yield: 37%).

¹H NMR (400 MHz, METHANOL-d₄) δ 8.95-9.00 (m, 1H), 7.91-7.98 (m, 1H), 7.63-7.69 (m, 1H), 7.56-7.62 (m, 1H), 7.14-7.20 (m, 1H), 7.07-7.11 (m, 1H), 3.82-3.93 (m, 4H), 3.68-3.77 (m, 2H), 3.38-3.52 (m, 4H), 3.13-3.21 (m, 4H), 2.88-3.00 (m, 1H), 1.90-2.02 (m, 2H). MS (ESI): [M+H]⁺=446.1

### Example 49: (3-(6-morpholino-1H-benzo[d]imidazol-2-yl)-1H-indazol-5-yl)(2,6-diazaspiro[3.3]heptan-2-yl)methanone (12h)

Compound (12h) of Example 49 was obtained from compound 11 h according to General Synthesis Procedure B (yellow oil, yield: 45%).

¹H NMR (400 MHz, METHANOL-d₄) δ 8.74-8.79 (m, 1H), 7.75-7.81 (m, 1H), 7.66-7.70 (m, 1H), 7.58-7.65 (m, 1H), 7.14-7.25 (m, 1H), 7.05-7.13 (m, 1H), 4.55-4.78 (m, 2H), 4.33-4.43 (m, 2H), 3.97-4.11 (m, 4H), 3.86-3.92 (m, 4H), 3.16-3.22 (m, 4H). MS (ESI): [M+H]⁺=444.1

### Example 50: N-((1S,3R)-3-aminocyclopentyl)-3-(6-morpholino-1H-benzo[d]imidazol-2-yl)-1H-indazole-5-carboxamide (12i)

Compound (12i) of Example 50 was obtained from compound 11i according to General Synthesis Procedure B (yellow oil, yield: 32%).

¹H NMR (400 MHz, METHANOL-d₄) δ 8.95-9.01 (m, 1H), 7.90-7.98 (m, 1H), 7.56-7.68 (m, 2H), 7.18 (d, J=2.06 Hz, 1H), 7.01-7.11 (m, 1H), 4.36-4.47 (m, 1H), 3.83-3.96 (m, 4H), 3.35-3.55 (m, 1H), 3.09-3.22 (m, 4H), 2.28-2.57 (m, 1H), 1.95-2.18 (m, 2H), 1.81-1.94 (m, 1H), 1.57-1.68 (m, 1H), 1.44-1.57 (m, 1H). MS (ESI): [M+H]⁺=446.6

### Example 51: N-((1S,3S)-3-aminocyclopentyl)-3-(6-morpholino-1H-benzo[d]imidazol-2-yl)-1H-indazole-5-carboxamide (12j)

Compound (12j) of Example 51 was obtained from compound 11j according to General Synthesis Procedure B (yellow oil, yield: 40%).

¹H NMR (400 MHz, METHANOL-d₄) δ 8.93-8.98 (m, 1H), 7.90-7.98 (m, 1H), 7.63-7.68 (m, 1H), 7.59-7.63 (m, 1H), 7.17-7.22 (m, 1H), 7.07-7.12 (m, 1H), 4.54-4.62 (m, 1H), 3.87-3.93 (m, 4H), 3.56-3.64 (m, 1H), 3.15-3.24 (m, 4H), 2.26-2.37 (m, 1H), 2.14-2.26 (m, 1H), 2.01-2.08 (m, 1H), 1.89-1.97 (m, 1H), 1.68-1.77 (m, 1H), 1.44-1.54 (m, 1H). MS (ESI): [M+H]⁺=446.5

### Example 52: (R)-3-(6-morpholino-1H-benzo[d]imidazol-2-yl)-N-(pyrrolidin-3-yl)-1H-indazole-5-carboxamide (12k)

Compound (12k) of Example 52 was obtained from compound 11k according to General Synthesis Procedure B (yellow oil, yield: 33%).

¹H NMR (400 MHz, METHANOL-d₄) δ 8.99 (dd, J=0.80, 1.72 Hz, 1H), 7.93-7.98 (m, 1H), 7.63-7.69 (m, 1H), 7.57-7.63 (m, 1H), 7.18-7.22 (m, 1H), 7.07-7.14 (m, 1H), 4.56 (tdd, J=4.92, 6.75, 7.67 Hz, 1H), 3.83-3.96 (m, 4H), 3.09-3.29 (m, 6H), 2.94-3.09 (m, 2H), 2.19-2.34 (m, 1H), 1.89-2.05 (m, 1H). MS (ESI): [M+H]⁺=432.7

### Example 53: (S)-3-(6-morpholino-1H-benzo[d]imidazol-2-yl)-N-(pyrrolidin-3-yl)-1H-indazole-5-carboxamide (121)

Compound (121) of Example 53 was obtained from compound 111 according to General Synthesis Procedure B (yellow oil, yield: 48%).

¹H NMR (400 MHz, METHANOL-d₄) δ 8.98 (dd, J=0.69, 1.60 Hz, 1H), 7.91-7.99 (m, 1H), 7.65 (dd, J=0.80, 8.82 Hz, 1H), 7.54-7.63 (m, 1H), 7.19 (s, 1H), 7.10 (dd, J=2.29, 8.93 Hz, 1H), 4.55 (tdd, J=4.75, 6.70, 7.84 Hz, 1H), 3.82-3.94 (m, 4H), 3.10-3.29 (m, 6H), 2.92-3.05 (m, 2H), 2.27 (dtd, J=6.41, 8.13, 13.28 Hz, 1H), 1.86-2.05 (m, 1H). MS (ESI): [M+H]⁺=432.7

### Example 54: (R)-3-(6-morpholino-1H-benzo[d]imidazol-2-yl)-N-(pyrrolidin-3-ylmethyl)-1H-indazole-5-carboxamide (12m)

Compound (12m) of Example 54 was obtained from compound 11m according to General Synthesis Procedure B (yellow oil, yield: 57%).

¹H NMR (400 MHz, METHANOL-d₄) δ 8.98-9.01 (m, 1H), 7.96 (dd, J=1.60, 8.70 Hz, 1H), 7.67 (dd, J=0.80, 8.82 Hz, 1H), 7.61 (d, J=8.70 Hz, 1H), 7.19 (d, J=2.06 Hz, 1H), 7.10 (dd, J=2.29, 8.93 Hz, 1H), 3.85-3.93 (m, 4H), 3.46-3.52 (m, 2H), 3.10-3.23 (m, 6H), 3.03 (td, J=7.56, 11.22 Hz, 1H), 2.85 (dd, J=7.10, 11.45 Hz, 1H), 2.61 (td, J=7.36, 14.83 Hz, 1H), 2.10 (dtd, J=5.15, 7.80, 12.91 Hz, 1H), 1.70 (qd, J=7.58, 12.99 Hz, 1H). MS (ESI): [M+H]⁺=446.4

### Example 55: (S)-3-(6-morpholino-1H-benzo[d]imidazol-2-yl)-N-(pyrrolidin-3-ylmethyl)-1H-indazole-5-carboxamide (12n)

Compound (12n) of Example 55 was obtained from compound 1in according to General Synthesis Procedure B (yellow oil, yield: 42%).

¹H NMR (400 MHz, METHANOL-d₄) δ 8.94-9.02 (m, 1H), 7.92-7.97 (m, 1H), 7.66 (dd, J=0.92, 8.93 Hz, 1H), 7.60 (d, J=8.93 Hz, 1H), 7.19 (d, J=2.06 Hz, 1H), 7.10 (dd, J=2.29, 8.93 Hz, 1H), 3.86-3.93 (m, 4H), 3.44-3.52 (m, 2H), 3.16-3.23 (m, 4H), 2.89-3.13 (m, 3H), 2.74 (dd, J=6.53, 11.33 Hz, 1H), 2.49-2.60 (m, 1H), 1.95-2.10 (m, 1H), 1.62 (tdd, J=7.07, 8.01, 12.88 Hz, 1H). MS (ESI): [M+H]⁺=446.7

### Example 56: 3-(6-morpholino-1H-benzo[d]imidazol-2-yl)-N-(piperidin-3-ylmethyl)-1H-indazole-5-carboxamide (120)

Compound (12o) of Example 56 was obtained from compound 11o according to General Synthesis Procedure B (yellow oil, yield: 32%).

¹H NMR (400 MHz, METHANOL-d₄) δ 8.96-9.00 (m, 1H), 7.92-7.97 (m, 1H), 7.66 (dd, J=0.80, 8.82 Hz, 1H), 7.60 (d, J=8.70 Hz, 1H), 7.19 (d, J=2.06 Hz, 1H), 7.10 (dd, J=2.29, 8.70 Hz, 1H), 3.86-3.92 (m, 4H), 3.34-3.38 (m, 1H), 3.11-3.23 (m, 5H), 2.94-3.05 (m, 1H), 2.58 (dt, J=3.09, 12.08 Hz, 1H), 2.41 (dd, J=10.53, 12.36 Hz, 1H), 1.81-2.01 (m, 2H), 1.70-1.81 (m, 1H), 1.48-1.64 (m, 1H), 1.15-1.42 (m, 2H). MS (ESI): [M+H]⁺=460.4

### Example 57: 3-(6-morpholino-1H-benzo[d]imidazol-2-yl)-N-(piperidin-4-ylmethyl)-1H-indazole-5-carboxamide (12p)

Compound (12p) of Example 57 was obtained from compound 11p according to General Synthesis Procedure B (yellow solid, yield: 45%).

¹H NMR (400 MHz, METHANOL-d₄) δ 8.98-9.00 (m, 1H), 7.95 (dd, J=1.72, 8.82 Hz, 1H), 7.66 (dd, J=0.80, 8.82 Hz, 1H), 7.57-7.63 (m, 1H), 7.17-7.22 (m, 1H), 7.10 (dd, J=2.29, 8.93 Hz, 1H), 3.86-3.92 (m, 4H), 3.34-3.39 (m, 2H), 3.17-3.22 (m, 4H), 3.07-3.15 (m, 2H), 2.58-2.68 (m, 2H), 1.80-1.93 (m, 3H), 1.25-1.35 (m, 2H). MS (ESI): [M+H]⁺=460.2

### Example 58: 3-(6-morpholino-1H-benzo[d]imidazol-2-yl)-N-(piperidin-4-yl)-1H-indazole-5-carboxamide (12q)

Compound (12q) of Example 58 was obtained from compound 11q according to General Synthesis Procedure B (yellow oil, yield: 50%).

¹H NMR (400 MHz, METHANOL-d₄) δ 8.98-9.01 (m, 1H), 7.94-8.00 (m, 1H), 7.58-7.69 (m, 2H), 7.15-7.24 (m, 1H), 7.05-7.15 (m, 1H), 4.09-4.23 (m, 1H), 3.81-3.95 (m, 4H), 3.33-3.40 (m, 2H), 3.16-3.24 (m, 4H), 2.92-3.02 (m, 2H), 2.10-2.20 (m, 2H), 1.72-1.87 (m, 2H). MS (ESI): [M+H]⁺=446.5

### Example 59: N-((1s,4s)-4-aminocyclohexyl)-3-(6-morpholino-1H-benzo[d]imidazol-2-yl)-1H-indazole-5-carboxamide (12r)

Compound (12r) of Example 59 was obtained from compound 11r according to General Synthesis Procedure B (yellow solid, yield: 42%).

¹H NMR (400 MHz, METHANOL-d₄) δ 8.93-9.00 (m, 1H), 7.95 (dd, J=1.60, 8.70 Hz, 1H), 7.58-7.69 (m, 2H), 7.19 (d, J=2.29 Hz, 1H), 7.09 (dd, J=2.29, 8.70 Hz, 1H), 4.02-4.11 (m, 1H), 3.85-3.95 (m, 4H), 3.14-3.21 (m, 4H), 2.89-3.01 (m, 1H), 1.86-1.96 (m, 2H), 1.65-1.84 (m, 6H). MS (ESI): [M+H]⁺=460.1

### Example 60: 1-(6-(3-(6-morpholino-1H-benzo[d]imidazol-2-yl)-1H-indazole-5-carbonyl)-2,6-diazaspiro[3.3]heptan-2-yl)ethan-1-one (13h)

Compound (13h) of Example 60 was obtained from compound 12 h according to General Synthesis Procedure F (yellow solid, yield: 55%).

¹H NMR (400 MHz, METHANOL-d₄) δ 8.76 (s, 1H), 7.77 (dd, J=1.37, 8.70 Hz, 1H), 7.66 (d, J=8.70 Hz, 1H), 7.60 (d, J=9.16 Hz, 1H), 7.18 (br. s., 1H), 7.07 (dd, J=2.29, 8.70 Hz, 1H), 4.67 (br. s., 2H), 4.38 (br. s., 4H), 4.16 (s, 2H), 3.78-3.96 (m, 4H), 3.09-3.20 (m, 4H), 1.83 (s, 3H). MS (ESI): [M+H]⁺=486.5

### Example 61: N-((1-acetylazetidin-3-yl)methyl)-3-(6-morpholino-1H-benzo[d]imidazol-2-yl)-1H-indazole-5-carboxamide (13f)

Compound (13f) of Example 61 was obtained from compound 12f according to General Synthesis Procedure F (yellow solid, yield: 42%).

¹H NMR (400 MHz, METHANOL-d₄) δ 8.94-8.99 (m, 1H), 7.91-7.97 (m, 1H), 7.55-7.68 (m, 2H), 7.16 (s, 1H), 7.07 (dd, J=2.29, 9.16 Hz, 1H), 4.31 (t, J=8.47 Hz, 1H), 4.00-4.14 (m, 2H), 3.77-3.95 (m, 5H), 3.59-3.77 (m, 2H), 3.11-3.21 (m, 4H), 2.93-3.05 (m, 1H), 1.85 (s, 3H). MS (ESI): [M+H]⁺=474.4

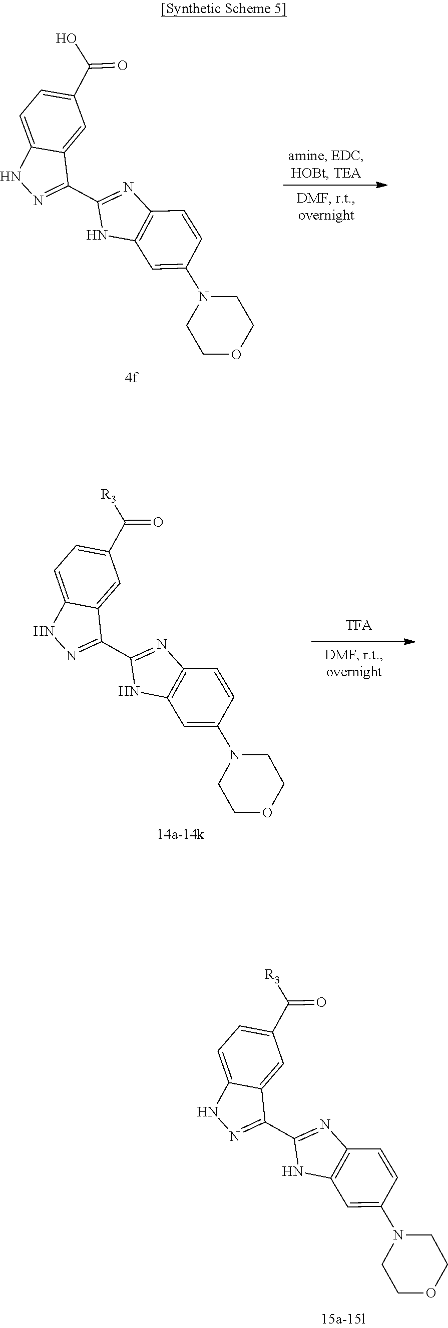

### tert-butyl 4-(3-(6-morpholino-1H-benzo[d]imidazol-2-yl)-1H-indazole-5-carbonyl)piperazine-1-carboxylate (14a)

Compound 14a was obtained from compound 4f by using tert-butyl piperazine-1-carboxylate according to General Synthesis Procedure A (brown solid, yield: 52%).

¹H NMR (400 MHz, METHANOL-d₄) δ 8.55-8.62 (m, 1H), 7.67-7.73 (m, 1H), 7.50-7.64 (m, 2H), 7.02-7.28 (m, 2H), 3.85-3.89 (m, 4H), 3.46-3.76 (m, 8H), 3.15-3.21 (m, 4H), 1.47 (s, 9H). MS (ESI): [M+H]⁺=532.4

### tert-butyl (1R,4R)-5-(3-(6-morpholino-1H-benzo[d]imidazol-2-yl)-1H-indazole-5-carbonyl)-2,5-diazabicyclo[2.2.1]heptane-2-carboxylate (14b)

Compound 14b was obtained from compound 4f by using tert-butyl (1R,4R)-2,5-diazabicyclo[2.2.1]heptane-2-carboxylate according to General Synthesis Procedure A (yellow oil, yield: 57%).

¹H NMR (400 MHz, ACETONE-d₆) δ 8.90-8.97 (m, 1H), 7.48-7.74 (m, 3H), 7.09-7.26 (m, 1H), 6.97-7.09 (m, 1H), 4.53-4.67 (m, 1H), 3.66-3.88 (m, 6H), 3.37-3.64 (m, 3H), 3.14-3.19 (m, 4H), 1.86-2.03 (m, 2H), 1.36-1.54 (m, 9H). MS (ESI): [M+H]⁺=544.1

### tert-butyl (1S,4S)-5-(3-(6-morpholino-1H-benzo[d]imidazol-2-yl)-1H-indazole-5-carbonyl)-2,5-diazabicyclo[2.2.1]heptane-2-carboxylate (14c)

Compound 14c was obtained from compound 4f by using tert-butyl (1S,4S)-2,5-diazabicyclo[2.2.1]heptane-2-carboxylate according to General Synthesis Procedure A (yellow oil, yield: 45%).

¹H NMR (400 MHz, DMSO-d₆) δ 8.63-8.77 (m, 1H), 7.50-7.77 (m, 3H), 6.84-7.10 (m, 2H), 4.31-4.57 (m, 2H), 3.76-3.82 (m, 4H), 3.48-3.71 (m, 2H), 3.36-3.45 (m, 2H), 3.08-3.15 (m, 4H), 1.81-2.02 (m, 2H), 1.32-1.52 (m, 9H). MS (ESI): [M+H]⁺=544.4

### tert-butyl 4-(3-(6-morpholino-1H-benzo[d]imidazol-2-yl)-1H-indazole-5-carbonyl)-1,4-diazepane-1-carboxylate (14d)

Compound 14d was obtained from compound 4f by using tert-butyl 1,4-diazepane-1-carboxylate according to General Synthesis Procedure A (brown oil, yield: 43%).

¹H NMR (400 MHz, METHANOL-d₄) δ 8.53-8.59 (m, 1H), 7.65-7.74 (m, 1H), 7.44-7.63 (m, 2H), 6.99-7.19 (m, 2H), 3.84-3.93 (m, 5H), 3.74-3.82 (m, 1H), 3.64-3.73 (m, 2H), 3.40-3.62 (m, 4H), 3.15-3.20 (m, 4H), 1.91-2.04 (m, 1H), 1.62-1.72 (m, 1H), 1.46 (s, 9H). MS (ESI): [M+H]⁺=546.8

### tert-butyl ((1-(3-(6-morpholino-1H-benzo[d]imidazol-2-yl)-1H-indazole-5-carbonyl)piperidin-3-yl)methyl)carbamate (14e)

Compound 14e was obtained from compound 4f by using tert-butyl (piperidin-3-ylmethyl)carbamate according to General Synthesis Procedure A (yellow oil, yield: 37%).

¹H NMR (400 MHz, DMSO-d₆) δ 8.44-8.57 (m, 1H), 7.34-7.64 (m, 3H), 7.03-7.29 (m, 1H), 6.81-7.03 (m, 2H), 4.19-4.46 (m, 1H), 3.70-3.79 (m, 4H), 3.05-3.12 (m, 4H), 2.55-2.95 (m, 4H), 1.54-1.76 (m, 3H), 1.25-1.44 (m, 5H), 0.97-1.23 (m, 7H). MS (ESI): [M+H]⁺=560.5

### tert-butyl (1-(3-(6-morpholino-1H-benzo[d]imidazol-2-yl)-1H-indazole-5-carbonyl)piperidin-4-yl)carbamate (14f)

Compound 14f was obtained from compound 4f by using tert-butyl piperidin-4-ylcarbamate according to General Synthesis Procedure A (yellow oil, yield: 40%).

¹H NMR (400 MHz, METHANOL-d₄) δ 8.53-8.58 (m, 1H), 7.56-7.81 (m, 2H), 7.46-7.53 (m, 1H), 6.94-7.18 (m, 2H), 4.38-4.65 (m, 1H), 3.76-3.92 (m, 5H), 3.58-3.72 (m, 1H), 3.02-3.29 (m, 6H), 1.78-2.08 (m, 2H), 1.42-1.52 (m, 11H). MS (ESI): [M+H]⁺=546.1

### tert-butyl methyl(1-(3-(6-morpholino-1H-benzo[d]imidazol-2-yl)-1H-indazole-5-carbonyl)piperidin-4-yl)carbamate (14g)

Compound 14g was obtained from compound 4f by using tert-butyl methyl(piperidin-4-yl)carbamate according to General Synthesis Procedure A (yellow oil, yield: 39%).

¹H NMR (400 MHz, METHANOL-d₄) δ 8.56-8.61 (m, 1H), 7.69 (dd, J=0.80, 8.59 Hz, 1H), 7.51-7.64 (m, 2H), 7.13-7.20 (m, 1H), 7.08 (dd, J=2.18, 8.82 Hz, 1H), 4.67-4.85 (m, 1H), 4.06-4.26 (m, 1H), 3.94-4.06 (m, 1H), 3.84-3.91 (m, 4H), 3.06-3.29 (m, 5H), 2.85-3.06 (m, 1H), 2.83 (s, 3H), 1.72-1.89 (m, 3H), 1.59-1.68 (m, 1H), 1.45 (s, 9H). MS (ESI): [M+H]⁺=560.4

### tert-butyl (1R,5S,6r)-6-(3-(6-morpholino-1H-benzo[d]imidazol-2-yl)-1H-indazole-5-carboxamido)-3-azabicyclo[3.1.0]hexane-3-carboxylate (14h)

Compound 14 h was obtained from compound 4f by using tert-butyl (1R,5S,6r)-6-amino-3-azabicyclo[3.1.0]hexane-3-carboxylate according to General Synthesis Procedure A (yellow oil, yield: 45%).

¹H NMR (400 MHz, METHANOL-d₄) δ 8.92-8.97 (m, 1H), 7.89-7.98 (m, 1H), 7.53-7.68 (m, 2H), 7.09 (d, J=8.70 Hz, 2H), 3.81-3.93 (m, 4H), 3.74 (dd, J=7.10, 10.76 Hz, 2H), 3.40-3.52 (m, 2H), 3.18 (dd, J=3.89, 5.72 Hz, 4H), 2.59 (t, J=2.29 Hz, 1H), 1.87-1.98 (m, 2H), 1.46 (s, 9H). MS (ESI): [M+H]⁺=544.1

### tert-butyl (4-(3-(6-morpholino-1H-benzo[d]imidazol-2-yl)-1H-indazole-5-carboxamido)phenyl)carbamate (14i)

Compound 14i was obtained from compound 4f by using tert-butyl (4-aminophenyl)carbamate according to General Synthesis Procedure A (brown oil, yield: 42%).

¹H NMR (400 MHz, METHANOL-d₄) δ 9.08-9.14 (m, 1H), 7.99-8.09 (m, 1H), 7.52-7.73 (m, 4H), 7.38-7.49 (m, 2H), 7.00-7.24 (m, 2H), 3.82-3.92 (m, 4H), 3.11-3.23 (m, 4H), 1.52 (s, 9H). MS (ESI): [M+H]⁺=554.1

### Example 62: N-(2-(dimethylamino)ethyl)-3-(6-morpholino-1H-benzo[d]imidazol-2-yl)-1H-indazole-5-carboxamide (14j)

Compound (14j) of Example 62 was obtained from compound 4f by using N,N-dimethylethane-1,2-diamine according to General Synthesis Procedure A (yellow oil, yield: 55%).

¹H NMR (400 MHz, METHANOL-d₄) δ 8.96-9.01 (m, 1H), 7.92-7.97 (m, 1H), 7.65 (s, 1H), 7.54-7.63 (m, 1H), 7.15-7.21 (m, 1H), 7.09 (dd, J=2.18, 8.83 Hz, 1H), 3.83-3.93 (m, 4H), 3.56-3.63 (m, 2H), 3.15-3.22 (m, 4H), 2.62-2.70 (m, 2H), 2.36 (s, 6H). MS (ESI): [M+H]⁺=434.5

### Example 63: N-(3-(dimethylamino)propyl)-3-(6-morpholino-1H-benzo[d]imidazol-2-yl)-1H-indazole-5-carboxamide (14k)

Compound (14k) of Example 63 was obtained from compound 4f by using N,N-dimethylpropane-1,3-diamine according to General Synthesis Procedure A (yellow oil, yield: 33%).

¹H NMR (400 MHz, METHANOL-d₄) δ 8.98 (dd, J=0.92, 1.60 Hz, 1H), 7.95 (dd, J=1.60, 8.71 Hz, 1H), 7.66 (dd, J=0.92, 8.94 Hz, 1H), 7.54-7.63 (m, 1H), 7.15-7.26 (m, 1H), 7.09 (dd, J=2.29, 8.71 Hz, 1H), 3.82-3.92 (m, 4H), 3.45-3.54 (m, 2H), 3.14-3.26 (m, 4H), 2.49-2.59 (m, 2H), 2.33 (s, 6H), 1.83-1.91 (m, 2H). MS (ESI): [M+H]⁺=448.8

### Example 64: (3-(6-morpholino-1H-benzo[d]imidazol-2-yl)-1H-indazol-5-yl)(piperazin-1-yl)methanone (15a)

Compound (15a) of Example 64 was obtained from compound 14a according to General Synthesis Procedure B (yellow oil, yield: 40%).

¹H NMR (400 MHz, METHANOL-d₄) δ 8.55-8.57 (m, 1H), 7.70 (dd, J=0.92, 8.70 Hz, 1H), 7.49-7.67 (m, 2H), 7.04-7.23 (m, 2H), 3.85-3.92 (m, 4H), 3.54-3.84 (m, 4H), 3.13-3.21 (m, 4H), 2.76-3.06 (m, 4H). MS (ESI): [M+H]⁺=431.9

### Example 65: ((1R,4R)-2,5-diazabicyclo[2.2.1]heptan-2-yl)(3-(6-morpholino-1H-benzo[d]imidazol-2-yl)-1H-indazol-5-yl)methanone (15b)

Compound (15b) of Example 65 was obtained from compound 14b according to General Synthesis Procedure B (yellow oil, yield: 46%).

¹H NMR (400 MHz, METHANOL-d₄) δ 8.62-8.72 (m, 1H), 7.52-7.76 (m, 3H), 7.02-7.27 (m, 2H), 4.53 (s, 1H), 3.78-3.92 (m, 5H), 3.65-3.78 (m, 1H), 3.47-3.57 (m, 1H), 3.11-3.29 (m, 5H), 2.95-3.11 (m, 1H), 1.70-2.13 (m, 3H). MS (ESI): [M+H]⁺=443.7

### Example 66: ((1S,4S)-2,5-diazabicyclo[2.2.1]heptan-2-yl)(3-(6-morpholino-1H-benzo[d]imidazol-2-yl)-1H-indazol-5-yl)methanone (15c)

Compound (15c) of Example 66 was obtained from compound 14c according to General Synthesis Procedure B (yellow oil, yield: 39%).

¹H NMR (400 MHz, METHANOL-d₄) δ 8.60-8.73 (m, 1H), 7.58-7.77 (m, 3H), 6.99-7.26 (m, 2H), 4.55 (s, 1H), 3.68-3.93 (m, 6H), 3.39-3.57 (m, 1H), 3.10-3.29 (m, 5H), 2.95-3.10 (m, 1H), 1.92-2.10 (m, 1H), 1.77 (d, J=10.53 Hz, 1H). MS (ESI): [M+H]⁺=444.5

### Example 67: (1,4-diazepan-1-yl)(3-(6-morpholino-1H-benzo[d]imidazol-2-yl)-1H-indazol-5-yl)methanone (15d)

Compound (15d) of Example 67 was obtained from compound 14d according to General Synthesis Procedure B (yellow solid, yield: 43%).

¹H NMR (400 MHz, METHANOL-d₄) δ 8.52-8.60 (m, 1H), 7.68 (d, J=8.47 Hz, 1H), 7.50-7.63 (m, 2H), 7.11-7.20 (m, 1H), 7.06 (dd, J=2.18, 8.82 Hz, 1H), 3.76-3.90 (m, 6H), 3.56-3.68 (m, 2H), 3.11-3.21 (m, 4H), 3.04-3.11 (m, 1H), 2.83-3.02 (m, 3H), 1.93-2.06 (m, 1H), 1.73-1.88 (m, 1H). MS (ESI): [M+H]⁺=446.0

### Example 68: (3-(aminomethyl)piperidin-1-yl)(3-(6-morpholino-1H-benzo[d]imidazol-2-yl)-1H-indazol-5-yl)methanone (15e)

Compound (15e) of Example 68 was obtained from compound 14e according to General Synthesis Procedure B (yellow oil, yield: 53%).

¹H NMR (400 MHz, DMSO-d₆) δ 8.53-8.60 (m, 1H), 7.64-7.73 (m, 1H), 7.43-7.64 (m, 2H), 6.87-7.09 (m, 2H), 4.22-4.50 (m, 1H), 3.73-3.84 (m, 4H), 3.26-3.38 (m, 4H), 3.08-3.16 (m, 4H), 1.80-1.88 (m, 1H), 1.62-1.72 (m, 1H), 1.40-1.60 (m, 2H), 1.05-1.33 (m, 2H). MS (ESI): [M+H]⁺=460.5

### Example 69: (4-aminopiperidin-1-yl)(3-(6-morpholino-1H-benzo[d]imidazol-2-yl)-1H-indazol-5-yl)methanone (15f)

Compound (15f) of Example 69 was obtained from compound 14f according to General Synthesis Procedure B (yellow oil, yield: 47%).

¹H NMR (400 MHz, METHANOL-d₄) δ 8.50-8.59 (m, 1H), 7.66-7.71 (m, 1H), 7.58 (d, J=9.16 Hz, 1H), 7.48-7.54 (m, 1H), 7.16 (d, J=1.83 Hz, 1H), 7.06 (dd, J=2.29, 8.70 Hz, 1H), 4.47-4.72 (m, 1H), 3.72-4.01 (m, 5H), 3.10-3.27 (m, 5H), 2.88-3.07 (m, 2H), 1.74-2.04 (m, 2H), 1.25-1.52 (m, 2H). MS (ESI): [M+H]⁺=445.8

### Example 70: (4-(methylamino)piperidin-1-yl)(3-(6-morpholino-1H-benzo[d]imidazol-2-yl)-1H-indazol-5-yl)methanone (15g)

Compound (15g) of Example 70 was obtained from compound 14g according to General Synthesis Procedure B (yellow oil, yield: 59%).

¹H NMR (400 MHz, METHANOL-d₄) δ 8.54-8.58 (m, 1H), 7.67-7.71 (m, 1H), 7.56-7.61 (m, 1H), 7.49-7.56 (m, 1H), 7.14-7.22 (m, 1H), 7.04-7.11 (m, 1H), 4.57-4.78 (m, 1H), 3.76-3.99 (m, 5H), 3.12-3.29 (m, 5H), 2.96-3.10 (m, 1H), 2.63-2.76 (m, 1H), 2.35-2.44 (m, 3H), 1.85-2.14 (m, 2H), 1.27-1.47 (m, 2H). MS (ESI): [M+H]⁺=460.4

### Example 71: N-((1R,5S,6r)-3-azabicyclo[3.1.0]hexan-6-yl)-3-(6-morpholino-1H-benzo[d]imidazol-2-yl)-1H-indazole-5-carboxamide (15h)

Compound (15h) of Example 71 was obtained from compound 14 h according to General Synthesis Procedure B (yellow oil, yield: 34%).

¹H NMR (400 MHz, DMSO-d₆) δ 8.90-8.98 (m, 1H), 8.46-8.55 (m, 1H), 7.81-7.92 (m, 1H), 7.57-7.68 (m, 2H), 6.89-7.06 (m, 2H), 3.75-3.82 (m, 4H), 3.07-3.17 (m, 4H), 3.02 (d, J=10.99 Hz, 2H), 2.73-2.83 (m, 3H), 1.66 (s, 2H). MS (ESI): [M+H]⁺=444.1

### Example 72: N-(4-aminophenyl)-3-(6-morpholino-1H-benzo[d]imidazol-2-yl)-1H-indazole-5-carboxamide (15i)

Compound (15i) of Example 72 was obtained from compound 14i according to General Synthesis Procedure B (yellow oil, yield: 49%).

¹H NMR (400 MHz, METHANOL-d₄) δ 9.04-9.12 (m, 1H), 8.02 (dd, J=1.60, 8.70 Hz, 1H), 7.64-7.71 (m, 1H), 7.54-7.64 (m, 1H), 7.45-7.52 (m, 2H), 7.12-7.24 (m, 1H), 7.03-7.12 (m, 1H), 6.74-6.85 (m, 2H), 3.85-3.92 (m, 4H), 3.13-3.22 (m, 4H). MS (ESI): [M+H]⁺=454.5

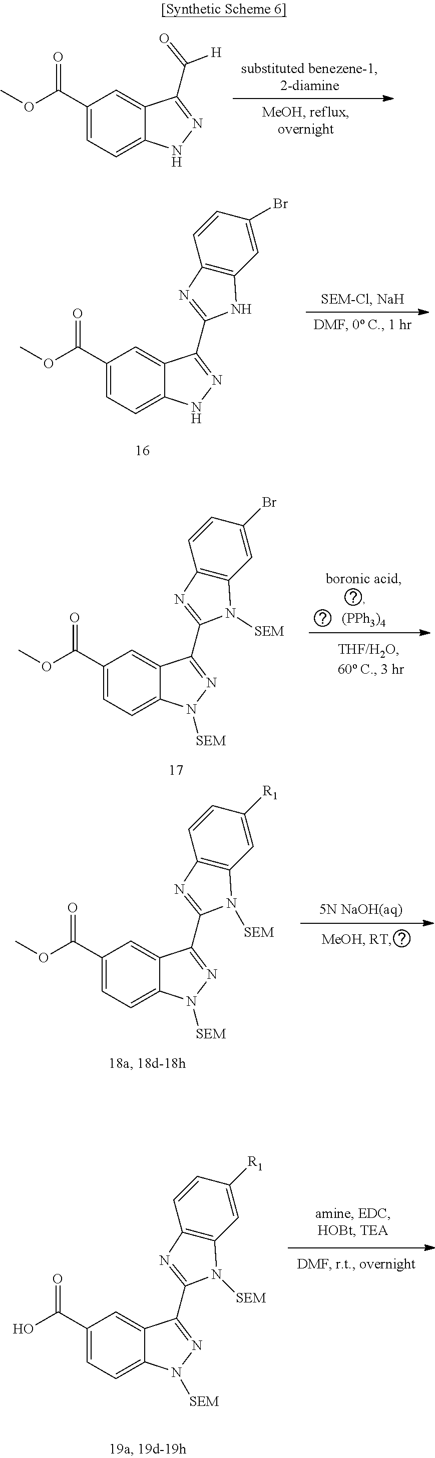

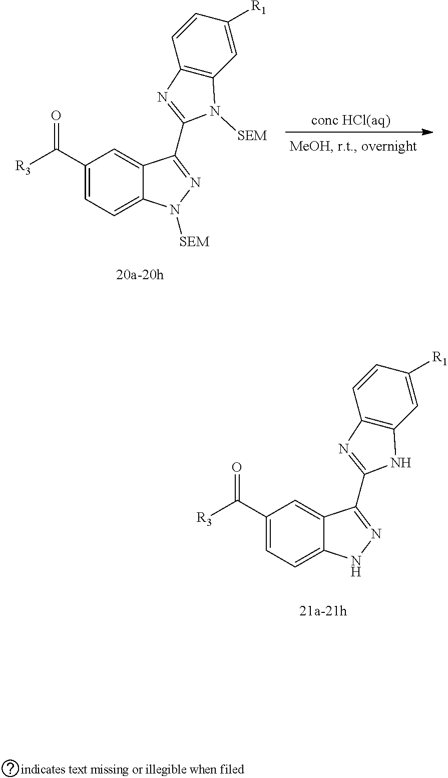

### methyl 3-(6-bromo-1H-benzo[d]imidazol-2-yl)-1H-indazole-5-carboxylate (16)

Compound 16 was obtained by using 4-bromobenzene-1,2-diamine according to General Synthesis Procedure C (yellow oil, yield: 32%).

¹H NMR (400 MHz, METHANOL-d₄) δ 9.14-9.22 (m, 1H), 8.03-8.13 (m, 1H), 7.74-7.98 (m, 1H), 7.49-7.70 (m, 2H), 7.33-7.43 (m, 1H), 3.94 (s, 3H). MS (ESI): [M+H]⁺=371.2

### methyl 3-(6-bromo-1-((2-(trimethylsilyl)ethoxy)methyl)-1H-benzo[d]imidazol-2-yl)-1-((2-(trimethylsilyl)ethoxy)methyl)-1H-indazole-5-carboxylate (17)

Sodium hydride (60%) (2.5 eq) was added to compound 16, and the solution was dissolved in DMF and cooled to 0° C. (2-Chloromethoxyethyl)trimethylsilane (2.5 eq) was then added, and the solution was stirred for 1 hour. Upon completion of the reaction, the solution was extracted with water and ethyl acetate, dried over Na₂SO₄ and filtered. The solvent was removed under reduced pressure, and the residue was purified by column chromatography to afford compound 17 (colorless oil, yield: 70%).

¹H NMR (400 MHz, DMSO-d₆) δ 9.11-9.20 (m, 1H), 8.07-8.18 (m, 1H), 7.98-8.07 (m, 2H), 7.84 (d, J=8.70 Hz, 1H), 7.46-7.56 (m, 1H), 6.23-6.34 (m, 2H), 5.95 (s, 2H), 3.94 (s, 3H), 3.59-3.75 (m, 2H), 3.53 (t, J=7.79 Hz, 2H), 0.84-0.89 (m, 2H), 0.72-0.77 (m, 2H), −0.10 (s, 9H), −0.23 (s, 9H).

### methyl 3-(6-(furan-3-yl)-1-((2-(trimethylsilyl)ethoxy)methyl)-1H-benzo[d]imidazol-2-yl)-1-((2-(trimethylsilyl)ethoxy)methyl)-1H-indazole-5-carboxylate (18a)

Compound 18a was obtained by using furan-3-ylboronic acid according to General Synthesis Procedure G (colorless oil, yield: 60%).

¹H NMR (400 MHz, DMSO-d₆) δ 9.21-9.25 (m, 1H), 8.26-8.31 (m, 1H), 8.07-8.20 (m, 1H), 7.96-8.07 (m, 2H), 7.87 (d, J=8.24 Hz, 1H), 7.74-7.83 (m, 1H), 7.61-7.71 (m, 1H), 7.09 (d, J=1.83 Hz, 1H), 6.27-6.35 (m, 2H), 5.96 (s, 2H), 3.94 (s, 3H), 3.65-3.74 (m, 2H), 3.55 (t, J=8.01 Hz, 2H), 0.82-0.95 (m, 2H), 0.69-0.82 (m, 2H), 0.09 (d, J=1.37 Hz, 9H), 0.28-0.21 (m, 9H). MS (ESI): [M+H]⁺=619.5

### methyl 3-(6-phenyl-1-((2-(trimethylsilyl)ethoxy)methyl)-1H-benzo[d]imidazol-2-yl)-1-((2-(trimethylsilyl)ethoxy)methyl)-1H-indazole-5-carboxylate (18d)

Compound 18d was obtained by using phenylboronic acid according to General Synthesis Procedure G (colorless oil, 71%).

¹H NMR (400 MHz, DMSO-d₆) δ 9.22-9.27 (m, 1H), 8.13-8.17 (m, 1H), 7.92-8.11 (m, 3H), 7.78-7.85 (m, 2H), 7.65-7.74 (m, 1H), 7.46-7.56 (m, 2H), 7.33-7.42 (m, 1H), 6.37 (s, 2H), 5.97 (s, 2H), 3.95 (s, 3H), 3.64-3.74 (m, 2H), 3.47-3.61 (m, 2H), 0.81-0.95 (m, 2H), 0.68-0.80 (m, 2H), 0.10-0.08 (m, 9H), 0.25-0.21 (m, 9H).

### methyl 3-(6-(pyridin-4-yl)-1-((2-(trimethylsilyl)ethoxy)methyl)-1H-benzo[d]imidazol-2-yl)-1-((2-(trimethylsilyl)ethoxy)methyl)-1H-indazole-5-carboxylate (18e)

Compound 18e was obtained by using pyridin-4-ylboronic acid according to General Synthesis Procedure G (colorless oil, yield: 58%).

### methyl 3-(6-(4-(dimethylamino)phenyl)-1-((2-(trimethylsilyl)ethoxy)methyl)-1H-benzo[d]imidazol-2-yl)-1-((2-(trimethylsilyl)ethoxy)methyl)-1H-indazole-5-carboxylate (18f)

Compound 18f was obtained by using (4-(dimethylamino)phenyl)boronic acid according to General Synthesis Procedure G (colorless oil, yield: 52%).

¹H NMR (400 MHz, DMSO-d₆) δ 9.23-9.26 (m, 1H), 8.10-8.19 (m, 1H), 7.97-8.08 (m, 2H), 7.77 (d, J=8.24 Hz, 1H), 7.58-7.70 (m, 3H), 6.79-6.89 (m, 2H), 6.28 (s, 2H), 5.96 (s, 2H), 3.95 (s, 3H), 3.64-3.73 (m, 2H), 3.52-3.60 (m, 2H), 2.96 (s, 6H), 0.80-0.93 (m, 2H), 0.67-0.79 (m, 2H), 0.13-0.06 (m, 9H), 0.25-0.20 (m, 9H).

### methyl 3-(6-(4-methoxyphenyl)-1-((2-(trimethylsilyl)ethoxy)methyl)-1H-benzo[d]imidazol-2-yl)-1-((2-(trimethylsilyl)ethoxy)methyl)-1H-indazole-5-carboxylate (18g)

Compound 18g was obtained by using (4-methoxyphenyl)boronic acid according to General Synthesis Procedure G (colorless oil, yield: 54%).

¹H NMR (400 MHz, DMSO-d₆) δ 9.22-9.27 (m, 1H), 8.06-8.18 (m, 1H), 7.97-8.06 (m, 2H), 7.57-7.81 (m, 4H), 7.00-7.12 (m, 2H), 6.28-6.39 (m, 2H), 5.96 (s, 2H), 3.95 (s, 3H), 3.82 (s, 3H), 3.65-3.71 (m, 2H), 3.52-3.60 (m, 2H), 0.84-0.93 (m, 2H), 0.74-0.81 (m, 2H), 0.13-0.07 (m, 9H), 0.26-0.18 (m, 9H).

### methyl 3-(6-(furan-2-yl)-1-((2-(trimethylsilyl)ethoxy)methyl)-1H-benzo[d]imidazol-2-yl)-1-((2-(trimethylsilyl)ethoxy)methyl)-1H-indazole-5-carboxylate (18h)

Compound 18 h was obtained by using furan-2-ylboronic acid according to General Synthesis Procedure G (light brown solid, yield: 75%).

Mixture of two regioisomers. ¹H NMR (400 MHz, DMSO-d₆) δ 9.21-9.25 (m, 1H), 8.13 (dd, J=1.15, 8.93 Hz, 1H), 8.04-8.21 (m, 1H), 8.00 (d, J=8.93 Hz, 1H), 7.79-7.92 (m, 1H), 7.78-7.79 (m, 1H), 7.70-7.78 (m, 1H), 7.01-7.06 (m, 1H), 6.60-6.66 (m, 1H), 6.25-6.35 (m, 2H), 5.95 (s, 2H), 3.92-3.96 (m, 3H), 3.67 (t, J=7.90 Hz, 2H), 3.52-3.60 (m, 2H), 0.87 (t, J=8.00 Hz, 2H), 0.74-0.80 (m, 2H), 0.10 (s, 9H), 0.25-0.22 (m, 9H). MS (ESI): [M+H]⁺=MS (ESI): [M+H]⁺=619.7

### 3-(6-(furan-3-yl)-1-((2-(trimethylsilyl)ethoxy)methyl)-1H-benzo[d]imidazol-2-yl)-1-((2-(trimethylsilyl)ethoxy)methyl)-1H-indazole-5-carboxylic acid (19a)

Compound 19a was obtained from compound 18a according to General Synthesis Procedure D (colorless oil, yield: 52%).

¹H NMR (400 MHz, DMSO-d₆) δ 9.06 (d, J=14.20 Hz, 1H), 8.25-8.28 (m, 1H), 8.14 (td, J=1.37, 8.70 Hz, 1H), 7.94-7.99 (m, 1H), 7.89 (d, J=8.24 Hz, 1H), 7.59-7.83 (m, 4H), 7.16 (d, J=1.37 Hz, 1H), 7.06-7.12 (m, 1H), 6.30 (s, 1H), 6.33 (s, 1H), 5.89 (s, 2H), 3.65 (dt, J=2.98, 7.90 Hz, 2H), 3.55 (dt, J=3.21, 7.79 Hz, 2H), 0.82-0.90 (m, 2H), 0.71-0.79 (m, 2H), −0.08 (d, J=1.37 Hz, 9H), −0.25-−0.21 (m, 9H). MS (ESI): [M+H]⁺=605.2

### 3-(6-phenyl-1-((2-(trimethylsilyl)ethoxy)methyl)-1H-benzo[d]imidazol-2-yl)-1-((2-(trimethylsilyl)ethoxy)methyl)-1H-indazole-5-carboxylic acid (19d)

Compound 19d was obtained from compound 18d according to General Synthesis Procedure D (brown solid, yield: 84%).

¹H NMR (400 MHz, DMSO-d₆) δ 9.13 (s, 1H), 8.08-8.24 (m, 1H), 8.03 (d, J=1.37 Hz, 1H), 7.97 (d, J=8.70 Hz, 1H), 7.77-7.83 (m, 3H), 7.63-7.73 (m, 1H), 7.43-7.55 (m, 2H), 7.33-7.43 (m, 1H), 6.26-6.43 (m, 2H), 5.92 (s, 2H), 3.64-3.70 (m, 2H), 3.54-3.60 (m, 2H), 0.86-0.91 (m, 2H), 0.74-0.80 (m, 2H), 0.09-0.07 (m, 9H), 0.24-0.20 (m, 9H).

### 3-(6-(pyridin-4-yl)-1-((2-(trimethylsilyl)ethoxy)methyl)-1H-benzo[d]imidazol-2-yl)-1-((2-(trimethylsilyl)ethoxy)methyl)-1H-indazole-5-carboxylic acid (19e)

Compound 19e was obtained from compound 18e according to General Synthesis Procedure D (brown solid, yield: 72%). 3-(6-(4-(dimethylamino)phenyl)-1-((2-(trimethylsilyl)ethoxy)methyl)-1H-benzo[d]imidazol-2-yl)-1-((2-(trimethylsilyl)ethoxy)methyl)-1H-indazole-5-carboxylic acid (19f)

Compound 19f was obtained from compound 18f according to General Synthesis Procedure D (brown solid, yield: 85%).

¹H NMR (400 MHz, DMSO-d₆) δ 9.19-9.24 (m, 1H), 8.09-8.18 (m, 1H), 7.89-7.99 (m, 2H), 7.60-7.68 (m, 3H), 7.52-7.60 (m, 1H), 6.80-6.88 (m, 2H), 6.27-6.37 (m, 2H), 5.91-5.98 (m, 2H), 3.63-3.73 (m, 2H), 3.50-3.61 (m, 2H), 2.96 (d, J=3.21 Hz, 6H), 0.82-0.91 (m, 2H), 0.68-0.80 (m, 2H), 0.09 (s, 9H), 0.23-0.22 (m, 9H).

### 3-(6-(4-methoxyphenyl)-1-((2-(trimethylsilyl)ethoxy)methyl)-1H-benzo[d]imidazol-2-yl)-1-((2-(trimethylsilyl)ethoxy)methyl)-1H-indazole-5-carboxylic acid (19g)

Compound 19g was obtained from compound 18g according to General Synthesis Procedure D (brown solid, yield: 46%).

¹H NMR (400 MHz, DMSO-d₆) δ 9.06-9.15 (m, 1H), 8.14-8.16 (m, 1H), 7.91-8.00 (m, 1H), 7.68-7.86 (m, 4H), 7.56-7.65 (m, 1H), 7.02-7.12 (m, 2H), 6.29-6.40 (m, 2H), 5.86-5.95 (m, 2H), 3.76-3.85 (m, 3H), 3.59-3.72 (m, 2H), 3.49-3.59 (m, 2H), 0.83-0.94 (m, 2H), 0.69-0.80 (m, 2H), 0.13-−0.03 (m, 9H), 0.27-0.18 (m, 9H).

### 3-(6-(furan-2-yl)-1-((2-(trimethylsilyl)ethoxy)methyl)-1H-benzo[d]imidazol-2-yl)-1-((2-(trimethylsilyl)ethoxy)methyl)-1H-indazole-5-carboxylic acid (19h)

Compound 19 h was obtained from compound 18 h according to General Synthesis Procedure D (light orange solid, yield: 50%).

Mixture of two regioisomers. ¹H NMR (400 MHz, CHLOROFORM-d) δ 9.60 (d, J=0.69 Hz, 1H), 8.23-8.28 (m, 1H), 7.98-8.32 (m, 1H), 7.70-7.78 (m, 1H), 7.67-7.70 (m, 1H), 7.84 (d, J=0.46 Hz, OH), 7.47-7.53 (m, 1H), 6.68-6.75 (m, 1H), 6.48-6.54 (m, 1H), 6.27-6.33 (m, 1H), 5.84-5.89 (m, 1H), 3.60-3.71 (m, 4H), 0.92-0.99 (m, 2H), 0.84-0.92 (m, 2H), 0.04-0.02 (m, 9H), 0.13-−0.11 (m, 9H). MS (ESI): [M+H]⁺=605.3

### tert-butyl (3-(3-(6-(furan-3-yl)-1-((2-(trimethylsilyl)ethoxy)methyl)-1H-benzo[d]imidazol-2-yl)-1-((2-(trimethylsilyl)ethoxy)methyl)-1H-indazole-5-carboxamido)propyl)carbamate (20a)

Compound 20a was obtained from compound 19a by using tert-butyl (3-aminopropyl)carbamate according to General Synthesis Procedure A (yellow oil, yield: 65%).

¹H NMR (400 MHz, METHANOL-d₄) δ 8.94-8.98 (m, 1H), 7.86-8.04 (m, 3H), 7.79-7.86 (m, 2H), 7.56-7.62 (m, 2H), 6.86-6.92 (m, 1H), 6.18-6.29 (m, 2H), 5.83-5.95 (m, 2H), 3.70 (t, J=8.01 Hz, 2H), 3.56-3.64 (m, 2H), 3.49 (t, J=6.87 Hz, 2H), 3.18 (t, J=6.64 Hz, 2H), 1.82 (t, J=6.87 Hz, 2H), 1.41-1.45 (m, 9H), 0.86-0.94 (m, 2H), 0.79 (t, J=8.01 Hz, 2H), 0.09-0.05 (m, 9H), 0.25-0.18 (m, 9H).

### tert-butyl ((1S,3S)-3-(3-(6-(furan-3-yl)-1-((2-(trimethylsilyl)ethoxy)methyl)-1H-benzo[d]imidazole-2-yl)-1-((2-(trimethylsilyl)ethoxy)methyl)-1H-indazole-5-carboxamido)cyclopentyl) carbamate (20b)

Compound 20b was obtained from compound 19a by using (1S,3S)-3-aminocyclopentyl tert-butylcarbamate according to General Synthesis Procedure A (white solid, yield: 70%).

Mixture of two regioisomers. ¹H NMR (400 MHz, DMSO-d₆) δ 8.98-9.04 (m, 1H), 8.50-8.57 (m, 1H), 8.24-8.29 (m, 1H), 8.03 (dd, J=1.37, 8.70 Hz, 1H), 7.98-8.18 (m, 1H), 7.93 (d, J=8.93 Hz, 1H), 7.75-7.80 (m, 1H), 7.73-7.89 (m, 1H), 7.59-7.69 (m, 1H), 7.05-7.16 (m, 1H), 6.95 (d, J=7.33 Hz, 1H), 6.25-6.34 (m, 2H), 5.94 (s, 2H), 4.37-4.47 (m, 1H), 3.95-4.06 (m, 1H), 3.62-3.71 (m, 2H), 3.54 (t, J=7.90 Hz, 2H), 1.94-2.12 (m, 2H), 1.76-1.90 (m, 2H), 1.50-1.61 (m, 1H), 1.41-1.46 (m, 1H), 1.39 (s, 9H), 0.85-0.90 (m, 2H), 0.76 (t, J=7.60 Hz, 2H), 0.10-0.08 (m, 9H), 0.25-0.21 (m, 9H). MS (ESI): [M+H]⁺=787.4.

### tert-butyl 3-((3-(6-(furan-3-yl)-1H-benzo[d]imidazol-2-yl)-1H-indazole-5-carboxamido) methyl)azetidine-1-carboxylate (20c)

Compound 20c was obtained from compound 19a by using tert-butyl 3-(aminomethyl)azetidine-1-carboxylate according to General Synthesis Procedure A (white solid, yield: 57%).

Mixture of two regioisomers. ¹H NMR (400 MHz, DMSO-d₆) δ 9.00-9.07 (m, 1H), 8.76-8.85 (m, 1H), 8.24-8.29 (m, 1H), 8.01-8.05 (m, 1H), 7.99-8.19 (m, 1H), 7.94-7.98 (m, 1H), 7.76-7.80 (m, 1H), 7.74-7.89 (m, OH), 7.62-7.69 (m, 1H), 7.07-7.14 (m, 1H), 6.25-6.34 (m, 2H), 5.94 (s, 2H), 3.90 (br. s., 2H), 3.63-3.72 (m, 4H), 3.48-3.58 (m, 4H), 2.78 (br. s., 1H), 1.35 (s, 9H), 0.85-0.90 (m, 2H), 0.76 (t, J=8.01 Hz, 2H), −0.10-−0.08 (m, 9H), −0.25-−0.22 (m, 9H). MS (ESI): [M+H]⁺=773.4

### tert-butyl (3-(3-(6-phenyl-1-((2-(trimethylsilyl)ethoxy)methyl)-1H-benzo[d]imidazol-2-yl)-1-((2-(trimethylsilyl)ethoxy)methyl)-1H-indazole-5-carboxamido)propyl)carbamate (20d)

Compound 20d was obtained from compound 19d by using tert-butyl (3-aminopropyl)carbamate according to General Synthesis Procedure A (yellow oil, yield: 53%).

¹H NMR (400 MHz, METHANOL-d₄) δ 8.97-9.00 (m, 1H), 8.04-8.07 (m, 1H), 7.90-7.95 (m, 2H), 7.84-7.88 (m, 1H), 7.72-7.76 (m, 2H), 7.67 (dd, J=1.60, 8.47 Hz, 1H), 7.46-7.49 (m, 2H), 7.34-7.39 (m, 1H), 6.32 (s, 2H), 5.94 (s, 2H), 3.69-3.74 (m, 2H), 3.59-3.64 (m, 2H), 3.48-3.52 (m, 2H), 3.17-3.20 (m, 2H), 1.83 (t, J=6.87 Hz, 2H), 1.43 (s, 9H), 0.91-0.96 (m, 2H), 0.79-0.83 (m, 2H), −0.06 (s, 9H), −0.21 (s, 9H).

### tert-butyl (3-(3-(6-(pyridin-4-yl)-1-((2-(trimethylsilyl)ethoxy)methyl)-1H-benzo[d]imidazol-2-yl)-1-((2-(trimethylsilyl)ethoxy)methyl)-1H-indazole-5-carboxamido)propyl)carbamate (20e)

Compound 20e was obtained from compound 19e by using tert-butyl (3-aminopropyl)carbamate according to General Synthesis Procedure A (yellow oil, yield: 43%).

¹H NMR (400 MHz, METHANOL-d₄) δ 8.97-9.06 (m, 1H), 8.60-8.66 (m, 2H), 8.15 (s, 1H), 8.03-8.10 (m, 1H), 7.96-8.03 (m, 1H), 7.78-7.92 (m, 4H), 6.26-6.45 (m, 2H), 5.88-6.05 (m, 2H), 3.72 (t, J=8.01 Hz, 2H), 3.59-3.66 (m, 2H), 3.45-3.51 (m, 2H), 3.15-3.23 (m, 2H), 1.81-1.91 (m, 2H), 1.39-1.48 (m, 9H), 0.90-0.99 (m, 2H), 0.70-0.87 (m, 2H), −0.08-0.02 (m, 9H), −0.30-−0.11 (m, 9H).

### tert-butyl (3-(3-(6-(4-(dimethylamino)phenyl)-1-((2-(trimethylsilyl)ethoxy)methyl)-1H-benzo[d]imidazol-2-yl)-1-((2-(trimethylsilyl)ethoxy)methyl)-1H-indazole-5-carboxamido)propyl)carbamate (20f)

Compound 20f was obtained from compound 19f by using tert-butyl (3-aminopropyl)carbamate according to General Synthesis Procedure A (yellow oil, yield: 32%).

¹H NMR (400 MHz, METHANOL-d₄) δ 8.93-8.99 (m, 1H), 8.02-8.09 (m, 1H), 7.79-7.88 (m, 2H), 7.70 (dd, J=0.57, 8.59 Hz, 1H), 7.50-7.66 (m, 3H), 6.84-6.94 (m, 2H), 6.24 (d, J=13.28 Hz, 2H), 5.92 (d, J=2.52 Hz, 2H), 3.66-3.75 (m, 2H), 3.59 (dt, J=5.04, 7.90 Hz, 2H), 3.49 (dt, J=1.37, 6.87 Hz, 2H), 3.13-3.23 (m, 2H), 2.99 (d, J=1.60 Hz, 6H), 1.76-1.90 (m, 2H), 1.39-1.45 (m, 9H), 0.90-0.95 (m, 2H), 0.75-0.86 (m, 2H), −0.09-−0.03 (m, 8H), −0.22-−0.16 (m, 8H).

### tert-butyl (3-(3-(6-(4-methoxyphenyl)-1-((2-(trimethylsilyl)ethoxy)methyl)-1H-benzo[d]imidazol-2-yl)-1-((2-(trimethylsilyl)ethoxy)methyl)-1H-indazole-5-carboxamido)propyl)carbamate (20g)

Compound 20g was obtained from compound 19g by using tert-butyl (3-aminopropyl)carbamate according to General Synthesis Procedure A (yellow oil, yield: 51%).

¹H NMR (400 MHz, METHANOL-d₄) δ 8.94-8.99 (m, 1H), 8.03-8.09 (m, 1H), 7.99 (dd, J=0.57, 1.72 Hz, 1H), 7.82-7.90 (m, 2H), 7.60-7.68 (m, 3H), 6.99-7.07 (m, 2H), 6.25-6.32 (m, 2H), 5.87-5.96 (m, 2H), 3.83-3.93 (m, 3H), 3.63-3.76 (m, 2H), 3.53-3.63 (m, 2H), 3.44-3.52 (m, 2H), 3.15-3.22 (m, 2H), 1.75-1.87 (m, 2H), 1.42 (d, J=7.79 Hz, 9H), 0.92 (t, J=7.90 Hz, 2H), 0.77-0.84 (m, 2H), −0.09-−0.01 (m, 9H), −0.25-−0.15 (m, 9H).

### tert-butyl ((1S,3S)-3-(3-(6-(furan-2-yl)-1-((2-(trimethylsilyl)ethoxy)methyl)-1H-benzo[d]imidazole-2-yl)-1-((2-(trimethylsilyl)ethoxy)methyl)-1H-indazole-5-carboxamido)cyclopentyl)carbamate (20h)

Compound 20 h was obtained from compound 19 h by using tert-butyl ((1S,3S)-3-aminocyclopentyl)carbamate according to General Synthesis Procedure A (light-brown solid, yield: 82%).

Mixture of two regioisomers. ¹H NMR (400 MHz, DMSO-d₆) δ 8.94-8.99 (m, 1H), 8.50 (d, J=7.33 Hz, 1H), 8.02-8.17 (m, 1H), 7.96-8.02 (m, 1H), 7.88-7.93 (m, 1H), 7.75-7.88 (m, 1H), 7.73-7.75 (m, 1H), 7.66-7.73 (m, 1H), 6.96-7.02 (m, 1H), 6.91 (d, J=7.79 Hz, 1H), 6.55-6.62 (m, 1H), 6.21-6.32 (m, 2H), 5.91 (s, 2H), 4.32-4.43 (m, 1H), 3.91-4.03 (m, 1H), 3.63 (t, J=7.80 Hz, 2H), 3.47-3.54 (m, 2H), 1.91-2.08 (m, 2H), 1.72-1.89 (m, 2H), 1.46-1.60 (m, 1H), 1.38-1.45 (m, 1H), 1.35 (s, 9H), 0.83 (t, J=8.20 Hz, 2H), 0.71-0.77 (m, 2H), −0.13 (s, 9H), −0.28-−0.24 (m, 9H). MS (ESI): [M+H]⁺=787.4

### Example 73: N-(3-aminopropyl)-3-(6-(furan-3-yl)-1H-benzo[d]imidazol-2-yl)-1H-indazole-5-carboxamide (21a)

Compound (21a) of Example 73 was obtained from compound 20a according to General Synthesis Procedure H (yellow oil, yield: 35%).

¹H NMR (400 MHz, METHANOL-d₄) δ 8.98-9.04 (m, 1H), 7.88-7.98 (m, 2H), 7.77-7.83 (m, 1H), 7.67 (dd, J=4.81, 8.47 Hz, 2H), 7.58 (t, J=1.60 Hz, 1H), 7.48-7.54 (m, 1H), 6.87 (dd, J=0.92, 1.83 Hz, 1H), 3.55 (t, J=6.64 Hz, 2H), 2.83 (t, J=7.10 Hz, 2H), 1.87 (quin, J=6.87 Hz, 2H). MS (ESI): [M+H]⁺=401.1

### Example 74: N-((1S,3S)-3-aminocyclopentyl)-3-(6-(furan-3-yl)-1H-benzo[d]imidazol-2-yl)-1H-indazole-5-carboxamide (21b)

Compound (21b) of Example 74 was obtained from compound 20b according to General Synthesis Procedure H (white solid, yield: 49%).

¹H NMR (400 MHz, METHANOL-d₄) δ 8.99 (dd, J=0.92, 1.60 Hz, 1H), 7.96 (dd, J=1.60, 8.70 Hz, 1H), 7.93 (dd, J=0.92, 1.60 Hz, 1H), 7.83 (s, 1H), 7.65-7.72 (m, 2H), 7.58 (t, J=1.72 Hz, 1H), 7.53 (dd, J=1.60, 8.24 Hz, 1H), 6.88 (dd, J=0.92, 1.83 Hz, 1H), 4.56-4.64 (m, 1H), 3.61 (quin, J=6.93 Hz, 1H), 2.28-2.37 (m, 1H), 2.17-2.26 (m, 1H), 2.03-2.11 (m, 1H), 1.91-2.00 (m, 1H), 1.70-1.81 (m, 1H), 1.46-1.56 (m, 1H). MS (ESI): [M+H]⁺=427.7

### Example 75: N-(azetidin-3-ylmethyl)-3-(6-(furan-3-yl)-1H-benzo[d]imidazol-2-yl)-1H-indazole-5-carboxamide (21c)

Compound (21c) of Example 75 was obtained from compound 20c according to General Synthesis Procedure H (white solid, yield: 23%).

¹H NMR (400 MHz, METHANOL-d₄) δ 9.08 (dd, J=0.80, 1.49 Hz, 1H), 8.00 (dd, J=1.60, 8.93 Hz, 1H), 7.95 (t, J=1.14 Hz, 1H), 7.85 (s, 1H), 7.69-7.75 (m, 2H), 7.59-7.61 (m, 1H), 7.57 (dd, J=1.60, 8.47 Hz, 1H), 6.88 (dd, J=0.92, 1.83 Hz, 1H), 4.15-4.22 (m, 2H), 4.04-4.12 (m, 2H), 3.69-3.75 (m, 2H), 2.03 (d, J=9.16 Hz, 1H). MS (ESI): [M+H]⁺=413.4

### Example 76: N-(3-aminopropyl)-3-(6-phenyl-1H-benzo[d]imidazol-2-yl)-1H-indazole-5-carboxamide (21d)

Compound (21d) of Example 76 was obtained from compound 20d according to General Synthesis Procedure H (yellow solid, yield: 32%).

¹H NMR (400 MHz, METHANOL-d₄) δ 9.05-9.09 (m, 1H), 7.98 (dd, J=1.60, 8.70 Hz, 1H), 7.87-7.93 (m, 1H), 7.76 (d, J=8.24 Hz, 1H), 7.65-7.72 (m, 3H), 7.59 (dd, J=1.72, 8.36 Hz, 1H), 7.43-7.52 (m, 2H), 7.30-7.37 (m, 1H), 3.60 (t, J=6.53 Hz, 2H), 3.08 (t, J=7.21 Hz, 2H), 2.04 (quin, J=6.87 Hz, 2H). MS (ESI): [M+H]⁺=411.1

### Example 77: N-(3-aminopropyl)-3-(6-(pyridin-4-yl)-1H-benzo[d]imidazol-2-yl)-1H-indazole-5-carboxamide (21e)

Compound (21e) of Example 77 was obtained from compound 20e according to General Synthesis Procedure H (yellow solid, yield: 42%).

¹H NMR (400 MHz, METHANOL-d₄) δ 9.09 (dd, J=0.92, 1.60 Hz, 1H), 8.62-8.67 (m, 2H), 8.10-8.15 (m, 1H), 7.97-8.01 (m, 1H), 7.90-7.96 (m, 2H), 7.82-7.88 (m, 1H), 7.75-7.82 (m, 1H), 7.73 (dd, J=0.80, 8.82 Hz, 1H), 3.57-3.65 (m, 2H), 3.08 (t, J=7.33 Hz, 2H), 2.02-2.09 (m, 2H). MS (ESI): [M+H]⁺=412.1

### Example 78: N-(3-aminopropyl)-3-(6-(4-(dimethylamino)phenyl)-1H-benzo[d]imidazol-2-yl)-1H-indazole-5-carboxamide (21f)

Compound (21f) of Example 78 was obtained from compound 20f according to General Synthesis Procedure H (yellow solid, yield: 32%).

¹H NMR (400 MHz, DMSO-d₆) δ 9.07-9.17 (m, 1H), 7.96-8.04 (m, 1H), 7.65-7.76 (m, 2H), 7.55-7.65 (m, 2H), 7.48-7.54 (m, 1H), 7.21-7.40 (m, 1H), 6.79-6.96 (m, 2H), 3.38-3.48 (m, 2H), 2.92-3.03 (m, 6H), 2.84-2.91 (m, 2H), 1.91 (quin, J=7.04 Hz, 2H). MS (ESI): [M+H]⁺=454.2

### Example 79: N-(3-aminopropyl)-3-(6-(4-methoxyphenyl)-1H-benzo[d]imidazol-2-yl)-1H-indazole-5-carboxamide (21g)

Compound (21g) of Example 79 was obtained from compound 20g according to General Synthesis Procedure H (yellow solid, yield: 33%).

¹H NMR (400 MHz, METHANOL-d₄) δ 9.01-9.05 (m, 1H), 7.93-7.99 (m, 1H), 7.83 (d, J=1.37 Hz, 1H), 7.72 (d, J=8.47 Hz, 1H), 7.67-7.70 (m, 1H), 7.58-7.64 (m, 2H), 7.54 (dd, J=1.72, 8.36 Hz, 1H), 6.97-7.06 (m, 2H), 3.85 (s, 3H), 3.56 (t, J=6.75 Hz, 2H), 2.84 (t, J=6.98 Hz, 2H), 1.82-1.96 (m, 2H). MS (ESI): [M+H]⁺=444.1

### Example 80: N-((1S,3S)-3-aminocyclopentyl)-3-(6-(furan-2-yl)-1H-benzo[d]imidazol-2-yl)-1H-indazole-5-carboxamide (21h)

Compound (21h) of Example 80 was obtained from compound 20 h according to General Synthesis Procedure H (white solid, yield: 21%).

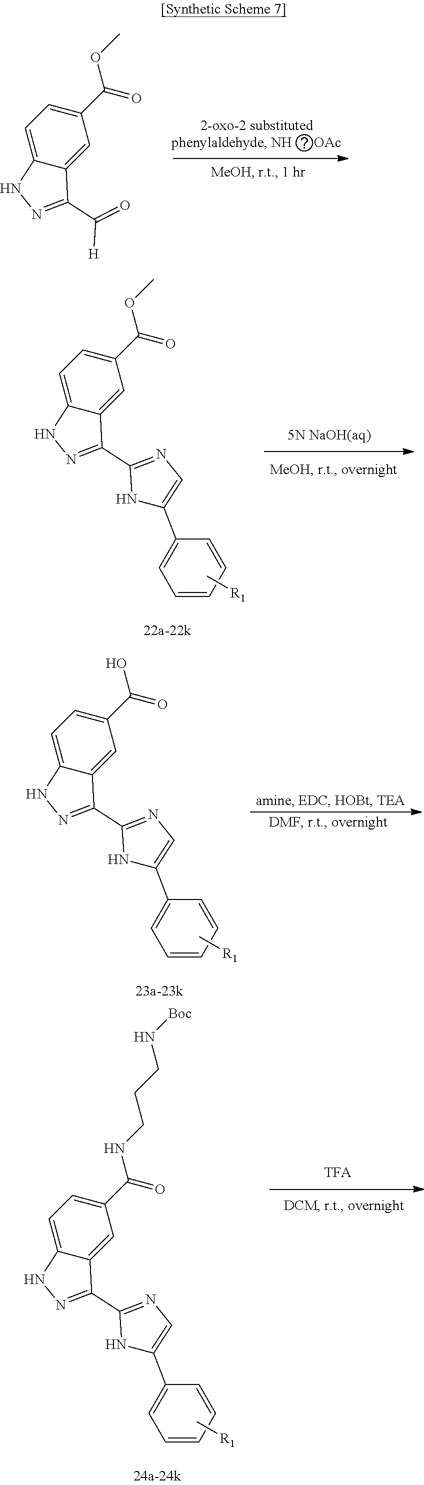

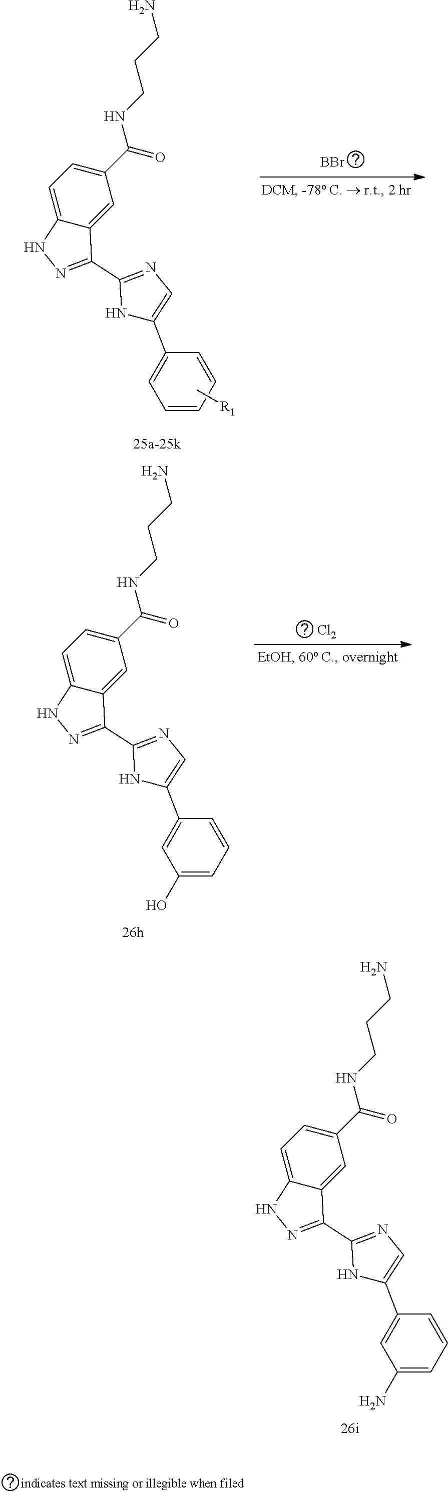

### methyl 3-(5-(4-fluorophenyl)-1H-imidazol-2-yl)-1H-indazole-5-carboxylate (22a)

Compound 22a was obtained by using 2-(4-fluorophenyl)-2-oxoacetaldehyde according to General Synthesis Procedure I (brown oil, yield: 22%).

¹H NMR (400 MHz, METHANOL-d₄) δ 9.11 (s, 1H), 7.98-8.09 (m, 1H), 7.82-7.93 (m, 2H), 7.57 (dd, J=0.69, 8.70 Hz, 1H), 7.47-7.55 (m, 1H), 7.05-7.17 (m, 2H), 3.93 (s, 3H). MS (ESI): [M+H]⁺=336.99

### methyl 3-(5-(3-fluorophenyl)-1H-imidazol-2-yl)-1H-indazole-5-carboxylate (22b)

Compound 22b was obtained by using 2-(3-fluorophenyl)-2-oxoacetaldehyde according to General Synthesis Procedure I (brown solid, yield: 48%).

¹H NMR (400 MHz, DMSO-d₆) δ 9.19 (s, 1H), 7.98-8.04 (m, 1H), 7.89 (d, J=2.14 Hz, 1H), 7.79 (td, J=1.11, 7.86 Hz, 1H), 7.65-7.76 (m, 2H), 7.47 (dt, J=6.41, 7.93 Hz, 1H), 7.02-7.11 (m, 1H), 3.93 (s, 3H). MS (ESI): [M+H]⁺=337.20

### methyl 3-(5-(2-fluorophenyl)-1H-imidazol-2-yl)-1H-indazole-5-carboxylate (22c)

Compound 22c was obtained by using 2-(2-fluorophenyl)-2-oxoacetaldehyde according to General Synthesis Procedure I (brown solid, yield: 38%).

¹H NMR (400 MHz, DMSO-d₆) δ 9.16-9.22 (m, 1H), 8.24 (dt, J=1.98, 7.55 Hz, 1H), 8.02 (dd, J=1.83, 8.85 Hz, 1H), 7.70 (dd, J=0.92, 8.85 Hz, 1H), 7.57 (dd, J=2.14, 3.97 Hz, 1H), 7.27-7.37 (m, 3H), 3.94 (s, 3H). MS (ESI): [M+H]⁺=337.61

### methyl 3-(5-(3,4-difluorophenyl)-1H-imidazol-2-yl)-1H-indazole-5-carboxylate (22d)

Compound 22d was obtained by using 2-(3,4-difluorophenyl)-2-oxoacetaldehyde according to General Synthesis Procedure I (brown solid, yield: 42%).

¹H NMR (400 MHz, DMSO-d₆) δ 9.18 (s, 1H), 7.98-8.03 (m, 1H), 7.91 (ddd, J=2.14, 7.93, 12.21 Hz, 1H), 7.87 (d, J=2.14 Hz, 1H), 7.76-7.82 (m, 1H), 7.67-7.71 (m, 1H), 7.45-7.55 (m, 1H), 3.92 (s, 3H). MS (ESI): [M+H]⁺=355.12

### methyl 3-(5-(4-chloro-3-fluorophenyl)-1H-imidazol-2-yl)-1H-indazole-5-carboxylate (22e)

Compound 22e was obtained by using 2-(4-chloro-3-fluorophenyl)-2-oxoacetaldehyde according to General Synthesis Procedure I (brown solid, yield: 28%).

¹H NMR (400 MHz, DMSO-d₆) δ 9.17 (dd, J=0.76, 1.68 Hz, 1H), 8.01 (dd, J=1.53, 8.85 Hz, 1H), 7.95 (s, 1H), 7.90 (dd, J=1.83, 10.99 Hz, 1H), 7.82 (dd, J=1.83, 8.24 Hz, 1H), 7.69 (dd, J=0.61, 8.85 Hz, 1H), 7.64 (t, J=8.09 Hz, 1H), 3.93 (s, 3H). MS (ESI): [M+H]⁺=371.41

### methyl 3-(5-(4-(trifluoromethyl)phenyl)-1H-imidazol-2-yl)-1H-indazole-5-carboxylate (22f)

Compound 22f was obtained by using 2-oxo-2-(4-(trifluoromethyl)phenyl)acetaldehyde according to General Synthesis Procedure I (red solid, yield: 39%).

¹H NMR (400 MHz, DMSO-d₆) δ 9.19 (dd, J=0.61, 1.53 Hz, 1H), 8.11-8.17 (m, J=7.93 Hz, 2H), 7.98-8.03 (m, 2H), 7.76-7.83 (m, J=7.93 Hz, 2H), 7.70 (dd, J=0.92, 8.85 Hz, 1H), 3.92 (s, 3H). MS (ESI): [M+H]⁺=387.03

### methyl 3-(5-(3,5-dichloro-2-methoxyphenyl)-1H-imidazol-2-yl)-1H-indazole-5-carboxylate (22g)

Compound 22g was obtained by using 2-(3,5-dichloro-2-methoxyphenyl)-2-oxoacetaldehyde according to General Synthesis Procedure I (yellowish oil, yield: 84%).

¹H NMR (400 MHz, DMSO-d₆) δ 9.21 (s, 1H), 8.19 (d, J=2.75 Hz, 1H), 8.02 (dd, J=1.37, 8.70 Hz, 1H), 7.75-7.79 (m, 1H), 7.70 (dd, J=0.80, 8.82 Hz, 1H), 7.55 (d, J=2.75 Hz, 1H), 3.92 (s, 3H), 3.85 (s, 3H). MS (ESI): [M+H]⁺=417.2.

### methyl 3-(5-(3-methoxyphenyl)-1H-imidazol-2-yl)-1H-indazole-5-carboxylate (22h)

Compound 22 h was obtained by using 2-(3-methoxyphenyl)-2-oxoacetaldehyde according to General Synthesis Procedure I (brown solid, yield: 42%).

¹H NMR (400 MHz, DMSO-d₆) δ 9.23 (s, 1H), 7.97-8.03 (m, 1H), 7.80 (s, 1H), 7.68 (dd, J=0.92, 8.85 Hz, 1H), 7.49-7.56 (m, 2H), 7.33 (t, J=7.93 Hz, 1H), 6.82 (ddd, J=0.92, 2.52, 8.16 Hz, 1H), 3.92 (s, 3H), 3.34 (s, 3H). MS (ESI): [M+H]⁺=349.21

### methyl 3-(5-(3-nitrophenyl)-1H-imidazol-2-yl)-1H-indazole-5-carboxylate (22i)

Compound 22i was obtained by using 2-(3-nitrophenyl)-2-oxoacetaldehyde according to General Synthesis Procedure I (brown solid, yield: 31%).

¹H NMR (400 MHz, DMSO-d₆) δ 9.23 (s, 1H), 8.79 (t, J=1.83 Hz, 1H), 8.33-8.39 (m, 1H), 8.10 (ddd, J=0.92, 2.44, 8.24 Hz, 1H), 8.03-8.07 (m, 1H), 8.01 (dd, J=1.68, 8.70 Hz, 1H), 7.67-7.76 (m, 2H), 3.94 (s, 3H). MS (ESI): [M+H]⁺=363.94

### methyl 3-(5-(4-morpholinophenyl)-1H-imidazol-2-yl)-1H-indazole-5-carboxylate (22j)

Compound 22j was obtained by using 2-(4-morpholinophenyl)-2-oxoacetaldehyde according to General Synthesis Procedure I (yellow oil, yield: 24%).

¹H NMR (400 MHz, DMSO-d₆) δ 9.20 (s, 1H), 7.97-8.02 (m, 1H), 7.77-7.83 (m, 2H), 7.65-7.69 (m, 1H), 7.60 (d, J=2.29 Hz, 1H), 6.99-7.05 (m, 2H), 3.92 (s, 3H), 3.75-3.78 (m, 4H), 3.11-3.17 (m, 4H). MS (ESI): [M+H]⁺=404.0

### methyl 3-(5-(3-morpholinophenyl)-1H-imidazol-2-yl)-1H-indazole-5-carboxylate (22k)

Compound 22k was obtained by using 2-(3-morpholinophenyl)-2-oxoacetaldehyde according to General Synthesis Procedure I (light brown solid, yield: 39%).

¹H NMR (400 MHz, DMSO-d₆) δ 9.21 (br. s., 1H), 8.00 (dd, J=1.72, 8.82 Hz, 1H), 7.66 (d, J=8.70 Hz, 2H), 7.54 (br. s., 1H), 7.35 (br. s., 1H), 7.27 (t, J=7.79 Hz, 1H), 6.84 (dd, J=2.18, 8.59 Hz, 1H), 3.93 (s, 3H), 3.76-3.85 (m, 4H), 3.21-3.25 (m, 4H). MS (ESI): [M+H]⁺=403.8.

### 3-(5-(4-fluorophenyl)-1H-imidazol-2-yl)-1H-indazole-5-carboxylic acid (23a)

Compound 23a was obtained from compound 22a according to General Synthesis Procedure D (black solid, yield: 83%).

¹H NMR (400 MHz, METHANOL-d₄) δ 9.05 (s, 1H), 8.15 (dd, J=1.53, 8.85 Hz, 1H), 7.86-7.93 (m, 2H), 7.80 (s, 1H), 7.70 (d, J=8.85 Hz, 1H), 7.19-7.26 (m, 2H). MS (ESI): [M+H]⁺=322.95

### 3-(5-(3-fluorophenyl)-1H-imidazol-2-yl)-1H-indazole-5-carboxylic acid (23b)

Compound 23b was obtained from compound 22b according to General Synthesis Procedure D (light brown solid, yield: 54%).

¹H NMR (400 MHz, DMSO-d₆) δ 9.10 (s, 1H), 8.00-8.13 (m, 2H), 7.72-7.85 (m, 3H), 7.52 (dt, J=6.26, 8.01 Hz, 1H), 7.05-7.20 (m, 1H). MS (ESI): [M+H]⁺=323.08

### 3-(5-(2-fluorophenyl)-1H-imidazol-2-yl)-1H-indazole-5-carboxylic acid (23c)

Compound 23c was obtained from compound 22c according to General Synthesis Procedure D (brown solid, yield: 42%).

¹H NMR (400 MHz, DMSO-d₆) δ 9.09 (s, 1H), 8.15-8.23 (m, 1H), 8.04 (dd, J=1.53, 8.55 Hz, 1H), 7.71-7.85 (m, 2H), 7.31-7.46 (m, 3H). MS (ESI): [M+H]⁺=323.01

### 3-(5-(3,4-difluorophenyl)-1H-imidazol-2-yl)-1H-indazole-5-carboxylic acid (23d)

Compound 23d was obtained from compound 22d according to General Synthesis Procedure D (brown solid, yield: 32%).

¹H NMR (400 MHz, DMSO-d₆) δ 9.08 (s, 1H), 7.99-8.14 (m, 3H), 7.78-7.90 (m, 1H), 7.74 (d, J=8.85 Hz, 1H), 7.53-7.63 (m, 1H). MS (ESI): [M+H]⁺=341.07

### 3-(5-(4-chloro-3-fluorophenyl)-1H-imidazol-2-yl)-1H-indazole-5-carboxylic acid (23e)

Compound 23e was obtained from compound 22e according to General Synthesis Procedure D (red solid, yield: 37%).

¹H NMR (400 MHz, DMSO-d₆) δ 9.10 (s, 1H), 8.00-8.10 (m, 2H), 7.97 (dd, J=1.53, 10.99 Hz, 1H), 7.83 (dd, J=1.83, 8.24 Hz, 1H), 7.65-7.74 (m, 2H). MS (ESI): [M+H]⁺=356.82

### 3-(5-(4-(trifluoromethyl)phenyl)-1H-imidazol-2-yl)-1H-indazole-5-carboxylic acid (23f)

Compound 23f was obtained from compound 22f according to General Synthesis Procedure D (brown solid, yield: 27%).

¹H NMR (400 MHz, DMSO-d₆) δ 9.09-9.16 (m, 1H), 8.09-8.21 (m, J=7.93 Hz, 2H), 7.99-8.07 (m, 2H), 7.76-7.84 (m, J=8.24 Hz, 2H), 7.69 (dd, J=0.61, 8.85 Hz, 1H).

MS (ESI): [M+H]⁺=372.97

### 3-(5-(3,5-dichloro-2-methoxyphenyl)-1H-imidazol-2-yl)-1H-indazole-5-carboxylic acid (23g)

Compound 23g was obtained from compound 22g according to General Synthesis Procedure D (brown solid, yield: 95%).

¹H NMR (400 MHz, DMSO-d₆) δ 9.12-9.15 (m, 1H), 8.16 (d, J=2.52 Hz, 1H), 8.02 (dd, J=1.60, 8.93 Hz, 1H), 7.82 (s, 1H), 7.69 (dd, J=0.80, 8.82 Hz, 1H), 7.57 (d, J=2.75 Hz, 1H), 3.84 (s, 3H). MS (ESI): [M+H]⁺=403.1.

### 3-(5-(3-methoxyphenyl)-1H-imidazol-2-yl)-1H-indazole-5-carboxylic acid (23h)

Compound 23 h was obtained from compound 22 h according to General Synthesis Procedure D (brown solid, yield: 34%).

¹H NMR (400 MHz, DMSO-d₆) δ 9.07 (s, 1H), 8.09-8.24 (m, 1H), 8.01-8.07 (m, 1H), 7.73-7.80 (m, 1H), 7.50-7.61 (m, 2H), 7.37-7.45 (m, 1H), 6.88-6.98 (m, 1H), 3.84 (s, 3H). MS (ESI): [M+H]⁺=335.03

### 3-(5-(3-nitrophenyl)-1H-imidazol-2-yl)-1H-indazole-5-carboxylic acid (23i)

Compound 23i was obtained from compound 22i according to General Synthesis Procedure D (black solid, yield: 64%).

¹H NMR (400 MHz, DMSO-d₆) δ 9.16 (s, 1H), 8.78 (s, 1H), 8.37 (d, J=7.63 Hz, 1H), 8.07-8.16 (m, 2H), 8.02 (dd, J=1.53, 8.55 Hz, 1H), 7.65-7.77 (m, 2H). MS (ESI): [M+H]⁺=349.89

### 3-(5-(4-morpholinophenyl)-1H-imidazol-2-yl)-1H-indazole-5-carboxylic acid (23j)

Compound 23j was obtained from compound 22j according to General Synthesis Procedure D (brown solid, yield: 47%).

¹H NMR (400 MHz, DMSO-d₆) δ 9.09 (s, 1H), 7.97-8.08 (m, 2H), 7.78-7.82 (m, 2H), 7.65-7.74 (m, 1H), 7.04 (d, J=8.93 Hz, 2H), 3.72-3.79 (m, 4H), 3.13-3.20 (m, 4H).

MS (ESI): [M+H]⁺=399.9

### 3-(5-(3-morpholinophenyl)-1H-imidazol-2-yl)-1H-indazole-5-carboxylic acid (23k)

Compound 23k was obtained from compound 22k according to General Synthesis Procedure D (brown solid, yield: 60%).

¹H NMR (400 MHz, DMSO-d₆) δ 8.99-9.16 (m, 1H), 8.00 (dd, J=1.03, 8.59 Hz, 1H), 7.74 (s, 1H), 7.51-7.63 (m, 2H), 7.39-7.46 (m, 1H), 7.21-7.32 (m, 1H), 6.79-6.85 (m, 1H), 3.75-3.83 (m, 4H), 3.16-3.25 (m, 4H). MS (ESI): [M+H]⁺=389.9

### tert-butyl (3-(3-(5-(4-fluorophenyl)-1H-imidazol-2-yl)-1H-indazole-5-carboxamido)propyl)carbamate (24a)

Compound 24a was obtained from compound 23a by using tert-butyl (3-aminopropyl)carbamate according to General Synthesis Procedure A (brown oil, yield: 34%).

¹H NMR (400 MHz, METHANOL-d₄) δ 8.89 (s, 1H), 7.83-8.01 (m, 3H), 7.56-7.65 (m, 1H), 7.54 (s, 1H), 7.06-7.20 (m, 2H), 3.48 (t, J=6.71 Hz, 2H), 3.20 (t, J=6.56 Hz, 2H), 1.81 (quin, J=6.71 Hz, 2H), 1.43 (s, 9H). MS (ESI): [M+H]⁺=479.04

### tert-butyl (3-(3-(5-(3-fluorophenyl)-1H-imidazol-2-yl)-1H-indazole-5-carboxamido)propyl)carbamate (24b)

Compound 24b was obtained from compound 23b by using tert-butyl (3-aminopropyl)carbamate according to General Synthesis Procedure A (brown oil, yield: 27%).

¹H NMR (400 MHz, METHANOL-d₄) δ 8.90-8.99 (m, 1H), 7.85-7.95 (m, 1H), 7.56-7.81 (m, 4H), 7.36-7.46 (m, 1H), 6.92-7.03 (m, 1H), 3.45-3.51 (m, 2H), 3.16-3.24 (m, 2H), 1.82 (quin, J=6.56 Hz, 2H), 1.42 (s, 9H). MS (ESI): [M+H]⁺=478.91 tert-butyl (3-(3-(5-(2-fluorophenyl)-1H-imidazol-2-yl)-1H-indazole-5-carboxamido)propyl) carbamate (24c)

Compound 24c was obtained from compound 23c by using tert-butyl (3-aminopropyl)carbamate according to General Synthesis Procedure A (brown oil, yield: 45%).

¹H NMR (400 MHz, METHANOL-d₄) δ 8.93-9.01 (m, 1H), 8.28-8.40 (m, 1H), 7.88-7.94 (m, 1H), 7.59-7.64 (m, 2H), 7.12-7.32 (m, 3H), 3.43-3.54 (m, 2H), 3.21 (t, J=6.56 Hz, 2H), 1.76-1.85 (m, 2H), 1.44 (s, 9H). MS (ESI): [M+H]⁺=479.04 tert-butyl (3-(3-(5-(3,4-difluorophenyl)-1H-imidazol-2-yl)-1H-indazole-5-carboxamido)propyl)carbamate (24d)

Compound 24d was obtained from compound 23d by using tert-butyl (3-aminopropyl)carbamate according to General Synthesis Procedure A (red solid, yield: 66%).

¹H NMR (400 MHz, METHANOL-d₄) δ 8.96 (s, 1H), 7.89-7.95 (m, 1H), 7.80-7.89 (m, 1H), 7.67-7.77 (m, 1H), 7.62 (dd, J=0.92, 8.85 Hz, 2H), 7.25-7.36 (m, 1H), 3.49 (t, J=6.70 Hz, 2H), 3.21 (t, J=6.71 Hz, 2H), 1.76-1.85 (m, 2H), 1.44 (s, 9H). MS (ESI): [M+H]⁺=496.97

### tert-butyl (3-(3-(5-(4-chloro-3-fluorophenyl)-1H-imidazol-2-yl)-1H-indazole-5-carboxamido)propyl)carbamate (24e)

Compound 24e was obtained from compound 23e by using tert-butyl (3-aminopropyl)carbamate according to General Synthesis Procedure A (yellow oil, yield: 42%).

¹H NMR (400 MHz, METHANOL-d₄) δ 8.94 (br. s., 1H), 7.88-7.91 (m, 1H), 7.77-7.86 (m, 1H), 7.68-7.75 (m, 1H), 7.63-7.68 (m, 1H), 7.60 (dd, J=0.92, 8.85 Hz, 1H), 7.46 (t, J=8.09 Hz, 1H), 3.49 (t, J=6.71 Hz, 2H), 3.21 (t, J=6.71 Hz, 2H), 1.82 (t, J=6.71 Hz, 2H), 1.43 (s, 9H). MS (ESI): [M+H]⁺=512.87

### tert-butyl (3-(3-(5-(4-(trifluoromethyl)phenyl)-1H-imidazol-2-yl)-1H-indazole-5-carboxamido)propyl)carbamate (24f)

Compound 24f was obtained from compound 23f by using tert-butyl (3-aminopropyl)carbamate according to General Synthesis Procedure A (red solid, yield: 43%).

¹H NMR (400 MHz, METHANOL-d₄) δ 8.94-9.02 (m, 1H), 8.07-8.20 (m, 2H), 7.92 (dd, J=1.53, 8.85 Hz, 1H), 7.68-7.77 (m, 3H), 7.63 (dd, J=0.76, 8.70 Hz, 1H), 3.49 (t, J=6.40 Hz, 2H), 3.22 (t, J=6.41 Hz, 2H), 1.79-1.86 (m, 2H), 1.42 (s, 9H). MS (ESI): [M+H]⁺=529.04

### tert-butyl (3-(3-(5-(3,5-dichloro-2-methoxyphenyl)-1H-imidazol-2-yl)-1H-indazole-5-carboxamido)propyl)carbamate (24g)

Compound 24g was obtained from compound 23g by using tert-butyl (3-aminopropyl)carbamate according to General Synthesis Procedure A (yellowish solid, yield: 72%).

¹H NMR (400 MHz, METHANOL-d₄) δ 8.97 (s, 1H), 8.26 (s, 1H), 7.91 (d, J=8.70 Hz, 1H), 7.82 (s, 1H), 7.62 (dd, J=0.80, 8.82 Hz, 1H), 7.33-7.39 (m, 1H), 3.86 (s, 3H), 3.49 (t, J=6.75 Hz, 2H), 3.21 (t, J=6.64 Hz, 2H), 1.82 (quin, J=6.64 Hz, 2H), 1.42 (s, 9H). MS (ESI): [M+H]⁺=559.0.

### tert-butyl (3-(3-(5-(3-methoxyphenyl)-1H-imidazol-2-yl)-1H-indazole-5-carboxamido)propyl)carbamate (24h)

Compound 24 h was obtained from compound 23 h by using tert-butyl (3-aminopropyl)carbamate according to General Synthesis Procedure A (yellow solid, yield: 32%).

¹H NMR (400 MHz, METHANOL-d₄) δ 8.90 (s, 1H), 7.88-7.96 (m, 1H), 7.60-7.64 (m, 1H), 7.58 (s, 1H), 7.36-7.54 (m, 2H), 7.28-7.36 (m, 1H), 6.80-6.88 (m, 1H), 3.88 (s, 3H), 3.48 (t, J=6.87 Hz, 2H), 3.20 (t, J=6.90 Hz, 2H), 1.81 (quin, J=6.71 Hz, 2H), 1.40 (s, 9H). MS (ESI): [M+H]⁺=491.13

### tert-butyl (3-(3-(5-(3-nitrophenyl)-1H-imidazol-2-yl)-1H-indazole-5-carboxamido)propyl)carbamate (24i)

Compound 24i was obtained from compound 23i by using tert-butyl (3-aminopropyl)carbamate according to General Synthesis Procedure A (yellow oil, yield: 25%).

¹H NMR (400 MHz, METHANOL-d₄) δ 8.96 (s, 1H), 8.70-8.81 (m, 1H), 8.28-8.39 (m, 1H), 8.08-8.16 (m, 1H), 7.91-7.94 (m, 1H), 7.78-7.82 (m, 1H), 7.61-7.70 (m, 2H), 3.47-3.52 (m, 2H), 3.18-3.24 (m, 2H), 1.81-1.86 (m, 2H), 1.42 (s, 9H). MS (ESI): [M+H]⁺=505.94

### tert-butyl (3-(3-(5-(4-morpholinophenyl)-1H-imidazol-2-yl)-1H-indazole-5-carboxamido)propyl)carbamate (24j)

Compound 24j was obtained from compound 23j by using tert-butyl (3-aminopropyl)carbamate according to General Synthesis Procedure A (yellow oil, yield: 50%).

¹H NMR (400 MHz, METHANOL-d₄) δ 8.81-8.97 (m, 1H), 7.88-7.95 (m, 1H), 7.59-7.88 (m, 3H), 7.39-7.48 (m, 1H), 7.01-7.08 (m, 2H), 3.81-3.90 (m, 4H), 3.44-3.52 (m, 2H), 3.14-3.26 (m, 6H), 1.77-1.88 (m, 2H), 1.43 (s, 9H). MS (ESI): [M+H]⁺=546.2

### tert-butyl (3-(3-(5-(3-morpholinophenyl)-1H-imidazol-2-yl)-1H-indazole-5-carboxamido) propyl)carbamate (24k)

Compound 24k was obtained from compound 23k by using tert-butyl (3-aminopropyl)carbamate according to General Synthesis Procedure A (yellowish oil, yield: 59%).

¹H NMR (400 MHz, METHANOL-d₄) δ 8.90 (br. s., 1H), 7.92 (d, J=8.93 Hz, 1H), 7.63 (dd, J=0.92, 8.70 Hz, 1H), 7.57 (br. s, 1H), 7.40 (br. s., 1H), 7.31 (t, J=7.56 Hz, 2H), 6.92 (dd, J=1.03, 7.67 Hz, 1H), 3.85-3.92 (m, 4H), 3.48 (t, J=6.87 Hz, 2H), 3.22-3.27 (m, 4H), 3.19 (t, J=6.64 Hz, 2H), 1.82 (quin, J=6.70 Hz, 2H), 1.43 (s, 9H). MS (ESI): [M+H]⁺=546.5

### Example 81: N-(3-aminopropyl)-3-(5-(4-fluorophenyl)-1H-imidazol-2-yl)-1H-indazole-5-carboxamide (25a)

Compound (25a) of Example 81 was obtained from compound 24a according to General Synthesis Procedure B (yellowish oil, yield: 45%).

¹H NMR (400 MHz, METHANOL-d₄) δ 8.90-8.94 (m, 1H), 7.85-7.96 (m, 3H), 7.60-7.67 (m, 1H), 7.55 (s, 1H), 7.12-7.21 (m, 2H), 3.54 (t, J=6.71 Hz, 2H), 2.83 (t, J=7.02 Hz, 2H), 1.82-1.91 (m, 2H). MS (ESI): [M+H]⁺=378.95

### Example 82: N-(3-aminopropyl)-3-(5-(3-fluorophenyl)-1H-imidazol-2-yl)-1H-indazole-5-carboxamide (25b)

Compound (25b) of Example 82 was obtained from compound 24b according to General Synthesis Procedure B (yellowish oil, yield: 47%).

¹H NMR (400 MHz, METHANOL-d₄) δ 8.88 (dd, J=0.92, 1.83 Hz, 1H), 7.85-7.89 (m, 1H), 7.61-7.69 (m, 2H), 7.56-7.61 (m, 2H), 7.38 (dt, J=5.80, 7.93 Hz, 1H), 6.92-6.99 (m, 1H), 3.49 (t, J=7.00 Hz, 2H), 2.76 (t, J=7.02 Hz, 2H), 1.80 (quin, J=6.87 Hz, 2H). MS (ESI): [M+H]⁺=379.01

### Example 83: N-(3-aminopropyl)-3-(5-(2-fluorophenyl)-1H-imidazol-2-yl)-1H-indazole-5-carboxamide (25c)

Compound (25c) of Example 83 was obtained from compound 24c according to General Synthesis Procedure B (white solid, yield: 44%).

¹H NMR (400 MHz, DMSO-d₆) δ 8.98-9.05 (m, 1H), 8.67-8.82 (m, 1H), 8.27-8.42 (m, 1H), 7.89-8.00 (m, 1H), 7.61-7.78 (m, 1H), 7.53-7.59 (m, 1H), 7.22-7.37 (m, 3H), 3.39 (q, J=6.49 Hz, 2H), 2.70-2.89 (m, 2H), 1.69-1.85 (m, 2H). MS (ESI): [M+H]⁺=378.95

### Example 84: N-(3-aminopropyl)-3-(5-(3,4-difluorophenyl)-1H-imidazol-2-yl)-1H-indazole-5-carboxamide (25d)

Compound (25d) of Example 84 was obtained from compound 24d according to General Synthesis Procedure B (yellowish oil, yield: 31%).

¹H NMR (400 MHz, METHANOL-d₄) δ 8.90-8.98 (m, 1H), 7.92-7.96 (m, 1H), 7.84 (ddd, J=1.83, 7.78, 12.05 Hz, 1H), 7.63-7.71 (m, 3H), 7.26-7.36 (m, 1H), 3.58 (t, J=6.41 Hz, 2H), 3.06 (t, J=7.32 Hz, 2H), 1.90-2.12 (m, 2H). MS (ESI): [M+H]⁺=396.87

### Example 85: N-(3-aminopropyl)-3-(5-(4-chloro-3-fluorophenyl)-1H-imidazol-2-yl)-1H-indazole-5-carboxamide (25e)

Compound (25e) of Example 85 was obtained from compound 24e according to General Synthesis Procedure B (yellowish oil, yield: 43%).

¹H NMR (400 MHz, DMSO-d₆) δ 8.97 (s, 1H), 8.70 (t, J=5.65 Hz, 1H), 7.97 (dd, J=1.98, 11.14 Hz, 1H), 7.90 (dd, J=1.68, 8.70 Hz, 1H), 7.85 (dd, J=1.83, 8.55 Hz, 1H), 7.60-7.66 (m, 2H), 3.35-3.41 (m, 2H), 2.67 (t, J=6.71 Hz, 2H), 1.61-1.70 (m, 2H). MS (ESI): [M+H]⁺=412.90

### Example 86: N-(3-aminopropyl)-3-(5-(4-(trifluoromethyl)phenyl)-1H-imidazol-2-yl)-1H-indazole-5-carboxamide (25f)

Compound (25f) of Example 86 was obtained from compound 24f according to General Synthesis Procedure B (yellowish oil, yield: 41%).

¹H NMR (400 MHz, METHANOL-d₄) δ 8.97 (dd, J=0.92, 1.83 Hz, 1H), 8.10 (d, J=7.93 Hz, 2H), 7.94 (dd, J=1.83, 8.85 Hz, 1H), 7.77 (s, 1H), 7.71 (dd, J=0.61, 8.85 Hz, 2H), 7.65 (dd, J=0.92, 8.85 Hz, 1H), 3.58 (t, J=6.56 Hz, 2H), 3.05 (t, J=7.32 Hz, 2H), 1.95-2.06 (m, 2H). MS (ESI): [M+H]⁺=428.92

### Example 87: N-(3-aminopropyl)-3-(5-(3,5-dichloro-2-methoxyphenyl)-1H-imidazol-2-yl)-1H-indazole-5-carboxamide (25g)

Compound (25g) of Example 87 was obtained from compound 24g according to General Synthesis Procedure B (white solid, yield: 22%).

¹H NMR (400 MHz, METHANOL-d₄) δ 8.96 (td, J=0.80, 1.60 Hz, 1H), 8.20 (s, 1H), 7.88-7.94 (m, 1H), 7.82 (d, J=0.69 Hz, 1H), 7.64 (td, J=0.77, 8.76 Hz, 1H), 7.37 (dd, J=0.57, 2.63 Hz, 1H), 3.86 (d, J=0.69 Hz, 3H), 3.54 (t, J=6.64 Hz, 2H), 2.83 (t, J=6.98 Hz, 2H), 1.87 (quin, J=6.90 Hz, 2H). MS (ESI): [M+H]⁺=459.1

### Example 88: N-(3-aminopropyl)-3-(5-(3-methoxyphenyl)-1H-imidazol-2-yl)-1H-indazole-5-carboxamide (25h)

Compound (25h) of Example 88 was obtained from compound 24 h according to General Synthesis Procedure B (yellowish oil, yield: 33%).

¹H NMR (400 MHz, METHANOL-d₄) δ 8.89 (dd, J=0.92, 1.83 Hz, 1H), 7.88-7.92 (m, 1H), 7.61 (dd, J=0.76, 8.70 Hz, 1H), 7.57 (s, 1H), 7.40-7.47 (m, 2H), 7.32 (t, J=8.09 Hz, 1H), 6.85 (ddd, J=0.92, 2.75, 8.24 Hz, 1H), 3.88 (s, 3H), 3.52 (t, J=6.87 Hz, 2H), 2.77 (t, J=6.87 Hz, 2H), 1.81 (quin, J=6.87 Hz, 2H). MS (ESI): [M+H]⁺=390.96

### Example 89: N-(3-aminopropyl)-3-(5-(3-nitrophenyl)-1H-imidazol-2-yl)-1H-indazole-5-carboxamide (25i)

Compound (25i) of Example 89 was obtained from compound 24i according to General Synthesis Procedure B (yellowish oil, yield: 21%).

¹H NMR (400 MHz, METHANOL-d₄) δ 9.00 (dd, J=0.92, 1.83 Hz, 1H), 8.82-8.85 (m, 1H), 8.29 (ddd, J=0.92, 1.75, 7.71 Hz, 1H), 8.15 (ddd, J=1.07, 2.37, 8.16 Hz, 1H), 7.94 (dd, J=1.53, 8.85 Hz, 1H), 7.85 (s, 1H), 7.64-7.71 (m, 2H), 3.58-3.59 (m, 1H), 3.59 (t, J=7.40 Hz, 2H), 3.11 (t, J=7.30 Hz, 2H), 2.00-2.08 (m, 2H). MS (ESI): [M+H]⁺=405.90

### Example 90: N-(3-aminopropyl)-3-(5-(4-morpholinophenyl)-1H-imidazol-2-yl)-1H-indazole-5-carboxamide (25j)

Compound (25j) of Example 90 was obtained from compound 24j according to General Synthesis Procedure B (yellowish oil, yield: 26%).

¹H NMR (400 MHz, METHANOL-d₄) δ 8.88 (dd, J=0.92, 1.60 Hz, 1H), 7.91 (dd, J=1.60, 8.93 Hz, 1H), 7.70-7.78 (m, 2H), 7.62 (dd, J=0.80, 8.82 Hz, 1H), 7.44 (s, 1H), 6.98-7.09 (m, 2H), 3.80-3.91 (m, 4H), 3.52 (t, J=6.60 Hz, 2H), 3.13-3.22 (m, 4H), 2.77 (t, J=6.90 Hz, 2H), 1.83 (quin, J=6.87 Hz, 2H). MS (ESI): [M+H]⁺=446.0

### Example 91: N-(3-aminopropyl)-3-(5-(3-morpholinophenyl)-1H-imidazol-2-yl)-1H-indazole-5-carboxamide (25k)

Compound (25k) of Example 91 was obtained from compound 24k according to General Synthesis Procedure B (light brown solid, yield: 38.9%).

¹H NMR (400 MHz, METHANOL-d₄) δ 8.91 (dd, J=0.80, 1.49 Hz, 1H), 7.93 (dd, J=1.72, 8.82 Hz, 1H), 7.64 (dd, J=0.80, 8.82 Hz, 1H), 7.57 (s, 1H), 7.49 (br. s., 1H), 7.29-7.38 (m, 2H), 6.90-6.95 (m, 1H), 3.86-3.91 (m, 4H), 3.55 (t, J=6.64 Hz, 2H), 3.22-3.27 (m, 4H), 2.96 (t, J=7.10 Hz, 2H), 1.95 (quin, J=6.93 Hz, 2H). MS (ESI): [M+H]⁺=446.5

### Example 92: N-(3-aminopropyl)-3-(5-(3-hydroxyphenyl)-1H-imidazol-2-yl)-1H-indazole-5-carboxamide (26h)

Compound 25 h was dissolved in DCM and cooled to −78° C., and BBr₃ in DCM (2.0 eq) was slowly added, and the solution was stirred at room temperature for 2 hours. Upon completion of the reaction, the solution was extracted with saturated aqueous NaHCO₃ solution and DCM, dried over Na₂SO₄, and filtered. The solvent was removed under reduced pressure, and the residue was purified by column chromatography to afford compound (26h) of Example 92 (light brown oil, yield: 25%).

¹H NMR (400 MHz, METHANOL-d₄) δ 8.88 (dd, J=0.92, 1.83 Hz, 1H), 7.87 (dd, J=1.83, 8.85 Hz, 1H), 7.57 (dd, J=0.92, 8.85 Hz, 1H), 7.50 (s, 1H), 7.18-7.36 (m, 3H), 6.72 (ddd, J=1.22, 2.52, 7.86 Hz, 1H), 3.51 (t, J=6.71 Hz, 2H), 2.80 (t, J=7.02 Hz, 2H), 1.82 (quin, J=6.79 Hz, 2H). MS (ESI): [M+H]⁺=376.84

### Example 93: 3-(5-(3-aminophenyl)-1H-imidazol-2-yl)-N-(3-aminopropyl)-1H-indazole-5-carboxamide (26i)

Compound 25i was dissolved in ethanol, and SnCl₂—H₂O (5.0 eq) was added, and the solution was heated to 60° C. and stirred for 5 hours. Upon completion of the reaction, the solvent was removed under reduced pressure, and the residue was extracted with TN aqueous NaOH solution and ethyl acetate, dried over Na₂SO₄, and filtered. The solvent was removed under reduced pressure, and the residue was purified by column chromatography to afford compound (26i) of Example 93 (yellowish solid, yield: 25%).

¹H NMR (400 MHz, METHANOL-d₄) δ 8.86-8.93 (m, 1H), 7.87-7.98 (m, 1H), 7.62 (dd, J=0.92, 8.85 Hz, 1H), 7.49 (s, 1H), 7.12-7.27 (m, 3H), 6.65-6.74 (m, 1H), 3.48-3.56 (m, 2H), 2.85 (t, J=7.02 Hz, 2H), 1.84-1.91 (m, 2H). MS (ESI): [M+H]⁺=375.89

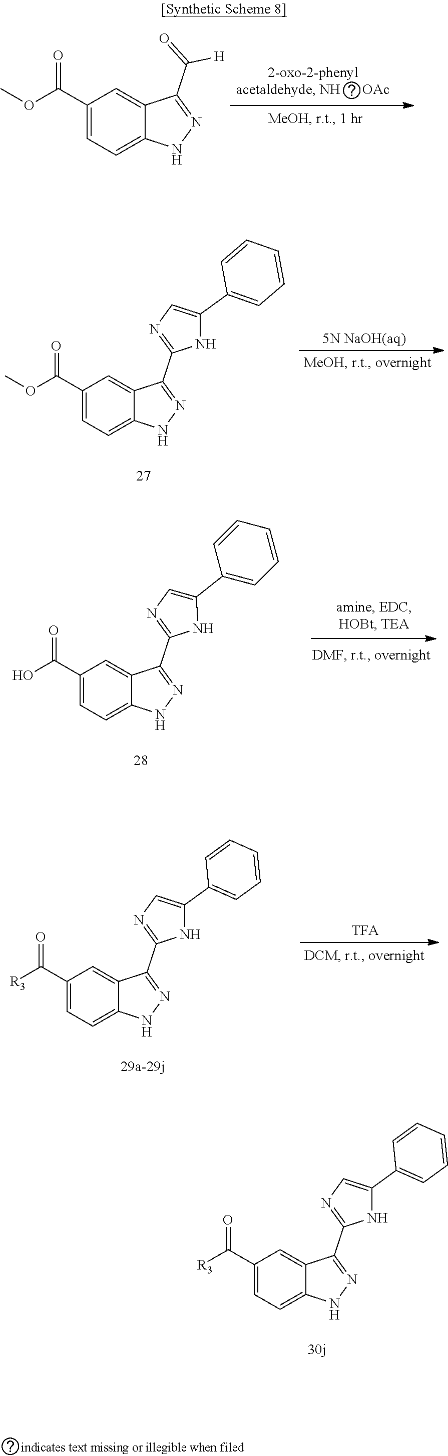

### methyl 3-(4-phenyl-1H-imidazol-2-yl)-1H-indazole-5-carboxylate (27)

Compound 27 was obtained by using 2-oxo-2-phenylacetaldehyde according to General Synthesis Procedure I (brown solid, yield: 20%).

¹H NMR (400 MHz, DMSO-d₆) δ 9.17-9.22 (m, 1H), 8.01 (dd, J=1.60, 8.93 Hz, 1H), 7.90-7.96 (m, 2H), 7.72-7.79 (m, 1H), 7.67-7.70 (m, 1H), 7.40-7.45 (m, 2H), 7.22-7.28 (m, 1H), 3.92 (s, 3H). MS (ESI): [M+H]⁺=319.69

### 3-(4-phenyl-1H-imidazol-2-yl)-1H-indazole-5-carboxylic acid (28)

Compound 28 was obtained from compound 27 according to General Synthesis Procedure D (yellowish solid, yield: 80%).

¹H NMR (400 MHz, DMSO-d₆) δ 13.61-13.77 (m, 1H), 12.78-12.93 (m, 1H), 9.12-9.19 (m, 1H), 7.98-8.06 (m, 1H), 7.89-7.98 (m, 2H), 7.73-7.83 (m, 1H), 7.62-7.70 (m, 1H), 7.38-7.48 (m, 2H), 7.22-7.30 (m, 1H). MS (ESI): [M+H]⁺=305.03

### Example 94: N-(2-(methylsulfonamido)ethyl)-3-(4-phenyl-1H-imidazol-2-yl)-1H-indazole-5-carboxamide (29a)

Compound (29a) of Example 94 was obtained from compound 28 by using N-(2-aminoethyl)methanesulfonamide according to General Synthesis Procedure A (yellowish oil, yield: 91%).

¹H NMR (400 MHz, DMSO-d₆) δ 9.01 (s, 1H), 8.64 (t, J=5.50 Hz, 1H), 7.98 (d, J=7.33 Hz, 2H), 7.91 (dd, J=1.37, 8.70 Hz, 1H), 7.76 (s, 1H), 7.61-7.65 (m, 1H), 7.42 (t, J=7.56 Hz, 2H), 7.21-7.27 (m, 1H), 7.16-7.21 (m, 1H), 3.45 (q, J=5.95 Hz, 2H), 3.18 (q, J=6.26 Hz, 2H), 2.94 (s, 3H). MS (ESI): [M+H]⁺=424.91

### Example 95: N-(2-(dimethylamino)ethyl)-3-(4-phenyl-1H-imidazol-2-yl)-1H-indazole-5-carboxamide (29b)

Compound (29b) of Example 95 was obtained from compound 28 by using N,N-dimethylethane-1,2-diamine according to General Synthesis Procedure A (yellowish oil, yield: 45%).

¹H NMR (400 MHz, DMSO-d₆) δ 8.92-9.03 (m, 1H), 8.44-8.49 (m, 1H), 7.93-8.01 (m, 2H), 7.86-7.90 (m, 1H), 7.71-7.80 (m, 1H), 7.60-7.64 (m, 1H), 7.38-7.44 (m, 2H), 7.21-7.27 (m, 1H), 3.39-3.42 (m, 2H), 2.43-2.48 (m, 2H), 2.21 (s, 6H). MS (ESI): [M+H]⁺=375.21

### Example 96: (3-(4-phenyl-1H-imidazol-2-yl)-1H-indazol-5-yl)(4-phenylpiperazin-1-yl)methanone (29c)

Compound (29c) of Example 96 was obtained from compound 28 by using 1-phenylpiperazine according to General Synthesis Procedure A (yellowish oil, yield: 37%).

¹H NMR (400 MHz, DMSO-d₆) δ 8.63 (s, 1H), 7.90 (d, J=7.33 Hz, 2H), 7.74 (s, 1H), 7.67 (dd, J=0.80, 8.59 Hz, 1H), 7.51-7.57 (m, 1H), 7.23-7.30 (m, 4H), 7.14-7.20 (m, 1H), 7.00 (d, J=8.01 Hz, 2H), 6.84 (t, J=7.21 Hz, 1H), 3.64-3.81 (m, 4H), 3.16-3.30 (m, 4H).

MS (ESI): [M+H]⁺=448.95

### Example 97: (4-(4-fluorophenyl)piperazin-1-yl)(3-(4-phenyl-1H-imidazol-2-yl)-1H-indazol-5-yl)methanone (29d)

Compound (29d) of Example 97 was obtained from compound 28 by using 1-(4-fluorophenyl)piperazine according to General Synthesis Procedure A (yellowish oil, yield: 68%).

¹H NMR (400 MHz, DMSO-d₆) δ 8.63 (s, 1H), 7.90 (d, J=7.33 Hz, 2H), 7.72-7.78 (m, 1H), 7.67 (dd, J=0.80, 8.59 Hz, 1H), 7.53 (dd, J=1.37, 8.70 Hz, 1H), 7.24-7.33 (m, 2H), 7.14-7.23 (m, 1H), 7.05-7.12 (m, 2H), 6.98-7.04 (m, 2H), 3.62-3.85 (m, 4H), 3.13-3.24 (m, 4H). MS (ESI): [M+H]⁺=466.95

### Example 98: (3-(4-phenyl-1H-imidazol-2-yl)-1H-indazol-5-yl)(4-(3-(trifluoromethyl)phenyl)piperazin-1-yl)methanone (29e)

Compound (29e) of Example 98 was obtained from compound 28 by using 1-(3-(trifluoromethyl)phenyl)piperazine according to General Synthesis Procedure A (yellowish oil, yield: 32%).

¹H NMR (400 MHz, METHANOL-d₄) δ 8.47-8.69 (m, 1H), 7.75-7.95 (m, 2H), 7.68 (d, J=8.24 Hz, 1H), 7.52-7.61 (m, 2H), 7.17-7.46 (m, 6H), 7.13 (d, J=7.79 Hz, 1H), 3.75-4.04 (m, 4H), 3.33-3.49 (m, 4H). MS (ESI): [M+H]⁺=517.5

### Example 99: (4-methylpiperazin-1-yl)(3-(4-phenyl-1H-imidazol-2-yl)-1H-indazol-5-yl)methanone (29f)

Compound (29f) of Example 99 was obtained from compound 28 by using 1-methylpiperazine according to General Synthesis Procedure A (yellowish oil, yield: 33%).

¹H NMR (400 MHz, METHANOL-d₄) δ 8.48-8.59 (m, 1H), 7.77-7.94 (m, 2H), 7.64-7.69 (m, 1H), 7.51-7.59 (m, 2H), 7.42 (t, J=7.56 Hz, 2H), 7.24-7.31 (m, 1H), 3.60-3.89 (m, 4H), 2.48-2.66 (m, 4H), 2.38 (s, 3H). MS (ESI): [M+H]⁺=386.91

### Example 100: (4-(2-fluorophenyl)piperazin-1-yl)(3-(4-phenyl-1H-imidazol-2-yl)-1H-indazol-5-yl)methanone (29g)

Compound (29g) of Example 100 was obtained from compound 28 by using 1-(2-fluorophenyl)piperazine according to General Synthesis Procedure A (yellowish oil, yield: 25%).

¹H NMR (400 MHz, DMSO-d₆) δ 8.64 (s, 1H), 7.92 (d, J=7.56 Hz, 2H), 7.75 (s, 1H), 7.67 (dd, J=0.80, 8.59 Hz, 1H), 7.52-7.57 (m, 1H), 7.25-7.34 (m, 2H), 6.99-7.22 (m, 5H), 3.64-3.89 (m, 4H), 3.02-3.20 (m, 4H). MS (ESI): [M+H]⁺=467.02

### Example 101: (4-(3-chlorophenyl)piperazin-1-yl)(3-(4-phenyl-1H-imidazol-2-yl)-1H-indazol-5-yl)methanone (29h)

Compound (29h) of Example 101 was obtained from compound 28 by using 1-(3-chlorophenyl)piperazine according to General Synthesis Procedure A (yellowish oil, yield: 22%).

¹H NMR (400 MHz, DMSO-d₆) δ 8.63 (s, 1H), 7.91 (d, J=7.56 Hz, 2H), 7.71-7.77 (m, 1H), 7.67 (dd, J=0.57, 8.59 Hz, 1H), 7.51-7.56 (m, 1H), 7.17-7.33 (m, 4H), 7.01 (s, 1H), 6.93-6.98 (m, 1H), 6.84-6.84 (m, 1H), 6.82-6.87 (m, 1H), 3.64-3.80 (m, 4H), 3.20-3.32 (m, 4H). MS (ESI): [M+H]⁺=482.91

### Example 102: (4-(2-chlorophenyl)piperazin-1-yl)(3-(4-phenyl-1H-imidazol-2-yl)-1H-indazol-5-yl)methanone (29i)

Compound (29i) of Example 102 was obtained from compound 28 by using 1-(2-chlorophenyl)piperazine according to General Synthesis Procedure A (yellowish oil, yield: 22%).

¹H NMR (400 MHz, DMSO-d₆) δ 8.64 (s, 1H), 7.89-7.95 (m, 2H), 7.74 (s, 1H), 7.67 (dd, J=0.80, 8.59 Hz, 1H), 7.52-7.57 (m, 1H), 7.45 (dd, J=1.60, 8.01 Hz, 1H), 7.28-7.36 (m, 3H), 7.17-7.24 (m, 2H), 7.09 (dt, J=1.49, 7.61 Hz, 1H), 3.64-3.89 (m, 4H), 3.00-3.15 (m, 4H). MS (ESI): [M+H]⁺=482.91

### tert-butyl (1-(2-(3-(4-phenyl-1H-imidazol-2-yl)-1H-indazole-5-carboxamido)ethyl)piperidin-4-yl)carbamate (29j)

Compound 29j was obtained from compound 28 by using tert-butyl (1-(2-aminoethyl)piperidin-4-yl)carbamate according to General Synthesis Procedure A (yellowish oil, yield: 83%).

¹H NMR (400 MHz, METHANOL-d₄) δ 8.91 (s, 1H), 7.86-7.95 (m, 3H), 7.60-7.65 (m, 1H), 7.58 (s, 1H), 7.38-7.47 (m, 2H), 7.23-7.31 (m, 1H), 3.57-3.66 (m, 2H), 3.34-3.41 (m, 1H), 2.99-3.07 (m, 2H), 2.65-2.72 (m, 2H), 2.21-2.30 (m, 2H), 1.85-1.92 (m, 2H), 1.49-1.58 (m, 2H), 1.43 (s, 9H). MS (ESI): [M+H]⁺=530.06

### Example 103: N-(2-(4-aminopiperidin-1-yl)ethyl)-3-(4-phenyl-1H-imidazol-2-yl)-1H-indazole-5-carboxamide (30j)

Compound (30j) of Example 103 was obtained from compound 29j according to General Synthesis Procedure B (white solid, yield: 52%).

¹H NMR (400 MHz, METHANOL-d₄) δ 8.87-8.94 (m, 1H), 7.82-7.94 (m, 3H), 7.59-7.65 (m, 1H), 7.57 (s, 1H), 7.42 (t, J=7.67 Hz, 2H), 7.24-7.32 (m, 1H), 3.60 (t, J=6.87 Hz, 2H), 3.03 (d, J=12.14 Hz, 2H), 2.61-2.73 (m, 3H), 2.12-2.24 (m, 2H), 1.80-1.90 (m, 2H), 1.40-1.52 (m, 2H). MS (ESI): [M+H]⁺=430.35

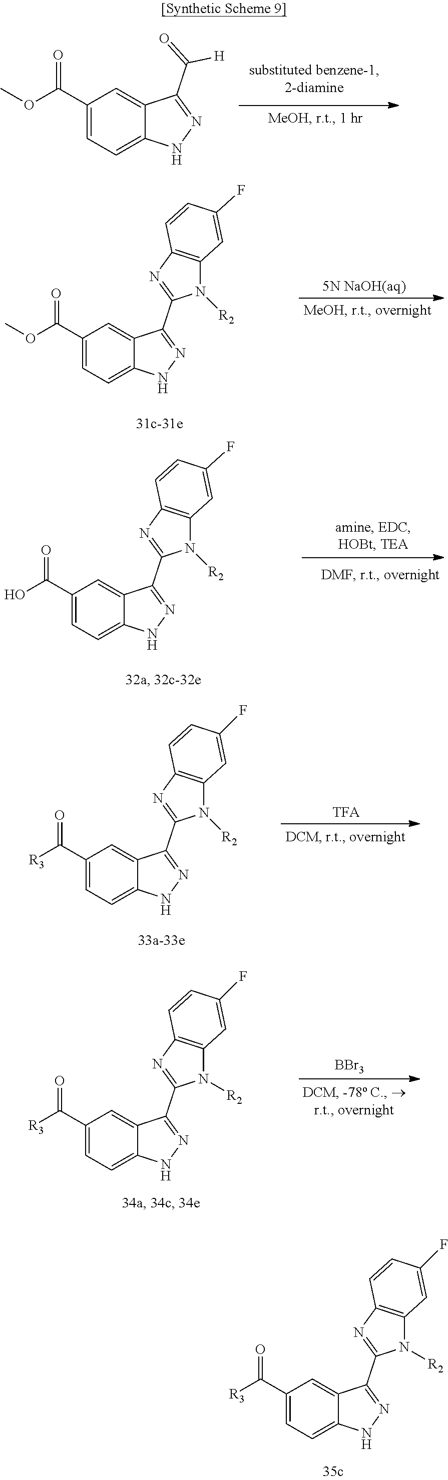

### methyl 3-(6-fluoro-1-(2-methoxyethyl)-1H-benzo[d]imidazol-2-yl)-1H-indazole-5-carboxylate (31c)

Compound 31c was obtained by using 5-fluoro-N-(2-methoxyethyl)benzene-1,2-diamine according to General Synthesis Procedure C (brown oil, yield: 31%).

¹H NMR (400 MHz, CHLOROFORM-d) δ 9.38 (s, TH), 8.07-8.14 (in, TH), 7.86 (dd, J=4.81, 8.70 Hz, TH), 7.44 (d, J=8.93 Hz, TH), 7.22-7.27 (mn, TH), 7.08 (dt, J=2.40, 9.22 Hz, TH), 4.86 (t, J=5.61 Hz, 2H), 3.94 (s, 3H), 3.86 (t, J=5.72 Hz, 2H), 3.32 (s, 3H). MS (ESI): [M+H]⁺=368.8

### methyl 3-(1-(2-(dimethylamino)ethyl)-6-fluoro-1H-benzo[d]imidazol-2-yl)-1H-indazole-5-carboxylate (31d)

Compound 31d was obtained by using N-(2-(dimethylamino)ethyl)-5-fluorobenzene-1,2-diamine according to General Synthesis Procedure C (orange oil, yield: 21%).

¹H NMR (400 MHz, DMSO-d₆) δ 9.23 (dd, J=0.80, 1.72 Hz, 1H), 8.03-8.10 (m, 1H), 7.84 (dd, J=4.81, 8.93 Hz, 1H), 7.75 (dd, J=0.80, 8.82 Hz, 1H), 7.60 (dd, J=2.40, 9.50 Hz, 1H), 7.14 (ddd, J=2.52, 8.93, 9.85 Hz, 1H), 4.91 (t, J=6.87 Hz, 2H), 3.93 (s, 3H), 2.69 (t, J=6.98 Hz, 2H), 2.19 (s, 6H). MS (ESI): [M+H]⁺=381.9

### methyl 3-(6-fluoro-1-(2-(4-methylpiperazin-1-yl)ethyl)-1H-benzo[d]imidazol-2-yl)-1H-indazole-5-carboxylate (31e)

Compound 31e was obtained by using 5-fluoro-N-(2-(4-methylpiperazin-1-yl)ethyl)benzene-1,2-diamine according to General Synthesis Procedure C (white solid, yield: 27%).

¹H NMR (400 MHz, DMSO-d₆) δ 9.20 (dd, J=0.80, 1.72 Hz, 1H), 8.05 (dd, J=1.60, 8.70 Hz, 1H), 7.83 (dd, J=4.92, 8.82 Hz, 1H), 7.72-7.77 (m, 1H), 7.59 (dd, J=2.52, 9.39 Hz, 1H), 7.13 (ddd, J=2.40, 8.87, 9.79 Hz, 1H), 4.90 (t, J=6.53 Hz, 2H), 3.90-3.96 (m, 3H), 2.70 (t, J=6.64 Hz, 2H), 2.25-2.49 (m, 4H), 1.96-2.25 (m, 7H). MS (ESI): [M+H]⁺=436.9

### 3-(6-fluoro-1-isopropyl-1H-benzo[d]imidazol-2-yl)-1H-indazole-5-carboxylic acid (32a)

Compound 32a was obtained by using 5-fluoro-N-isopropylbenzene-1,2-diamine according to General Synthesis Procedure E (red solid, yield: 82%).

¹H NMR (400 MHz, DMSO-d₆) δ 9.09-9.16 (m, 1H), 7.99-8.06 (m, 1H), 7.85 (dd, J=5.15, 8.82 Hz, 1H), 7.78 (dd, J=2.29, 9.85 Hz, 1H), 7.74 (dd, J=0.80, 8.82 Hz, 1H), 7.14 (ddd, J=2.40, 8.87, 9.79 Hz, 1H), 6.08-6.20 (m, 1H), 1.66 (d, J=7.10 Hz, 6H). MS (ESI): [M+H]⁺=339.2

### 3-(6-fluoro-1-(2-methoxyethyl)-1H-benzo[d]imidazol-2-yl)-1H-indazole-5-carboxylic acid (32c)

Compound 32c was obtained from compound 31c according to General Synthesis Procedure D (white solid, yield: 45%).

¹H NMR (400 MHz, DMSO-d₆) δ 9.24 (d, J=0.92 Hz, 1H), 8.03-8.06 (m, 1H), 7.85 (dd, J=4.81, 8.93 Hz, 1H), 7.70-7.76 (m, 1H), 7.55-7.62 (m, 1H), 7.09-7.16 (m, 1H), 4.99 (t, J=5.50 Hz, 2H), 3.80 (t, J=5.50 Hz, 2H), 3.17 (s, 3H). MS (ESI): [M+H]⁺=354.8

### 3-(1-(2-(dimethylamino)ethyl)-6-fluoro-1H-benzo[d]imidazol-2-yl)-1H-indazole-5-carboxylic acid (32d)

Compound 32d was obtained from compound 31d according to General Synthesis Procedure D (brown solid, yield: 45%).

¹H NMR (400 MHz, DMSO-d₆) δ 9.23 (d, J=0.92 Hz, 1H), 8.03-8.08 (m, 1H), 7.90 (dd, J=4.81, 8.93 Hz, 1H), 7.85 (dd, J=2.29, 9.16 Hz, 1H), 7.77 (d, J=8.24 Hz, 1H), 7.16-7.24 (m, 1H), 5.17-5.25 (m, 2H), 3.55-3.62 (m, 2H), 2.93 (d, J=4.58 Hz, 6H). MS (ESI): [M+H]⁺=367.8

### 3-(6-fluoro-1-(2-(4-methylpiperazin-1-yl)ethyl)-1H-benzo[d]imidazol-2-yl)-1H-indazole-5-carboxylic acid (32e)

Compound 32e was obtained from compound 31e according to General Synthesis Procedure D (brown solid, yield: 33%).

¹H NMR (400 MHz, DMSO-d₆) δ 9.19-9.25 (m, 1H), 8.02-8.08 (m, 1H), 7.88-7.93 (m, 1H), 7.79-7.83 (m, 1H), 7.73-7.78 (m, 1H), 7.13-7.23 (m, 1H), 5.04-5.21 (m, 2H), 3.42-3.72 (m, 6H), 3.12-3.41 (m, 4H), 2.74-2.84 (m, 3H). MS (ESI): [M+H]⁺=423.4

### tert-butyl (3-(3-(6-fluoro-1-isopropyl-1H-benzo[d]imidazol-2-yl)-1H-indazole-5-carboxamido) propyl)carbamate (33a)

Compound 33a was obtained from compound 32a by using tert-butyl (3-aminopropyl)carbamate according to General Synthesis Procedure A (yellow oil, yield: 38%).

¹H NMR (400 MHz, METHANOL-d₄) δ 8.71-8.78 (m, 1H), 7.97 (dd, J=1.60, 8.70 Hz, 1H), 7.78 (dd, J=4.81, 8.93 Hz, 1H), 7.70 (dd, J=0.92, 8.93 Hz, 1H), 7.61 (dd, J=2.18, 9.50 Hz, 1H), 7.08-7.17 (m, 1H), 5.87 (td, J=6.98, 13.97 Hz, 1H), 3.42-3.52 (m, 2H), 2.97-3.20 (m, 2H), 1.76-1.84 (m, 2H), 1.70 (d, J=7.10 Hz, 6H), 1.42 (s, 9H). MS (ESI): [M+H]⁺=495.3

### Example 104: N-(3-(dimethylamino)propyl)-3-(6-fluoro-1-isopropyl-1H-benzo[d]imidazol-2-yl)-1H-indazole-5-carboxamide (33b)

Compound (33b) of Example 104 was obtained from compound 32a by using N,N-dimethylpropane-1,3-diamine according to General Synthesis Procedure A (brown oil, yield: 38%).

¹H NMR (400 MHz, METHANOL-d₄) δ 8.74 (dd, J=0.92, 1.60 Hz, 1H), 7.97 (dd, J=1.60, 8.70 Hz, 1H), 7.74-7.81 (m, 1H), 7.70 (dd, J=0.80, 8.82 Hz, 1H), 7.56-7.64 (m, 1H), 6.98-7.18 (m, 1H), 5.78-5.92 (m, 1H), 3.46 (t, J=6.98 Hz, 2H), 2.47 (t, J=7.00 Hz, 2H), 2.28 (s, 6H), 1.75-1.93 (m, 2H), 1.71 (d, J=6.87 Hz, 6H). MS (ESI): [M+H]⁺=423.1

### tert-butyl (3-(3-(6-fluoro-1-(2-methoxyethyl)-1H-benzo[d]imidazol-2-yl)-1H-indazole-5-carboxamido)propyl)carbamate (33c)

Compound 33c was obtained from compound 32c by using tert-butyl (3-aminopropyl)carbamate according to General Synthesis Procedure A (brown oil, yield: 69%).

¹H NMR (400 MHz, METHANOL-d₄) δ 8.92 (dd, J=0.80, 1.72 Hz, 1H), 7.93-8.00 (m, 1H), 7.75 (dd, J=4.69, 8.82 Hz, 1H), 7.67 (dd, J=0.80, 8.82 Hz, 1H), 7.41 (dd, J=2.40, 9.04 Hz, 1H), 4.90-4.94 (m, 2H), 3.86 (t, J=5.38 Hz, 2H), 3.48 (t, J=6.87 Hz, 2H), 3.24 (s, 3H), 3.17 (t, J=6.75 Hz, 2H), 1.77-1.85 (m, 2H), 1.43 (s, 9H). MS (ESI): [M+H]⁺=511.2

### tert-butyl (3-(3-(1-(2-(dimethylamino)ethyl)-6-fluoro-1H-benzo[d]imidazol-2-yl)-1H-indazole-5-carboxamido)propyl)carbamate (33d)

Compound 33d was obtained from compound 32d by using tert-butyl (3-aminopropyl)carbamate according to General Synthesis Procedure A (brown oil, yield: 52%).

¹H NMR (400 MHz, DMSO-d₆) δ 9.01 (s, 1H), 8.58 (t, J=5.80 Hz, 1H), 7.90-7.95 (m, 1H), 7.83 (dd, J=5.19, 8.85 Hz, 1H), 7.69 (d, J=7.94 Hz, 1H), 7.59 (dd, J=2.44, 9.16 Hz, 1H), 7.08-7.18 (m, 1H), 6.79-6.87 (m, 1H), 4.90 (t, J=6.71 Hz, 2H), 3.27-3.32 (m, 2H), 3.00 (q, J=6.71 Hz, 2H), 2.67 (t, J=7.02 Hz, 2H), 2.17 (s, 6H), 1.66 (t, J=7.02 Hz, 2H), 1.38 (s, 9H). MS (ESI): [M+H]⁺=524.3

### tert-butyl (3-(3-(6-fluoro-1-(2-(4-methylpiperazin-1-yl)ethyl)-1H-benzo[d]imidazol-2-yl)-1H-indazole-5-carboxamido)propyl)carbamate (33e)

Compound 33e was obtained from compound 32e by using tert-butyl (3-aminopropyl)carbamate according to General Synthesis Procedure A (yellow oil, yield: 36%).

¹H NMR (400 MHz, METHANOL-d₄) δ 8.87-8.92 (m, 1H), 7.98 (dd, J=1.83, 8.70 Hz, 1H), 7.78 (dd, J=4.58, 8.70 Hz, 1H), 7.69 (dd, J=0.92, 8.70 Hz, 1H), 7.44 (dd, J=2.29, 9.16 Hz, 1H), 7.13 (dt, J=2.29, 9.39 Hz, 1H), 4.90-4.94 (m, 2H), 3.44-3.51 (m, 2H), 3.17 (t, J=6.64 Hz, 2H), 2.80 (t, J=6.87 Hz, 2H), 2.40-2.66 (m, 4H), 2.06-2.36 (m, 7H), 1.81 (quin, J=6.64 Hz, 2H), 1.43 (s, 9H). MS (ESI): [M+H]⁺=423.4 MS (ESI): [M+H]⁺=579.5

### Example 105: N-(3-aminopropyl)-3-(6-fluoro-1-isopropyl-1H-benzo[d]imidazol-2-yl)-1H-indazole-5-carboxamide (34a)

Compound (34a) of Example 105 was obtained from compound 33a according to General Synthesis Procedure B (white solid, yield: 37%).

¹H NMR (400 MHz, METHANOL-d₄) δ 8.71-8.77 (m, 1H), 7.94-8.00 (m, 1H), 7.72-7.81 (m, 1H), 7.67-7.72 (m, 1H), 7.58-7.65 (m, 1H), 7.09-7.18 (m, 1H), 5.81-5.94 (m, 1H), 3.40-3.55 (m, 2H), 2.75 (t, J=6.98 Hz, 2H), 1.77-1.86 (m, 2H), 1.62-1.73 (m, 6H) MS (ESI): [M+H]⁺=395.0

### Example 106: N-(3-aminopropyl)-3-(6-fluoro-1-(2-methoxyethyl)-1H-benzo[d]imidazol-2-yl)-1H-indazole-5-carboxamide (34c)

Compound (34c) of Example 106 was obtained from compound 33c according to General Synthesis Procedure B (yellow oil, yield: 42%).

¹H NMR (400 MHz, METHANOL-d₄) δ 8.92 (d, J=1.83 Hz, 1H), 7.94-7.98 (m, 1H), 7.75 (dd, J=4.88, 8.55 Hz, 1H), 7.65-7.71 (m, 1H), 7.42 (dd, J=2.14, 8.85 Hz, 1H), 7.09 (dd, J=2.14, 9.46 Hz, 1H), 4.91-4.94 (m, 2H), 3.86 (t, J=5.19 Hz, 2H), 3.52 (t, J=6.71 Hz, 2H), 3.23 (s, 3H), 2.76 (t, J=6.71 Hz, 2H), 1.77-1.87 (m, 2H). MS (ESI): [M+H]⁺=410.8

### Example 107: N-(3-aminopropyl)-3-(1-(2-(dimethylamino)ethyl)-6-fluoro-1H-benzo[d]imidazol-2-yl)-1H-indazole-5-carboxamide (34d)

Compound (34d) of Example 107 was obtained from compound 33d according to General Synthesis Procedure B (yellow oil, yield: 27%).

¹H NMR (400 MHz, METHANOL-d₄) δ 8.91-8.94 (m, 1H), 7.96 (dd, J=1.53, 8.85 Hz, 1H), 7.77 (dd, J=4.58, 8.85 Hz, 1H), 7.66-7.71 (m, 1H), 7.43 (dd, J=2.44, 8.55 Hz, 1H), 7.08-7.18 (m, 1H), 4.91-4.94 (m, 2H), 3.52 (t, J=7.02 Hz, 2H), 2.80-2.89 (m, 3H), 2.77 (t, J=7.02 Hz, 2H), 2.34 (s, 7H), 1.83 (t, J=6.71 Hz, 2H). MS (ESI): [M+H]⁺=424.5

### Example 108: N-(3-aminopropyl)-3-(6-fluoro-1-(2-(4-methylpiperazin-1-yl)ethyl)-1H-benzo[d]imidazol-2-yl)-1H-indazole-5-carboxamide (34e)

Compound (34e) of Example 108 was obtained from compound 33e according to General Synthesis Procedure B (yellow oil, yield: 37%).

¹H NMR (400 MHz, METHANOL-d₄) δ 8.90 (dd, J=0.80, 1.72 Hz, 1H), 7.96-8.01 (m, 1H), 7.74-7.80 (m, 1H), 7.66-7.71 (m, 1H), 7.40-7.46 (m, 1H), 7.08-7.16 (m, 1H), 4.89-4.95 (m, 2H), 3.48-3.57 (m, 2H), 2.72-2.88 (m, 4H), 2.36-2.60 (m, 4H), 2.07-2.36 (m, 7H), 1.80-1.88 (m, 2H). MS (ESI): [M+H]⁺=479.4

### Example 109: N-(3-aminopropyl)-3-(6-fluoro-1-(2-hydroxyethyl)-1H-benzo[d]imidazol-2-yl)-1H-indazole-5-carboxamide (35c)

Compound 34c was dissolved in DCM and cooled to −78° C., and BBr₃ in DCM (2.0 eq) was slowly added, and the solution was stirred at room temperature for 2 hours. Upon completion of the reaction, the solution was extracted with saturated aqueous NaHCO₃ solution and DCM, dried over Na₂SO₄, and filtered. The solvent was removed under reduced pressure, and the residue was purified by column chromatography to afford compound (35c) of Example 109 (white solid, yield: 27%)/

¹H NMR (400 MHz, DMSO-d₆) δ 9.02 (d, J=1.83 Hz, 1H), 7.92 (dd, J=1.53, 8.85 Hz, 1H), 7.82 (dd, J=5.19, 8.85 Hz, 1H), 7.69 (d, J=9.77 Hz, 1H), 7.57 (dd, J=2.44, 9.77 Hz, 1H), 7.12 (dt, J=2.44, 9.46 Hz, 1H), 4.83 (t, J=5.49 Hz, 2H), 3.86 (t, J=5.80 Hz, 2H), 3.35-3.39 (m, 2H), 2.64 (t, J=6.71 Hz, 2H), 1.64 (quin, J=6.71 Hz, 2H). MS (ESI): [M+H]⁺=396.8

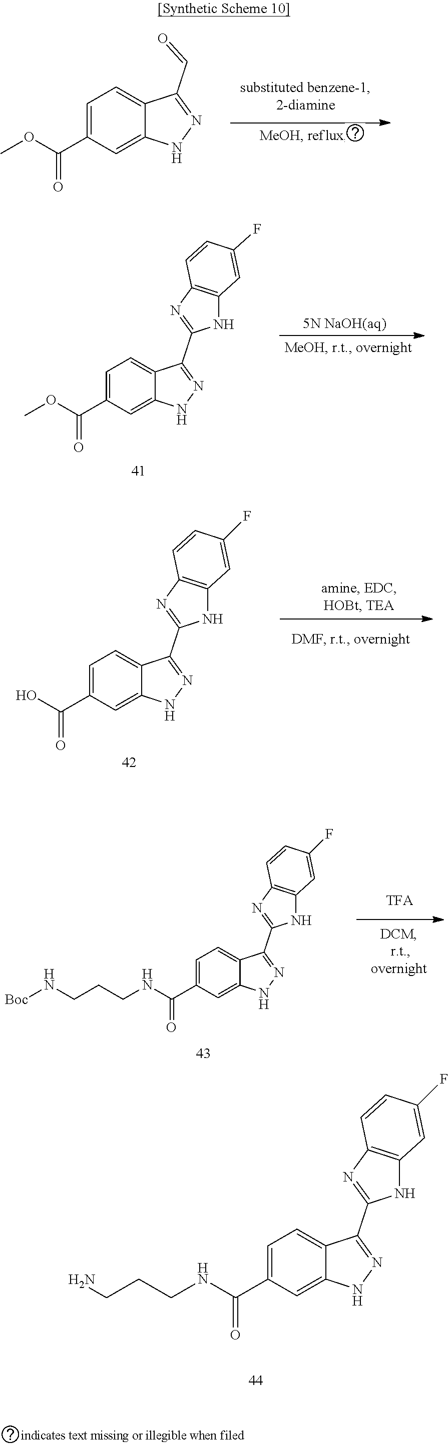 [SMILES: COC(=O)c1ccc2c(C=O)n[nH]c2c1 ; COC(=O)c1ccc2c(-c3nc4ccc(F)cc4[nH]3)n[nH]c2c1 ; O=C(O)c1ccc2c(-c3nc4ccc(F)cc4[nH]3)n[nH]c2c1 ; CC(C)(C)OC(=O)NCCCNC(=O)c1ccc2c(-c3nc4ccc(F)cc4[nH]3)n[nH]c2c1 ; NCCCNC(=O)c1ccc2c(-c3nc4ccc(F)cc4[nH]3)n[nH]c2c1]

### methyl 3-(6-fluoro-H-benzo[d]imidazol-2-yl)-1H-indazole-6-carboxylate (41)

methyl 3-formyl-1H-indazole-6-carboxylate (1.0 eq) and 4-fluorobenzene-1,2-diamine (1.2 eq) were dissolved in methanol, and the solution was stirred under reflux for 12 hours. Upon completion of the reaction, the solvent was evaporated under reduced pressure, and the residue was extracted with water and ethyl acetate. The organic layer was dried over Na₂SO₄ and filtered. The solvent was removed under reduced pressure, and the residue was purified by column chromatography to afford compound 41 (yellowish solid, yield: 38%).

¹H NMR (400 MHz, DMSO-d₆) δ 8.57-8.64 (m, 1H), 8.26 (s, 1H), 7.89 (dd, J=1.26, 8.59 Hz, 1H), 7.65-7.80 (m, 1H), 7.46-7.61 (m, 1H), 7.03-7.16 (m, 1H), 3.91-3.95 (m, 3H). MS (ESI): [M−H]⁻=309.3

### 3-(6-fluoro-1H-benzo[d]imidazol-2-yl)-1H-indazole-6-carboxylic acid (42)

Compound 42 was obtained from compound 41 according to General Synthesis Procedure D (brown solid, yield: 51%) ¹H NMR (400 MHz, DMSO-d₆) δ 8.59 (dd, J=0.80, 8.59 Hz, 1H), 8.27 (t, J=1.03 Hz, 1H), 7.90 (dd, J=1.37, 8.70 Hz, 1H), 7.68 (dd, J=4.81, 8.70 Hz, 1H), 7.43-7.50 (m, 1H), 7.13-7.21 (m, 1H). MS (ESI): [M+H]⁺=296.8.

### tert-butyl (3-(3-(6-fluoro-1H-benzo[d]imidazol-2-yl)-1H-indazole-6-carboxamido)propyl) carbamate (43)

Compound 43 was obtained from compound 42 by using tert-butyl (3-aminopropyl)carbamate according to General Synthesis Procedure A (brown solid, yield: 52%).

¹H NMR (400 MHz, METHANOL-d₄) δ 8.53 (d, J=8.55 Hz, 1H), 8.11-8.17 (m, 1H), 7.73-7.80 (m, 1H), 7.65 (br. s, 1H), 7.35 (br. s, 1H), 7.07 (dt, J=2.44, 9.16 Hz, 1H), 3.48 (t, J=7.02 Hz, 2H), 3.18 (t, J=6.71 Hz, 2H), 1.81 (quin, J=6.71 Hz, 2H), 1.45 (s, 9H). MS (ESI): [M+H]⁺=453.5.

### Example 110: N-(3-aminopropyl)-3-(6-fluoro-1H-benzo[d]imidazol-2-yl)-1H-indazole-6-carboxamide (44)

Compound (44) of Example 110 was obtained from compound 43 according to General Synthesis Procedure B (white solid, yield: 40%).

¹H NMR (400 MHz, DMSO-d₆) δ 8.74 (t, J=5.50 Hz, 1H), 8.50 (d, J=8.24 Hz, 1H), 8.10-8.15 (m, 1H), 7.77 (dd, J=1.37, 8.70 Hz, 1H), 7.62 (br. s, 1H), 7.40 (br. s, 1H), 7.04-7.12 (m, 1H), 3.34-3.40 (m, 2H), 2.63 (t, J=6.64 Hz, 2H), 1.64 (quin, J=6.76 Hz, 2H). MS (ESI): [M−H]⁻=351.3.

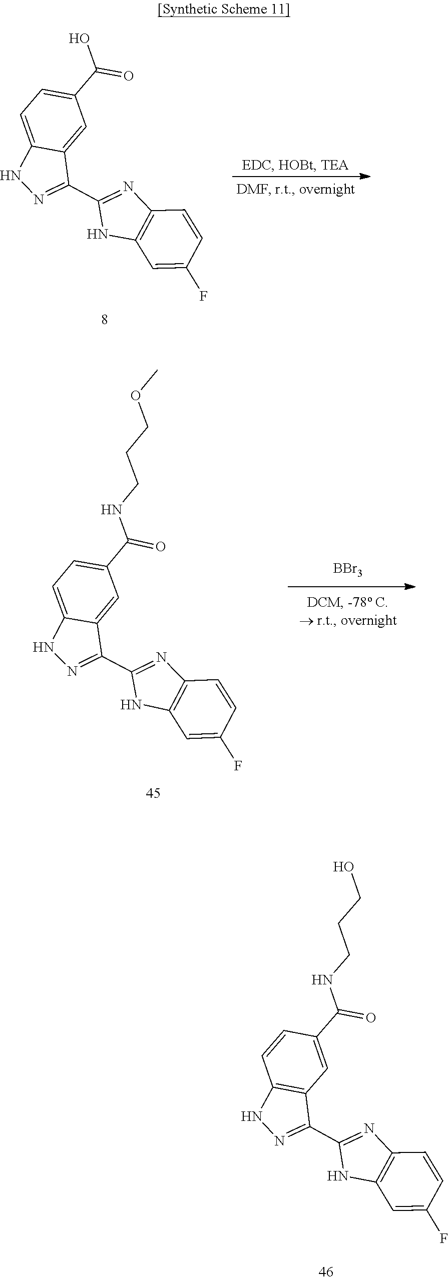 [SMILES: O=C(O)c1ccc2[nH]nc(-c3nc4ccc(F)cc4[nH]3)c2c1 ; COCCCNC(=O)c1ccc2[nH]nc(-c3nc4ccc(F)cc4[nH]3)c2c1 ; "BBr3" ; O=C(NCCCO)c1ccc2[nH]nc(-c3nc4ccc(F)cc4[nH]3)c2c1]

### 3-(6-fluoro-1H-benzo[d]imidazol-2-yl)-N-(3-methoxypropyl)-1H-indazole-5-carboxamide (45)

Compound 45 was obtained from compound 8 by using 3-methoxypropan-1-amine according to General Synthesis Procedure A (yellow oil, yield: 42%).

¹H NMR (400 MHz, METHANOL-d₄) δ 8.99 (s, 1H), 7.93-7.97 (m, 1H), 7.62-7.74 (m, 2H), 7.37-7.51 (m, 1H), 6.99-7.15 (m, 1H), 3.53-3.57 (m, 4H), 3.39 (s, 3H), 1.93-1.97 (m, 2H). MS (ESI): [M+H]⁺=368.0

### Example 111: 3-(6-fluoro-1H-benzo[d]imidazol-2-yl)-N-(3-hydroxypropyl)-1H-indazole-5-carboxamide (46)

Compound 45 was dissolved in DCM and cooled to −78° C., and BBr₃ in DCM (2.0 eq) was slowly added, and the solution was stirred at room temperature for 2 hours. Upon completion of the reaction, the solution was extracted with saturated aqueous NaHCO₃ solution and DCM, dried over Na2SO₄, and filtered. The solvent was removed under reduced pressure, and the residue was purified by column chromatography to afford compound (46) of Example 111 (white solid, yield: 23%).

¹H NMR (400 MHz, METHANOL-d₄) δ 8.99 (dd, J=0.80, 1.72 Hz, 1H), 7.91-7.99 (m, 1H), 7.62-7.80 (m, 2H), 7.37-7.51 (m, 1H), 7.23-7.37 (m, 1H), 7.01-7.14 (m, 1H), 3.71 (t, J=6.30 Hz, 2H), 3.58 (t, J=6.30 Hz, 2H), 1.85-1.96 (m, 2H). MS (ESI): [M+H]⁺=354.2

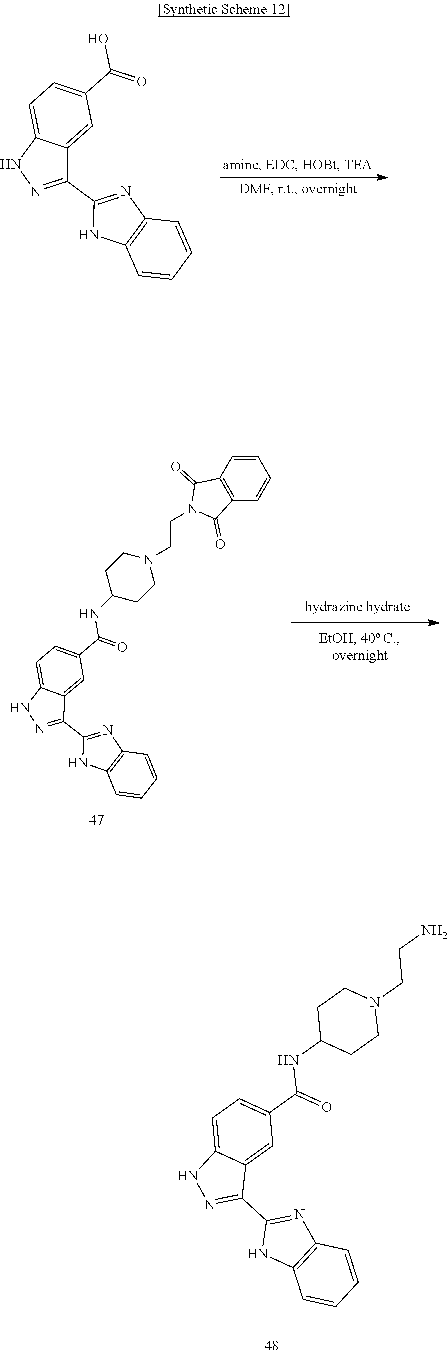 [SMILES: O=C(O)c1ccc2[nH]nc(-c3nc4ccccc4[nH]3)c2c1 ; O=C(NC1CCN(CCN2C(=O)c3ccccc3C2=O)CC1)c1ccc2[nH]nc(-c3nc4ccccc4[nH]3)c2c1 ; "hydrazine hydrate" ; NCCN1CCC(NC(=O)c2ccc3[nH]nc(-c4nc5ccccc5[nH]4)c3c2)CC1]

### 3-(1H-benzo[d]imidazol-2-yl)-N-(1-(2-(1,3-dioxoisoindolin-2-yl)ethyl)piperidin-4-yl)-1H-indazole-5-carboxamide (47)

Compound 47 was obtained by using 2-(2-(4-aminopiperidin-1-yl)ethyl)isoindoline-1,3-dione according to General Synthesis Procedure A (yellow oil, yield: 32%).

¹H NMR (400 MHz, METHANOL-d₄) δ 8.97 (dd, J=0.80, 1.72 Hz, 1H), 7.91-7.97 (m, 1H), 7.75-7.88 (m, 5H), 7.61-7.69 (m, 2H), 7.23-7.35 (m, 2H), 3.92-4.03 (m, 1H), 3.83-3.91 (m, 2H), 3.10-3.19 (m, 2H), 2.66-2.77 (m, 2H), 2.19-2.29 (m, 2H), 1.96-2.09 (m, 2H), 1.60-1.76 (m, 2H). MS (ESI): [M+H]⁺=534.06

### Example 112: N-(1-(2-aminoethyl)piperidin-4-yl)-3-(1H-benzo[d]imidazol-2-yl)-1H-indazole-5-carboxamide (48)

Compound 47 was dissolved in ethanol, and N₂H₄-H₂O (4.0 eq) was added. The solution was heated to 40° C. and stirred for 12 hours. Upon completion of the reaction, the solvent was completely removed under reduced pressure, and the residue was purified by column chromatography to afford compound (48) of Example 112 (yellow solid, yield: 23%).

¹H NMR (400 MHz, METHANOL-d₄) δ 9.00 (dd, J=0.80, 1.72 Hz, 1H), 7.96 (dd, J=1.60, 8.70 Hz, 1H), 7.63-7.74 (m, 3H), 7.25-7.36 (m, 2H), 3.90-4.01 (m, 1H), 2.98-3.06 (m, 2H), 2.74-2.83 (m, 2H), 2.46-2.56 (m, 2H), 2.15-2.28 (m, 2H), 1.98-2.08 (m, 2H), 1.69-1.84 (m, 2H). MS (ESI): [M−H]⁺=402.51

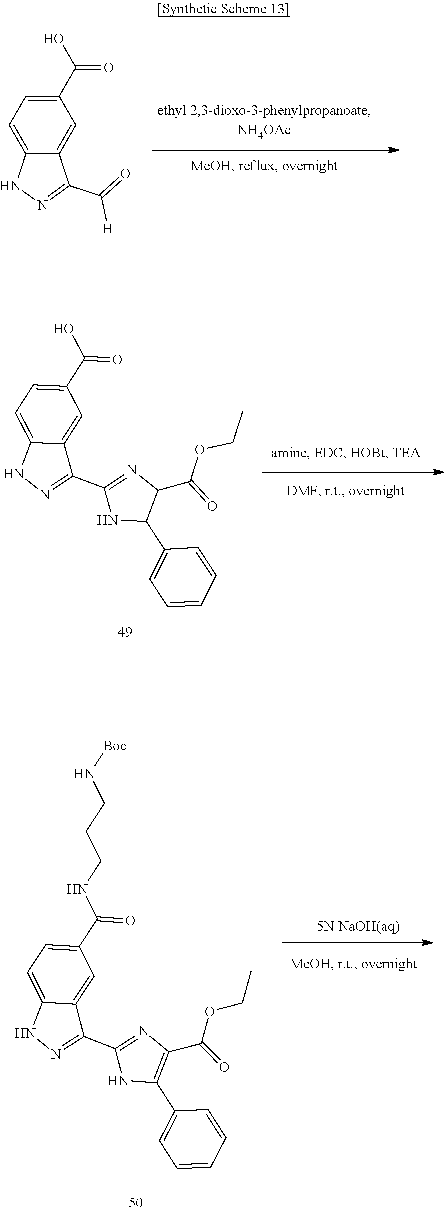 [SMILES: O=Cc1n[nH]c2ccc(C(=O)O)cc12 ; CCOC(=O)C1N=C(c2n[nH]c3ccc(C(=O)O)cc23)NC1c1ccccc1 ; CCOC(=O)c1nc(-c2n[nH]c3ccc(C(=O)NCCCNC(=O)OC(C)(C)C)cc23)[nH]c1-c1ccccc1]

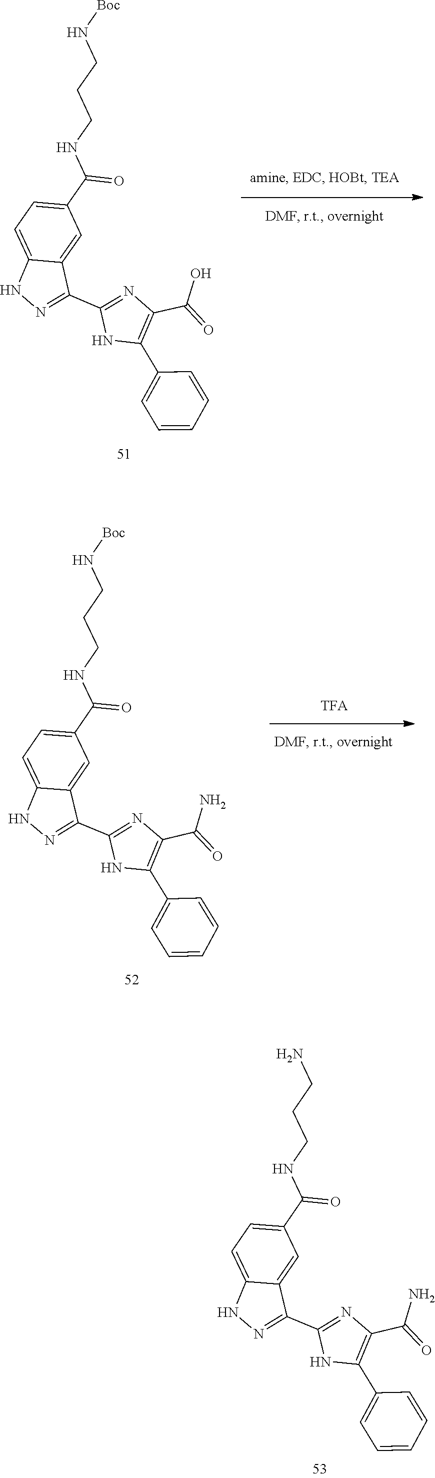 [SMILES: CC(C)(C)OC(=O)NCCCNC(=O)c1ccc2[nH]nc(-c3nc(C(=O)O)c(-c4ccccc4)[nH]3)c2c1 ; CC(C)(C)OC(=O)NCCCNC(=O)c1ccc2[nH]nc(-c3nc(C(N)=O)c(-c4ccccc4)[nH]3)c2c1 ; NCCCNC(=O)c1ccc2[nH]nc(-c3nc(C(N)=O)c(-c4ccccc4)[nH]3)c2c1]

### 3-(4-(ethoxycarbonyl)-5-phenyl-1H-imidazol-2-yl)-1H-indazole-5-carboxylic acid (49)

The starting material 3-formyl-1H-indazole-5-carboxylic acid was dissolved in methanol, and ethyl 2,3-dioxo-3-phenylpropanoate (1.0 eq) and NH₄OAc (5.0 eq) were added. The solution was stirred under reflux for 12 hours, and completion of the reaction was confirmed. The solvent was completely removed under reduced pressure, and the residue was extracted with water and ethyl acetate, dried over Na₂SO₄, and filtered. The solvent was removed under reduced pressure, and the residue was purified by column chromatography to afford compound 49 (brown solid, yield: 43%).

¹H NMR (400 MHz, DMSO-d₆) δ 9.02 (s, 1H), 7.84-8.10 (m, 2H), 7.66-7.79 (m, 1H), 7.55-7.66 (m, 1H), 7.36-7.49 (m, 3H), 4.14-4.33 (m, 2H), 1.20-1.26 (m, 3H). MS (ESI): [M+H]⁺=376.8

### ethyl 2-(5-((3-((tert-butoxycarbonyl)amino)propyl)carbamoyl)-1H-indazol-3-yl)-5-phenyl-1H-imidazole-4-carboxylate(50)

Compound 50 was obtained from compound 49 by using tert-butyl (3-aminopropyl)carbamate according to General Synthesis Procedure A (brown oil, yield: 23%).

¹H NMR (400 MHz, DMSO-d₆) δ 8.78-8.89 (m, 1H), 8.47-8.57 (m, 1H), 8.00-8.13 (m, 1H), 7.80-7.94 (m, 1H), 7.62-7.73 (m, 2H), 7.33-7.51 (m, 3H), 6.79-6.89 (m, 1H), 4.12-4.33 (m, 2H), 3.24-3.29 (m, 2H), 2.98-3.05 (m, 2H), 1.61-1.70 (m, 2H), 1.38 (d, J=1.60 Hz, 9H), 1.14-1.28 (m, 3H). MS (ESI): [M+H]⁺=533.3

### 2-(5-((3-((tert-butoxycarbonyl)amino)propyl)carbamoyl)-1H-indazol-3-yl)-5-phenyl-1H-imidazole-4-carboxylic acid (51)

Compound 51 was obtained from compound 50 according to General Synthesis Procedure D (brown solid, yield: 43%).

¹H NMR (400 MHz, DMSO-d₆) δ 8.84-8.96 (m, 1H), 8.54-8.60 (m, 1H), 7.86-8.03 (m, 1H), 7.73-7.84 (m, 1H), 7.60-7.69 (m, 1H), 7.28-7.51 (m, 2H), 6.78-6.92 (m, 1H), 2.95-3.07 (m, 2H), 1.58-1.74 (m, 2H), 1.33 (s, 9H), 1.07-1.28 (m, 2H). MS (ESI): [M+H]⁺=505.3

### tert-butyl (3-(3-(4-carbamoyl-5-phenyl-1H-imidazol-2-yl)-1H-indazole-5-carboxamido)propyl)carbamate (52)

Compound 51 was dissolved in DMF, and EDC-HCl(1.2 eq) and HOBt (1.3 eq) were added, and ammonium hydroxide solution (2.0 eq) was added, and the solution was stirred at room temperature for 12 hours. Upon confirming completion of the reaction, the solution was extracted with water and ethyl acetate, dried over Na₂SO₄, and filtered. Thereafter, the solvent was removed under reduced pressure, and the residue was purified by column chromatography to afford compound 52 (brown oil, yield: 52%).

¹H NMR (400 MHz, DMSO-d₆) δ 8.94-8.99 (m, 1H), 8.60-8.67 (m, 1H), 7.90-7.98 (m, 3H), 7.66-7.72 (m, 1H), 7.36-7.46 (m, 3H), 7.20-7.26 (m, 1H), 6.78-6.88 (m, 1H), 2.99-3.04 (m, 2H), 1.64-1.70 (m, 2H), 1.38 (s, 9H), 1.20-1.25 (m, 2H). MS (ESI): [M+H]⁺=504.3

### Example 113: N-(3-aminopropyl)-3-(4-carbamoyl-5-phenyl-1H-imidazol-2-yl)-1H-indazole-5-carboxamide (53)

Compound (53) of Example 113 was obtained from compound 52 according to General Synthesis Procedure B (white solid, yield: 44%).

¹H NMR (400 MHz, DMSO-d₆) δ 8.93-9.00 (m, 1H), 8.67-8.75 (m, 1H), 7.89-7.99 (m, 3H), 7.66-7.72 (m, 1H), 7.59-7.66 (m, 1H), 7.39-7.44 (m, 2H), 7.31-7.39 (m, 1H), 7.20-7.28 (m, 1H), 3.35-3.40 (m, 2H), 2.60-2.69 (m, 2H), 1.59-1.68 (m, 2H). MS (ESI): [M+H]⁺=404.3

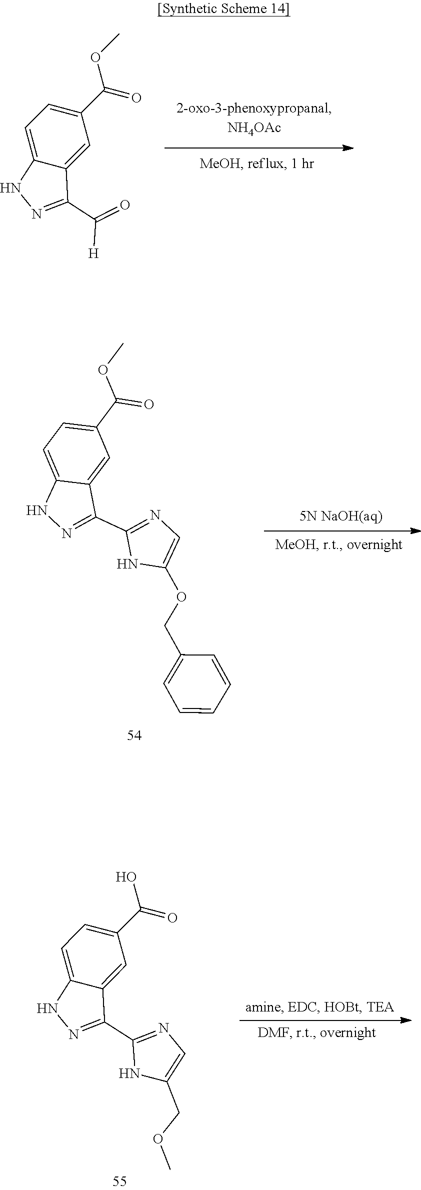 [SMILES: COC(=O)c1ccc2[nH]nc(C=O)c2c1 ; COC(=O)c1ccc2[nH]nc(-c3ncc(OCc4ccccc4)[nH]3)c2c1 ; COCc1cnc(-c2n[nH]c3ccc(C(=O)O)cc23)[nH]1]

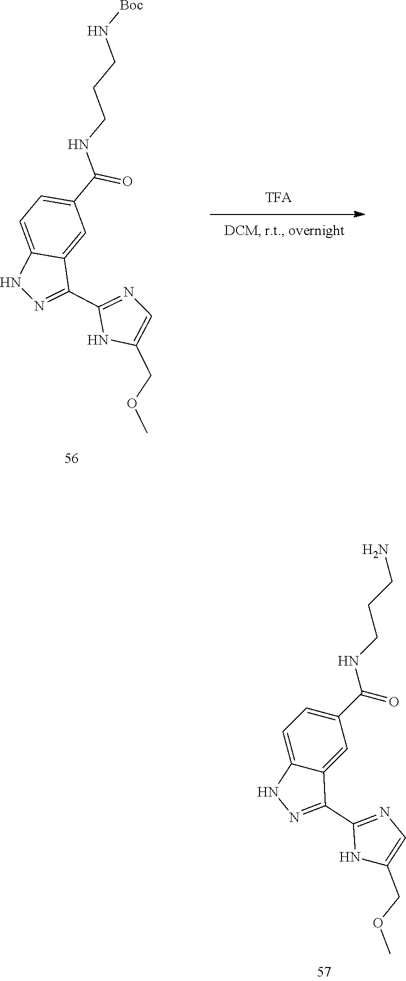 [SMILES: COCc1cnc(-c2n[nH]c3ccc(C(=O)NCCCNC(=O)OC(C)(C)C)cc23)[nH]1 ; COCc1cnc(-c2n[nH]c3ccc(C(=O)NCCCN)cc23)[nH]1]

### methyl 3-(5-(phenoxymethyl)-1H-imidazol-2-yl)-1H-indazole-5-carboxylate (54)

Compound 54 was obtained by using 2-oxo-3-phenoxypropanal according to General Synthesis Procedure I (brown oil, yield: 31%).

¹H NMR (400 MHz, DMSO-d₆) δ 9.07 (s, 1H), 7.99 (dd, J=1.60, 8.93 Hz, 1H), 7.64-7.70 (m, 1H), 7.23-7.38 (m, 3H), 7.11 (d, J=7.79 Hz, 2H), 6.91-7.00 (m, 1H), 5.04-5.12 (m, 2H), 3.91 (s, 3H). MS (ESI): [M+H]⁺=349.3

### 3-(5-(methoxymethyl)-1H-imidazol-2-yl)-1H-indazole-5-carboxylic acid (55)

Compound 54 was dissolved in MeOH, and an equal volume of 5N NaOH(aq) was added. The solution was stirred at room temperature for 12 hours, and completion of the reaction was confirmed. The solution was neutralized with 1N HCl(aq), and the solvent was completely removed under reduced pressure. The residue was dissolved in chloroform/Methanol (3:1) and filtered. The filtrate was collected, and the solvent was completely removed under reduced pressure to afford compound 55 (brown solid, yield: 21%).

### tert-butyl (3-(3-(5-(methoxymethyl)-1H-imidazol-2-yl)-1H-indazole-5-carboxamido)propyl)carbamate (56)

Compound 56 was obtained from compound 55 by using tert-butyl (3-aminopropyl)carbamate according to General Synthesis Procedure A (yellow oil, yield: 37%).

¹H NMR (400 MHz, METHANOL-d₄) δ 8.85 (br. s., 1H), 7.92 (dd, J=1.60, 8.70 Hz, 1H), 7.61 (dd, J=0.80, 8.82 Hz, 1H), 7.22 (s, 1H), 4.53 (s, 2H), 3.46-3.51 (m, 2H), 3.42 (s, 3H), 3.15-3.21 (m, 2H), 1.78-1.86 (m, 2H), 1.43 (s, 9H). MS (ESI): [M+H]⁺=429.3

### Example 114: N-(3-aminopropyl)-3-(5-(methoxymethyl)-1H-imidazol-2-yl)-1H-indazole-5-carboxamide (57)

Compound (57) of Example 114 was obtained from compound 56 according to General Synthesis Procedure B (colorless oil, yield: 32%).

¹H NMR (400 MHz, METHANOL-d₄) δ 8.78-8.82 (m, 1H), 7.96-8.02 (m, 1H), 7.74 (dd, J=0.80, 8.82 Hz, 1H), 7.52-7.57 (m, 1H), 4.52-4.64 (m, 2H), 3.49-3.61 (m, 2H), 3.46 (s, 3H), 2.99-3.10 (m, 2H), 1.95-2.08 (m, 2H). MS (ESI): [M+H]⁺=329.6

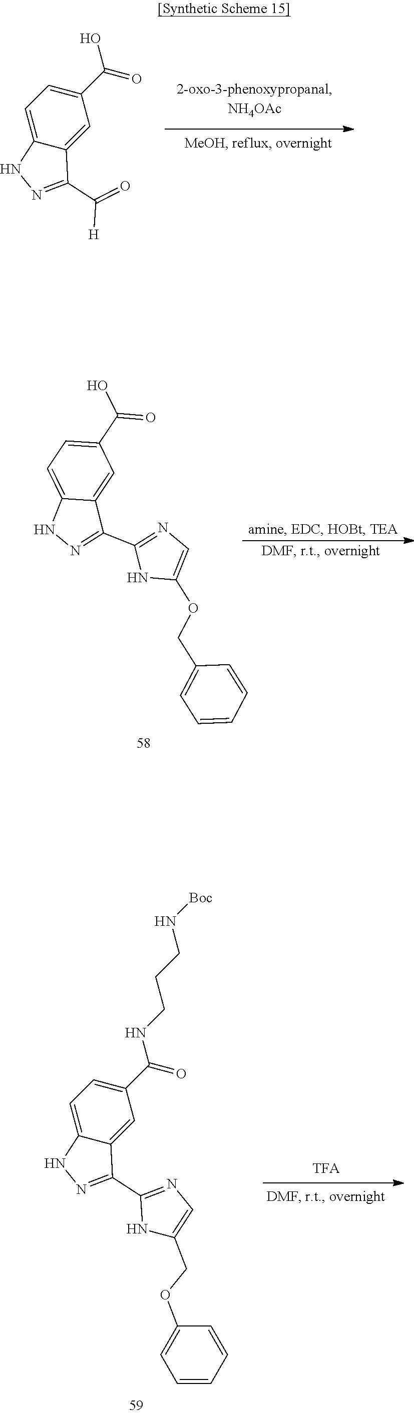 [SMILES: O=Cc1n[nH]c2ccc(C(=O)O)cc12 ; O=C(O)c1ccc2[nH]nc(-c3ncc(OCc4ccccc4)[nH]3)c2c1 ; CC(C)(C)OC(=O)NCCCNC(=O)c1ccc2[nH]nc(-c3ncc(COc4ccccc4)[nH]3)c2c1]

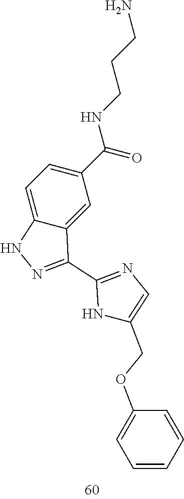 [SMILES: NCCCNC(=O)c1ccc2[nH]nc(-c3ncc(COc4ccccc4)[nH]3)c2c1]

### (5-(benzyloxy)-1H-imidazol-2-yl)-1H-indazole-5-carboxylic acid (58)

3-3-formyl-1H-indazole-5-carboxylic acid was dissolved in methanol, and 2-oxo-3-phenoxypropanal (1.0 eq) and NH₄OAc (5.0 eq) were added. The solution was stirred under reflux for 12 hours, and completion of the reaction was confirmed. The solution was filtered and washed with water and a small amount of cold methanol. Thereafter, the solid was dried in a vacuum oven to afford compound 58 (brown solid, yield: 32%).

### tert-butyl (3-(3-(5-(phenoxymethyl)-1H-imidazol-2-yl)-1H-indazole-5-carboxamido)propyl)carbamate (59)

Compound 59 was obtained from compound 58 by using tert-butyl (3-aminopropyl)carbamate according to General Synthesis Procedure A (brown oil, yield: 27%). MS (ESI): [M+H]⁺=491.2

### Example 115: N-(3-aminopropyl)-3-(5-(phenoxymethyl)-1H-imidazol-2-yl)-1H-indazole-5-carboxamide (60)

Compound (60) of Example 115 was obtained from compound 59 according to General Synthesis Procedure B (yellow oil, yield: 27%).

¹H NMR (400 MHz, METHANOL-d₄) δ 8.87 (dd, J=0.92, 1.60 Hz, 1H), 7.90-7.95 (m, 1H), 7.57-7.65 (m, 1H), 7.24-7.34 (m, 3H), 7.06 (quind, J=1.14, 7.82 Hz, 2H), 6.95 (tt, J=0.97, 7.39 Hz, 1H), 5.14 (s, 2H), 3.48-3.55 (m, 2H), 2.81 (t, J=6.99 Hz, 2H), 1.79-1.90 (m, 2H). MS (ESI): [M+H]⁺=391.3

In experiment examples, anti-cancer effect of Compound of the present invention was confirmed by enzyme inhibition test and cell proliferation inhibition test in cancer cell line.

### Experimental Example 1. Evaluation of Various Kinase Inhibitory Activities of a Compound According to the Present Invention

The following assay method was performed to evaluate the inhibitory activities of a compound according to the present invention with respect to various enzymes. Kinase selectivity was measured for the compound of Example 32, selected from the examples of the present invention, and the experiment was performed using the scanMax® kinase panel from Eurofins. The compound was applied to the enzyme at a concentration of 1 μM, dissolved in DMSO, and the % control was determined according to Equation 1 below.

$$( Example Compound - Positive Control ) / ( Negative Control - Positive Control ) * 100 \qquad [ Equation 1 ]$$

Here, the positive control refers to a compound corresponding to 0% control, and the negative control refers to a compound corresponding to 100% control.

As a result, the compound of Example 32 showed a % control value of less than 10 against the following enzymes:

ABL1(E255K)-phosphorylated, ABL1(F317I)-phosphorylated, ABL1(F317L)-phosphorylated, ABL1(H396P)-nonphosphorylated, ABL1(H396P)-phosphorylated, ABL1(M351T)-phosphorylated, ABL1(Q252H)-nonphosphorylated, ABL1(Q252H)-phosphorylated, ABL1(T315I)-nonphosphorylated, ABL1(T315I)-phosphorylated, ABL1(Y253F)-phosphorylated, ABL1-nonphosphorylated, ABL1-phosphorylated, ARK5, AURKA, AURKB, AURKC, AXL, CDK2, CDK3, CDK4-cycllinDl, CDK4-cyclinD3, CDK5, CDK7, DYRKIA, DYRK1B, DYRK2, ERK1, ERK2, ERK3, ERK4, ERK8, FLT3, FLT3(D835H), FLT3(D835V), FLT3(D835Y), FLT3(ITD), FLT3(ITD, D835V), FLT3(ITD, F691L), FLT3(K663Q), FLT3(N841I), FLT3(R834Q), FLT3-autoinhibited, GSK3A, GSK3B, HASPIN, HIPK1, HIPK2, HIPK3, HIPK4, IRAKI, IRAK3, IRAK4, JAKI(JH1domain-catalytic), JAK1(JH2domain-pseudokinase), JAK2(JH1domain-catalytic), JAK3(JH1domain-catalytic), KIT, KIT(A829P), KIT(D816H), KIT(D816V), KIT(L576P), KIT(V559D), KIT(V559D, T670I), KIT(V559D, V654A), MAP3K1, MAP3K15, MAP3K2, MAP3K3, MEKI, MEK2, MEK3, MEK4, MEK5, MEK6, PAKI, PAK2, PAK3, PAK4, PAK4, PAK5, PAK6, PAK7, PIM1, PIM2, PIM3, RET, RET(M918T), RET(V804L), RET(V804M), ROCKI, ROCK2, SRC, TRKA, TRKB, TRKC, YES1.

### Experimental Example 2. Evaluation of the Kinase Inhibitory Activity of a Compound According to the Present Invention

FLT3 activity inhibition was measured using the ADP-Glo kinase assay method (Promega, cat #V9101). The ADP-Glo kinase assay method is a luminescent ADP detection assay method that provides a homogeneous, high-throughput screening method for measuring kinase activity by quantifying the amount of ADP produced during the kinase reaction.

The assay was performed in two steps. First, after the kinase reaction, an equal volume of ADP-Glo™ reagent was added to terminate the kinase reaction and deplete any remaining ATP. Second, a kinase detection reagent was added to convert ADP to ATP, and simultaneously the newly synthesized ATP was measured via the luciferase/luciferin reaction. The light produced was measured using a luminometer. The measured amount of luminescence was plotted on the ATP-ADP standard conversion curve to confirm the correlation with ADP concentration.

Recombinant proteins containing FLT3 and FLT3(D835Y) domains were purchased from Promega (cat #V4064, cat #V4514), respectively. According to the manufacturer's instructions, the optimal enzyme, ATP, and substrate concentrations were optimized using the ADP-Glo™ kinase assay method (Promega, USA). The 5× kinase reaction buffer (200 mM Tris-HCl, pH 7.5, 100 mM MgCl₂, 0.5 mg/ml BSA) was diluted and used, and a diluted compound of the present invention (5×, 1 μl), FLT3 enzyme or FLT3/D835Y enzyme (2.5×, 2 μl) and substrate/ATP mixture (2.5×, 2 μl) were sequentially mixed and reacted at room temperature for 120 minutes. Thereafter, ADP-Glo™ reagent (5 μl) was added and reacted for an additional 40 minutes at room temperature. Finally, the detection reagent (kinase detection reagent, 10 μl) was added, followed by reaction at room temperature for 30 minutes, and the phosphorylation of the substrate peptide was measured by detecting the luciferase signal (integration time 0.5~1 sec) using a SpectraMax ID5 reader (Molecular Devices LLC., San Jose, CA, USA). The IC₅₀was calculated by nonlinear regression analysis using Prism version 5.01 (GraphPad).

The measured enzyme activity (IC₅₀, nM) results are shown in Table 5 below.

TABLE 5
Evaluation of FLT3 WT/D835Y
Kinase Inhibitory Activity

| | Enzyme (IC₅₀) | |
|---|---|---|
| | FLT3 | FLT3 |
| | WT | D835Y |
| Example 7 (2g) | ++++ | — |
| Example 11 (5r) | ++++ | — |
| Example 12 (5s) | ++++ | — |
| Example 15 (6a) | ++++ | — |
| Example 16 (6b) | ++++ | ++++ |
| Example 17 (6c) | ++++ | ++++ |
| Example 18 (6d) | ++++ | — |
| Example 20 (6f) | ++++ | ++++ |
| Example 21 (6g) | ++++ | ++++ |
| Example 22 (6h) | ++++ | ++++ |
| Example 23 (6i) | ++++ | — |
| Example 26 (6l) | ++++ | — |
| Example 30 (6t) | ++++ | ++++ |
| Example 31 (6u) | ++++ | — |
| Example 32 (6v) | ++++ | — |
| Example 42 (12a) | ++++ | — |
| Example 43 (12b) | ++++ | — |
| Example 45 (12d) | ++++ | — |
| Example 47 (12f) | ++++ | ++++ |
| Example 50 (12i) | ++++ | ++++ |
| Example 51 (12j) | ++++ | ++++ |
| Example 52 (12k) | ++++ | — |
| Example 53 (12l) | ++++ | — |
| Example 54 (12m) | ++++ | — |
| Example 55 (12n) | ++++ | — |
| Example 56 (12o) | ++++ | — |
| Example 57 (12p) | ++++ | — |
| Example 58 (12q) | ++++ | — |
| Example 59 (12r) | ++++ | — |
| Example 63 (14k) | ++++ | — |
| Example 73 (21a) | ++++ | ++++ |
| Example 74 (21b) | ++++ | ++++ |
| Example 77 (21e) | ++++ | ++++ |
| Example 78 (21f) | ++++ | — |
| Example 76 (21d) | ++++ | — |
| Example 79 (21g) | ++++ | — |

(++++ < 10 nM < +++ < 100 nM < ++ < 1,000 nM < +)

As shown in Table 5, the compound according to the present invention exhibited excellent inhibitory activity against FLT3 kinase and FLT3 mutant kinase.

### Experimental Example 3. Evaluation of Cell Proliferation Inhibition in AML Cell Lines

The cell proliferation inhibition experiment on acute myeloid leukemia (AML) cell lines was performed using FL3-ITD-expressing AML cell line MV4-11, BaF3-ITD-FLT3 F691L cell line, and BaF3-ITD-FLT3 D835Y cell line to test FLT3 inhibitory activity. All cell lines were human-derived cell lines, and the sources and culture conditions for each cell line are as follows.

MV4-11 cells were purchased from the American Type Culture Collection (ATCC, Rockville, MD, USA, cat #CRL-9591), and BaF3-ITD-FLT3 F691L and BaF3-ITD-FLT3-D835Y were purchased from Creative Biogene (Shirley, NY, USA, cat #CSC—R00739 and CSC—R00241), respectively. Cells were cultured in RPMI 1640 medium (Coring Co. 10-040-CV) supplemented with 10% fetal bovine serum, 1% penicillin/streptomycin, and 4 mM L-glutamine (Life Technology, GrandIsland, NY).

Cell viability was assessed by a resazurin redox-based assay using the Alamar Blue Assay kit (Invitrogen, USA). Specifically, MV4-11, BaF3-ITD-FLT3-F691L, and BaF3-ITD-FLT3-D835Y cells were plated in 384-well plates at 8,000 cells in 45 μL of medium. As a negative control, dimethyl sulfoxide (DMSO) was added together with the compound. After three days (72 hours) of compound treatment, 5 μL of the Alamar Blue kit reagent was added to each well of the 384-well plates, followed by incubation at 37° C. for 2 hours in a humidified CO₂ incubator. After incubation, fluorescence intensity (Flu) was measured at 544 nm and 590 nm using a SpectraMax iD3 Multi Mode Microplate Reader (Molecular Devices, ID3-STD). The GI₅₀ was calculated by nonlinear regression analysis using Prism version 5.01 (GraphPad, LaJolla, CA, USA).

The measured cell proliferation inhibitory activity (GI₅₀, nM) results are shown in Table 6 below.

TABLE 6
Cell proliferation inhibitory
activity in AML cell lines (GI₅₀, nM)

| | Cellular (GI₅₀) | | |
|---|---|---|---|
| | | BaF3-ITD- | BaF3-ITD- |
| | | FLT3 | FLT3 |
| | MV4-11 | F691L | D835Y |
| Example 2 | ++ | — | — |
| (1b) | | | |
| Example 3 | + | — | — |
| (1c) | | | |
| Example 7 | +++ | — | — |
| (2g) | | | |
| Example 8 | +++ | — | — |
| (2h) | | | |
| Example 9 | +++ | — | — |
| (5p) | | | |
| Example | ++ | — | — |
| 10 (5q) | | | |
| Example | ++++ | — | — |
| 11 (5r) | | | |
| Example | ++++ | — | — |
| 12 (5s) | | | |
| Example | +++ | +++ | +++ |
| 13 (5w) | | | |
| Example | +++ | ++ | +++ |
| 14 (5x) | | | |
| Example | ++++ | — | — |
| 15 (6a) | | | |
| Example | ++++ | +++ | ++++ |
| 16 (6b) | | | |
| Example | ++++ | +++ | ++++ |
| 17 (6c) | | | |
| Example | +++ | — | — |
| 18 (6d) | | | |
| Example | ++ | — | — |
| 19 (6e) | | | |
| Example | ++++ | +++ | ++++ |
| 20 (6f) | | | |
| Example | ++ | — | — |
| 21 (6g) | | | |
| Example | ++++ | +++ | ++++ |
| 22 (6h) | | | |
| Example | ++++ | — | — |
| 23 (6i) | | | |
| Example | ++ | — | — |
| 24 (6j) | | | |
| Example | +++ | — | — |
| 25 (6k) | | | |
| Example | ++ | — | — |
| 26 (6l) | | | |
| Example | +++ | — | — |
| 27 (6m) | | | |
| Example | +++ | — | — |
| 28 (6n) | | | |
| Example | +++ | — | — |
| 29 (60) | | | |
| Example | ++++ | ++++ | ++++ |
| 30 (6t) | | | |
| Example | ++++ | +++ | ++++ |
| 31 (6u) | | | |
| Example | ++++ | ++++ | ++++ |
| 32 (6v) | | | |
| Example | ++++ | — | — |
| 33 (36c) | | | |
| Example | + | — | — |
| 34 (9a) | | | |
| Example | +++ | — | — |
| 35 (9b) | | | |
| Example | +++ | — | — |
| 36 (9c) | | | |
| Example | +++ | — | — |
| 37 (10d) | | | |
| Example | +++ | — | — |
| 38 (10e) | | | |
| Example | ++ | — | — |
| 39 (10f) | | | |
| Example | +++ | — | — |
| 40 (10g) | | | |
| Example | + | — | — |
| 41 (10h) | | | |
| Example | +++ | — | — |
| 42 (12a) | | | |
| Example | +++ | — | — |
| 43 (12b) | | | |
| Example | ++++ | — | — |
| 44 (12c) | | | |
| Example | ++++ | — | — |
| 45 (12d) | | | |
| Example | +++ | — | — |
| 46 (12e) | | | |
| Example | +++ | +++ | — |
| 47 (12f) | | | |
| Example | ++++ | — | — |
| 48 (12g) | | | |
| Example | ++ | — | — |
| 49 (12h) | | | |
| Example | ++++ | +++ | +++ |
| 50 (12i) | | | |
| Example | ++++ | +++ | ++++ |
| 51 (12j) | | | |
| Example | +++ | — | — |
| 52 (12k) | | | |
| Example | -+++ | — | — |
| 53 (12l) | | | |
| Example | -+++ | — | — |
| 54 (12m) | | | |
| Example | ++++ | — | — |
| 55 (12n) | | | |
| Example | ++++ | — | — |
| 56 (120) | | | |
| Example | ++++ | — | — |
| 57 (12p) | | | |
| Example | +++ | — | — |
| 58 (12q) | | | |
| Example | ++++ | ++ | ++ |
| 59 (12r) | | | |
| Example | + | — | — |
| 60 (13h) | | | |
| Example | + | — | — |
| 61 (13f) | | | |
| Example | ++++ | — | — |
| 62 (14j) | | | |
| Example | ++++ | — | — |
| 63 (14k) | | | |
| Example | ++ | — | — |
| 64 (15a) | | | |
| Example | +++ | — | — |
| 65 (15b) | | | |
| Example | +++ | — | — |
| 66 (15c) | | | |
| Example | +++ | — | — |
| 67 (15d) | | | |
| Example | +++ | — | — |
| 68 (15e) | | | |
| Example | +++ | — | — |
| 69 (15f) | | | |
| Example | +++ | — | — |
| 70 (15g) | | | |
| Example | +++ | — | — |
| 71 (15h) | | | |
| Example | +++ | — | — |
| 72 (15i) | | | |
| Example | ++++ | +++ | +++ |
| 73 (21 a) | | | |
| Example | ++++ | ++++ | ++++ |
| 74 (21b) | | | |
| Example | +++ | +++ | ++++ |
| 75 (21c) | | | |
| Example | +++ | — | — |
| 77 (21e) | | | |
| Example | +++ | +++ | — |
| 78 (21f) | | | |
| Example | ++++ | +++ | ++++ |
| 76 (21d) | | | |
| Example | ++++ | +++ | — |
| 79 (21g) | | | |
| Example | ++++ | +++ | ++++ |
| 80 (21h) | | | |
| Example | + | — | — |
| 110 (44) | | | |
| Example | ++ | — | — |
| 111 (46) | | | |
| Example | +++ | — | — |
| 112 (48) | | | |
| Example | + | — | — |
| 105 (34a) | | | |
| Example | + | — | — |
| 106 (34c) | | | |
| Example | + | — | — |
| 107 (34d) | | | |
| Example | + | — | — |
| 108 (34e) | | | |
| Example | + | — | — |
| 109 (35c) | | | |
| Example | +++ | — | — |
| 81 (25a) | | | |
| Example | +++ | — | — |
| 82 (25b) | | | |
| Example | +++ | — | — |
| 83 (25c) | | | |
| Example | +++ | — | — |
| 85 (25e) | | | |
| Example | +++ | — | — |
| 86 (25f) | | | |
| Example | +++ | — | — |
| 87 (25g) | | | |
| Example | +++ | — | — |
| 88 (25h) | | | |
| Example | ++ | — | — |
| 89 (25i) | | | |
| Example | ++ | — | — |
| 90 (25j) | | | |
| Example | +++ | — | — |
| 91 (25k) | | | |
| Example | ++ | — | — |
| 92 (26h) | | | |
| Example | ++ | — | — |
| 93 (26i) | | | |
| Example | + | — | — |
| 94 (29a) | | | |
| Example | ++ | — | — |
| 95 (29b) | | | |
| Example | + | — | — |
| 97 (29d) | | | |
| Example | + | — | — |
| 99 (29f) | | | |
| Example | + | — | — |
| 103 (30j) | | | |
| Example | ++ | — | — |
| 113 (53) | | | |
| Example | + | — | — |
| 114 (57) | | | |
| Example | + | — | — |
| 115 (60) | | | |

(++++ < 10 nM < +++ < 100 nM < ++ < 1,000 nM < +)

As shown in Table 6, it was confirmed that the compound of the present invention exhibited potent anti-proliferative activity in acute myeloid leukemia cell lines. In particular, it was confirmed that it exhibited potent anti-proliferative activity in the FLT3-ITD expressing MV4-11 cell line, BaF3-ITD-FLT3 F691L cell line and BaF3-ITD-FLT3 D835Y cell line, which express mutant kinase. From these results, it was confirmed that the compound of the present invention exhibits excellent inhibitory activity against FLT3 kinase, especially FLT3 mutant kinase, and preventive or therapeutic effects on leukemia associated therewith.

1. A compound or a pharmaceutically acceptable salt thereof, wherein the compound is represented by Formula 1 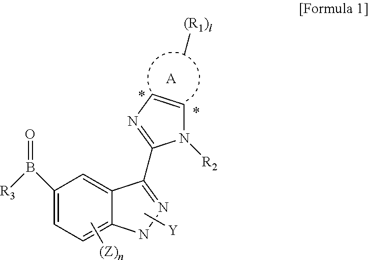
In Formula 1,
wherein A is absent, or if present, is a (6-14 membered) aryl or a (5-12 membered) heteroaryl,
if A is absent, Ri is optionally bonded to the “*” position,
if A is present, R₁ is optionally substituted on A,
R₁ is each independently selected from the group consisting of hydrogen, halogen, C₁₋₁₀ alkyl, C₁₋₁₀ haloalkyl, (3-10 membered) cycloalkyl, C₁₋₁₀ alkylene-(3-10 membered) cycloalkyl, (3-10 membered) heterocyclyl, C₁₋₁₀ alkylene-(3-10 membered) heterocyclyl, (6-14 membered) aryl, C₀₋₁₀ alkylene-(6-14 membered) aryl, (5-12 membered) heteroaryl, C₁₋₁₀ alkylene-(5-12 membered) heteroaryl, C₁₋₁₀ alkoxy, C₁₋₁₀ alkylene-C₁₋₆ alkoxy, 0-(6-14 membered) aryl, C₁₋₁₀ alkylene-O-(6-14 membered) aryl, O—C₁₋₁₀ alkylene-C₁₋₆ alkoxy, O—C₁₋₁₀ alkylene-NR₁ₐR_{1b}, C(O)—NR₁ₐR_{1b}, C₁₋₁₀ alkylene-C(O)—NR₁ₐR_{1b}, C₁₋₆ haloalkoxy, C₁₋₁₀ alkylene-C₁₋₆ haloalkoxy, CN, C₁₋₁₀ alkylene-CN, OH, NO₂, NR₁ₐR_{1b}, OC(O)—NR₁ₐR_{1b}, C(O)R₁ₐ, C(O)OR₁ₐ, NR₁ₐC(O)R_{1b}, NR₁ₐC(O)OR_{1b}, NR₁ₐSO₂R_{1b} and SO₂NR₁ₐR_{1b},
wherein (3-10 membered) cycloalkyl, (3-10 membered) heterocyclyl, (6-14 membered) aryl, and (5-12 membered) heteroaryl are each independently unsubstituted or optionally substituted with at least one of (3-10 membered) cycloalkyl, (3-10 membered) heterocyclyl, C₁₋₆ alkyl, C₁₋₆ alkoxy, halogen, C₁₋₆ haloalkyl, NH₂, NO₂, OH, or NR₁ₐR_{1b},
wherein R₁ₐ and R_{1b} are each independently selected from hydrogen, C₁₋₆ alkyl, or C₁₋₆ haloalkyl, or the R₁ₐ and R_{1b} are optionally bonded together to the same nitrogen atom to form a (3-10 membered) heterocyclyl or a (5-12 membered) heteroaryl within the substituent,
1 is an integer from 0 to 2 when A is absent, and an integer from 0 to 6 when A is present,
R₂ is independently selected from the group consisting of hydrogen, C₁₋₁₀ alkyl, halogen, C₁₋₆ haloalkyl, C₁₋₆ alkoxy, C₁₋₁₀ alkylene-C₁₋₆ alkoxy, C₁₋₆ haloalkoxy, CN, OH, C₁₋₁₀ alkylene-OH, NO₂, C₁₋₆ alkylamino, C₁₋₁₀ alkylene-NR₂ₐR_{2b}, (6-14 membered) aryl, (5-12 membered) heteroaryl, (3-10 membered) cycloalkyl, (3-10 membered) heterocyclyl and C₁₋₁₀ alkylene-(3-10 membered) heterocyclyl,
wherein (6-14 membered) aryl, (5-12 membered) heteroaryl, (3-10 membered) cycloalkyl and (3-10 membered) heterocyclyl are each independently unsubstituted or optionally substituted with at least one of C₁₋₆ alkyl, halogen, C₁₋₆ haloalkyl, C₁₋₆ alkoxy or C₁₋₆ haloalkoxy,
wherein R₂ₐ and R_{2b} are each independently selected from hydrogen, C₁₋₆ alkyl or C₁₋₆ haloalkyl, or R₂ₐ and R_{2b} are optionally bonded together to the same nitrogen atom to form a (3-10 membered) heterocyclyl or a (5-12 membered) heteroaryl within the substituent,
B is carbon or sulfur,
R₃ is NR₃ₐR_{3b},
wherein R₃ₐ and R_{3b} are each independently selected from the group consisting of hydrogen, OH, C₁₋₁₀ alkyl, C₁₋₆ haloalkyl, C(O)C₁₋₆ alkyl, 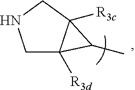
(3-10 membered) cycloalkyl, (6-14 membered) aryl, (5-12 membered) heteroaryl and (3-10 membered) heterocyclyl,
or wherein R₃ₐ and R_{3b} are optionally bonded together to the same nitrogen atom to form a (3-10 membered) heterocyclyl or a (5-12 membered) heteroaryl within the substituent,
wherein R_{3c} and R_{3d} are each independently selected from hydrogen, C₁₋₆ alkyl or C₁₋₆ haloalkyl,
wherein C₁₋₁₀ alkyl, (3-10 membered) cycloalkyl, (3-10 membered) heterocyclyl, (6-14 membered) aryl, and (5-12 membered) heteroaryl are each independently unsubstituted or optionally substituted with R₄,
R₄ is selected from the group consisting of OH, C₁₋₆ alkyl, C₁₋₆ alkoxy, halogen, C₁₋₆ haloalkyl, C(O)C₁₋₆ alkyl, (6-14 membered) aryl, (3-10 membered) cycloalkyl, (3-10 membered) heterocyclyl, (5-12 membered) heteroaryl and NR₄ₐR_{4b},
wherein R₄ₐ and R_{4b} are each independently selected from hydrogen, C₁₋₆ alkyl, C₁₋₆ haloalkyl, C(O)C₁₋₆ alkyl, SO₂(C₁₋₆)alkyl, or C(O)C₁₋₆ haloalkyl, or R₄ₐ and R_{4b} are optionally bonded together to the same nitrogen atom to form a (3-10 membered) heterocyclyl or a (5-12 membered) heteroaryl within the substituent,
wherein C₁₋₆ alkyl, (6-14 membered) aryl, (3-10 membered) cycloalkyl, and (3-10 membered) heterocyclyl are each independently unsubstituted or optionally substituted with at least one of C₁₋₆ alkyl, amino, halogen, C₁₋₆ haloalkyl, C(O)C₁₋₆ alkyl, C₁₋₆ alkoxy or C₁₋₆ haloalkoxy,
Y is selected from the group consisting of hydrogen, C₁₋₆ alkyl, C₁₋₆ haloalkyl, C₁₋₆ alkoxy, C(O)C₁₋₆ alkyl, C(O)OC₁₋₆ alkyl and C(O)N—C₁₋₆ alkyl,
Z is selected from the group consisting of hydrogen, C₁₋₆ alkyl, halogen, C₁₋₆ haloalkyl, C₁₋₆ alkoxy, NH₂, OH, CN, COOH, C(O)C₁₋₆ alkyl, C(O)OC₁₋₆ alkyl and C(O)N—C₁₋₆ alkyl,
wherein heterocyclyl and heteroaryl are each independently comprise at least one of N, O or S in the ring, and
n is an integer from 0 to 3.

2. The compound or the pharmaceutically acceptable salt thereof according to claim 1, characterized in that
1 is an integer from 0 to 2 both when A is absent and when A is present,
R₁ is each independently selected from the group consisting of hydrogen, halogen, C₁₋₁₀ alkyl, C₁₋₁₀ haloalkyl, (3-10 membered) heterocyclyl, C₁₋₁₀ alkylene-(3-10 membered) heterocyclyl, (6-14 membered) aryl, (5-12 membered) heteroaryl, C₁₋₁₀ alkoxy, C₁₋₁₀ alkylene-C₁₋₆ alkoxy, C₁₋₁₀ alkylene-O-(6-14 membered) aryl, O—C₁₋₁₀ alkylene-C₁₋₆ alkoxy, O—C₁₋₁₀ alkylene-NR₁ₐR_{1b}, C(O)—NR₁ₐR_{1b}, C₁₋₁₀ alkylene-C(O)—NR₁ₐR_{1b} and CN,
wherein (3-10 membered) heterocyclyl, (6-14 membered) aryl, and (5-12 membered) heteroaryl are each independently unsubstituted or optionally substituted with at least one of (3-10 membered) heterocyclyl, C₁₋₆ alkyl, C₁₋₆ alkoxy, halogen, C₁₋₆ haloalkyl, NH₂, NO₂, OH, or NR₁ₐR_{1b},
wherein R₁ₐ and R_{1b} are each independently hydrogen or C₁₋₆ alkyl, or R₁ₐ and R_{1b} are optionally bonded together to the same nitrogen atom to form a (3-10 membered) heterocyclyl within the substituent,
R₂ is each independently selected from the group consisting of hydrogen, C₁₋₁₀ alkyl, C₁₋₁₀ alkylene-C₁₋₆ alkoxy, C₁₋₁₀ alkylene-OH, C₁₋₁₀ alkylene-NR₂ₐR_{2b} and C₁₋₁₀ alkylene-(3-10 membered) heterocyclyl,
wherein (3-10 membered) heterocyclyl is each independently unsubstituted or optionally substituted with C₁₋₆ alkyl,
wherein R₂ₐ and R_{2b} are each independently selected from hydrogen or C₁₋₆ alkyl, or the R₂ₐ and R_{2b} are optionally bonded together to the same nitrogen atom to form a (3-10 membered) heterocyclyl within the substituent,
B is carbon or sulfur,
R₃ is —NR₃ₐR_{3b},
wherein R₃ₐ and R_{3b} are each independently selected from the group consisting of hydrogen, C₁₋₁₀ alkyl, 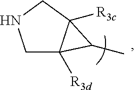
(3-10 membered) cycloalkyl, (6-14 membered) aryl, and (3-10 membered) heterocyclyl,
or wherein R₃ₐ and R_{3b} are optionally bonded together to the same nitrogen atom to form a (3-10 membered) heterocyclyl within the substituent,
wherein R₃, and R_{3d} are each independently selected from hydrogen or C₁₋₆ alkyl,
wherein the C₁₋₁₀ alkyl, (3-10 membered) cycloalkyl, (6-14 membered) aryl, and (3-10 membered) heterocyclyl are each independently unsubstituted or optionally substituted with R₄,
R₄ is selected from the group consisting of OH, C₁₋₆ alkyl, C₁₋₆ haloalkyl, C(O)C₁₋₆ alkyl, (6-14 membered) aryl, (3-10 membered) cycloalkyl, (3-10 membered) heterocyclyl and NR₄ₐR_{4b},
wherein R₄ₐ and R_{4b} are each independently selected from hydrogen, C₁₋₆ alkyl, C(O)C₁₋₆ alkyl, or SO₂(C₁₋₆)alkyl, or the R₄ₐ and R_{4b} are optionally bonded together to the same nitrogen atom to form a (3-10 membered) heterocyclyl within the substituent,
wherein C₁₋₆ alkyl, (6-14 membered) aryl, (3-10 membered) cycloalkyl, and (3-10 membered) heterocyclyl are each independently unsubstituted or optionally substituted with at least one of C₁₋₆ alkyl, amino, halogen, C₁₋₆ haloalkyl, or C(O)C₁₋₆ alkyl,
Y is hydrogen, n is 0.

3. The compound or the pharmaceutically acceptable salt thereof according to claim 1, wherein the compound is represented by Formula 2: 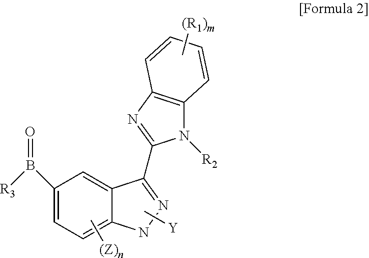
In Formula 2,
R₁ is each independently selected from the group consisting of hydrogen, halogen, C₁₋₁₀ alkyl, C₁₋₁₀ haloalkyl, (3-10 membered)cycloalkyl, C₁₋₁₀ alkylene-(3-10 membered)cycloalkyl, (3-10 membered) heterocyclyl, C₁₋₁₀ alkylene-(3-10 membered)heterocyclyl, (6-14 membered) aryl, C₀₋₁₀ alkylene-(6-14 membered) aryl, (5-12 membered) heteroaryl, C₁₋₁₀ alkylene-(5-12 membered)heteroaryl, C₁₋₁₀ alkoxy, C₁₋₁₀ alkylene-C₁₋₆ alkoxy, 0-(6-14 membered) aryl, C₁₋₁₀ alkylene-O-(6-14 membered) aryl, O—C₁₋₁₀ alkylene-C₁₋₆ alkoxy, O—C₁₋₁₀ alkylene-NR₁ₐR_{1b}, C(O)—NR₁ₐR_{1b}, C₁₋₁₀ alkylene-C(O)—NR₁ₐR_{1b}, C₁₋₆ haloalkoxy, C₁₋₁₀ alkylene-C₁₋₆ haloalkoxy, CN, C₁₋₁₀ alkylene-CN, OH, NO₂, NR₁ₐR_{1b}, OC(O)—NR₁ₐR_{1b}, C(O)R₁ₐ, C(O)OR₁ₐ, NR₁ₐC(O)R_{1b}, NR₁ₐC(O)OR_{1b}, NR₁ₐSO₂R_{1b} and SO₂NR₁ₐR_{1b},
wherein (3-10 membered) cycloalkyl, (3-10 membered) heterocyclyl, (6-14 membered) aryl, and (5-12 membered) heteroaryl are each independently unsubstituted or optionally substituted with at least one of (3-10 membered) cycloalkyl, (3-10 membered) heterocyclyl, C₁₋₆ alkyl, C₁₋₆ alkoxy, halogen, C₁₋₆ haloalkyl, NH₂, NO₂, OH, or NR₁ₐR_{1b},
wherein R₁ₐ and R_{1b} are each independently selected from hydrogen, C₁₋₆ alkyl or C₁₋₆ haloalkyl, or the R₁ₐ and R_{1b} are optionally bonded together to the same nitrogen atom to form a (3-10 membered) heterocyclyl or a (5-12 membered) heteroaryl within the substituent,
m is an integer from 0 to 4,
R₂ is each independently selected from the group consisting of hydrogen, C₁₋₁₀ alkyl, halogen, C₁₋₆ haloalkyl, C₁₋₆ alkoxy, C₁₋₁₀ alkylene-C₁₋₆ alkoxy, C₁₋₆ haloalkoxy, CN, OH, C₁₋₁₀ alkylene-OH, NO₂, C₁₋₆ alkylamino, C₁₋₁₀ alkylene-NR₂ₐR_{2b}, (6-14 membered) aryl, (5-12 membered) heteroaryl, (3-10 membered) cycloalkyl, (3-10 membered) heterocyclyl and C₁₋₁₀ alkylene-(3-10 membered) heterocyclyl,
wherein (6-14 membered) aryl, (5-12 membered) heteroaryl, (3-10 membered) cycloalkyl and (3-10 membered) heterocyclyl are each independently unsubstituted or optionally substituted with at least one of C₁₋₆ alkyl, halogen, C₁₋₆ haloalkyl, C₁₋₆ alkoxy or C₁₋₆ haloalkoxy,
wherein R₂ₐ and R_{2b} are each independently selected from hydrogen, C₁₋₆ alkyl or C₁₋₆ haloalkyl, or the R₂ₐ and R_{2b} are optionally bonded together to the same nitrogen atom to form a (3-10 membered) heterocyclyl or a (5-12 membered) heteroaryl within the substituent,
B is carbon or sulfur,
R₃ is —NR₃ₐR_{3b},
wherein R₃ₐ and R_{3b} are each independently selected from the group consisting of hydrogen, OH, C₁₋₁₀ alkyl, C₁₋₆ haloalkyl, C(O)C₁₋₆ alkyl, 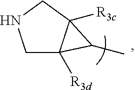
(3-10 membered) cycloalkyl, (6-14 membered) aryl, (5-12 membered) heteroaryl and (3-10 membered) heterocyclyl,
or wherein R₃ₐ and R_{3b} are optionally bonded together to the same nitrogen atom to form a (3-10 membered) heterocyclyl or a (5-12 membered) heteroaryl within the substituent,
wherein R_{3c} and R_{3d} are each independently selected from hydrogen, C₁₋₆ alkyl or C₁₋₆ haloalkyl,
wherein C₁₋₁₀ alkyl, (3-10 membered) cycloalkyl, (3-10 membered) heterocyclyl, (6-14 membered) aryl, and (5-12 membered) heteroaryl are each independently unsubstituted or optionally substituted with R₄,
R₄ is selected from the group consisting of OH, C₁₋₆ alkyl, C₁₋₆ alkoxy, halogen, C₁₋₆ haloalkyl, C(O)C₁₋₆ alkyl, (6-14 membered) aryl, (3-10 membered) cycloalkyl, (3-10 membered) heterocyclyl, (5-12 membered) heteroaryl and NR₄ₐR_{4b},
wherein R₄ₐ and R_{4b} are each independently selected from hydrogen, C₁₋₆ alkyl, C₁₋₆ haloalkyl, C(O)C₁₋₆ alkyl, SO₂(C₁₋₆)alkyl, or C(O)C₁₋₆ haloalkyl, or the R₄ₐ and R_{4b} are optionally bonded together to the same nitrogen atom to form a (3-10 membered) heterocyclyl or a (5-12 membered) heteroaryl within the substituent,
wherein C₁₋₆ alkyl, (6-14 membered) aryl, (3-10 membered) cycloalkyl, and (3-10 membered) heterocyclyl are each independently unsubstituted or optionally substituted with at least one of C₁₋₆ alkyl, amino, halogen, C₁₋₆ haloalkyl, C(O)C₁₋₆ alkyl, C₁₋₆ alkoxy or C₁₋₆ haloalkoxy,
Y is selected from the group consisting of hydrogen, C₁₋₆ alkyl, C₁₋₆ haloalkyl, C₁₋₆ alkoxy, C(O)C₁₋₆ alkyl, C(O)OC₁₋₆ alkyl and C(O)N—C₁₋₆ alkyl,
Z is selected from the group consisting of hydrogen, C₁₋₆ alkyl, halogen, C₁₋₆ haloalkyl, C₁₋₆ alkoxy, NH₂, OH, CN, COOH, C(O)C₁₋₆ alkyl, C(O)OC₁₋₆ alkyl and C(O)N—C₁₋₆ alkyl,
wherein heterocyclyl and heteroaryl are each independently comprise at least one of N, O or S in the ring,
n is an integer from 0 to 3.

4. The compound or the pharmaceutically acceptable salt thereof according to claim 3, characterized in that
R₁ is each independently selected from the group consisting of hydrogen, halogen, C₁₋₁₀ alkyl, C₁₋₁₀ haloalkyl, (3-10 membered) heterocyclyl, C₁₋₁₀ alkylene-(3-10 membered) heterocyclyl, (6-14 membered) aryl, (5-12 membered) heteroaryl, C₁₋₁₀ alkoxy, O—C₁₋₁₀ alkylene-C₁₋₆ alkoxy, O—C₁-0 alkylene-NR₁ₐR_{1b}, C(O)—NR₁ₐR_{1b} and CN,
wherein (3-10 membered) heterocyclyl, (6-14 membered) aryl, and (5-12 membered) heteroaryl are each independently unsubstituted or optionally substituted with at least one of (3-10 membered) heterocyclyl, C₁₋₆ alkyl, C₁₋₆ alkoxy, halogen, C₁₋₆ haloalkyl, NH₂, NO₂, OH, or NR₁ₐR_{1b},
wherein R₁ₐ and R_{1b} are each independently hydrogen or C₁₋₆ alkyl, or the R₁ₐ and R_{1b} are optionally bonded together to the same nitrogen atom to form a (3-10 membered) heterocyclyl within the substituent,
m is an integer from 0 to 2,
R₂ is each independently selected from the group consisting of hydrogen, C₁₋₁₀ alkyl, C₁₋₁₀ alkylene-C₁₋₆ alkoxy, C₁₋₁₀ alkylene-OH, C₁₋₁₀ alkylene-NR₂ₐR_{2b}, and C₁₋₁₀ alkylene-(3-10 membered) heterocyclyl, wherein (3-10 membered) heterocyclyl is unsubstituted or optionally substituted with C₁₋₆ alkyl,
wherein R₂ₐ and R_{2b} are each independently selected from hydrogen, C₁₋₆ alkyl or C₁₋₆ haloalkyl, or the R₂ₐ and R_{2b} are optionally bonded together to the same nitrogen atom to form a (3-10 membered) heterocyclyl within the substituent,
B is carbon,
R₃ is —NR₃ₐR_{3b},
wherein R₃ₐ and R_{3b} are each independently selected from the group consisting of hydrogen, C₁₋₁₀ alkyl, 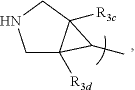
(3-10 membered) cycloalkyl, (6-14 membered) aryl, and (3-10 membered) heterocyclyl,
or the R₃ₐ and R_{3b} are optionally bonded together to the same nitrogen atom to form a (3-10 membered) heterocyclyl within the substituent,
wherein R_{3c} and R_{3d} are each independently selected from hydrogen or C₁₋₆ alkyl,
wherein C₁₋₁₀ alkyl, (3-10 membered) cycloalkyl, (6-14 membered) aryl, and (3-10 membered) heterocyclyl are each independently unsubstituted or optionally substituted with R₄,
R₄ is selected from the group consisting of OH, C₁₋₆ alkyl, C₁₋₆ haloalkyl, C(O)C₁₋₆ alkyl, (6-14 membered) aryl, (3-10 membered) cycloalkyl, (3-10 membered) heterocyclyl and NR₄ₐR_{4b},
wherein R₄ₐ and R_{4b} are each independently selected from hydrogen, C₁₋₆ alkyl or C(O)C₁₋₆ alkyl, or the R₄ₐ and R_{4b} are optionally bonded together to the same nitrogen atom to form a (3-10 membered) heterocyclyl within the substituent,
wherein C₁₋₆ alkyl, (6-14 membered) aryl, (3-10 membered) cycloalkyl, and (3-10 membered) heterocyclyl are each independently unsubstituted or optionally substituted with at least one of C₁₋₆ alkyl, amino, halogen, and C(O)C₁₋₆ alkyl,
Y is hydrogen, and n is 0.

5. The compound or the pharmaceutically acceptable salt thereof according to claim 3, characterized in that the compound of Formula 2 is selected from the group consisting of the compounds of Examples 1 to 40 below:
(3-(1H-benzo[d]imidazol-2-yl)-1H-indazol-5-yl)(4-phenylpiperazin-1-yl)methanone (Example 1),
(3-(1H-benzo[d]imidazol-2-yl)-1H-indazol-5-yl)(4-methylpiperazin-1-yl)methanone (Example 2),
(3-(1H-benzo[d]imidazol-2-yl)-1H-indazol-5-yl)(4-(4-fluorophenyl)piperazin-1-yl)methanone (Example 3),
(3-(1H-benzo[d]imidazol-2-yl)-1H-indazol-5-yl)(4-(2-fluorophenyl)piperazin-1-yl)methanone (Example 4),
(3-(1H-benzo[d]imidazol-2-yl)-1H-indazol-5-yl)(4-(3-chlorophenyl)piperazin-1-yl)methanone (Example 5),
(3-(1H-benzo[d]imidazol-2-yl)-1H-indazol-5-yl)(4-(2-chlorophenyl)piperazin-1-yl)methanone (Example 6),
N-(3-aminopropyl)-3-(1H-benzo[d]imidazol-2-yl)-1H-indazole-5-carboxamide (Example 7),
N-(2-(4-aminopiperidin-1-yl)ethyl)-3-(1H-benzo[d]imidazol-2-yl)-1H-indazole-5-carboxamide (Example 8),
3-(5,6-difluoro-1H-benzo[d]imidazol-2-yl)-N-(3-(dimethylamino)propyl)-1H-indazole-5-carboxamide (Example 9),
3-(6-cyano-1H-benzo[d]imidazol-2-yl)-N-(3-(dimethylamino)propyl)-1H-indazole-5-carboxamide (Example 10),
N-(3-(dimethylamino)propyl)-3-(6-methoxy-1H-benzo[d]imidazol-2-yl)-1H-indazole-5-carboxamide (Example 11),
N-(3-(dimethylamino)propyl)-3-(6-methyl-1H-benzo[d]imidazol-2-yl)-1H-indazole-5-carboxamide (Example 12),
3-(6-(pyrrolidin-1-yl)-1H-benzo[d]imidazol-2-yl)-N-(4,4,4-trifluorobutyl)-1H-indazole-5-carboxamide (Example 13),
N-((3,3-difluorocyclobutyl)methyl)-3-(6-(pyrrolidin-1-yl)-1H-benzo[d]imidazol-2-yl)-1H-indazole-5-carboxamide (Example 14),
N-(3-aminopropyl)-3-(6-methyl-1H-benzo[d]imidazol-2-yl)-1H-indazole-5-carboxamide (Example 15),
N-(3-aminopropyl)-3-(6-(pyrrolidin-1-yl)-1H-benzo[d]imidazol-2-yl)-1H-indazole-5-carboxamide (Example 16),
N-(3-aminopropyl)-3-(6-(piperidin-1-yl)-1H-benzo[d]imidazol-2-yl)-1H-indazole-5-carboxamide (Example 17),
N-(3-aminopropyl)-3-(6-(4-methylpiperazin-1-yl)-1H-benzo[d]imidazol-2-yl)-1H-indazole-5-carboxamide (Example 18),
N-(3-aminopropyl)-3-(6-((3S,5R)-3,5-dimethylpiperazin-1-yl)-1H-benzo[d]imidazol-2-yl)-1H-indazole-5-carboxamide (Example 19),
N-(3-aminopropyl)-3-(6-morpholino-1H-benzo[d]imidazol-2-yl)-1H-indazole-5-carboxamide (Example 20),
N-(3-aminopropyl)-3-(6-(morpholine-4-carbonyl)-1H-benzo[d]imidazol-2-yl)-1H-indazole-5-carboxamide (Example 21),
N-(3-aminopropyl)-3-(6-methoxy-1H-benzo[d]imidazol-2-yl)-1H-indazole-5-carboxamide (Example 22),
N-(3-aminopropyl)-3-(6-(2-methoxyethoxy)-1H-benzo[d]imidazol-2-yl)-1H-indazole-5-carboxamide (Example 23),
N-(3-aminopropyl)-3-(6-(2-(dimethylamino)ethoxy)-1H-benzo[d]imidazol-2-yl)-1H-indazole-5-carboxamide (Example 24),
N-(3-aminopropyl)-3-(7-fluoro-1H-benzo[d]imidazol-2-yl)-1H-indazole-5-carboxamide (Example 25),
N-(3-aminopropyl)-3-(6-(morpholinomethyl)-1H-benzo[d]imidazol-2-yl)-1H-indazole-5-carboxamide (Example 26),
N-(3-aminopropyl)-3-(6-chloro-1H-benzo[d]imidazol-2-yl)-1H-indazole-5-carboxamide (Example 27),
N-(3-aminopropyl)-3-(6-bromo-1H-benzo[d]imidazol-2-yl)-1H-indazole-5-carboxamide (Example 28),
N-(3-aminopropyl)-3-(6-(trifluoromethyl)-1H-benzo[d]imidazol-2-yl)-1H-indazole-5-carboxamide (Example 29),
N-((1S,3S)-3-aminocyclopentyl)-3-(6-(pyrrolidin-1-yl)-1H-benzo[d]imidazol-2-yl)-1H-indazole-5-carboxamide (Example 30),
N-((1S,3S)-3-aminocyclopentyl)-3-(6-(piperidin-1-yl)-1H-benzo[d]imidazol-2-yl)-1H-indazole-5-carboxamide (Example 31),
N-((1S,3S)-3-aminocyclopentyl)-3-(6-methoxy-1H-benzo[d]imidazol-2-yl)-1H-indazole-5-carboxamide (Example 32),
N-(3-acetamidopropyl)-3-(6-(piperidin-1-yl)-1H-benzo[d]imidazol-2-yl)-1H-indazole-5-carboxamide (Example 33),
(3-(6-fluoro-1H-benzo[d]imidazol-2-yl)-1H-indazol-5-yl)(4-(4-methylpiperazin-1-yl) piperidin-1-yl)methanone (Example 34),
N-(2-(dimethylamino)ethyl)-3-(6-fluoro-1H-benzo[d]imidazol-2-yl)-1H-indazole-5-carboxamide (Example 35),
N-(3-(dimethylamino)propyl)-3-(6-fluoro-1H-benzo[d]imidazol-2-yl)-1H-indazole-5-carboxamide (Example 36),
N-(2-aminoethyl)-3-(6-fluoro-1H-benzo[d]imidazol-2-yl)-1H-indazole-5-carboxamide (Example 37),
N-(3-aminopropyl)-3-(6-fluoro-1H-benzo[d]imidazol-2-yl)-1H-indazole-5-carboxamide (Example 38),
N-(4-aminobutyl)-3-(6-fluoro-1H-benzo[d]imidazol-2-yl)-1H-indazole-5-carboxamide (Example 39) and
3-(6-fluoro-1H-benzo[d]imidazol-2-yl)-N-(piperidin-4-yl)-1H-indazole-5-carboxamide (Example 40).

6. The compound or the pharmaceutically acceptable salt thereof according to claim 3, characterized in that the compound of Formula 2 is selected from the group consisting of the compounds of Examples 41 to 80 below:
(R)-3-(6-fluoro-1H-benzo[d]imidazol-2-yl)-N-(piperidin-3-yl)-1H-indazole-5-carboxamide (Example 41),
N-(2-aminoethyl)-3-(6-morpholino-1H-benzo[d]imidazol-2-yl)-1H-indazole-5-carboxamide (Example 42),
N-(4-aminobutyl)-3-(6-morpholino-1H-benzo[d]imidazol-2-yl)-1H-indazole-5-carboxamide (Example 43),
N-(3-(methylamino)propyl)-3-(6-morpholino-1H-benzo[d]imidazol-2-yl)-1H-indazole-5-carboxamide (Example 44),
N-(3-amino-2-methylpropyl)-3-(6-morpholino-1H-benzo[d]imidazol-2-yl)-1H-indazole-5-carboxamide (Example 45),
N-(azetidin-3-yl)-3-(6-morpholino-1H-benzo[d]imidazol-2-yl)-1H-indazole-5-carboxamide (Example 46),
N-(azetidin-3-ylmethyl)-3-(6-morpholino-1H-benzo[d]imidazol-2-yl)-1H-indazole-5-carboxamide (Example 47),
N-(2-(azetidin-3-yl)ethyl)-3-(6-morpholino-1H-benzo[d]imidazol-2-yl)-1H-indazole-5-carboxamide (Example 48),
(3-(6-morpholino-1H-benzo[d]imidazol-2-yl)-1H-indazol-5-yl)(2,6-diazaspiro[3.3]heptan-2-yl)methanone (Example 49),
N—((1S,3R)-3-aminocyclopentyl)-3-(6-morpholino-1H-benzo[d]imidazol-2-yl)-1H-indazole-5-carboxamide (Example 50),
N-((1S,3S)-3-aminocyclopentyl)-3-(6-morpholino-1H-benzo[d]imidazol-2-yl)-1H-indazole-5-carboxamide (Example 51),
(R)-3-(6-morpholino-1H-benzo[d]imidazol-2-yl)-N-(pyrrolidin-3-yl)-1H-indazole-5-carboxamide (Example 52),
(S)-3-(6-morpholino-1H-benzo[d]imidazol-2-yl)-N-(pyrrolidin-3-yl)-1H-indazole-5-carboxamide (Example 53),
(R)-3-(6-morpholino-1H-benzo[d]imidazol-2-yl)-N-(pyrrolidin-3-ylmethyl)-1H-indazole-5-carboxamide (Example 54),
(S)-3-(6-morpholino-1H-benzo[d]imidazol-2-yl)-N-(pyrrolidin-3-ylmethyl)-1H-indazole-5-carboxamide (Example 55),
3-(6-morpholino-1H-benzo[d]imidazol-2-yl)-N-(piperidin-3-ylmethyl)-1H-indazole-5-carboxamide (Example 56),
3-(6-morpholino-1H-benzo[d]imidazol-2-yl)-N-(piperidin-4-ylmethyl)-1H-indazole-5-carboxamide (Example 57),
3-(6-morpholino-1H-benzo[d]imidazol-2-yl)-N-(piperidin-4-yl)-1H-indazole-5-carboxamide (Example 58),
N-((1s,4s)-4-aminocyclohexyl)-3-(6-morpholino-1H-benzo[d]imidazol-2-yl)-1H-indazole-5-carboxamide (Example 59),
1-(6-(3-(6-morpholino-1H-benzo[d]imidazol-2-yl)-1H-indazole-5-carbonyl)-2,6-diazaspiro[3.3]heptan-2-yl)ethan-1-one (Example 60),
N-((1-acetylazetidin-3-yl)methyl)-3-(6-morpholino-1H-benzo[d]imidazol-2-yl)-1H-indazole-5-carboxamide (Example 61),
N-(2-(dimethylamino)ethyl)-3-(6-morpholino-1H-benzo[d]imidazol-2-yl)-1H-indazole-5-carboxamide (Example 62),
N-(3-(dimethylamino)propyl)-3-(6-morpholino-1H-benzo[d]imidazol-2-yl)-1H-indazole-5-carboxamide (Example 63),
(3-(6-morpholino-1H-benzo[d]imidazol-2-yl)-1H-indazol-5-yl)(piperazin-1-yl)methanone (Example 64),
((1R,4R)-2,5-diazabicyclo[2.2.1]heptan-2-yl)(3-(6-morpholino-1H-benzo[d]imidazol-2-yl)-1H-indazol-5-yl)methanone (Example 65),
((1S,4S)-2,5-diazabicyclo[2.2.1]heptan-2-yl)(3-(6-morpholino-1H-benzo[d]imidazol-2-yl)-1H-indazol-5-yl)methanone (Example 66),
(1,4-diazepan-1-yl)(3-(6-morpholino-1H-benzo[d]imidazol-2-yl)-1H-indazol-5-yl)methanone (Example 67),
(3-(aminomethyl)piperidin-1-yl)(3-(6-morpholino-1H-benzo[d]imidazol-2-yl)-1H-indazol-5-yl)methanone (Example 68),
(4-aminopiperidin-1-yl)(3-(6-morpholino-1H-benzo[d]imidazol-2-yl)-1H-indazol-5-yl)methanone (Example 69),
(4-(methylamino)piperidin-1-yl)(3-(6-morpholino-1H-benzo[d]imidazol-2-yl)-1H-indazol-5-yl)methanone (Example 70),
N-((1R,5S,6r)-3-azabicyclo[3.1.0]hexan-6-yl)-3-(6-morpholino-1H-benzo[d]imidazol-2-yl)-1H-indazole-5-carboxamide (Example 71),
N-(4-aminophenyl)-3-(6-morpholino-1H-benzo[d]imidazol-2-yl)-1H-indazole-5-carboxamide (Example 72),
N-(3-aminopropyl)-3-(6-(furan-3-yl)-1H-benzo[d]imidazol-2-yl)-1H-indazole-5-carboxamide (Example 73),
N-((1S,3S)-3-aminocyclopentyl)-3-(6-(furan-3-yl)-1H-benzo[d]imidazol-2-yl)-1H-indazole-5-carboxamide (Example 74),
N-(azetidin-3-ylmethyl)-3-(6-(furan-3-yl)-1H-benzo[d]imidazol-2-yl)-1H-indazole-5-carboxamide (Example 75),
N-(3-aminopropyl)-3-(6-phenyl-1H-benzo[d]imidazol-2-yl)-1H-indazole-5-carboxamide (Example 76),
N-(3-aminopropyl)-3-(6-(pyridin-4-yl)-1H-benzo[d]imidazol-2-yl)-1H-indazole-5-carboxamide (Example 77),
N-(3-aminopropyl)-3-(6-(4-(dimethylamino)phenyl)-1H-benzo[d]imidazol-2-yl)-1H-indazole-5-carboxamide (Example 78),
N-(3-aminopropyl)-3-(6-(4-methoxyphenyl)-1H-benzo[d]imidazol-2-yl)-1H-indazole-5-carboxamide (Example 79) and
N-((1S,3S)-3-aminocyclopentyl)-3-(6-(furan-2-yl)-1H-benzo[d]imidazol-2-yl)-1H-indazole-5-carboxamide (Example 80).

7. The compound or the pharmaceutically acceptable salt thereof according to claim 3, characterized in that the compound of Formula 2 is selected from the group consisting of the compounds of Examples 104 to 112 below:
N-(3-(dimethylamino)propyl)-3-(6-fluoro-1-isopropyl-1H-benzo[d]imidazol-2-yl)-1H-indazole-5-carboxamide (Example 104),
N-(3-aminopropyl)-3-(6-fluoro-1-isopropyl-1H-benzo[d]imidazol-2-yl)-1H-indazole-5-carboxamide (Example 105),
N-(3-aminopropyl)-3-(6-fluoro-1-(2-methoxyethyl)-1H-benzo[d]imidazol-2-yl)-1H-indazole-5-carboxamide (Example 106),
N-(3-aminopropyl)-3-(1-(2-(dimethylamino)ethyl)-6-fluoro-1H-benzo[d]imidazol-2-yl)-1H-indazole-5-carboxamide (Example 107),
N-(3-aminopropyl)-3-(6-fluoro-1-(2-(4-methylpiperazin-1-yl)ethyl)-1H-benzo[d]imidazol-2-yl)-1H-indazole-5-carboxamide (Example 108),
N-(3-aminopropyl)-3-(6-fluoro-1-(2-hydroxyethyl)-1H-benzo[d]imidazol-2-yl)-1H-indazole-5-carboxamide (Example 109),
N-(3-aminopropyl)-3-(6-fluoro-1H-benzo[d]imidazol-2-yl)-1H-indazole-6-carboxamide (Example 110),
3-(6-fluoro-1H-benzo[d]imidazol-2-yl)-N-(3-hydroxypropyl)-1H-indazole-5-carboxamide (Example 111) and
N-(1-(2-aminoethyl)piperidin-4-yl)-3-(1H-benzo[d]imidazol-2-yl)-1H-indazole-5-carboxamide (Example 112).

8. The compound or the pharmaceutically acceptable salt thereof according to claim 1, wherein the compound is represented by Formula 3 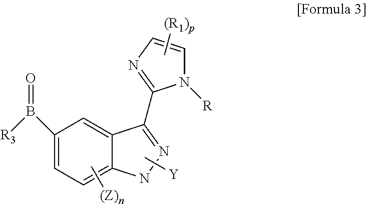
In formula 3,
R₁ is each independently selected from the group consisting of hydrogen, halogen, C₁₋₁₀ alkyl, C₁₋₁₀ haloalkyl, (3-10 membered) cycloalkyl, C₁₋₁₀ alkylene-(3-10 membered) cycloalkyl, (3-10 membered) heterocyclyl, C₁₋₁₀ alkylene-(3-10 membered) heterocyclyl, (6-14 membered) aryl, C₀₋₁₀ alkylene-(6-14 membered) aryl, (5-12 membered) heteroaryl, C₁₋₁₀ alkylene-(5-12 membered) heteroaryl, C₁₋₁₀ alkoxy, C₁₋₁₀ alkylene-C₁₋₆ alkoxy, 0-(6-14 membered) aryl, C₁₋₁₀ alkylene-O-(6-14 membered) aryl, O—C₁₋₁₀ alkylene-C₁₋₆ alkoxy, O—C₁₋₁₀ alkylene-NR₁ₐR_{1b}, C(O)—NR₁ₐR_{1b}, C₁₋₁₀ alkylene-C(O)—NR₁ₐR_{1b}, C₁₋₆ haloalkoxy, C₁₋₁₀ alkylene-C₁₋₆ haloalkoxy, CN, C₁₋₁₀ alkylene-CN, OH, NO₂, NR₁ₐR_{1b}, OC(O)—NR₁ₐR_{1b}, C(O)R₁ₐ, C(O)OR₁ₐ, NR₁ₐC(O)R_{1b}, NR₁ₐC(O)OR_{1b}, NR₁ₐSO₂R_{1b} and SO₂NR₁ₐR_{1b},
wherein (3-10 membered) cycloalkyl, (3-10 membered) heterocyclyl, (6-14 membered) aryl, and (5-12 membered) heteroaryl are each independently unsubstituted or optionally substituted with at least one of (3-10 membered) cycloalkyl, (3-10 membered) heterocyclyl, C₁₋₆ alkyl, C₁₋₆ alkoxy, halogen, C₁₋₆ haloalkyl, NH₂, NO₂, OH, or NR₁ₐR_{1b},
wherein R₁ₐ and R_{1b} are each independently selected from hydrogen, C₁₋₆ alkyl or C₁₋₆ haloalkyl, or the R₁ₐ and R_{1b} are optionally bonded together to the same nitrogen atom to form a (3-10 membered) heterocyclyl or a (5-12 membered) heteroaryl within the substituent,
p is an integer from 0 to 2,
R₂ is independently selected from the group consisting of hydrogen, C₁₋₁₀ alkyl, halogen, C₁₋₆ haloalkyl, C₁₋₆ alkoxy, C₁₋₁₀ alkylene-C₁₋₆ alkoxy, C₁₋₆ haloalkoxy, CN, OH, C₁₋₁₀ alkylene-OH, NO₂, C₁₋₆ alkylamino, C₁₋₁₀ alkylene-NR₂ₐR_{2b}, (6-14 membered) aryl, (5-12 membered) heteroaryl, (3-10 membered) cycloalkyl, (3-10 membered) heterocyclyl and C₁₋₁₀ alkylene-(3-10 membered) heterocyclyl,
wherein (6-14 membered) aryl, (5-12 membered) heteroaryl, (3-10 membered) cycloalkyl and (3-10 membered) heterocyclyl are each independently unsubstituted or optionally substituted with at least one of C₁₋₆ alkyl, halogen, C₁₋₆ haloalkyl, C₁₋₆ alkoxy or C₁₋₆ haloalkoxy,
wherein R₂ₐ and R_{2b} are each independently selected from hydrogen, C₁₋₆ alkyl or C₁₋₆ haloalkyl, or the R₂ₐ and R_{2b} are optionally bonded together to the same nitrogen atom to form a (3-10 membered) heterocyclyl or a (5-12 membered) heteroaryl within the substituent,
B is carbon or sulfur,
R₃ is —NR₃ₐR_{3b},
wherein R₃ₐ and R_{3b} are each independently selected from the group consisting of hydrogen, OH, C₁₋₁₀ alkyl, C₁₋₆ haloalkyl, C(O)C₁₋₆ alkyl, 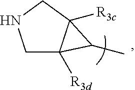
(3-10 membered) cycloalkyl, (6-14 membered) aryl, (5-12 membered) heteroaryl and (3-10 membered) heterocyclyl,
or the R₃ₐ and R_{3b} are optionally bonded together to the same nitrogen atom to form a (3-10 membered) heterocyclyl or a (5-12 membered) heteroaryl within the substituent,
wherein R_{3c} and R_{3d} are each independently selected from hydrogen, C₁₋₆ alkyl or C₁₋₆ haloalkyl,
wherein C₁₋₁₀ alkyl, (3-10 membered) cycloalkyl, (3-10 membered) heterocyclyl, (6-14 membered) aryl, and (5-12 membered) heteroaryl are each independently unsubstituted or optionally substituted with R₄,
R₄ is selected from the group consisting of OH, C₁₋₆ alkyl, C₁₋₆ alkoxy, halogen, C₁₋₆ haloalkyl, C(O)C₁₋₆alkyl, (6-14 membered) aryl, (3-10 membered) cycloalkyl, (3-10 membered) heterocyclyl, (5-12 membered) heteroaryl and NR₄ₐR_{4b},
wherein R₄ₐ and R_{4b} are each independently selected from hydrogen, C₁₋₆ alkyl, C₁₋₆ haloalkyl, C(O)C₁₋₆ alkyl, SO₂(C₁₋₆)alkyl, or C(O)C₁₋₆ haloalkyl, or the R₄ₐ and R_{4b} are optionally bonded together to the same nitrogen atom to form a (3-10 membered) heterocyclyl or a (5-12 membered) heteroaryl within the substituent,
wherein (6-14 membered) aryl, (3-10 membered) cycloalkyl, and (3-10 membered) heterocyclyl are each independently unsubstituted or optionally substituted with at least one of C₁₋₆ alkyl, amino, halogen, C₁₋₆ haloalkyl, C(O)C₁₋₆ alkyl, C₁₋₆ alkoxy or C₁₋₆ haloalkoxy,
Y is selected from the group consisting of hydrogen, C₁₋₆ alkyl, C₁₋₆ haloalkyl, C₁₋₆ alkoxy, C(O)C₁₋₆ alkyl, C(O)OC₁₋₆ alkyl, and C(O)N—C₁₋₆ alkyl,
Z is selected from the group consisting of hydrogen, C₁₋₆ alkyl, halogen, C₁₋₆ haloalkyl, C₁₋₆ alkoxy, NH₂, OH, CN, COOH, C(O)C₁₋₆ alkyl, C(O)OC₁₋₆ alkyl and C(O)N—C₁₋₆ alkyl,
wherein heterocyclyl and heteroaryl each independently comprise at least one of N, O, or S in the ring,
n is an integer from 0 to 3.

9. The compound or the pharmaceutically acceptable salt thereof according to claim 8, characterized in that
R₁ is independently selected from the group consisting of hydrogen, (6-14 membered) aryl, C₁₋₁₀ alkylene-C₁₋₆ alkoxy, C₁₋₁₀ alkylene-O-(6-14 membered) aryl, and C(O)—NR₁ₐR_{1b},
wherein (6-14 membered) aryl are each independently unsubstituted or optionally substituted with at least one of (3-10 membered) cycloalkyl, (3-10 membered) heterocyclyl, C₁₋₆ alkyl, C₁₋₆ alkoxy, halogen, C₁₋₆ haloalkyl, NH₂, NO₂, OH, or NR₁ₐR_{1b},
wherein R₁ₐ and R_{1b} are each independently hydrogen or C₁₋₆ alkyl, or the R₁ₐ and R_{1b} are optionally bonded together to the same nitrogen atom to form a (3-10 membered) heterocyclyl within the substituent,
p is an integer from 0 to 2,
R₂ is each independently hydrogen or C₁₋₁₀ alkyl,
B is carbon,
R₃ is —NR₃ₐR_{3b},
wherein R₃ₐ and R_{3b} are each independently hydrogen or C₁₋₁₀ alkyl,
or the R₃ₐ and R_{3b} are optionally bonded together to the same nitrogen atom to form a (3-10 membered) heterocyclyl within the substituent,
wherein C₁₋₁₀ alkyl and (3-10 membered) heterocyclyl are unsubstituted or optionally substituted with R₄,
R₄ is selected from the group consisting of C₁₋₆ alkyl, (6-14 membered) aryl, (3-10 membered) heterocyclyl, and NR₄ₐR_{4b},
wherein R₄ₐ and R_{4b} are each independently selected from hydrogen, C₁₋₆ alkyl, or SO₂(C₁₋₆)alkyl, or the R₄ₐ and R_{4b} are optionally bonded to the same nitrogen atom to form a (3-10 membered) heterocyclyl,
wherein (6-14 membered) aryl and (3-10 membered) heterocyclyl are each independently unsubstituted or optionally substituted with at least one of C₁₋₆ alkyl, amino, halogen, or C₁₋₆ haloalkyl,
Y is hydrogen, and n is 0.

10. The compound or the pharmaceutically acceptable salt thereof according to claim 8, characterized in that the compound of Formula 3 is selected from the group consisting of the compounds of Examples 81 to 103 and Examples 113 to 115 below:
N-(3-aminopropyl)-3-(5-(4-fluorophenyl)-1H-imidazol-2-yl)-1H-indazole-5-carboxamide (Example 81),
N-(3-aminopropyl)-3-(5-(3-fluorophenyl)-1H-imidazol-2-yl)-1H-indazole-5-carboxamide (Example 82),
N-(3-aminopropyl)-3-(5-(2-fluorophenyl)-1H-imidazol-2-yl)-1H-indazole-5-carboxamide (Example 83),
N-(3-aminopropyl)-3-(5-(3,4-difluorophenyl)-1H-imidazol-2-yl)-1H-indazole-5-carboxamide (Example 84),
N-(3-aminopropyl)-3-(5-(4-chloro-3-fluorophenyl)-1H-imidazol-2-yl)-1H-indazole-5-carboxamide (Example 85),
N-(3-aminopropyl)-3-(5-(4-(trifluoromethyl)phenyl)-1H-imidazol-2-yl)-1H-indazole-5-carboxamide (Example 86),
N-(3-aminopropyl)-3-(5-(3,5-dichloro-2-methoxyphenyl)-1H-imidazol-2-yl)-1H-indazole-5-carboxamide (Example 87),
N-(3-aminopropyl)-3-(5-(3-methoxyphenyl)-1H-imidazol-2-yl)-1H-indazole-5-carboxamide (Example 88),
N-(3-aminopropyl)-3-(5-(3-nitrophenyl)-1H-imidazol-2-yl)-1H-indazole-5-carboxamide (Example 89),
N-(3-aminopropyl)-3-(5-(4-morpholinophenyl)-1H-imidazol-2-yl)-1H-indazole-5-carboxamide (Example 90),
N-(3-aminopropyl)-3-(5-(3-morpholinophenyl)-1H-imidazol-2-yl)-1H-indazole-5-carboxamide (Example 91),
N-(3-aminopropyl)-3-(5-(3-hydroxyphenyl)-1H-imidazol-2-yl)-1H-indazole-5-carboxamide (Example 92),
3-(5-(3-aminophenyl)-1H-imidazol-2-yl)-N-(3-aminopropyl)-1H-indazole-5-carboxamide (Example 93),
N-(2-(methylsulfonamido)ethyl)-3-(4-phenyl-1H-imidazol-2-yl)-1H-indazole-5-carboxamide (Example 94),
N-(2-(dimethylamino)ethyl)-3-(4-phenyl-1H-imidazol-2-yl)-1H-indazole-5-carboxamide (Example 95),
(3-(4-phenyl-1H-imidazol-2-yl)-1H-indazol-5-yl)(4-phenylpiperazin-1-yl)methanone (Example 96),
(4-(4-fluorophenyl)piperazin-1-yl)(3-(4-phenyl-1H-imidazol-2-yl)-1H-indazol-5-yl)methanone (Example 97),
(3-(4-phenyl-1H-imidazol-2-yl)-1H-indazol-5-yl)(4-(3-(trifluoromethyl)phenyl)piperazin-1-yl)methanone (Example 98),
(4-methylpiperazin-1-yl)(3-(4-phenyl-1H-imidazol-2-yl)-1H-indazol-5-yl)methanone (Example 99),
(4-(2-fluorophenyl)piperazin-1-yl)(3-(4-phenyl-1H-imidazol-2-yl)-1H-indazol-5-yl)methanone (Example 100),
(4-(3-chlorophenyl)piperazin-1-yl)(3-(4-phenyl-1H-imidazol-2-yl)-1H-indazol-5-yl)methanone (Example 101),
(4-(2-chlorophenyl)piperazin-1-yl)(3-(4-phenyl-1H-imidazol-2-yl)-1H-indazol-5-yl)methanone (Example 102),
N-(2-(4-aminopiperidin-1-yl)ethyl)-3-(4-phenyl-1H-imidazol-2-yl)-1H-indazole-5-carboxamide (Example 103),
N-(3-aminopropyl)-3-(4-carbamoyl-5-phenyl-1H-imidazol-2-yl)-1H-indazole-5-carboxamide (Example 113),
N-(3-aminopropyl)-3-(5-(methoxymethyl)-1H-imidazol-2-yl)-1H-indazole-5-carboxamide (Example 114) and
N-(3-aminopropyl)-3-(5-(phenoxymethyl)-1H-imidazol-2-yl)-1H-indazole-5-carboxamide (Example 115).

11. A method for preventing or treating cancer diseases, comprising administering to a subject in need thereof the compound or the pharmaceutically acceptable salt thereof according to claim 1.

12. The method of claim 11, wherein the subject needs inhibiting the activity of protein kinase.

13. The method of claim 12, wherein the protein kinase is selected from the group consisting of ABL1(E255K)-phosphorylated, ABL1(F317I)-phosphorylated, ABL1(F317L)-phosphorylated, ABL1(H396P)-nonphosphorylated, ABL1(H396P)-phosphorylated, ABL1(M351T)-phosphorylated, ABL1(Q252H)-nonphosphorylated, ABL1(Q252H)-phosphorylated, ABL1(T315I)-nonphosphorylated, ABL1(T315I)-phosphorylated, ABL1(Y253F)-phosphorylated, ABL1-nonphosphorylated, ABL1-phosphorylated, ARK5, AURKA, AURKB, AURKC, AXL, CDK2, CDK3, CDK4-cyclinD1, CDK4-cyclinD3, CDK5, CDK7, DYRK1A, DYRK1B, DYRK2, ERK1, ERK2, ERK3, ERK4, ERK8, FLT3, FLT3(D835H), FLT3(D835V), FLT3(D835Y), FLT3(ITD), FLT3(ITD, D835V), FLT3(ITD, F691L), FLT3(K663Q), FLT3(N841I), FLT3(R834Q), FLT3-autoinhibited, GSK3A, GSK3B, HASPIN, HIPK1, HIPK2, HIPK3, HIPK4, IRAK1, IRAK3, IRAK4, JAK1(JHldomain-catalytic), JAK1(JH2domain-pseudokinase), JAK2(JHldomain-catalytic), JAK3 (JHldomain-catalytic), KIT, KIT (A829P), KIT (D816H), KIT (D816V), KIT (L576P), KIT (V559D), KIT (V559D, T670I), KIT (V559D, V654A), MAP3K1, MAP3K15, MAP3K2, MAP3K3, MEK1, MEK2, MEK3, MEK4, MEK5, MEK6, PAK1, PAK2, PAK3, PAK4, PAK4, PAK5, PAK6, PAK7, PIM1, PIM2, PIM3, RET, RET (M918T), RET (V804L), RET (V804M), ROCK1, ROCK2, SRC, TRKA, TRKB, TRKC, and YES1.

14. The method of claim 13, wherein the protein kinase is selected from the group consisting of ARK5, FLT3, YES1, ABL, KIT and RET.

15. The method of claim 14, wherein the protein kinase is Fms-like tyrosine kinase 3 (FLT3).

16. The method of claim 11, wherein the cancer diseases is multiple myeloma, malignant plasma cell neoplasm, Hodgkin's lymphoma, nodular lymphocyte-predominant Hodgkin's lymphoma, Kahler's disease and myelomatosis, plasma cell leukemia, plasmacytoma, B-cell prolymphocytic leukemia, hairy cell leukemia, B-cell non-Hodgkin's lymphoma (NHL), acute myelogenous leukemia (AML), chronic lymphocytic leukemia (CLL), acute lymphocytic leukemia (ALL), chronic myelogenous leukemia (CML), follicular lymphoma, Burkitt's lymphoma, marginal zone lymphoma, mantle cell lymphoma, large cell lymphoma, precursor B-lymphoblastic lymphoma, myeloid leukemia, Waldenstrom's macroglobulinemia, diffuse large B-cell lymphoma, follicular lymphoma, marginal zone lymphoma, mucosa-associated lymphoid tissue lymphoma, small cell lymphocytic lymphoma, mantle cell lymphoma, Burkitt's lymphoma, primary mediastinal (thymic) large B-cell lymphoma, lymphoplasmacytic lymphoma, Waldenstrom macroglobulinemia, nodal marginal zone B-cell lymphoma, splenic marginal zone lymphoma, intravascular large B-cell lymphoma, primary effusion lymphoma, lymphomatoid granulomatosis, T-cell/histiocytoid-rich large B-cell lymphoma, primary central nervous system lymphoma, primary cutaneous diffuse large B-cell lymphoma (leg type), EBV-positive diffuse large B-cell lymphoma in the elderly, inflammation-associated diffuse large B-cell lymphoma, intravascular large B-cell lymphoma, ALK-positive large B-cell lymphoma, plasmacytoid lymphoma, large B-cell lymphoma arising in HHV8-associated multicentric Castleman disease, unclassified B-cell lymphoma having intermediate features between diffuse large B-cell lymphoma and Burkitt lymphoma, or unclassified B-cell lymphoma having intermediate features between diffuse large B-cell lymphoma and traditional Hodgkin's lymphoma.

17. The method of claim 11, wherein the cancer diseases is leukemia.

18. The method of claim 17, wherein the leukemia is acute myeloid leukemia (AML).

19. The method of claim 18, wherein the acute myeloid leukemia (AML) is one in which mutant FLT3 is expressed.

20. A method for preventing or treating a disease associated with Fms-like tyrosine kinase 3 (FLT3), comprising administering to a subject in need thereof the compound or the pharmaceutically acceptable salt thereof of claim 1.
